(19) United States
(12) Patent Application Publication
(10) Pub. No.: US 2026/0033128 A1
(43) Pub. Date: Jan. 29, 2026

(54) COMPOUND AND ORGANIC LIGHT-EMITTING DEVICE COMPRISING SAME

(71) Applicant: LG CHEM, LTD., Seoul (KR)

(72) Inventors: Jae Seung HA, Daejeon (KR); Sujeong Geum, Daejeon (KR); Da Jung Lee, Daejeon (KR); Jiye Jeon, Daejeon (KR); Woo Jin Cho, Daejeon (KR); Ji Young Choi, Daejeon (KR); Woochul Lee, Daejeon (KR)

(21) Appl. No.: 19/339,981

(22) Filed: Sep. 25, 2025

Related U.S. Application Data
(63) Continuation-in-part of application No. PCT/KR2024/004097, filed on Mar. 29, 2024.

(30) Foreign Application Priority Data
Mar. 31, 2023 (KR) 10-2023-0043063

(51) Int. Cl. H10K 50/12; C07D 209/86; C09K 11/06; H10K 85/40; H10K 85/60; H10K 101/00

(52) U.S. Cl. CPC H10K 50/12; C07D 209/86; C09K 11/06; H10K 85/40; H10K 85/622; H10K 85/626; H10K 85/633; H10K 85/636; H10K 85/654; H10K 85/6572; H10K 85/6574; H10K 85/6576; H10K 85/658; C09K 2211/1088; H10K 2101/90

(57) ABSTRACT

The present specification relates to a compound of Chemical Formula 1:

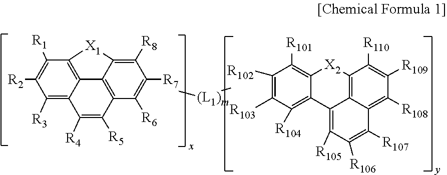

where X₁ and X₂ are the same as or different from each other, and are each independently O or S, L₁ is a direct bond, a substituted or unsubstituted arylene group or a substituted or unsubstituted divalent heterocyclic group, and the other substituents are as described in the specification; and an organic light-emitting device including the same.

### CROSS-REFERENCE TO RELATED APPLICATION(S)

This application is a continuation-in-part of, and claims priority to, International Application No. PCT/KR2024/004097 filed on Mar. 29, 2024, which claims priority to and the benefit of Korean Patent Application No. 10-2023-0043063 filed in the Korean Intellectual Property Office on Mar. 31, 2023, the entire contents of which are incorporated herein by reference.

### TECHNICAL FIELD

The present specification relates to a compound and an organic light emitting device including the same.

### BACKGROUND

An organic light emission phenomenon generally refers to a phenomenon converting electrical energy to light energy using an organic material. An organic light emitting device using the organic light emitting phenomenon usually has a structure including an anode, a cathode, and an organic material layer interposed therebetween. Here, the organic material layer has in many cases a multi-layered structure composed of different materials in order to improve the efficiency and stability of the organic light emitting device, and for example, may be composed of a hole injection layer, a hole transport layer, a light emitting layer, an electron transport layer, an electron injection layer, and the like. In the structure of the organic light emitting device, if a voltage is applied between two electrodes, holes are injected from an anode into the organic material layer and electrons are injected from a cathode into the organic material layer, and when the injected holes and electrons meet each other, an exciton is formed, and light is emitted when the exciton falls down again to a ground state.

There is a continuous need for developing a new material for the aforementioned organic light emitting device.

### BRIEF DESCRIPTION

### Technical Problem

The present specification has been made in an effort to provide a compound and an organic light emitting device including the same.

### Technical Solution

The present specification provides a compound of the following Chemical Formula 1.

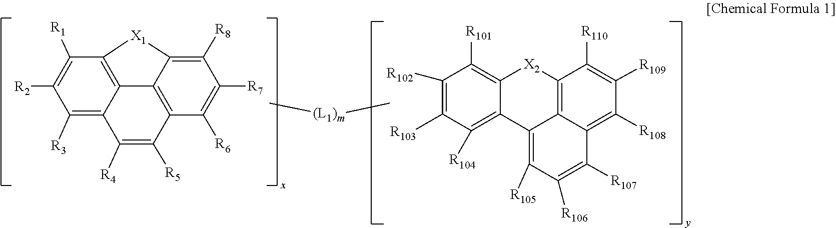

wherein, in Chemical Formula 1,
X₁ and X₂ are the same as or different from each other, and are each independently O or S,
L₁ is a direct bond, a substituted or unsubstituted arylene group or a substituted or unsubstituted divalent heterocyclic group,
here, L₁ is further unsubstituted or substituted with deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and optionally, at least one of the substituents of L₁ is optionally bonded to an adjacent substituent to form one or more substituted or unsubstituted rings,
one of R₁ to R₈ and one of R₁₀₁ to R₁₁₀ are each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring,
m is an integer from 1 to 4, and when m is 2 or higher, L₁'s are the same as or different from each other,
x is 1 or 2, and y is 1 or 2, and
when x and y are each 2, structures in the parenthesis are the same as or different from each other.

The another present specification provides a compound of any one of the following Chemical Formulae 11 to 13.

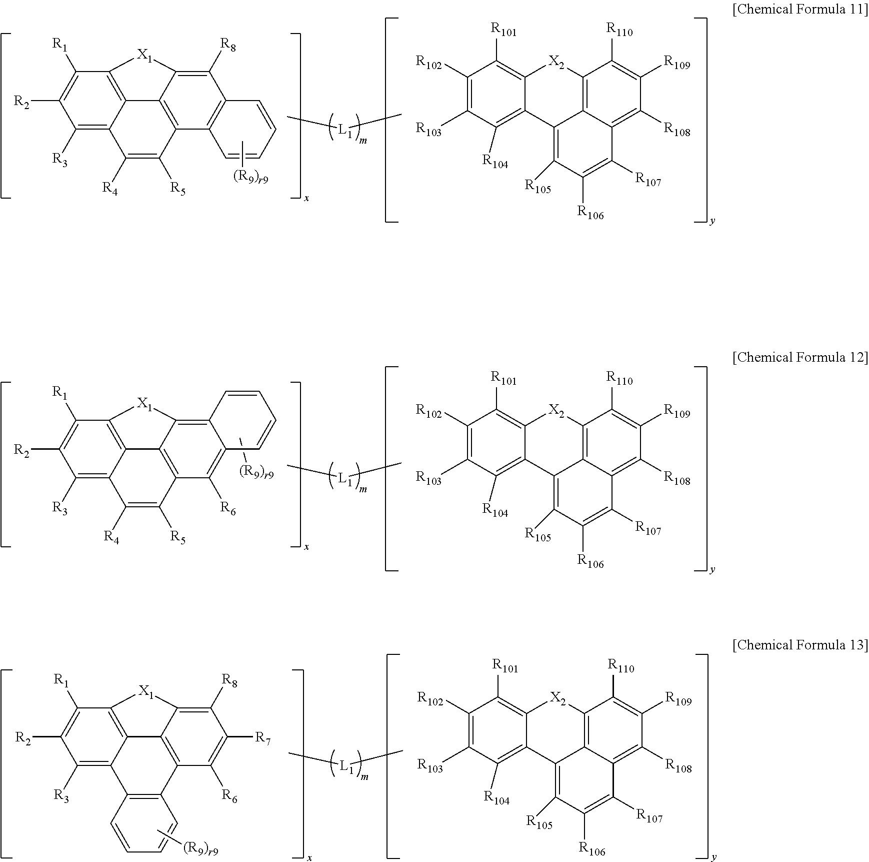

in Chemical Formulae 11 to 13,
X₁ and X₂ are the same as or different from each other, and are each independently O or S,
L₁ is a direct bond, a substituted or unsubstituted arylene group or a substituted or unsubstituted divalent heterocyclic group,
here, L₁ is further unsubstituted or substituted with deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and optionally, at least one of the substituents of L₁ is optionally bonded to an adjacent substituent to form one or more substituted or unsubstituted rings,
one of R₁ to R₉ and one of R₁₀₁ to R₁₁₀ are each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring,
m is an integer from 1 to 4, and when m is 2 or higher, L₁'s are the same as or different from each other,
x is 1 or 2, and y is 1 or 2,
when x and y are each 2, structures in the parenthesis are the same as or different from each other, and
r9 is an integer from 1 to 4, and when r9 is 2 or higher, R₉'s are the same as or different from each other.

Further, the present specification provides an organic light emitting device including: an anode; a cathode; and an organic material layer having one or more layers provided between the anode and the cathode, in which one or more layers of the organic material layer include the above-described compound.

### Advantageous Effects

An organic light emitting device using the compound according to an exemplary embodiment of the present application can have a low driving voltage, high light emitting efficiency, or a long service life.

### BRIEF DESCRIPTION OF DRAWINGS

FIG. 1 illustrates a structure of an organic light emitting device according to a first exemplary embodiment.

FIG. 2 illustrates a structure of an organic light emitting device according to a second exemplary embodiment.

FIG. 3 illustrates a structure of an organic light emitting device according to a third exemplary embodiment.

FIG. 4 illustrates a structure of an organic light emitting device according to a fourth exemplary embodiment.

FIG. 5 is a view illustrating the results of HPLC analysis of a film deposited using the mixture of Experimental Example 2.

FIG. 6 is a view illustrating the results of HPLC analysis of a film deposited using the mixture of Experimental Example 3.

### EXPLANATION OF REFERENCE NUMERALS AND SYMBOLS

101: Substrate
102: Anode
103: Hole injection layer
104: Hole Transport layer
105: Hole adjusting layer
106: Light Emitting Layer
106-1: First light emitting layer
106-2: Second light emitting layer
107: Electron adjusting layer
108: Electron Transport Layer
109: Electron injection layer
110: Cathode

### DETAILED DESCRIPTION

Hereinafter, the present specification will be described in detail.

The present specification provides the compound represented by Chemical Formula 1.

Chemical Formula 1 according to an exemplary embodiment of the present specification is a structure in which a benzo(thio)xanthine is bonded to a phenanthrofuran or phenanthrothiophene structure through an intermediate linking group L₁, and has excellent ability to transfer electrons and holes, and high quantum efficiency. Therefore, by applying the compound of Chemical Formula 1 to the organic material layer of an organic light emitting device, it is possible to obtain the effects of reducing the driving voltage, improving the efficiency, and prolonging the service life.

When one part “includes” one constituent element in the present specification, unless otherwise specifically described, this does not mean that another constituent element is excluded, but means that another constituent element may be further included.

When one member is disposed “on” another member in the present specification, this includes not only a case where the one member is brought into contact with another member, but also a case where still another member is present between the two members.

In the present specification, * or a dotted line means a site bonded or fused to another substituent or a bonding portion.

In the present specification, Cn means that the number of carbon atoms is n, and Cn-Cm means that the number of carbon atoms is n to m.

Examples of the substituents in the present specification will be described below, but are not limited thereto.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, the term “substituted or unsubstituted” means being substituted with one or two or more substituents selected from the group consisting of deuterium; a halogen group; a cyano group (—CN); a silyl group; a boron group; an alkyl group; a cycloalkyl group; an aryl group; and a heterocyclic group, being substituted with a substituent in which two or more substituents among the exemplified substituents are linked together, or having no substituent. For example, “the substituent in which two or more substituents are linked together” may be a biphenyl group. That is, the biphenyl group may also be an aryl group, and may be interpreted as a substituent in which two phenyl groups are linked together.

In an exemplary embodiment of the present invention, the “substituted or unsubstituted” means being substituted with one or more substituents selected from the group consisting of deuterium; a halogen group; a cyano group (—CN); a silyl group; a C1-C20 alkyl group; a C3-C60 cycloalkyl group; a C6-C60 aryl group; and a C2-C60 heterocyclic group, being substituted with a substituent in which two or more groups selected from the above group are linked together, or having no substituent.

In an exemplary embodiment of the present invention, the “substituted or unsubstituted” means being substituted with one or more substituents selected from the group consisting of deuterium; a halogen group; a cyano group (—CN); a silyl group; a C1-C10 alkyl group; a C3-C30 cycloalkyl group; a C6-C30 aryl group; and a C2-C30 heterocyclic group, being substituted with a substituent in which two or more groups selected from the above group are linked together, or having no substituent.

In an exemplary embodiment of the present invention, the “substituted or unsubstituted” means being substituted with one or more substituents selected from the group consisting of deuterium; a halogen group; a cyano group (—CN); a silyl group; a C1-C6 alkyl group; a C3-C20 cycloalkyl group; a C6-C20 aryl group; and a C2-C20 heterocyclic group, being substituted with a substituent in which two or more groups selected from the above group are linked together, or having no substituent.

In the present specification, the fact that two or more substituents are linked indicates that hydrogen of any one substituent is replaced with another substituent. For example, an isopropyl group and a phenyl group may be linked to each other to become a substituent of

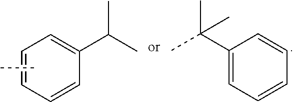

In the present specification, the fact that three substituents are linked to one another includes not only a case where (Substituent 1)-(Substituent 2)-(Substituent 3) are consecutively linked to one another, but also a case where (Substituent 2) and (Substituent 3) are linked to (Substituent 1). For example, two phenyl groups and an isopropyl group may be linked to each other to become a substituent of

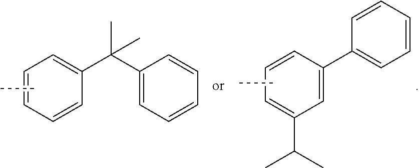

The same also applies to the case where four or more substituents are linked to one another.

In the present specification, “substituted with A or B” includes not only the case of being substituted with A alone or with B alone, but also the case of being substituted with A and B.

In the present specification, “substituted with at least one selected from the group consisting of A, B, and C” means being substituted with one or more substituents selected from the group, or being substituted with a substituent to which two or more groups selected from the group are linked.

Examples of the substituents will be described below; however, the substituents are not limited thereto.

In the present specification, examples of a halogen group include fluorine (—F), chlorine (—Cl), bromine (—Br) or iodine (—I).

In the present specification, a silyl group may be represented by a chemical formula of —SiYaYbYc, and the Ya, Yb, and Yc may be each hydrogen; a substituted or unsubstituted alkyl group; or a substituted or unsubstituted aryl group. Specific examples of the silyl group include a trimethylsilyl group, a triethylsilyl group, a tert-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a boron group may be represented by a chemical formula of —BYdYe, and the Yd and Ye may be each hydrogen; a substituted or unsubstituted alkyl group; or a substituted or unsubstituted aryl group. Specific examples of the boron group include a trimethylboron group, a triethylboron group, a tert-butyldimethylboron group, a triphenylboron group, a phenylboron group, and the like, but are not limited thereto.

In the present specification, the alkyl group may be straight-chained or branched, and the number of carbon atoms thereof is not particularly limited, but is preferably 1 to 60. According to an exemplary embodiment, the number of carbon atoms of the alkyl group is 1 to 30. According to another exemplary embodiment, the number of carbon atoms of the alkyl group is 1 to 20. According to still another exemplary embodiment, the number of carbon atoms of the alkyl group is 1 to 10. Specific examples of the alkyl group include a methyl group, an ethyl group, a propyl group, an n-propyl group, an isopropyl group, a butyl group, an n-butyl group, an isobutyl group, a tert-butyl group, a pentyl group, an n-pentyl group, a hexyl group, an n-hexyl group, a heptyl group, an n-heptyl group, an octyl group, an n-octyl group, and the like, but are not limited thereto.

Substituents including an alkyl group and other alkyl group moieties described in the present specification include both a straight-chained form and a branched form.

In the present specification, a cycloalkyl group is not particularly limited, but has preferably 3 to 60 carbon atoms, and according to an exemplary embodiment, the number of carbon atoms of the cycloalkyl group is 3 to 30. According to another exemplary embodiment, the number of carbon atoms of the cycloalkyl group is 3 to 20. According to another embodiment, the number of carbon atoms of the cycloalkyl group is from 3 to 6. Specific examples thereof may include a cyclopropyl group, a cyclobutyl group, a cyclopentyl group, a cyclohexyl group, a cycloheptyl group, a cyclooctyl group and the like, but are not limited thereto.

In the present specification, an aryl group is not particularly limited, but has preferably 6 to 60 carbon atoms, and may be a monocyclic aryl group or a polycyclic aryl group. According to an exemplary embodiment, the number of carbon atoms of the aryl group is from 6 to 30. According to an exemplary embodiment, the number of carbon atoms of the aryl group is from 6 to 20. Examples of a monocyclic aryl group as the aryl group include a phenyl group, a biphenyl group, a terphenyl group, and the like, but are not limited thereto. Examples of the polycyclic aryl group include a naphthyl group, an anthracenyl group, a phenanthrenyl group, a pyrenyl group, a perylenyl group, a triphenylenyl group, a chrysenyl group, and the like, but are not limited thereto.

In the present specification, the aryl group may also include a form in which an aliphatic ring is fused to an aromatic ring. For example, a fluorenyl group, a spirofluorenyl group, a spirobifluorenyl group, a tetrahydronaphthalenyl group, a dihydroindenyl group, a dihydroanthracenyl group, or the like are included in the aryl group. In the following structure, one of the carbons of a benzene ring may be linked to another position.

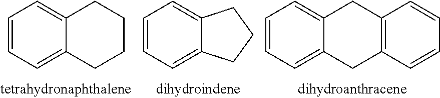

In the present specification, No. 9 carbon atom (C) of a fluorenyl group may be substituted with an alkyl group, an aryl group, or the like, and two substituents may be bonded to each other to form a spiro structure such as cyclopentane or fluorene.

In the present specification, the above-described description on the aryl group may be applied to an arylene group except for a divalent arylene group.

In the present specification, the alkylaryl group means an aryl group substituted with an alkyl group, and a substituent other than the alkyl group may be further linked.

In the present specification, an arylalkyl group means an alkyl group substituted with an aryl group, and a substituent other than the alkyl group may be further linked.

In the present specification, the aryloxy group is one in which an aryl group is linked to an oxygen atom, the arylthio group is one in which an aryl group is linked to a sulfur atom, and the above-described description on the aryl group may be applied to the aryl group of the aryloxy group and the arylthio group. An aryl group of an aryloxy group is the same as the above-described examples of the aryl group. Specifically, examples of the aryloxy group include a phenoxy group, a p-tolyloxy group, an m-tolyloxy group, a 3,5-dimethyl-phenoxy group, a 2,4,6-trimethylphenoxy group, a p-tert-butylphenoxy group, a 3-biphenyloxy group, a 4-biphenyloxy group, a 1-naphthyloxy group, a 2-naphthyloxy group, a 4-methyl-1-naphthyloxy group, a 5-methyl-2-naphthyloxy group, a 1-anthryloxy group, a 2-anthryloxy group, a 9-anthryloxy group, a 1-phenanthryloxy group, a 3-phenanthryloxy group, a 9-phenanthryloxy group, and the like, and examples of the arylthioxy group include a phenylthioxy group, a 2-methylphenylthioxy group, a 4-tert-butylphenylthioxy group, and the like, but the examples are not limited thereto.

In the present specification, a heterocyclic group is a cyclic group including one or more of N, O, P, S, Si, and Se as a heteroatom, and the number of carbon atoms thereof is not particularly limited, but is preferably 2 to 60. According to one embodiment, the number of carbon atoms of the heterocyclic group is from 2 to 30. According to one embodiment, the number of carbon atoms of the heterocyclic group is from 2 to 20. Examples of the heterocyclic group include a pyridyl group; a quinoline group; a thiophene group; a dibenzothiophene group; a furan group; a dibenzofuran group; a naphthobenzofuran group; a carbazole group; a benzocarbazole group; a naphthobenzothiophene group; a dibenzosilole group; a naphthobenzosilole group; a hexahydrocarbazole group; dihydroacridine group; a dihydrodibenzoazasiline group; a phenoxazine group; a phenothiazine group; a spiro(dibenzosilole-dibenzoazasiline) group; a spiro(acridine-fluorene) group, and the like, but are not limited thereto.

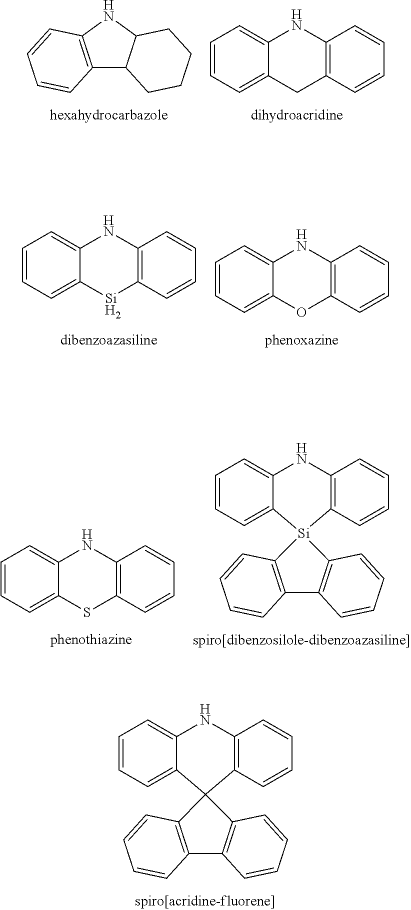

In the present specification, the above-described description on the heterocyclic group may be applied to a heteroaryl group except for an aromatic heteroaryl group.

In the present specification, an amine group may be selected from the group consisting of —NH2; an alkylamine group; an N-alkylarylamine group; an arylamine group; an N-arylheteroarylamine group; an N-alkylheteroarylamine group; and a heteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, an N-phenylnaphthylamine group, a ditolylamine group, an N-phenyltolylamine group, a triphenylamine group, an N-phenylbiphenylamine group, an N-biphenylnaphthylamine group, an N-naphthylfluorenylamine group, an N-phenylphenanthrenylamine group, an N-biphenylphenanthrenylamine group, an N-phenylfluorenylamine group, an N-phenyl terphenylamine group, an N-phenanthrenylfluorenylamine group, an N-biphenylfluorenylamine group, and the like, but are not limited thereto.

In the present specification, an N-alkylarylamine group means an amine group in which an alkyl group and an aryl group are substituted with N of the amine group.

In the present specification, an N-arylheteroarylamine group means an amine group in which an aryl group and a heteroaryl group are substituted with N of the amine group.

In the present specification, an N-alkylheteroarylamine group means an amine group in which an alkyl group and a heteroaryl group are substituted with N of the amine group.

In the present specification, an alkyl group, an aryl group, and a heteroaryl group in an alkylamine group; an N-alkylarylamine group; an arylamine group; an N-arylheteroarylamine group; an N-alkylheteroarylamine group, and a heteroarylamine group are each the same as the above-described examples of the alkyl group, the aryl group, and the heteroaryl group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other. In addition, substituents (four in total) linked to two consecutive carbons in an aliphatic ring may be interpreted as “adjacent” groups.

In the present specification, the “adjacent groups are bonded to each other to form a ring” among the substituents means that a substituent is bonded to an adjacent group to form a substituted or unsubstituted hydrocarbon ring; or a substituted or unsubstituted hetero ring.

In the present specification, “a five-membered or six-membered ring formed by bonding adjacent groups” means that a ring including a substituent participating in the ring formation is five-membered or six-membered. It is possible to include an additional ring condensed to the ring including the substituent participating in the ring formation.

In the present specification, the hydrocarbon ring may be an aromatic hydrocarbon ring, an aliphatic hydrocarbon ring, or a combination thereof.

In the present specification, a hetero ring is a ring including a heteroatom, and may be in the form in which an aromatic hydrocarbon ring or an aliphatic hydrocarbon ring fused with a ring having a heteroatom. The hetero ring may be an aromatic hetero ring, an aliphatic hetero ring or a combination thereof.

In the present specification, an aromatic hydrocarbon ring means a hydrocarbon ring in which pi electrons are completely conjugated and are planar, and the description on the aryl group may be applied to an aromatic hydrocarbon ring except for a divalent aromatic hydrocarbon ring.

In the present specification, an aliphatic hydrocarbon ring has a cyclically bonded structure, and means a non-aromatic ring. Examples of the aliphatic hydrocarbon ring include cycloalkyl or cycloalkene, and the above-described description on the cycloalkyl group or cycloalkenyl group may be applied to the aliphatic hydrocarbon ring except for a divalent aliphatic hydrocarbon ring. Further, a substituted aliphatic hydrocarbon ring also includes an aliphatic hydrocarbon ring in which aromatic rings are fused.

In the present specification, a fused ring of an aromatic hydrocarbon ring and an aliphatic hydrocarbon ring means that an aromatic hydrocarbon ring and an aliphatic hydrocarbon ring form a fused ring. Examples of the condensed ring of the aromatic ring and the aliphatic ring include a 1,2,3,4-tetrahydronaphthalene group, a 2,3-dihydro-1H-indene group, and the like, but are not limited thereto.

In the present specification, when adjacent groups among substituents are bonded to each other to form a ring, it is possible to form any one of the following structures.

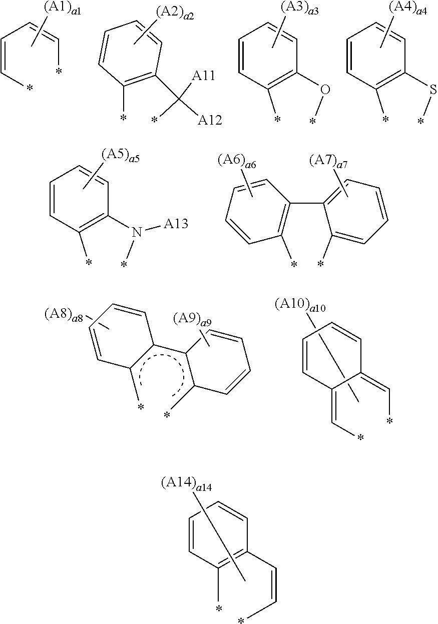

In the structures,
A1 to A14 are each independently hydrogen; deuterium; a halogen group; a cyano group; a substituted or unsubstituted alkyl group; a substituted or unsubstituted cycloalkyl group; a substituted or unsubstituted aryl group; or a substituted or unsubstituted heterocyclic group,
a1 to a9 are each an integer from 1 to 4,
a10 and a14 are each an integer from 1 to 6,
when a1 to a14 are each 2 or higher, two or more substituents in the parenthesis are the same as or different from each other, and
* denotes a position that is substituted.

According to an exemplary embodiment of the present specification, A1 is hydrogen; or a substituted or unsubstituted aryl group.

According to an exemplary embodiment of the present specification, A1 is hydrogen; or a substituted or unsubstituted aryl group having 6 to 30 carbon atoms.

According to an exemplary embodiment of the present specification, A1 is hydrogen; or a substituted or unsubstituted phenyl group.

According to an exemplary embodiment of the present specification, A1 is hydrogen or a phenyl group.

According to an exemplary embodiment of the present specification, A2 to A10 and A14 are hydrogen.

According to an exemplary embodiment of the present specification, A11 to A13 are each independently a substituted or unsubstituted alkyl group; or a substituted or unsubstituted aryl group.

According to an exemplary embodiment of the present specification, A11 to A13 are each independently a substituted or unsubstituted alkyl group having 1 to 30 carbon atoms; or a substituted or unsubstituted aryl group having 6 to 30 carbon atoms.

According to an exemplary embodiment of the present specification, A11 and A12 are a methyl group.

According to an exemplary embodiment of the present specification, A13 is a substituted or unsubstituted benzene group, or a substituted or unsubstituted naphthalene group.

In the present specification, x is 1, and y is 1.

In the present specification, x is 1, and y is 2.

In the present specification, x is 2, and y is 1.

In the present specification, x is 2, and y is 2.

In the present specification, x is 1, y is 1, and L₁ is a substituent of the following Chemical Formula A, or a substituent of the following Chemical Formula B.

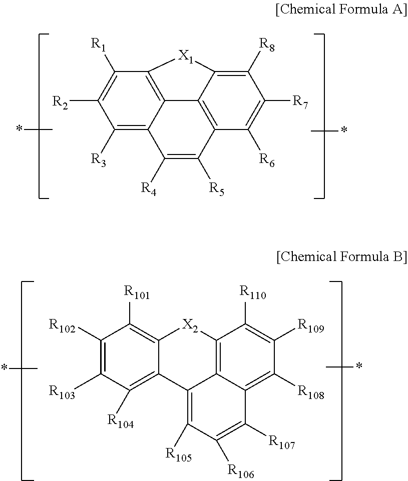

in Chemical Formulae A and B,
X₁ and X₂ are the same as or different from each other, and are each independently O or S,
two of R₁ to R₈ and two of R₁₀₁ to R₁₁₀ are each linked to an adjacent group, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring, and
* denotes a bonding position.

In the present specification, x is 1, y is 1, and one of R₁ to R₈ and R₁₀₁ to R₁₁₀ is a substituent of the following Chemical Formula A-1, or a substituent of the following Chemical Formula B-1.

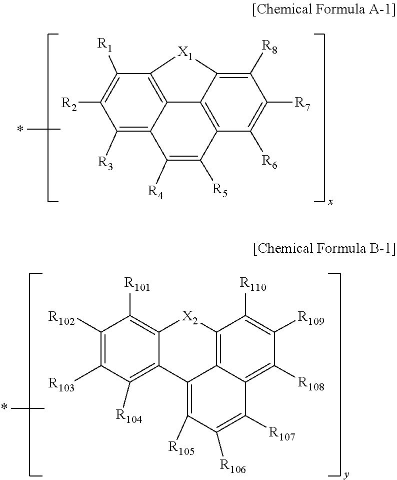

in Chemical Formulae A and B,
X₁ and X₂ are the same as or different from each other, and are each independently O or S,
one of R₁ to R₈ and one of R₁₀₁ to R₁₁₀ are each linked to an adjacent group, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring, and
* denotes a bonding position.

In the present specification, wherein at least one of R₁ and R₂, R₂ and R₃, R₄ and R₅, R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₈ and R₁₀₉, and R₁₀₉ and R₁₁₀ are bonded to form a substituted or unsubstituted hydrocarbon ring.

In the present specification, wherein at least one of R₁ and R₂, R₂ and R₃, R₄ and R₅, R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₇ and R₁₀₈, and R₁₀₉ and R₁₁₀ are bonded to form a substituted or unsubstituted benzene ring, or a substituted or unsubstituted naphthalene ring.

In the present specification, wherein at least one of R₁ and R₂, R₂ and R₃, R₄ and R₅, R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₈ and R₁₀₉, and R₁₀₉ and R₁₁₀ are bonded to form a benzene ring that is unsubstituted or substituted with deuterium, or a naphthalene ring that is unsubstituted or substituted with deuterium.

In the present specification, wherein at least one of R₁ and R₂, R₂ and R₃, R₄ and R₅, R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₈ and R₁₀₉, and R₁₀₉ and R₁₁₀ are bonded to form a substituted or unsubstituted benzene ring.

In the present specification, wherein at least one of R₁ and R₂, R₂ and R₃, R₄ and R₅, R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₈ and R₁₀₉, and R₁₀₉ and R₁₁₀ are bonded to form a benzene ring that is unsubstituted or substituted with deuterium.

The another present specification provides a compound of any one of the following Chemical Formulae 11 to 13.

In the present specification, Chemical Formula 1 is any one of the following Chemical Formulae 11 to 13.

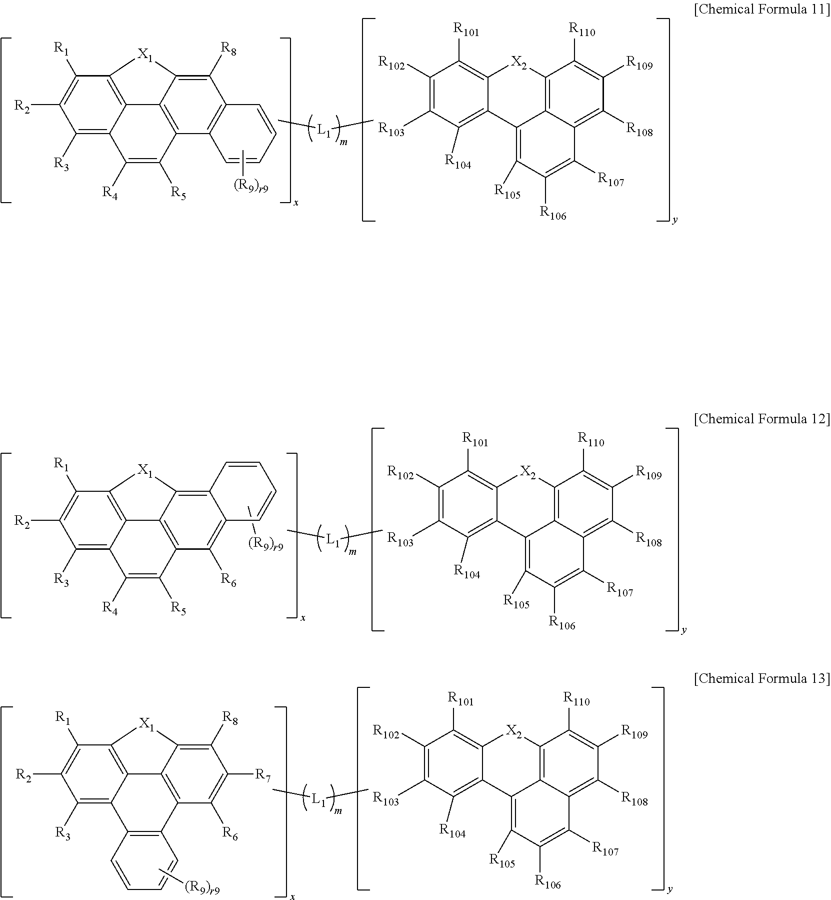

wherein in Chemical Formulae 11 to 13,
X₁ and X₂ are the same as or different from each other, and are each independently O or S,
L₁ is a direct bond, a substituted or unsubstituted arylene group or a substituted or unsubstituted divalent heterocyclic group,
here, L₁ is further unsubstituted or substituted with deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and optionally, at least one of the substituents of L₁ is optionally bonded to an adjacent substituent to form one or more substituted or unsubstituted rings,
one of R₁ to R₉ and one of R₁₀₁ to R₁₁₀ are each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring,
m is an integer from 1 to 4, and when m is 2 or higher, L₁'s are the same as or different from each other,
x is 1 or 2, and y is 1 or 2,
when x and y are each 2, structures in the parenthesis are the same as or different from each other, and
r9 is an integer from 1 to 4, and when r9 is 2 or higher, R₉'s are the same as or different from each other.

In the present specification, Chemical Formula 1 is any one of the following Chemical Formulae 11 to 13.

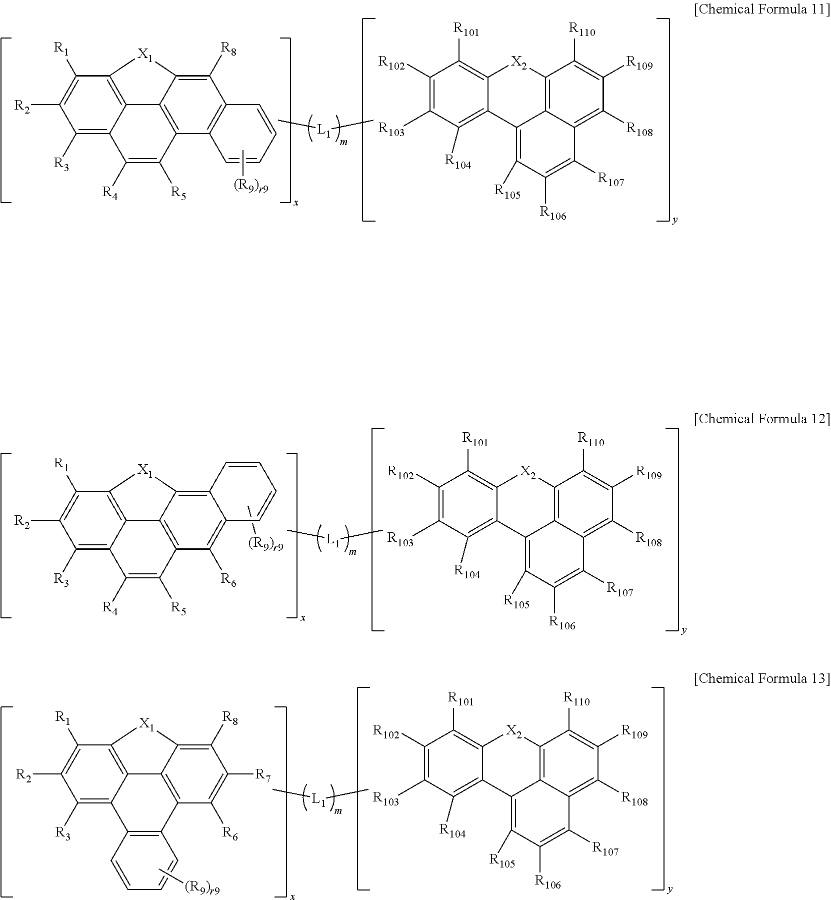

wherein in Chemical Formulae 11 to 13,
X₁ and X₂ are the same as or different from each other, and are each independently O or S,
L₁ is a direct bond, a substituted or unsubstituted arylene group or a substituted or unsubstituted divalent heterocyclic group,
here, L₁ is further unsubstituted or substituted with deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and optionally, at least one of the substituents of L₁ is optionally bonded to an adjacent substituent to form one or more substituted or unsubstituted rings,
one of R₁ to R₉ and one of R₁₀₁ to R₁₁₀ are each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group,
m is an integer from 1 to 4, and when m is 2 or higher, L₁'s are the same as or different from each other,
x is 1 or 2, and y is 1 or 2,
when x and y are each 2, structures in the parenthesis are the same as or different from each other, and
r9 is an integer from 1 to 4, and when r9 is 2 or higher, R₉'s are the same as or different from each other.

In the present specification, Chemical Formula 1 is any one of the following Chemical Formula 11 or 12.

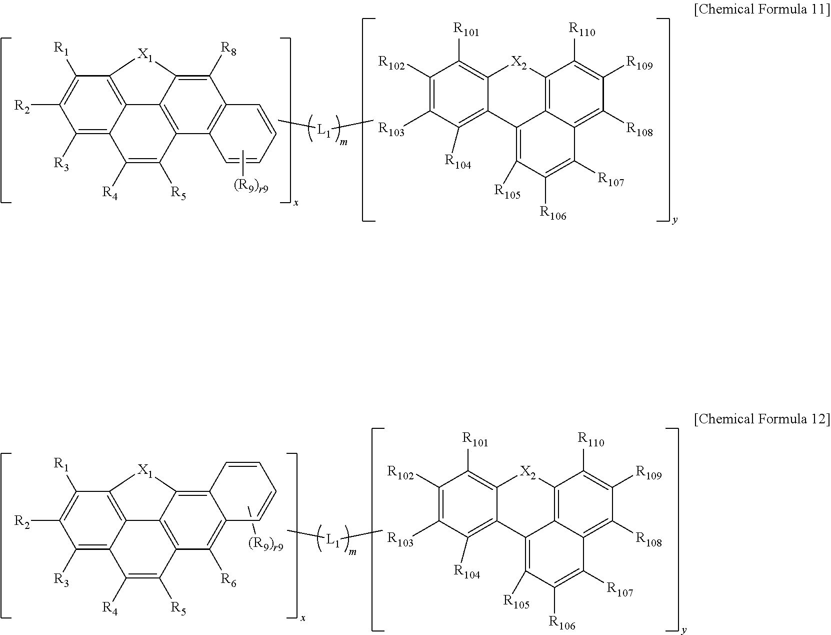

wherein in Chemical Formulae 11 and 12,
X₁, X₂, L₁, x, m, R₁₀₁ to R₁₁₀ and y are the same as the definitions of Chemical Formula 1,
one of R₁ to R₆, R₈ and R₉ is each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and
r9 is an integer from 1 to 4, and when r9 is 2 or higher, R₉'s are the same as or different from each other.

In the present specification, Chemical Formula 11 is any one of the following Chemical Formulae 111 to 114.

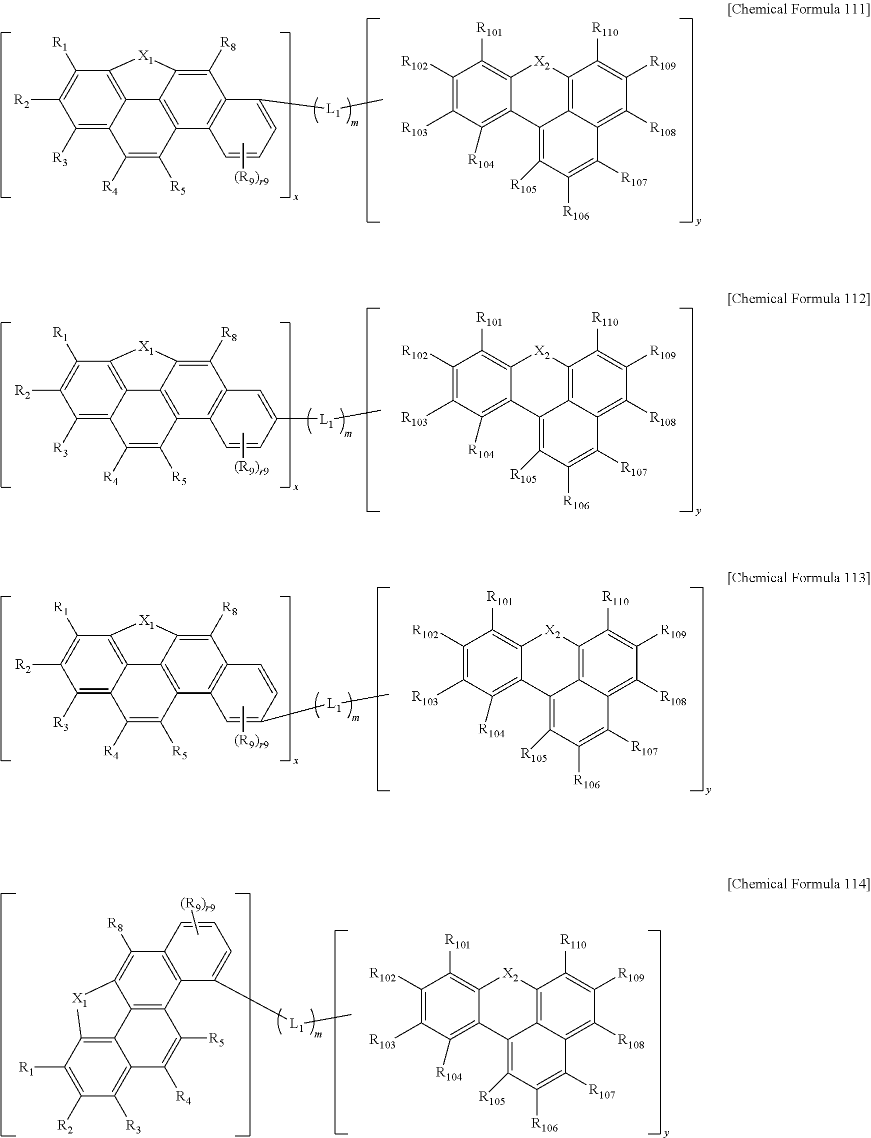

wherein in Chemical Formulae 111 to 114,
X₁, X₂, L₁, x, m, R₁₀₁ to R₁₁₀ and y are the same as the definitions of Chemical Formula 11,
R₁ to R₅, R₈ and R₉ are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and

r9 is an integer from 1 to 3, and when r9 is 2 or higher, R₉'s are the same as or different from each other.

In the present specification, Chemical Formula 12 is any one of the following Chemical Formulae 121 to 124.

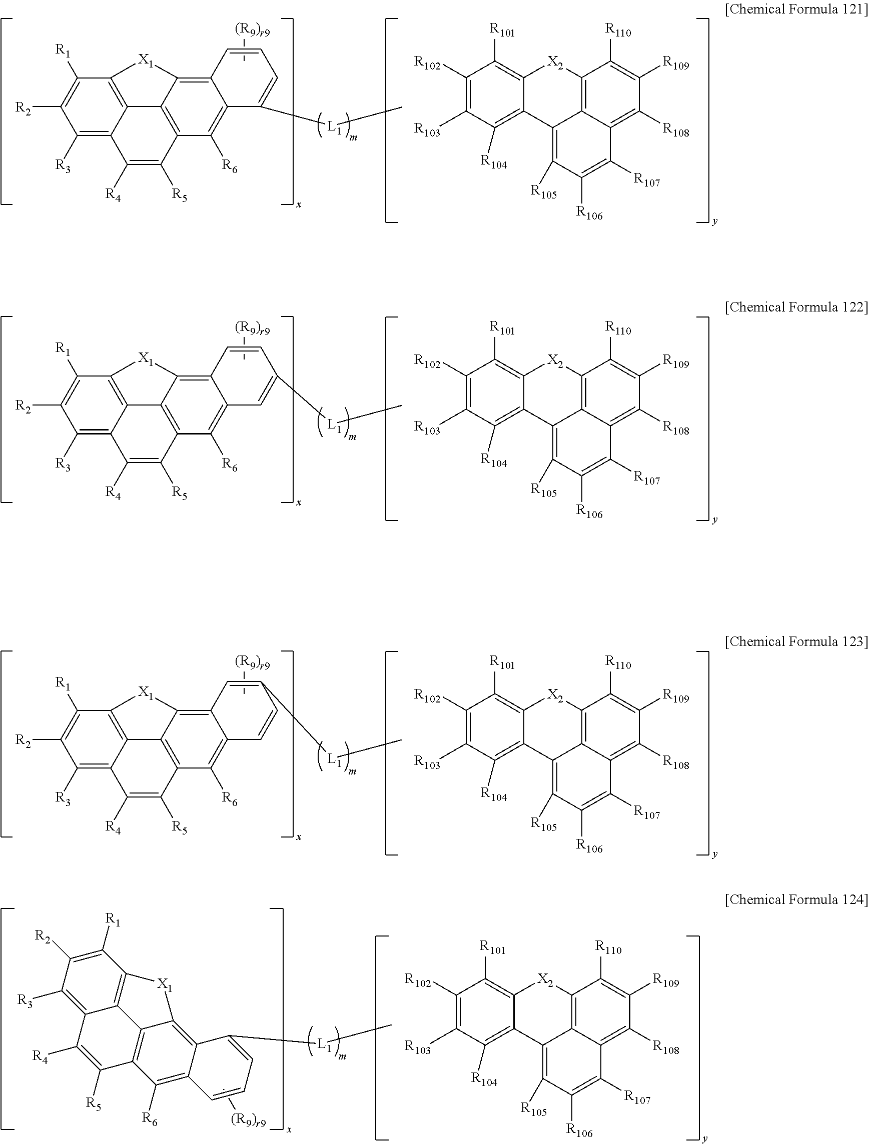

wherein in Chemical Formulae 121 to 124,
X₁, X₂, L₁, x, m, R₁₀₁ to R₁₁₀ and y are the same as the definitions of Chemical Formula 12,
R₁ to R₆ and R₉ are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and
r9 is an integer from 1 to 3, and when r9 is 2 or higher, R₉'s are the same as or different from each other.

In the present specification, Chemical Formula 13 is any one of the following Chemical Formulae 131 to 134.

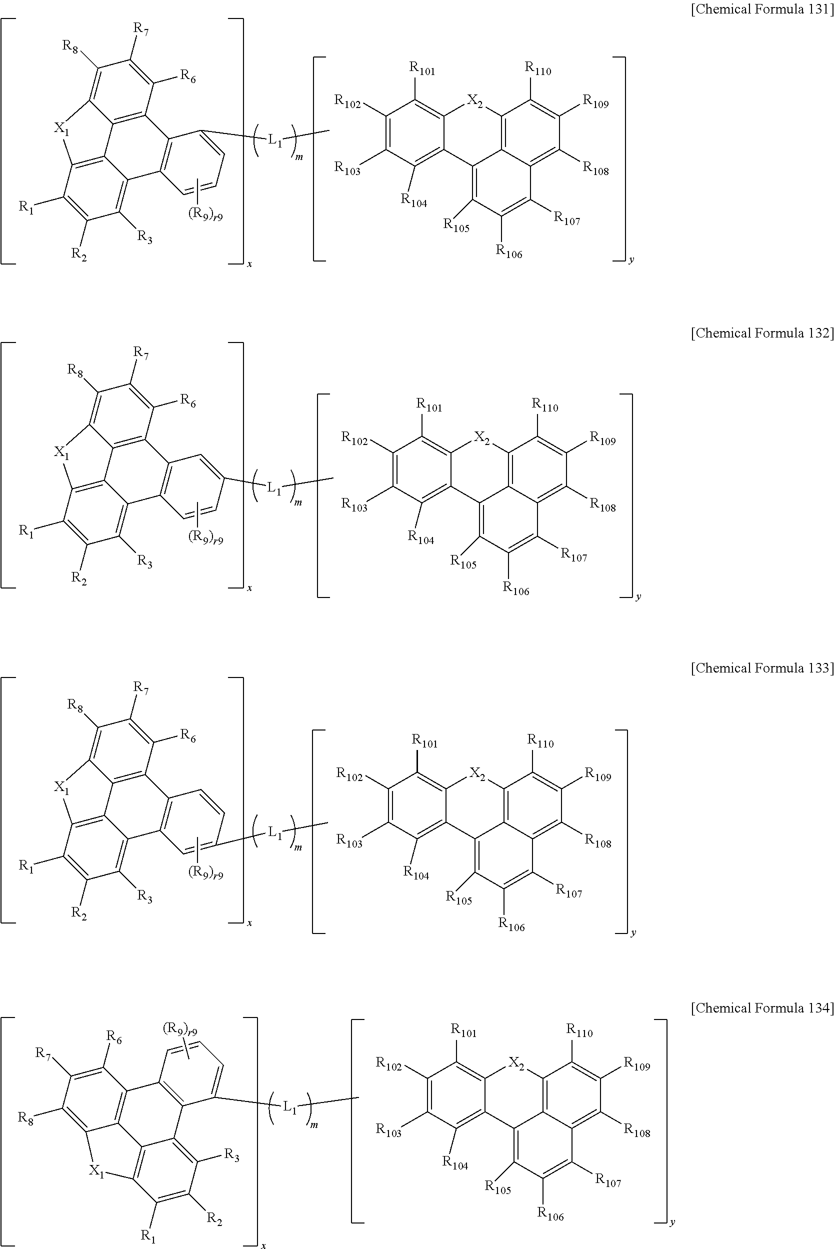

wherein in Chemical Formulae 131 to 134,
X₁, X₂, L₁, x, m, R₁₀₁ to R₁₁₀ and y are the same as the definitions of Chemical Formula 13,
R₁ to R₃ and R₆ to R₉ are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and
r9 is an integer from 1 to 3, and when r9 is 2 or higher, R₉'s are the same as or different from each other.

In an exemplary embodiment of the present specification, the compound may have a band gap energy of 2.9 eV or more, and 2.9 eV or more and 4.0 eV or less. The organic compound used in the organic light emitting device may serve as an organic semiconductor only when the organic compound needs to have a band gap of 0.5 eV to 4.0 eV, and in order to exhibit blue color, the organic compound needs to have a band gap energy of at least 2.9 eV. According to the principle of fluorescent blue light emission in the host-dopant system, energy transfer occurs smoothly when the band gap of the blue fluorescent host is larger than the band gap of the blue fluorescent dopant. Therefore, in order for the compound to be used as a blue fluorescent host, it is preferred that the compound has an energy band gap of 2.9 eV to 4.0 eV.

Various methods are known to determine band gap energy, and particularly, cyclic voltammetry and absorption spectroscopy are representative.

Cyclic voltammetry is an electrochemical technique that measures the band gap through the oxidation and reduction reactions of compounds, and the technique measures current under conditions of cyclical voltage changes, and measures the current caused by oxidation-reduction reactions that occur according to changes in potential energy.

Absorption spectroscopy measures UV-vis spectra, and is exemplified in Korean Patent Publication No. 2009-0051787 (KR 2009-0051787 A).

In the present specification, “including deuterium,” “deuteration” or “deuterated” means that hydrogen at a substitutable position of a compound is substituted with deuterium.

In the present specification, “overdeuterated” means a compound or group in which all hydrogens in the molecule are substituted with deuterium, and has the same meaning as “100% deuterated”.

In the present specification, “X % deuterated”, “X % degree of deuteration”, or “X % deuterium substitution rate” means that X % of the hydrogens at substitutable positions in the corresponding structure are substituted with deuterium. For example, when the corresponding structure is dibenzofuran, the dibenzofuran being “25% deuterated”, “25% degree of deuteration” of the dibenzofuran, or “25% deuterium substitution rate” of the dibenzofuran means that 2 of the 8 hydrogens at the substitutable positions of the dibenzofuran are substituted with deuterium.

In an exemplary embodiment of the present specification, the deuterium substitution rate of the compound may be 1% to 100%, specifically, 40% to 99%. The deuterium substitution rate of the compound may be 5% or more, 10% or more, 20% or more, 30% or more, 40% or more, 50% or more, 60% or more, 70% or more, or 80% or more, and 100% or less, or 99% or less. The physicochemical properties associated with deuterium, such as chemical bond length, appear to be different from those of hydrogen. In particular, it can be shown that the stretching amplitude of C-D bonds is smaller than that of C—H bonds, and the van der Waals radius of deuterium is smaller than that of hydrogen, and that C-D bonds are generally shorter and stronger than C—H bonds. When substituted with deuterium, the energy of the ground state becomes lower and the bond length between deuterium and carbon becomes shorter, so that the molecular hardcore volume decreases. Accordingly, the electrical polarizability may be reduced and the intermolecular interaction may be weakened, so that the volume of the thin film may be increased.

These characteristics may have an effect of reducing the crystallinity of the thin film, that is, create an amorphous state, and may be generally effective for increasing the service life and driving characteristics of an OLED, and the heat resistance may be more improved.

As a related art related to the organic light emitting compound including deuterium, Korean Patent No. 10-1111406 describes a technology for providing a device with low voltage driving and long service life by substituting an amine-based compound including carbazole with deuterium or mixing compounds substituted with deuterium, and Korean Patent No. 10-1068224 describes a technology for using an anthracene derivative including a phenyl group in which hydrogen in the phenyl group is substituted with deuterium as a host.

Here, the deuterium substitution rate may be calculated using a spectrogram obtained by mass spectrometry of a material separated by chromatography. Specifically, the deuterium substitution rate may be calculated based on the maximum value of the molecular weight distribution in the spectrogram obtained by mass spectrometry of a material separated by liquid chromatography or gas chromatography according to a sample to be measured. For example, when the degree of deuteration of the following Compound A is analyzed, the molecular weight of the following starting material is set to 506 and the maximum molecular weight (median value) of the following Compound A is set to 527 in the MS graph, then it may be calculated that about 81% of the hydrogen has been deuterated because 21 of the 26 hydrogens at the substitutable positions in the following starting material have been substituted with deuterium.

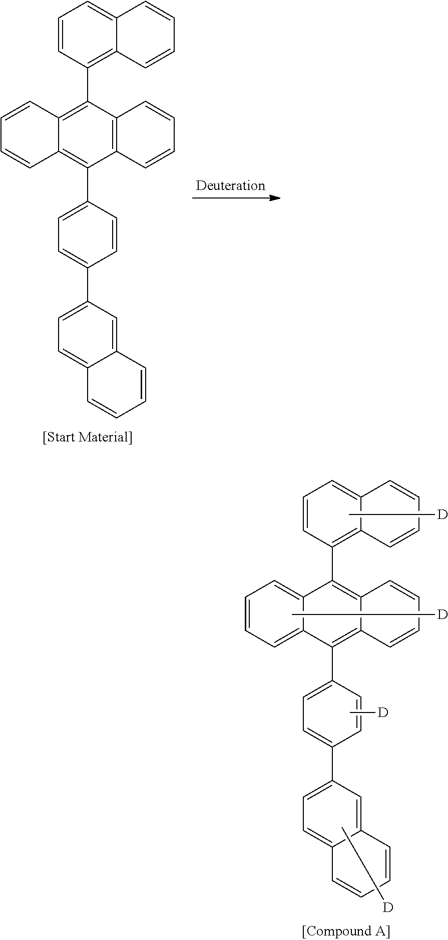

In the present specification, D means deuterium.

Another method for determining the deuterium substitution rate may use nuclear magnetic resonance (NMR), and specifically by adding dimethylformamide (DMF) as an internal standard and using the integration ratio in the 1H NMR graph, the deuterium substitution rate may be calculated from the integrated amount of the total peak.

In an exemplary embodiment of the present specification, the compound of Chemical Formula 1 may be any one of the following structures.

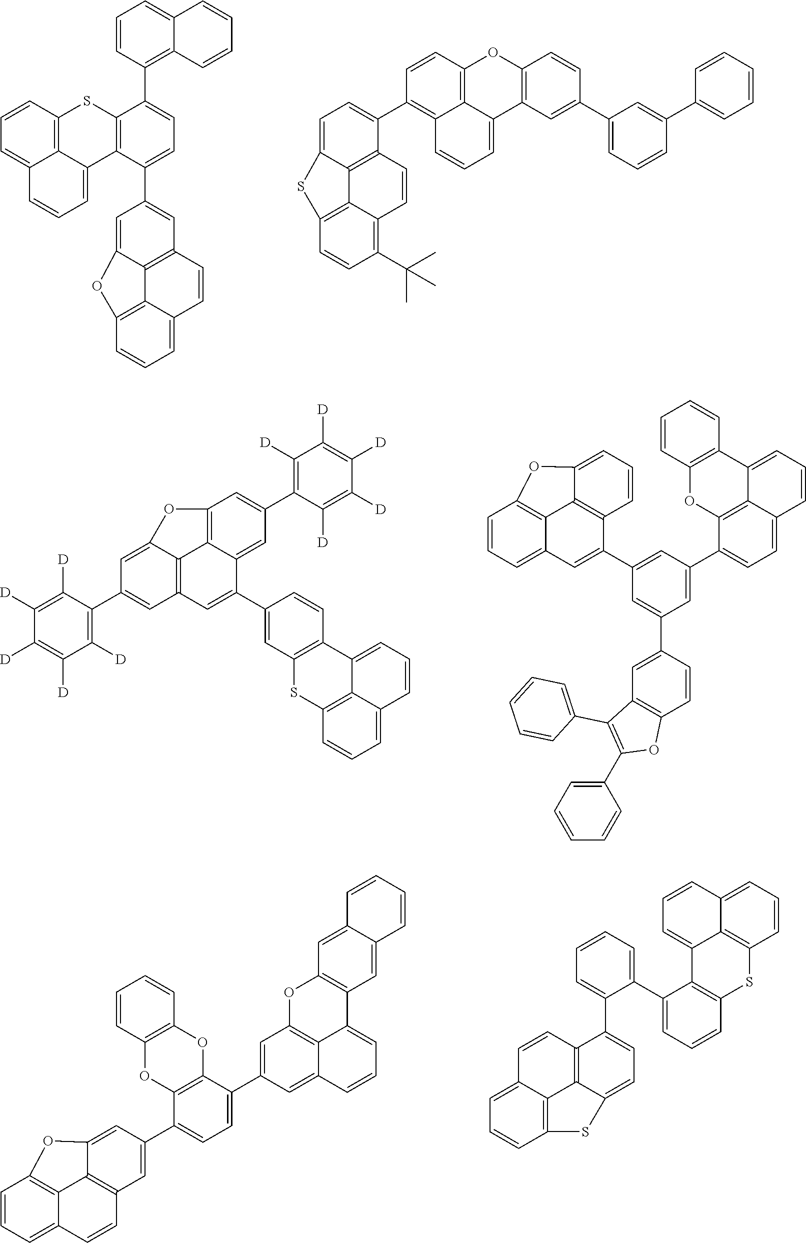

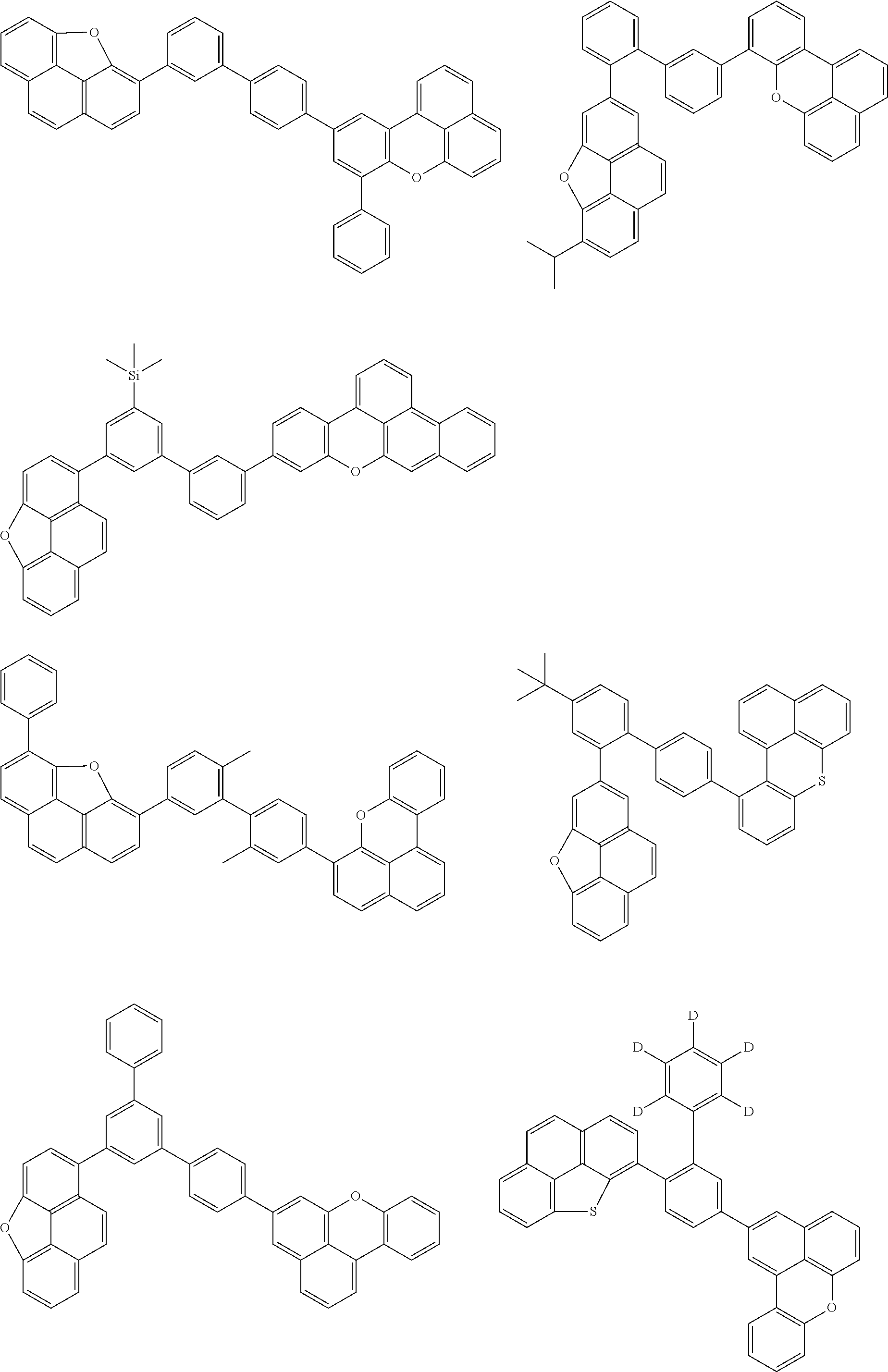

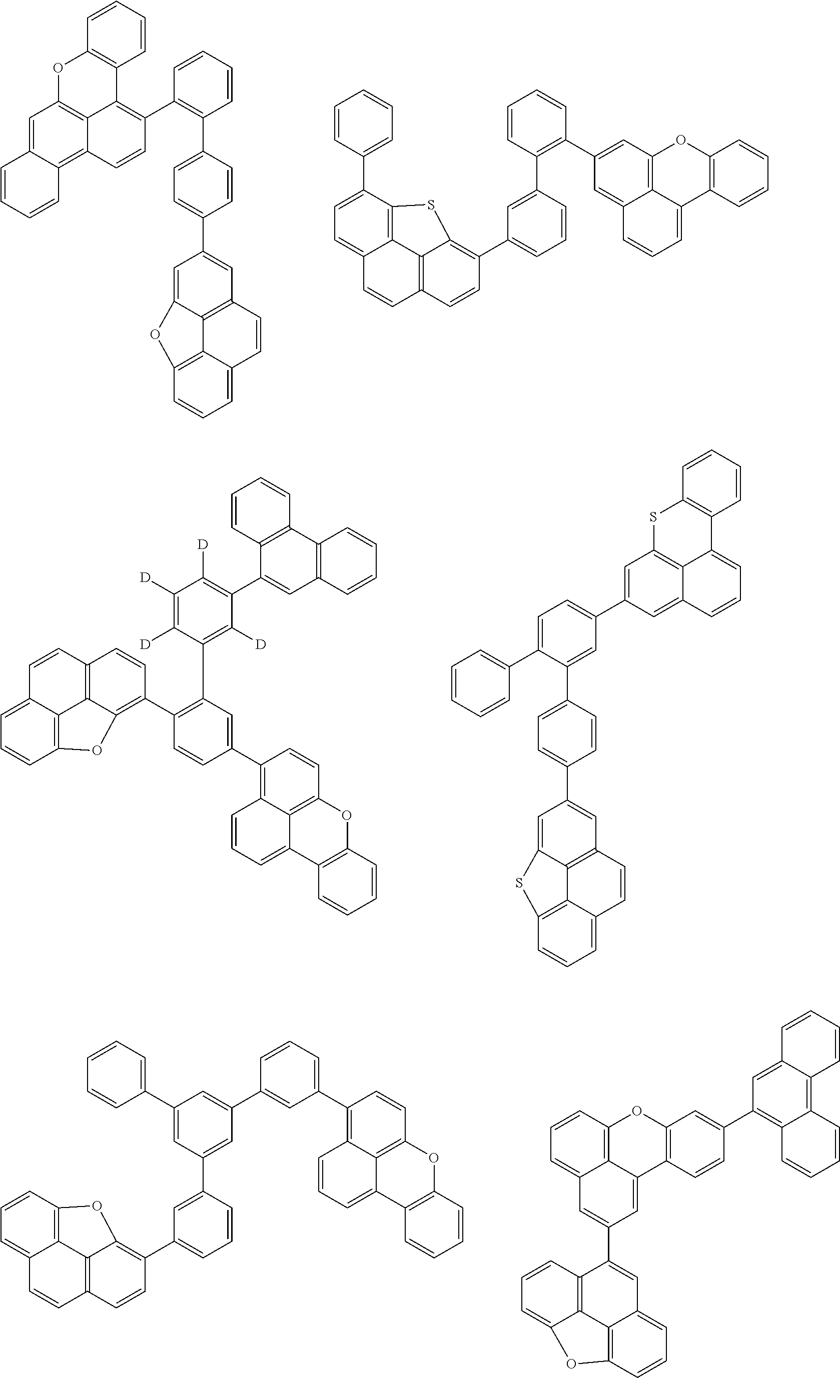

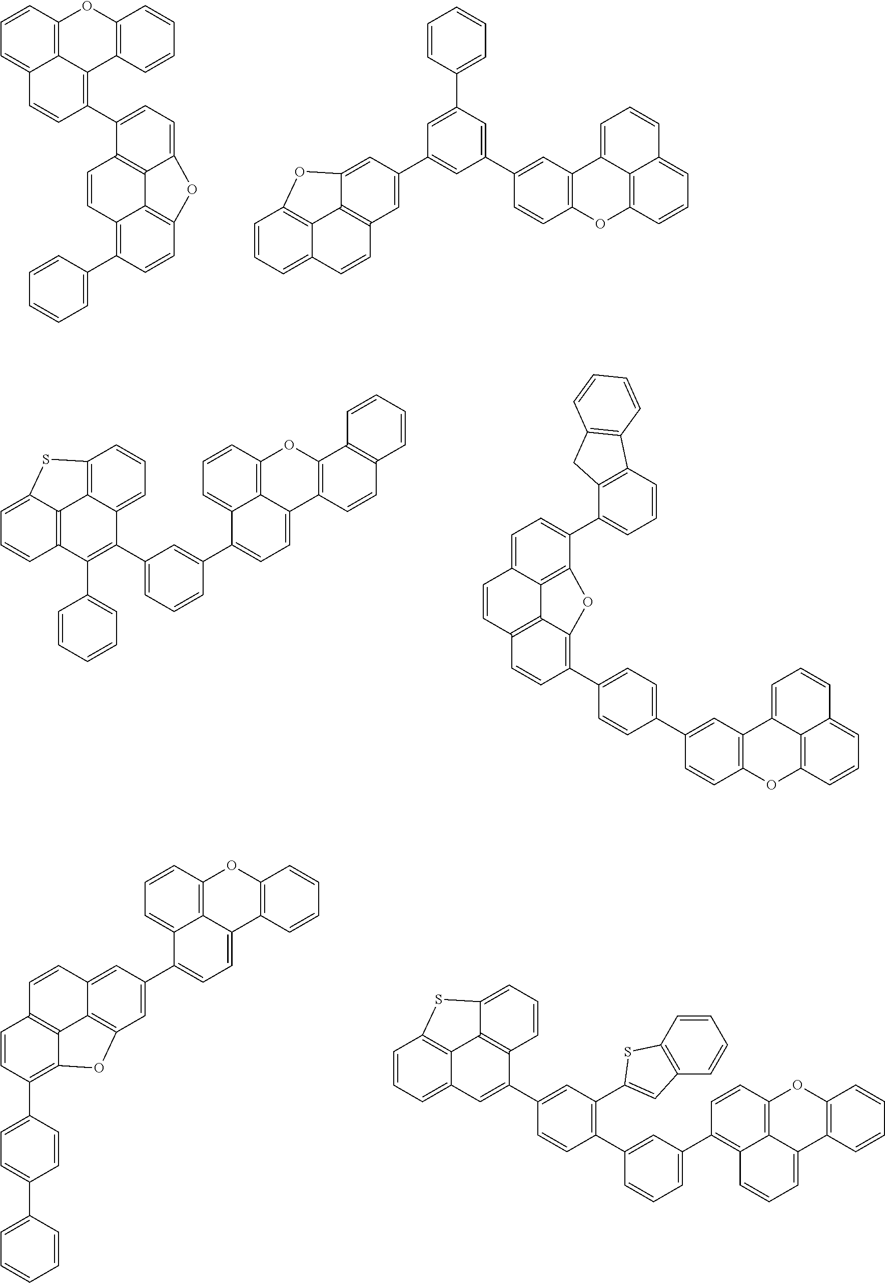 [SMILES: c1ccc(-c2ccc3oc4ccc(-c5ccc6cccc7c6c5-c5ccccc5O7)c5ccc2c3c45)cc1 ; c1ccc(-c2cc(-c3ccc4c(c3)-c3cccc5cccc(c35)O4)cc(-c3cc4ccc5cccc6oc(c3)c4c56)c2)cc1 ; c1ccc(-c2c(-c3cccc(-c4ccc5c6c(cccc46)Oc4c-5ccc5ccccc45)c3)c3cccc4sc5cccc2c5c43)cc1 ; c1ccc2c(c1)Cc1c-2cccc1-c1ccc2ccc3ccc(-c4ccc(-c5ccc6c(c5)-c5cccc7cccc(c57)O6)cc4)c4oc1c2c34 ; c1ccc(-c2ccc(-c3ccc4ccc5cc(-c6ccc7c8c(cccc68)Oc6ccccc6-7)cc6oc3c4c56)cc2)cc1 ; c1cc(-c2ccc(-c3cc4cccc5sc6cccc3c6c45)cc2-c2cc3ccccc3s2)cc(-c2ccc3c4c(cccc24)-c2ccccc2O3)c1]

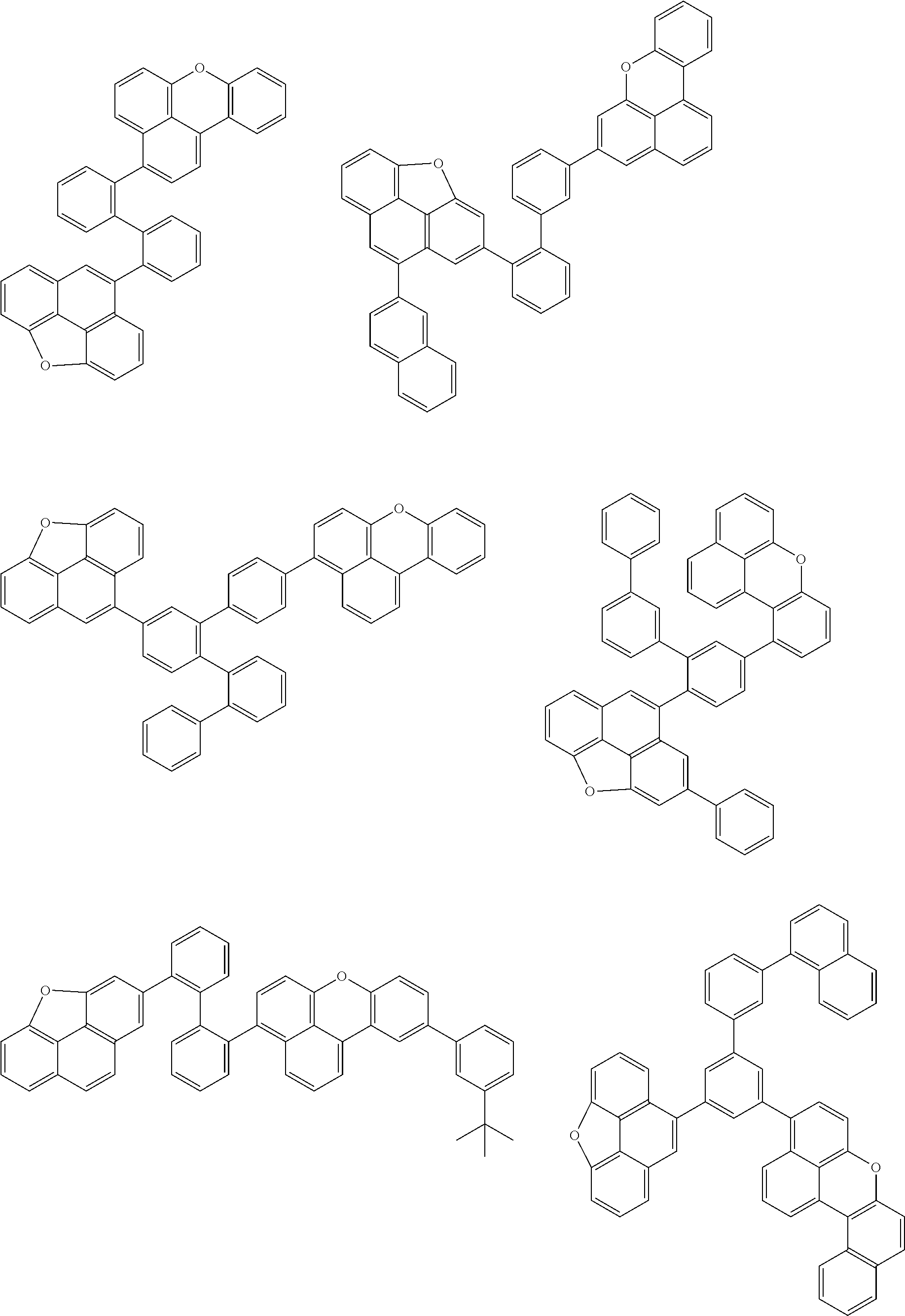

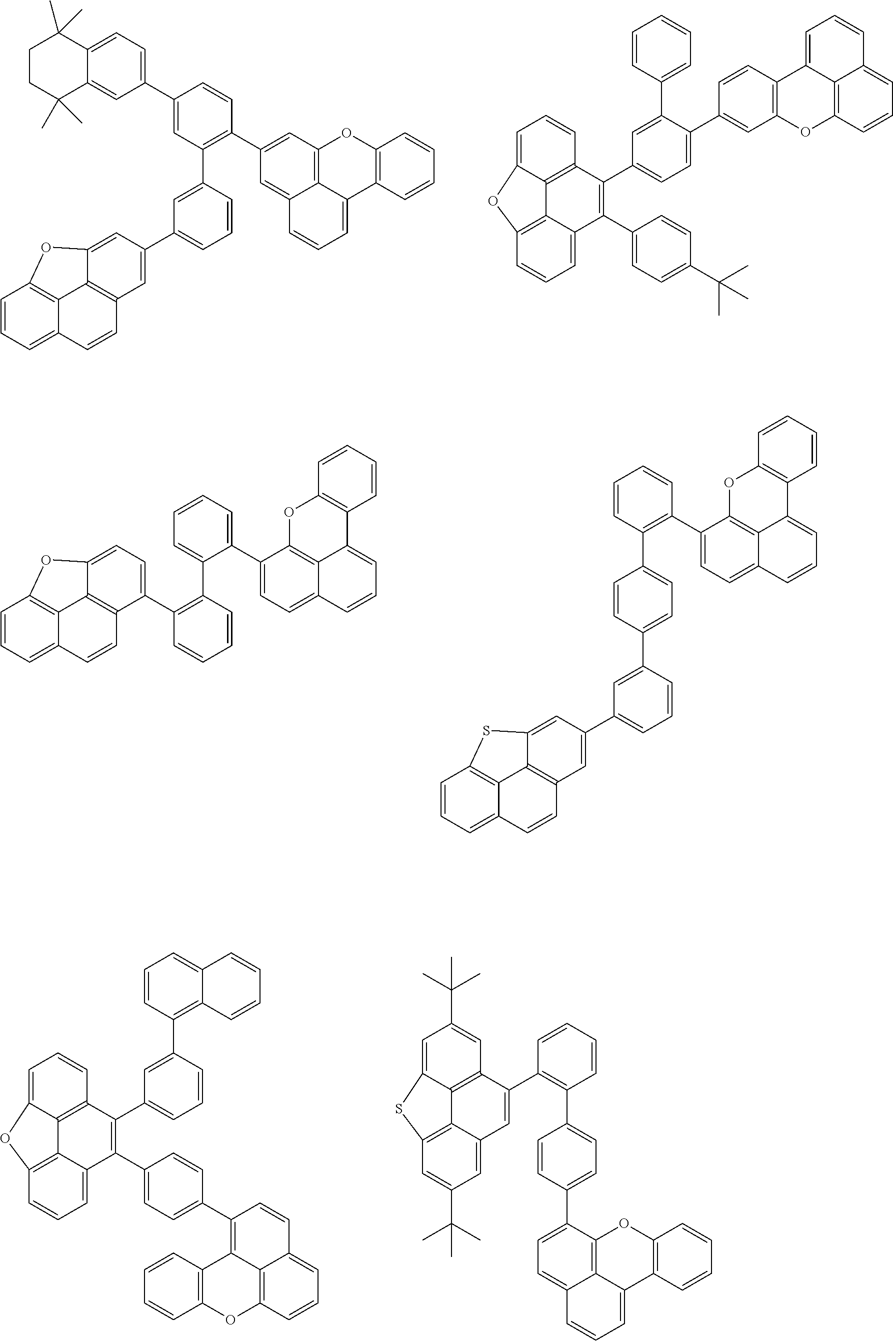

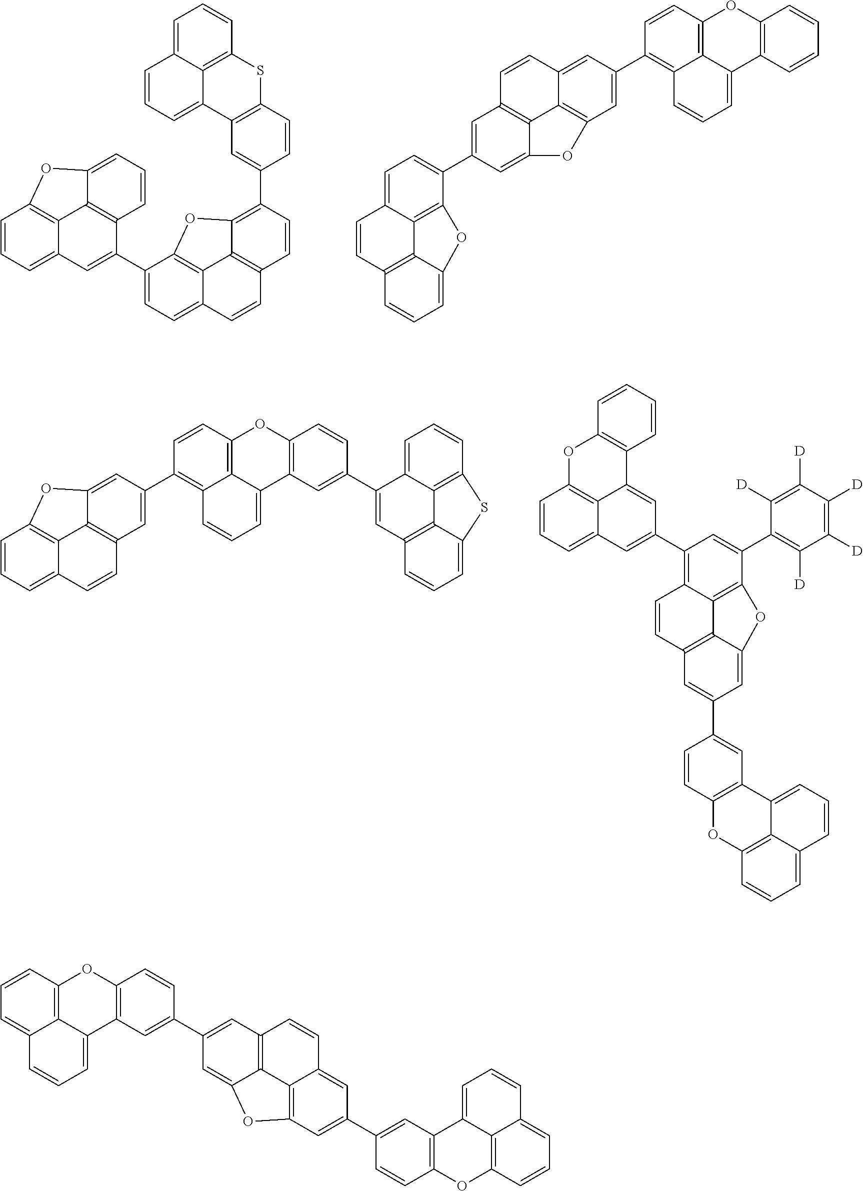 [SMILES: c1cc2c3c(cccc3c1)-c1cc(-c3ccc4ccc5ccc(-c6cc7cccc8oc9cccc6c9c78)c6oc3c4c56)ccc1S2 ; c1ccc2c(c1)Oc1ccc(-c3cc4ccc5cc(-c6ccc7ccc8cccc9oc6c7c89)cc6oc(c3)c4c56)c3cccc-2c13 ; c1cc2ccc3cc(-c4ccc5c6c(cccc46)-c4cc(-c6cc7cccc8sc9cccc6c9c78)ccc4O5)cc4oc(c1)c2c34 ; [2H]c1c([2H])c([2H])c(-c2cc(-c3cc4c5c(cccc5c3)Oc3ccccc3-4)c3ccc4cc(-c5ccc6c(c5)-c5cccc7cccc(c57)O6)cc5oc2c3c45)c([2H])c1[2H] ; c1cc2c3c(cccc3c1)-c1cc(-c3cc4ccc5cc(-c6ccc7c(c6)-c6cccc8cccc(c68)O7)cc6oc(c3)c4c56)ccc1O2]

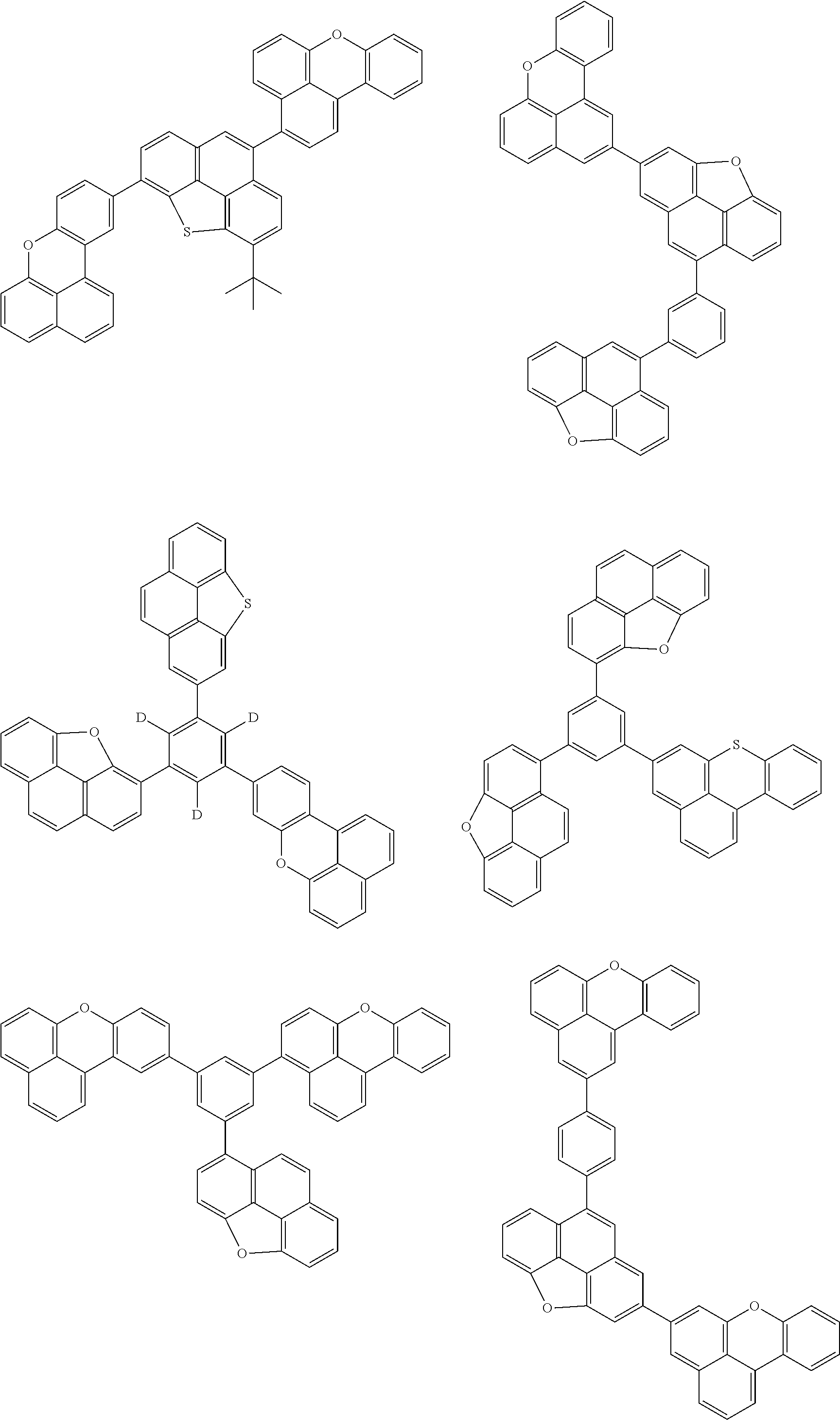

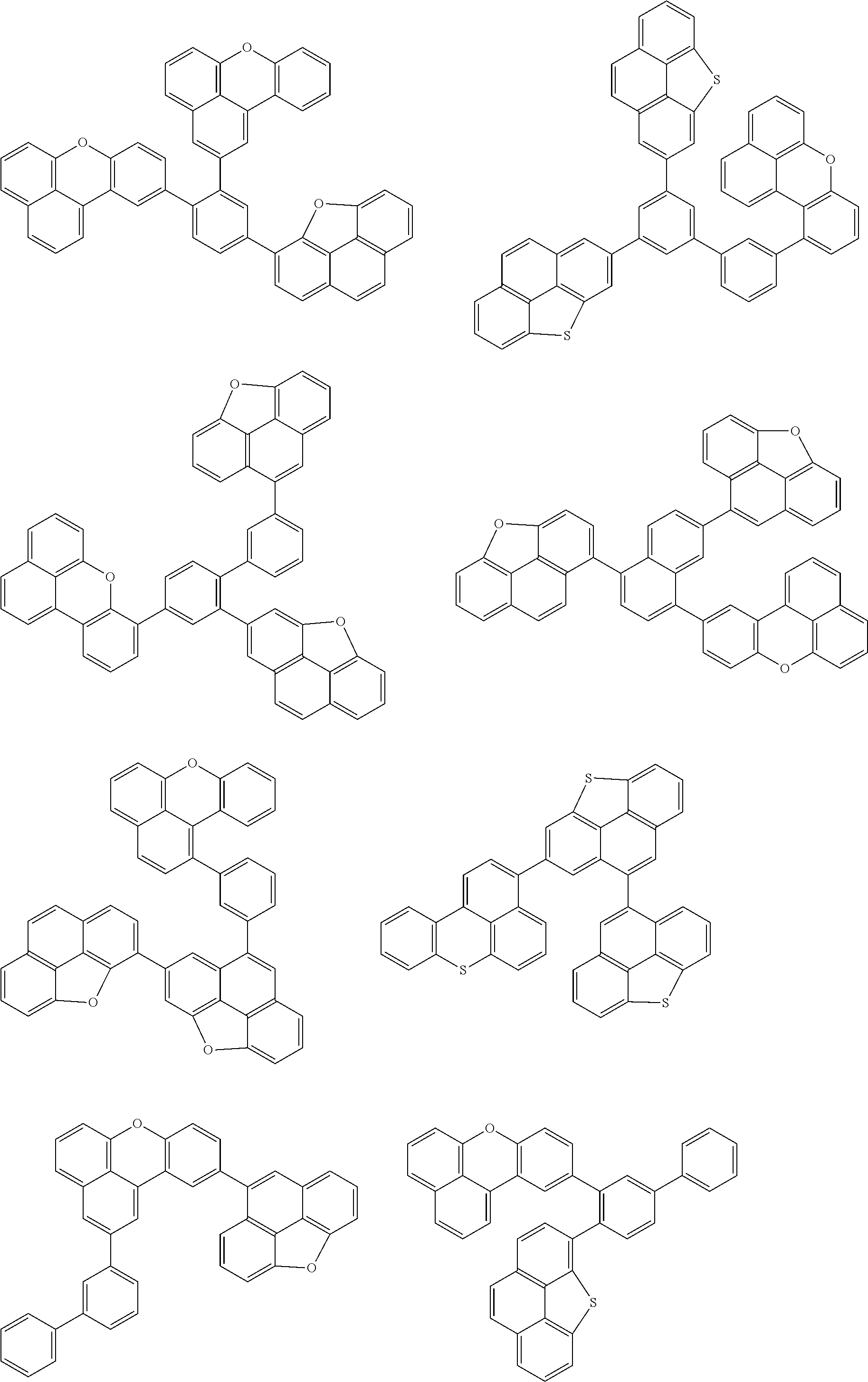

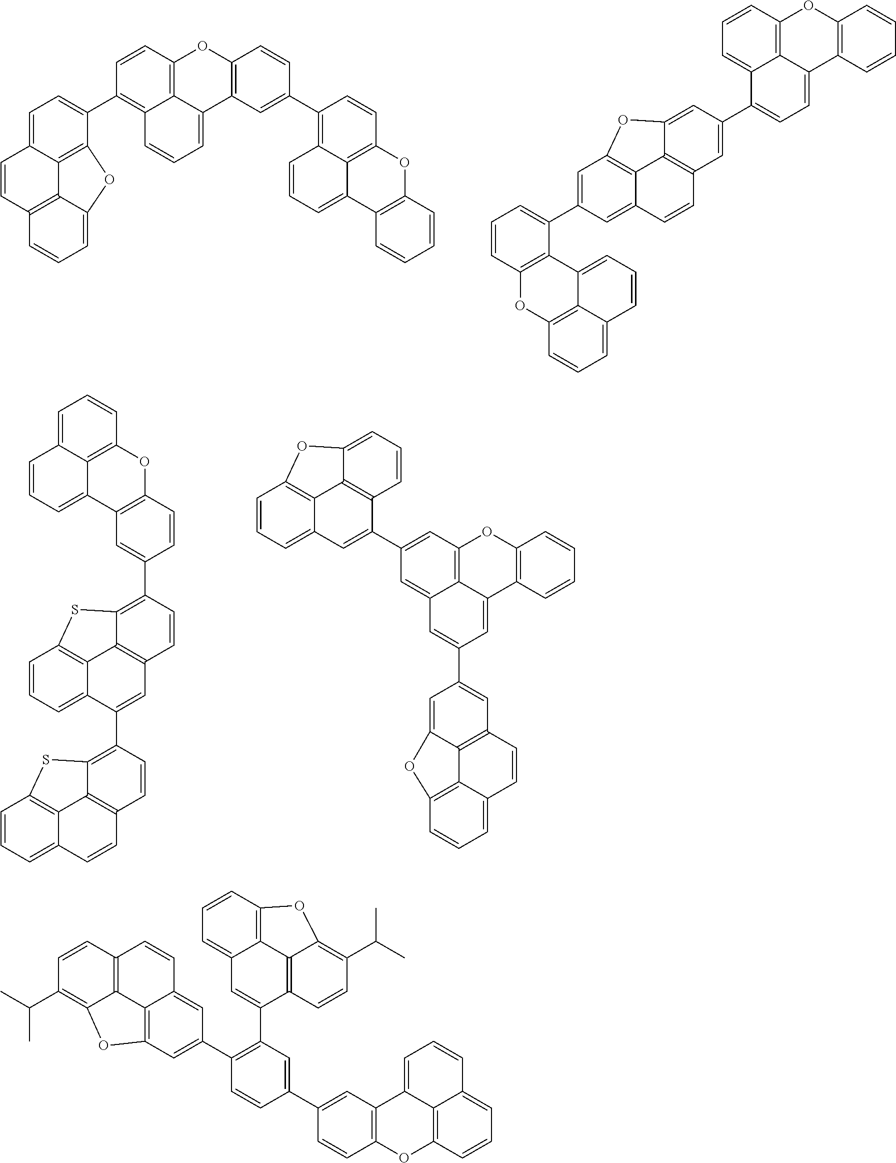 [SMILES: c1ccc2c(c1)Oc1ccc(-c3ccc4c(c3)-c3cccc5c(-c6ccc7ccc8cccc9oc6c7c89)ccc(c35)O4)c3cccc-2c13 ; c1ccc2c(c1)Oc1cccc3c(-c4cc5ccc6cc(-c7cccc8c7-c7cccc9cccc(c79)O8)cc7oc(c4)c5c67)ccc-2c13 ; c1cc2c3c(cccc3c1)-c1cc(-c3ccc4cc(-c5ccc6ccc7cccc8sc5c6c78)c5cccc6sc3c4c65)ccc1O2 ; c1ccc2c(c1)Oc1cc(-c3cc4cccc5oc6cccc3c6c45)cc3cc(-c4cc5ccc6cccc7oc(c4)c5c67)cc-2c13 ; CC(C)c1ccc2ccc3cc(-c4ccc(-c5ccc6c(c5)-c5cccc7cccc(c57)O6)cc4-c4cc5cccc6oc7c(C(C)C)ccc4c7c56)cc4oc1c2c34]

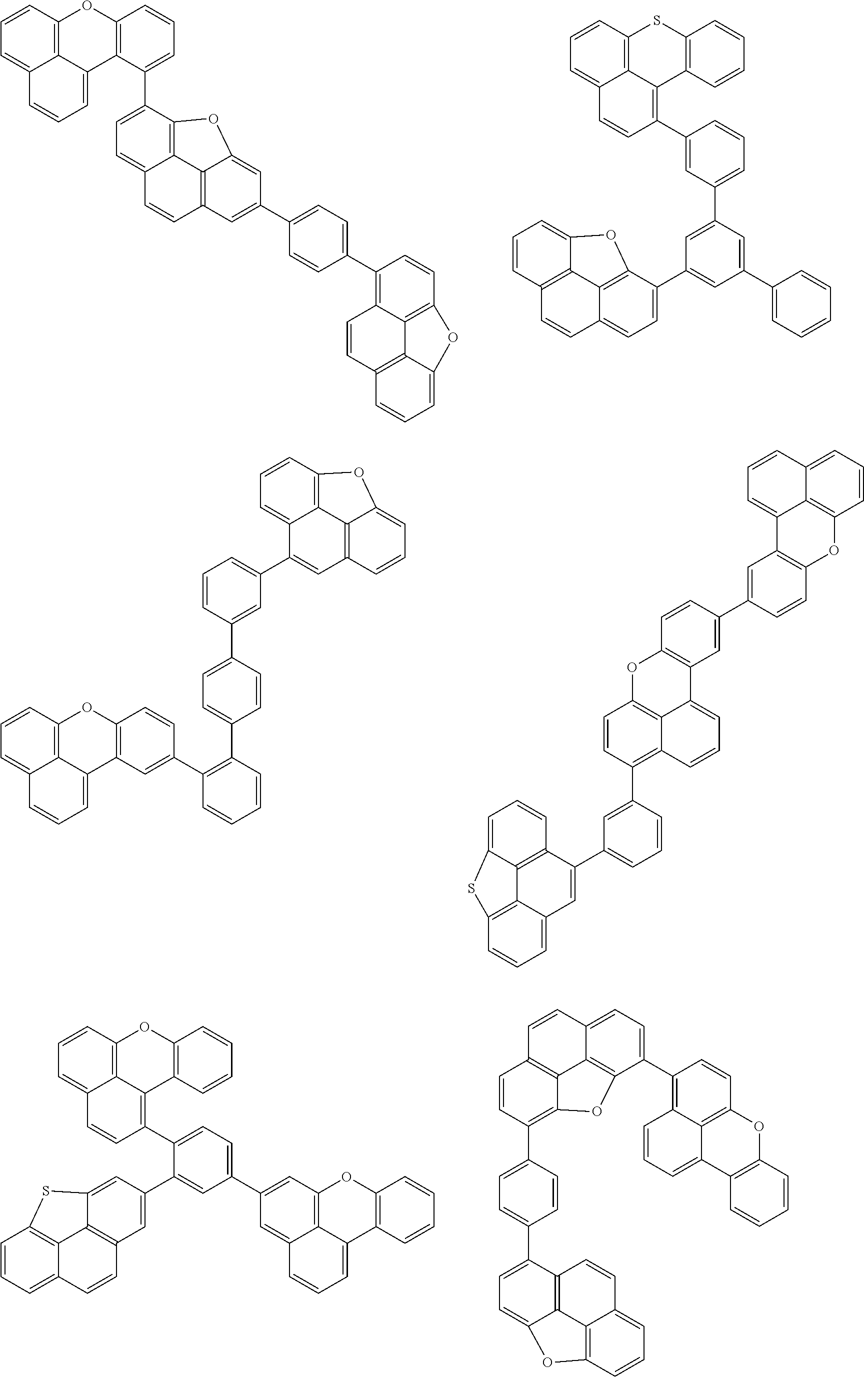

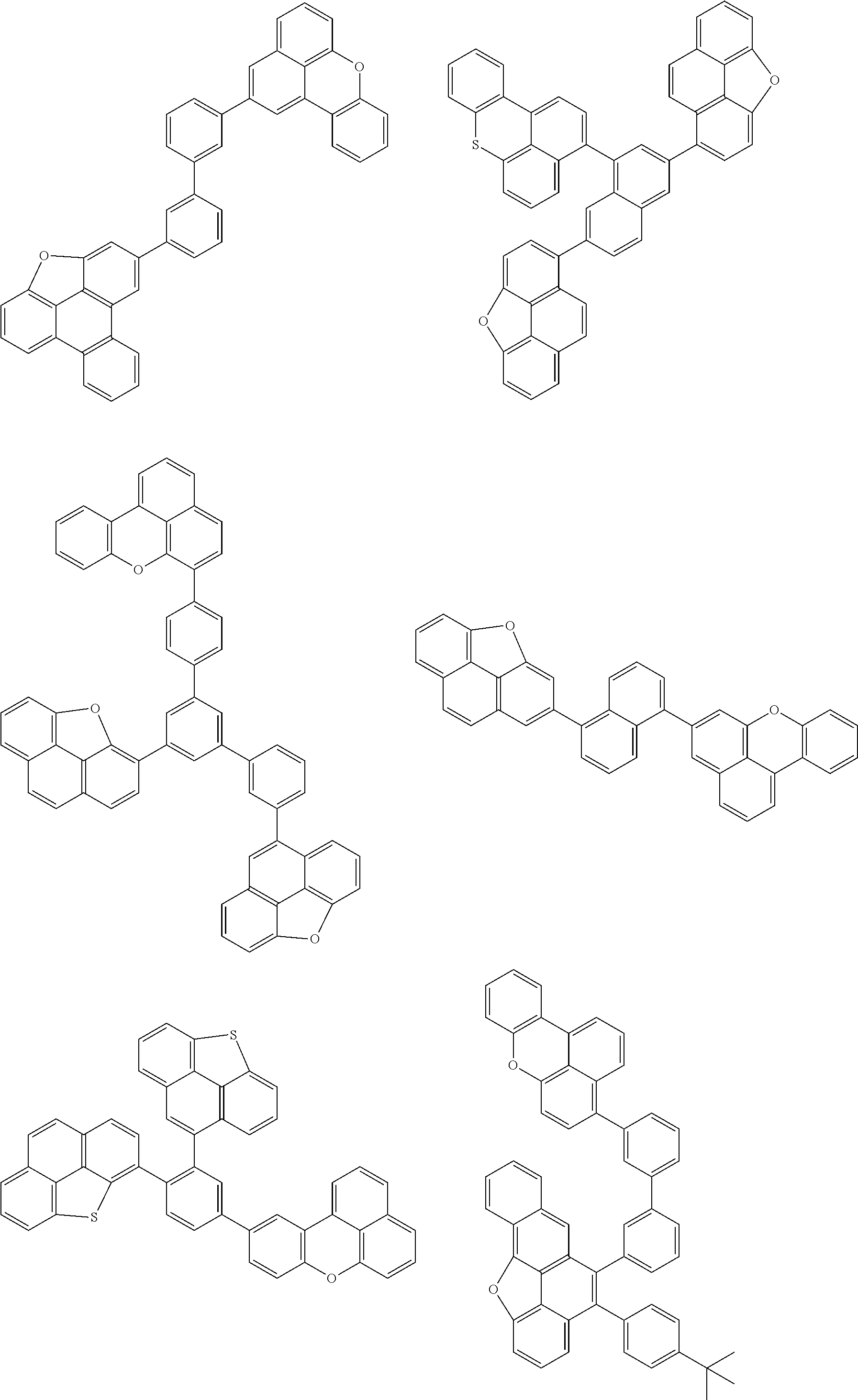

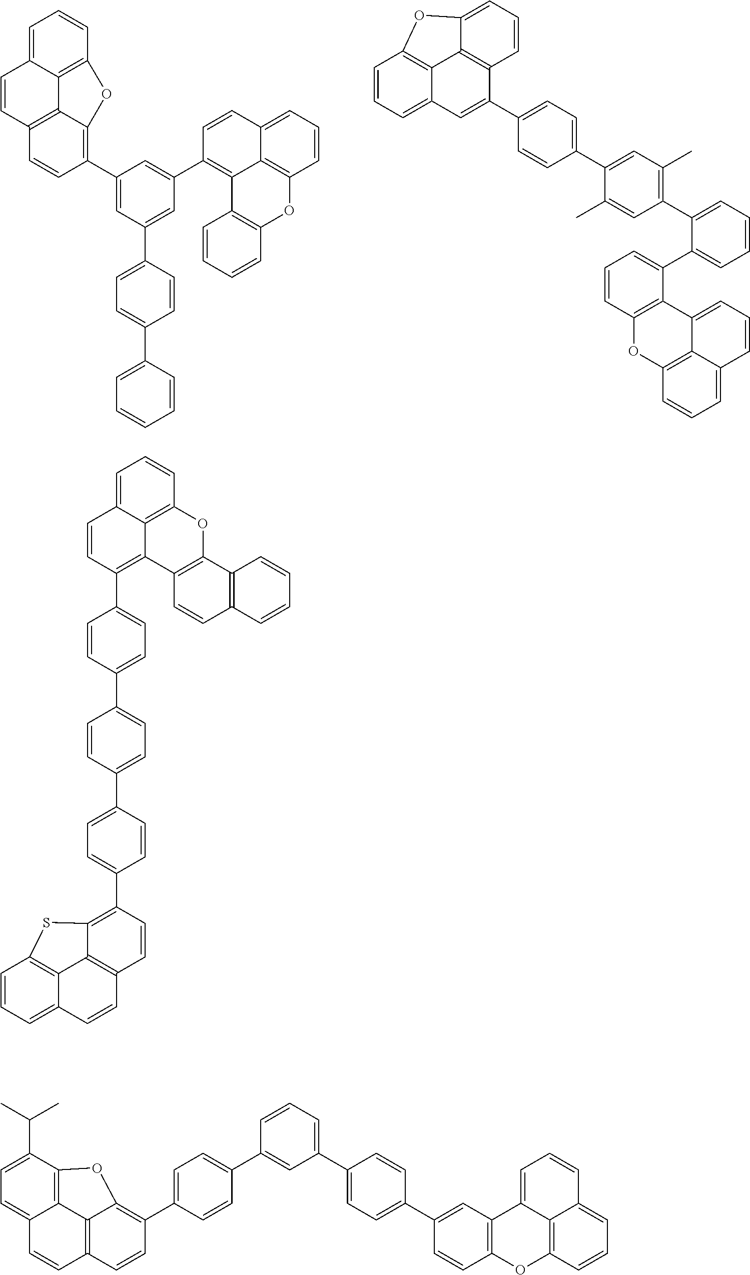

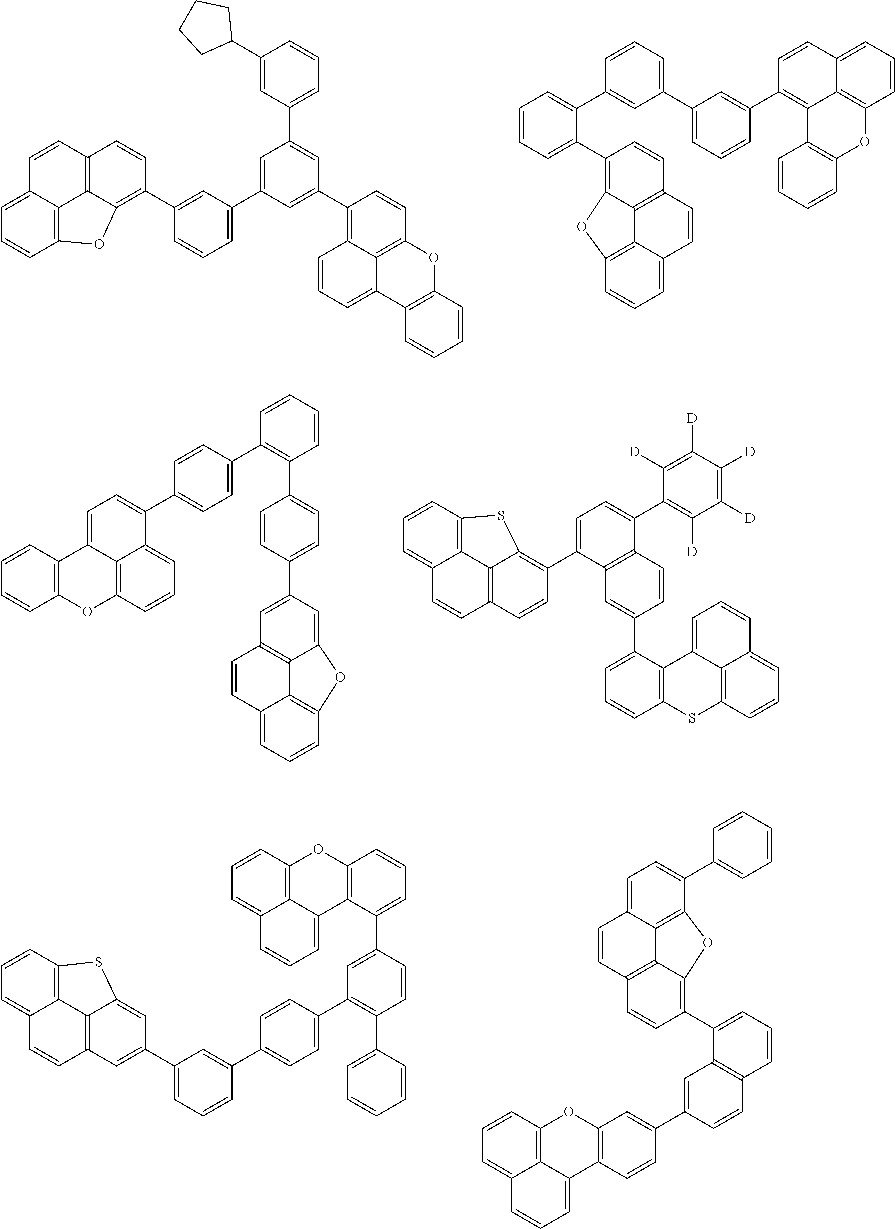

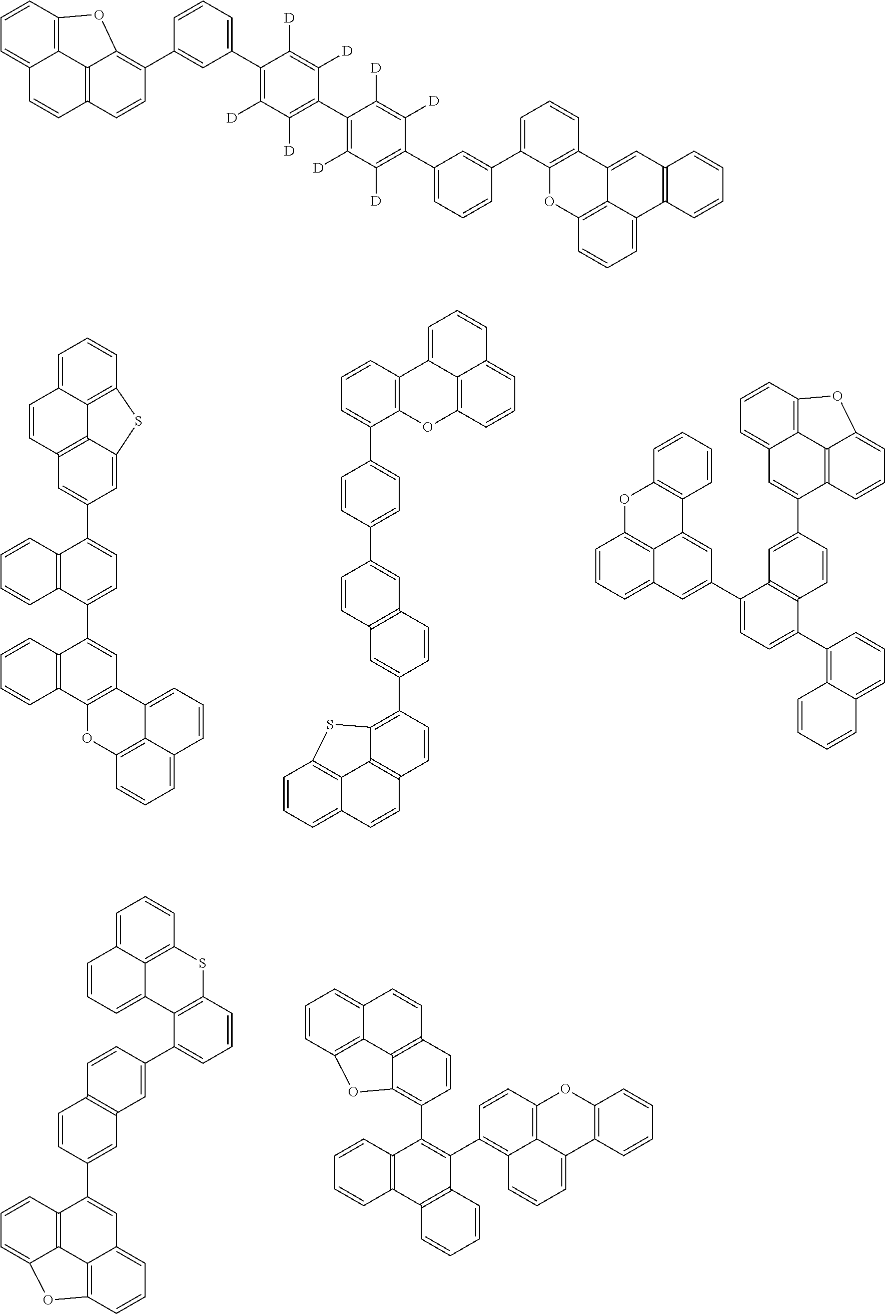

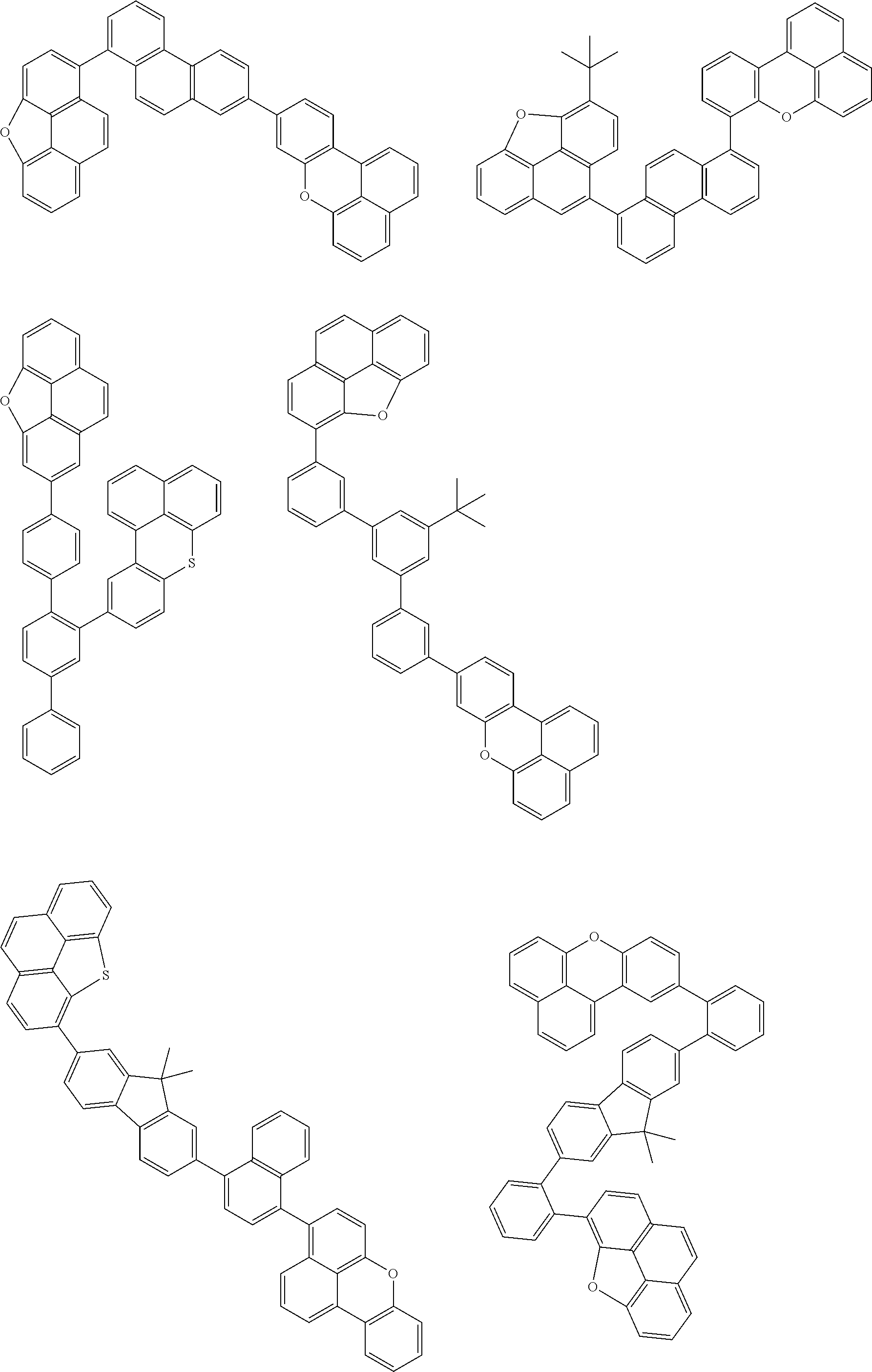

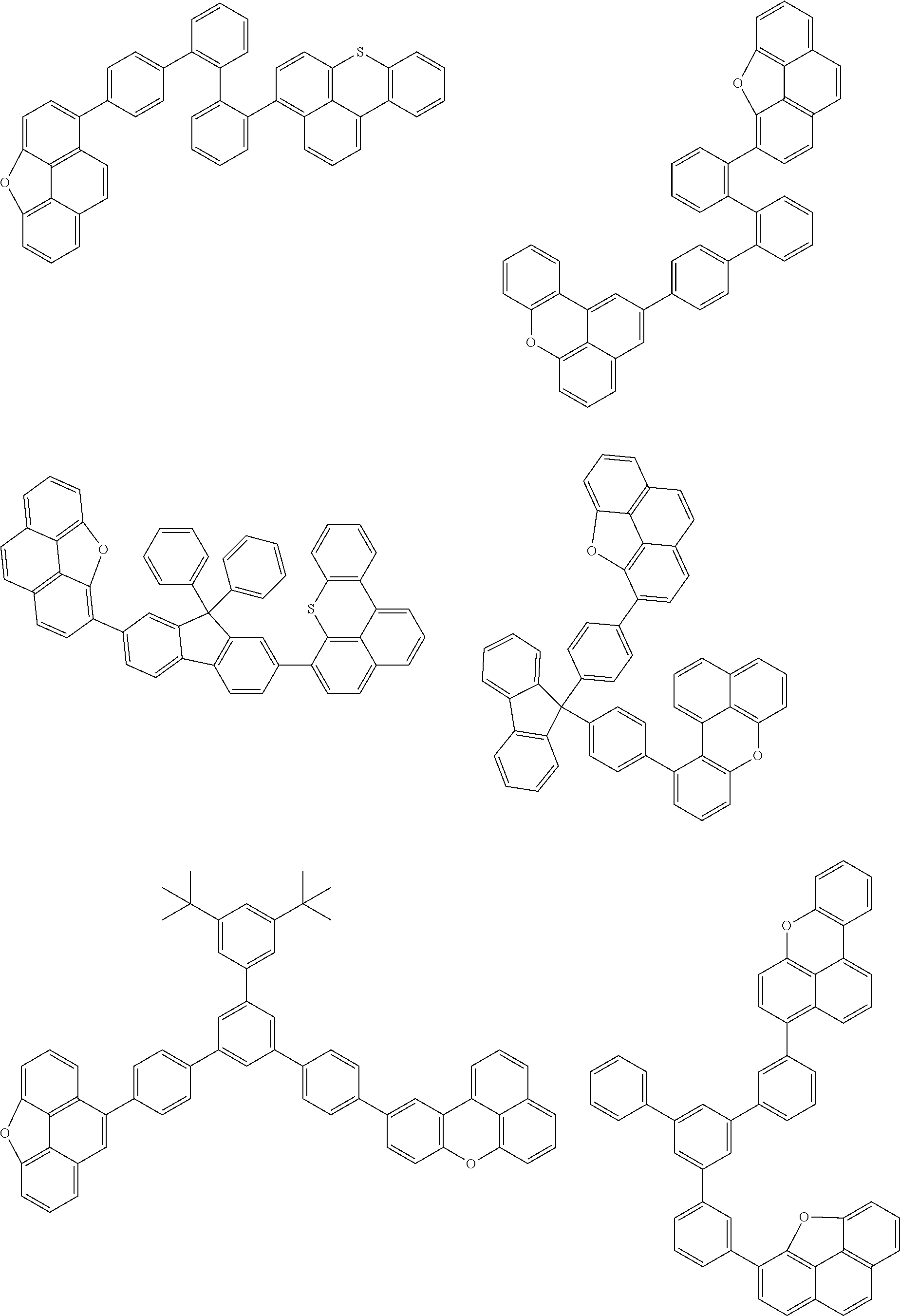

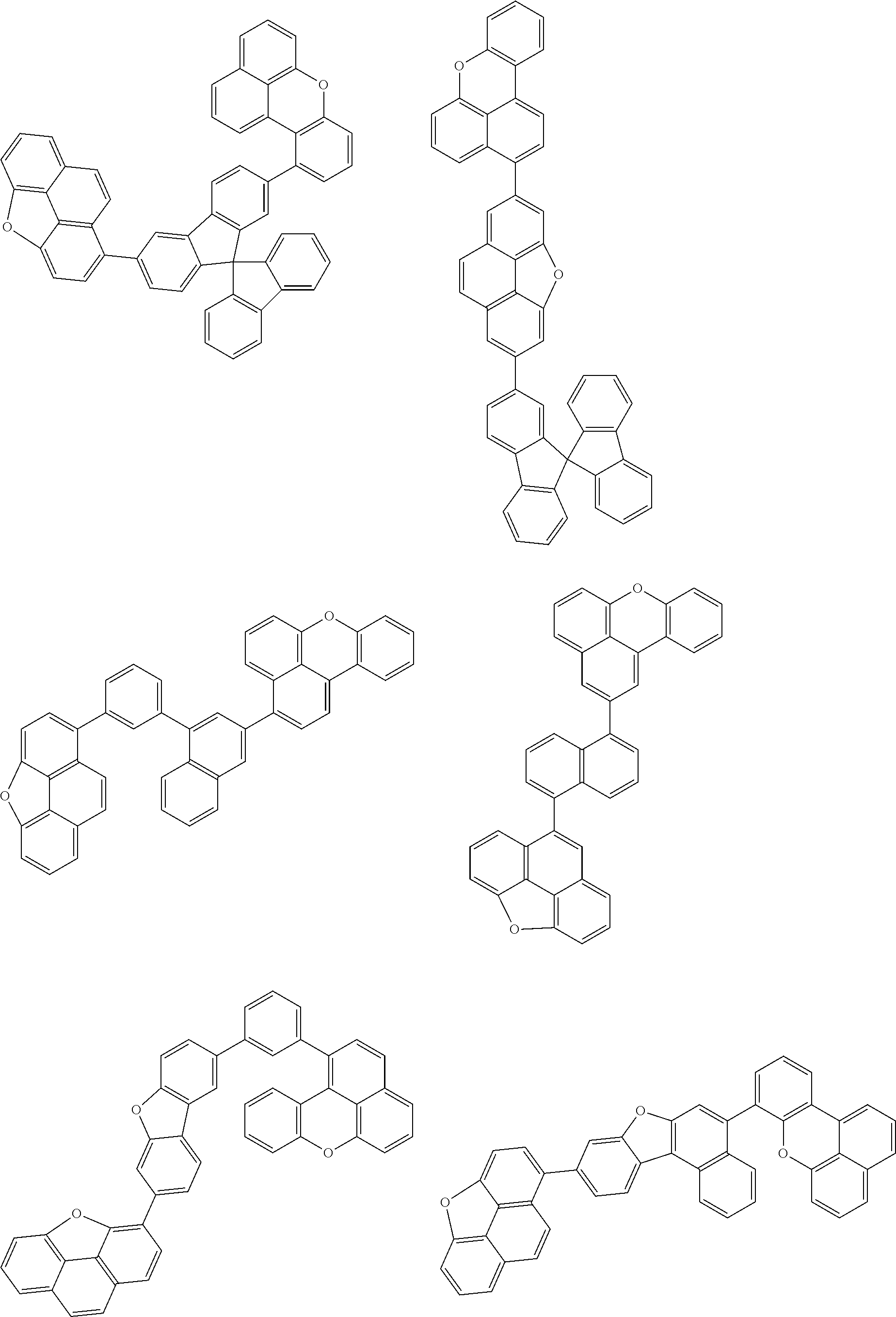

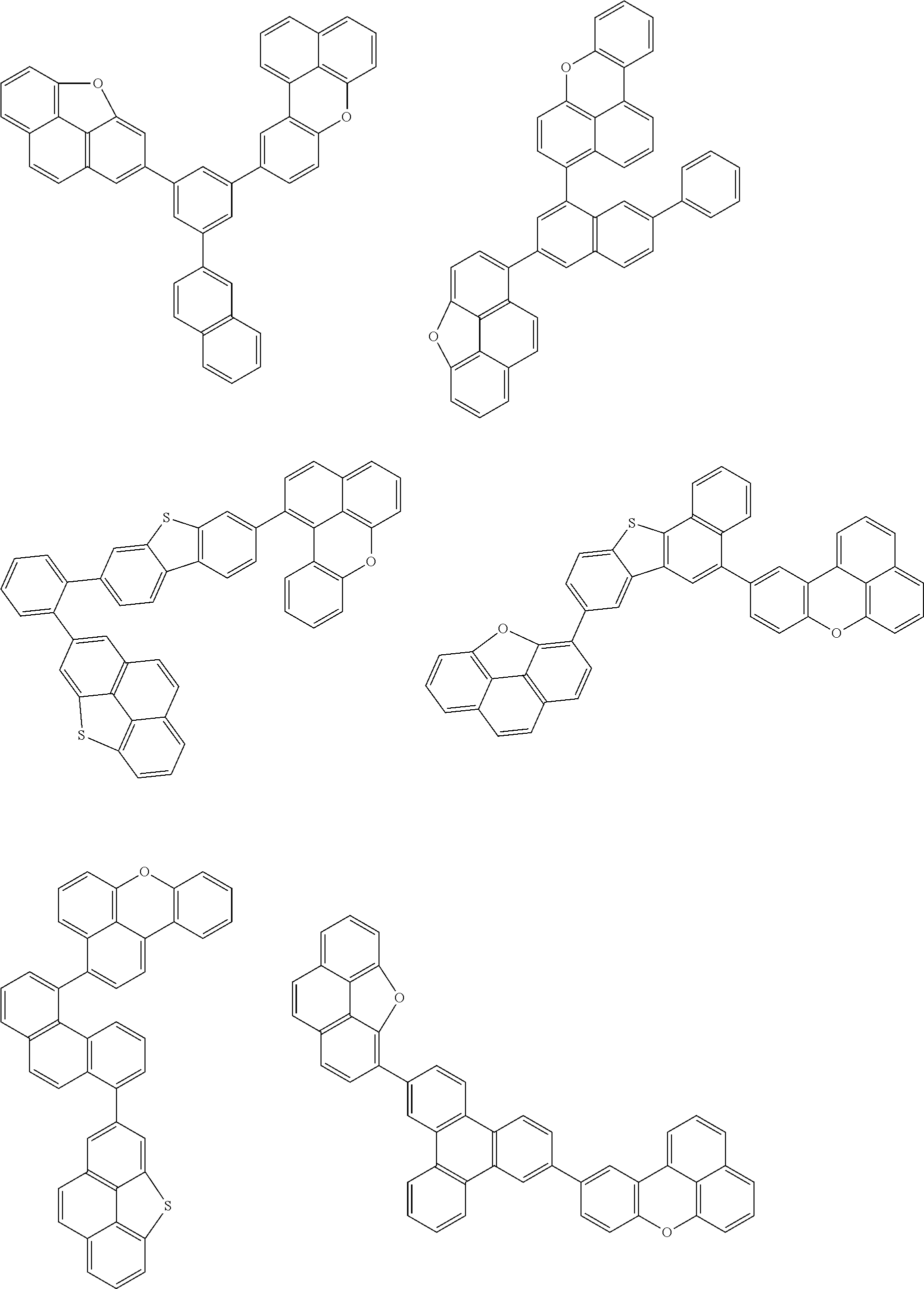

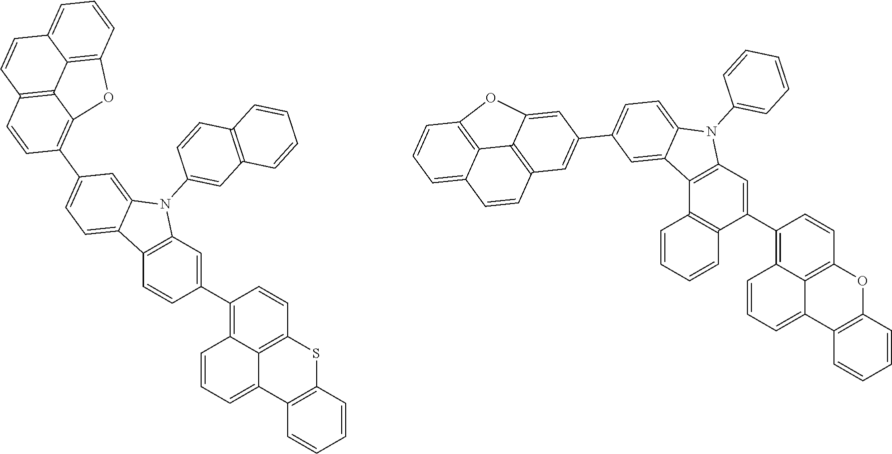 [SMILES: c1ccc2c(c1)Sc1ccc(-c3ccc4c5ccc(-c6ccc7ccc8cccc9oc6c7c89)cc5n(-c5ccc6ccccc6c5)c4c3)c3cccc-2c13 ; c1ccc(-n2c3ccc(-c4cc5ccc6cccc7oc(c4)c5c67)cc3c3c4ccccc4c(-c4ccc5c6c(cccc46)-c4ccccc4O5)cc32)cc1]

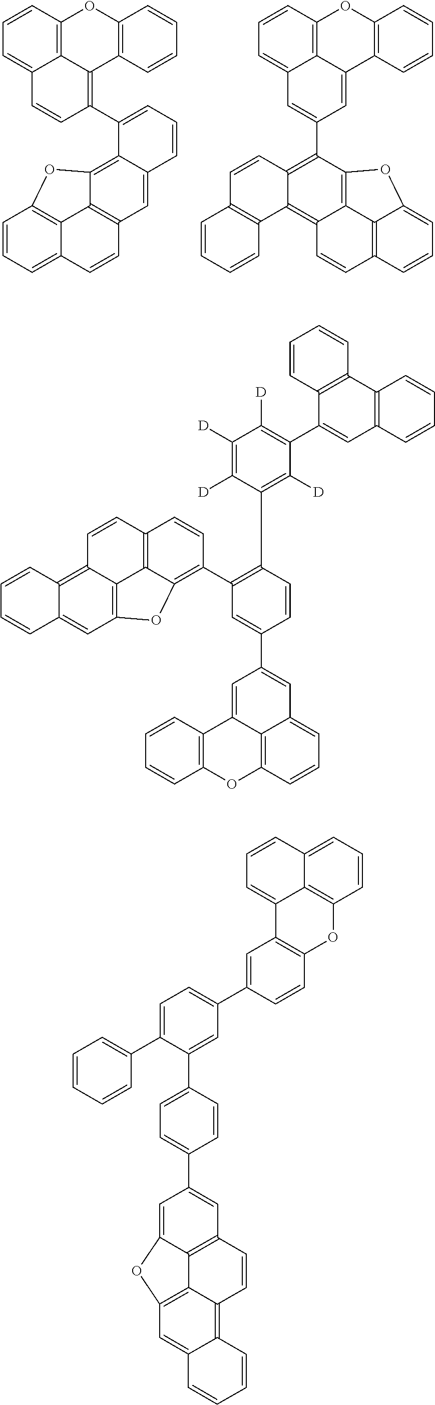 [SMILES: c1ccc2c(c1)Oc1cccc3ccc(-c4cccc5cc6ccc7cccc8oc(c45)c6c78)c-2c13 ; c1ccc2c(c1)Oc1cccc3cc(-c4c5ccc6ccccc6c5c5ccc6cccc7oc4c5c67)cc-2c13 ; [2H]c1c([2H])c(-c2ccc(-c3cc4c5c(cccc5c3)Oc3ccccc3-4)cc2-c2ccc3ccc4c5ccccc5cc5oc2c3c54)c([2H])c(-c2cc3ccccc3c3ccccc23)c1[2H] ; c1ccc(-c2ccc(-c3ccc4c(c3)-c3cccc5cccc(c35)O4)cc2-c2ccc(-c3cc4ccc5c6ccccc6cc6oc(c3)c4c65)cc2)cc1]

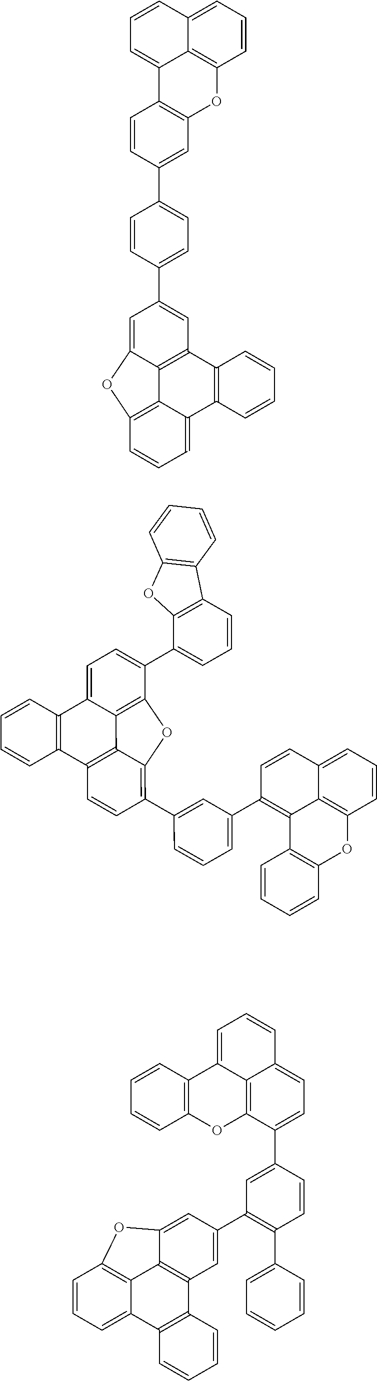

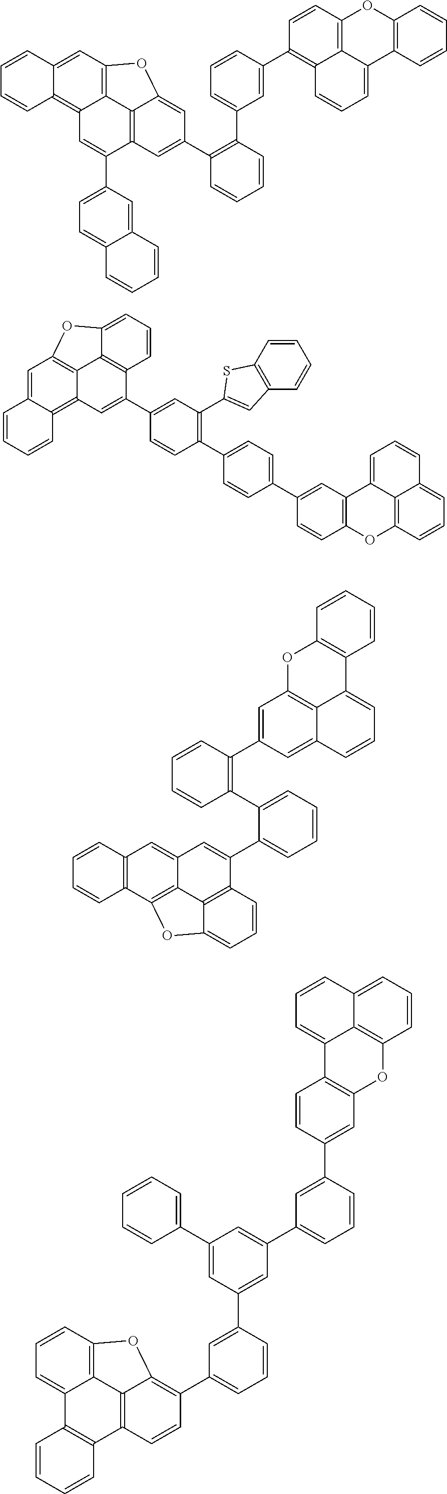

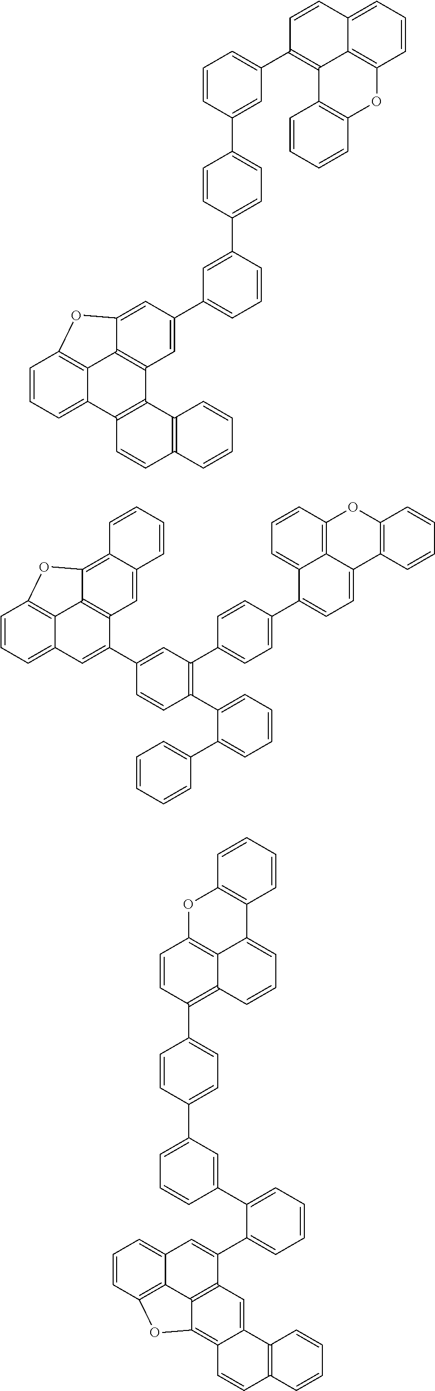

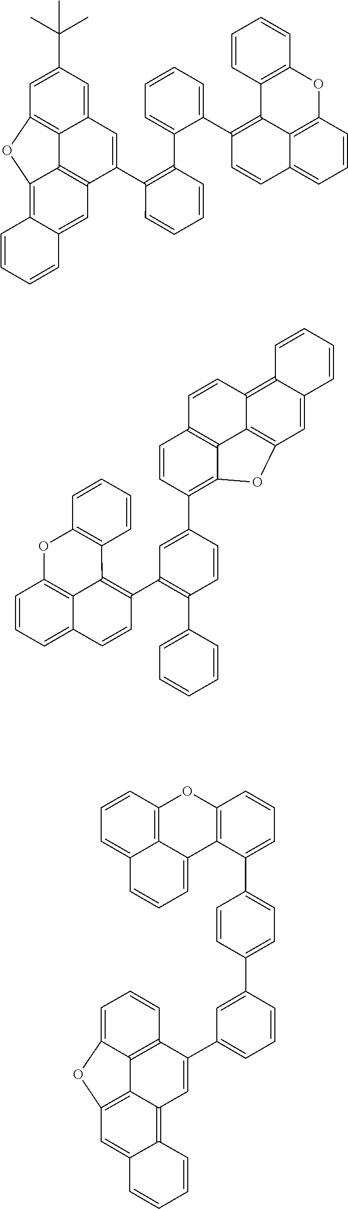 [SMILES: CC(C)(C)c1cc2cc(-c3ccccc3-c3ccccc3-c3ccc4cccc5c4c3-c3ccccc3O5)c3cc4ccccc4c4oc(c1)c2c34 ; c1ccc(-c2ccc(-c3ccc4ccc5c6ccccc6cc6oc3c4c65)cc2-c2ccc3cccc4c3c2-c2ccccc2O4)cc1 ; c1cc(-c2ccc(-c3cccc4c3-c3cccc5cccc(c35)O4)cc2)cc(-c2cc3c4ccccc4cc4oc5cccc2c5c43)c1]

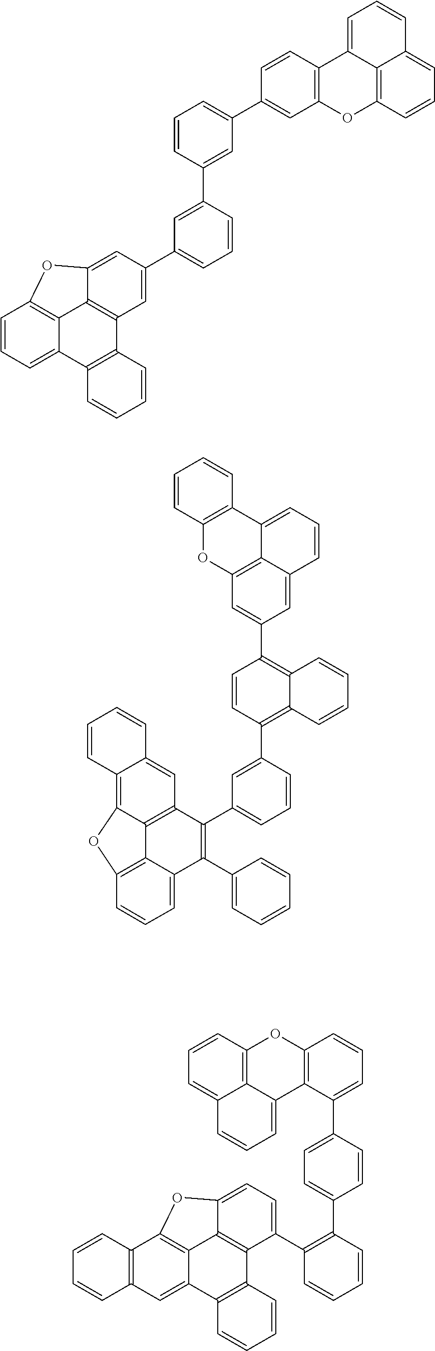 [SMILES: c1cc(-c2cccc(-c3cc4oc5cccc6c7ccccc7c(c3)c4c56)c2)cc(-c2ccc3c(c2)Oc2cccc4cccc-3c24)c1 ; c1ccc(-c2c(-c3cccc(-c4ccc(-c5cc6c7c(cccc7c5)-c5ccccc5O6)c5ccccc45)c3)c3cc4ccccc4c4oc5cccc2c5c34)cc1 ; c1ccc(-c2ccc3oc4c5ccccc5cc5c6ccccc6c2c3c54)c(-c2ccc(-c3cccc4c3-c3cccc5cccc(c35)O4)cc2)c1]

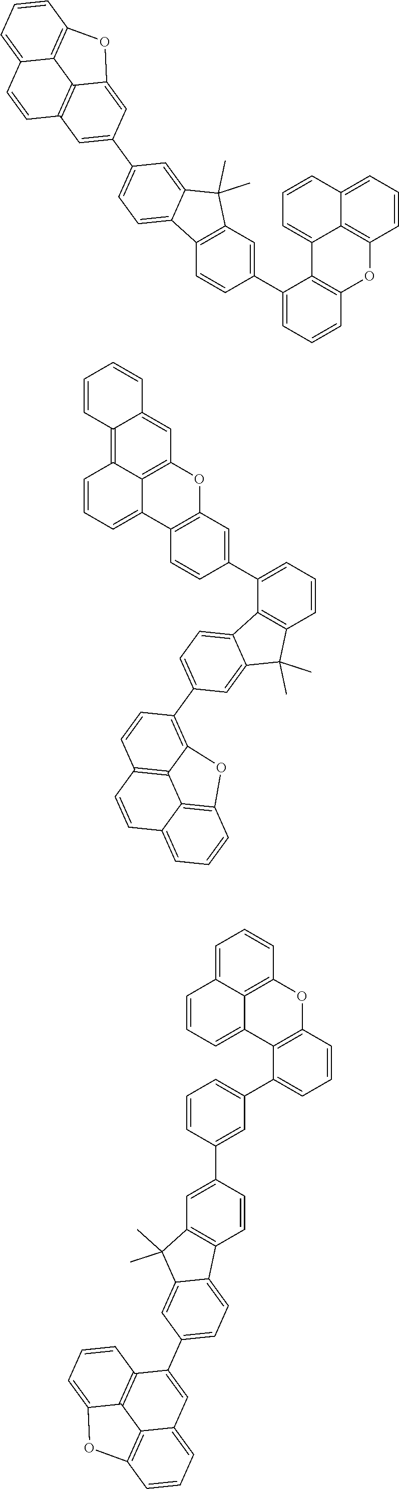 [SMILES: CC1(C)c2cc(-c3cc4ccc5cccc6oc(c3)c4c56)ccc2-c2ccc(-c3cccc4c3-c3cccc5cccc(c35)O4)cc21 ; CC1(C)c2cc(-c3ccc4ccc5cccc6oc3c4c56)ccc2-c2c(-c3ccc4c(c3)Oc3cc5ccccc5c5cccc-4c35)cccc21 ; CC1(C)c2cc(-c3cccc(-c4cccc5c4-c4cccc6cccc(c46)O5)c3)ccc2-c2ccc(-c3cc4cccc5oc6cccc3c6c45)cc21]

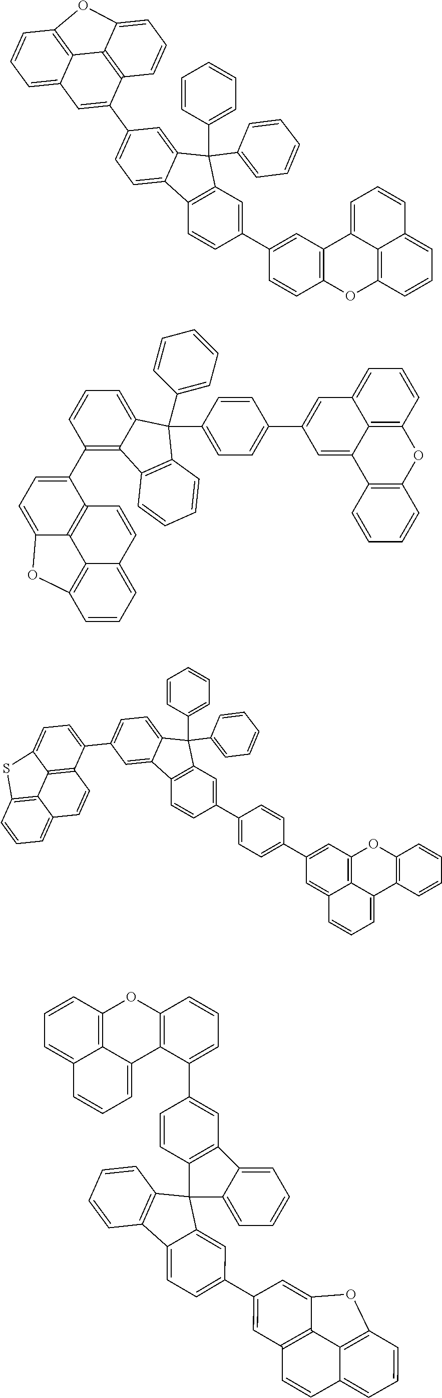 [SMILES: c1ccc(C2(c3ccccc3)c3cc(-c4ccc5c(c4)-c4cccc6cccc(c46)O5)ccc3-c3ccc(-c4cc5cccc6oc7cccc4c7c56)cc32)cc1 ; c1ccc(C2(c3ccc(-c4cc5c6c(cccc6c4)Oc4ccccc4-5)cc3)c3ccccc3-c3c(-c4ccc5oc6cccc7ccc4c5c76)cccc32)cc1 ; c1ccc(C2(c3ccccc3)c3ccc(-c4ccc5sc6cccc7ccc4c5c76)cc3-c3ccc(-c4ccc(-c5cc6c7c(cccc7c5)-c5ccccc5O6)cc4)cc32)cc1 ; c1ccc2c(c1)-c1cc(-c3cccc4c3-c3cccc5cccc(c35)O4)ccc1C21c2ccccc2-c2ccc(-c3cc4ccc5cccc6oc(c3)c4c56)cc21]

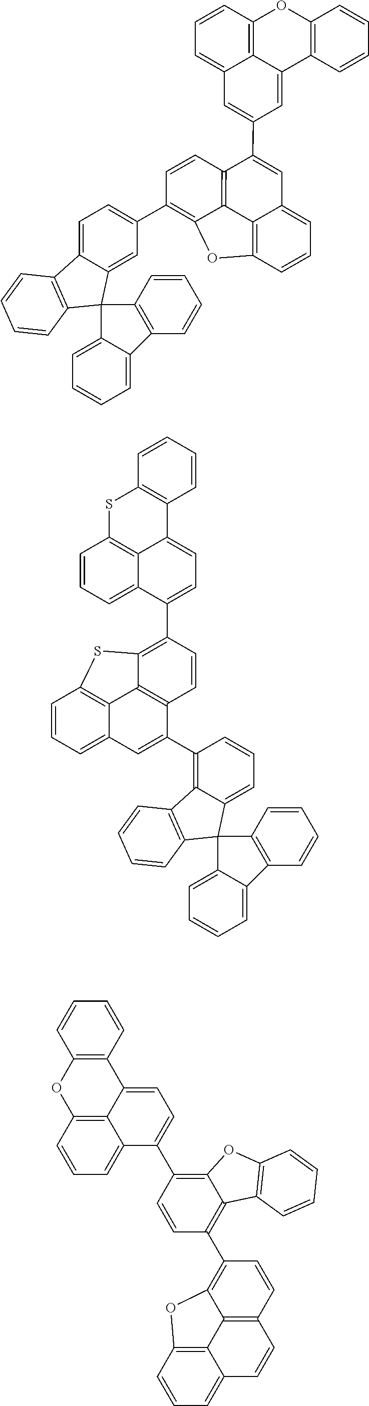 [SMILES: c1ccc2c(c1)Oc1cccc3cc(-c4cc5cccc6oc7c(-c8ccc9c(c8)C8(c%10ccccc%10-c%10ccccc%108)c8ccccc8-9)ccc4c7c56)cc-2c13 ; c1ccc2c(c1)Sc1cccc3c(-c4ccc5c(-c6cccc7c6-c6ccccc6C76c7ccccc7-c7ccccc76)cc6cccc7sc4c5c67)ccc-2c13 ; c1ccc2c(c1)Oc1cccc3c(-c4ccc(-c5ccc6ccc7cccc8oc5c6c78)c5c4oc4ccccc45)ccc-2c13]

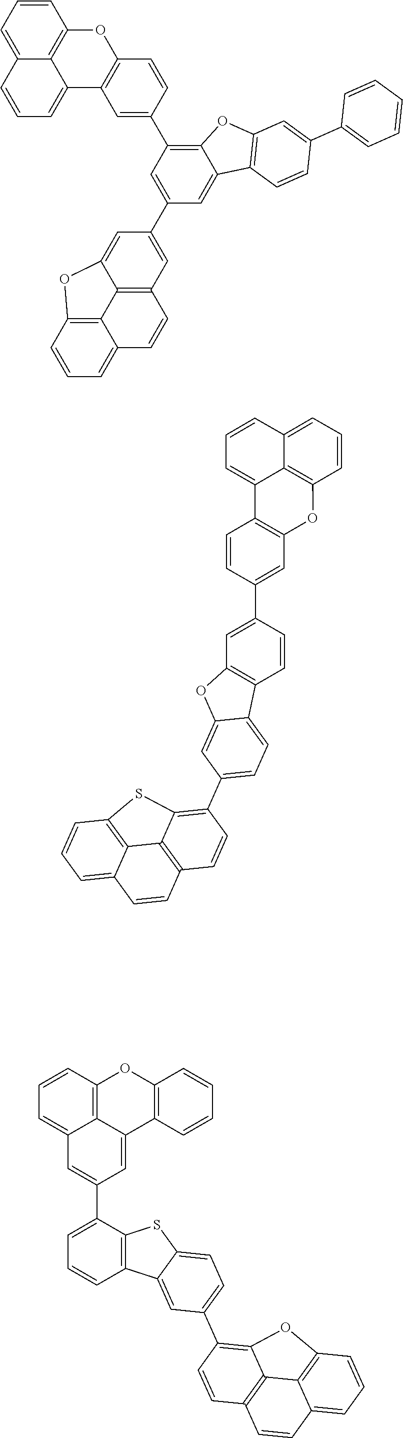 [SMILES: c1ccc(-c2ccc3c(c2)oc2c(-c4ccc5c(c4)-c4cccc6cccc(c46)O5)cc(-c4cc5ccc6cccc7oc(c4)c5c67)cc23)cc1 ; c1cc2c3c(cccc3c1)-c1ccc(-c3ccc4c(c3)oc3cc(-c5ccc6ccc7cccc8sc5c6c78)ccc34)cc1O2 ; c1ccc2c(c1)Oc1cccc3cc(-c4cccc5c4sc4ccc(-c6ccc7ccc8cccc9oc6c7c89)cc45)cc-2c13]

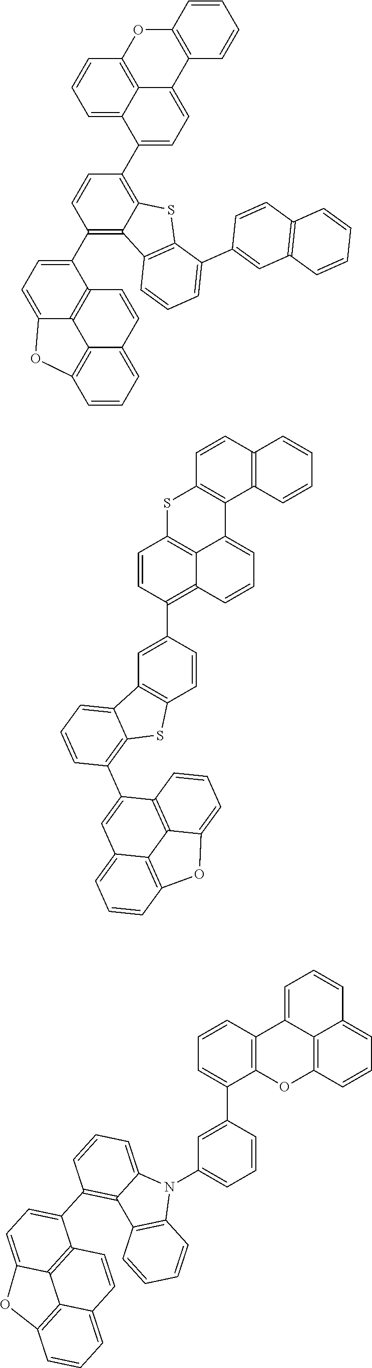 [SMILES: c1ccc2c(c1)Oc1cccc3c(-c4ccc(-c5ccc6oc7cccc8ccc5c6c87)c5c4sc4c(-c6ccc7ccccc7c6)cccc45)ccc-2c13 ; c1ccc2c3c(ccc2c1)Sc1ccc(-c2ccc4sc5c(-c6cc7cccc8oc9cccc6c9c78)cccc5c4c2)c2cccc-3c12 ; c1cc(-c2cccc3c2Oc2cccc4cccc-3c24)cc(-n2c3ccccc3c3c(-c4ccc5oc6cccc7ccc4c5c76)cccc32)c1]

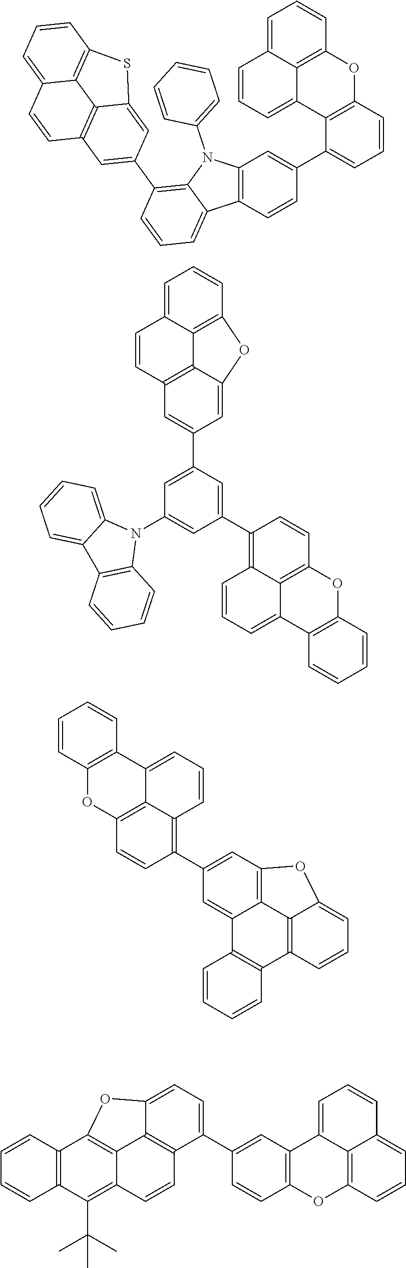

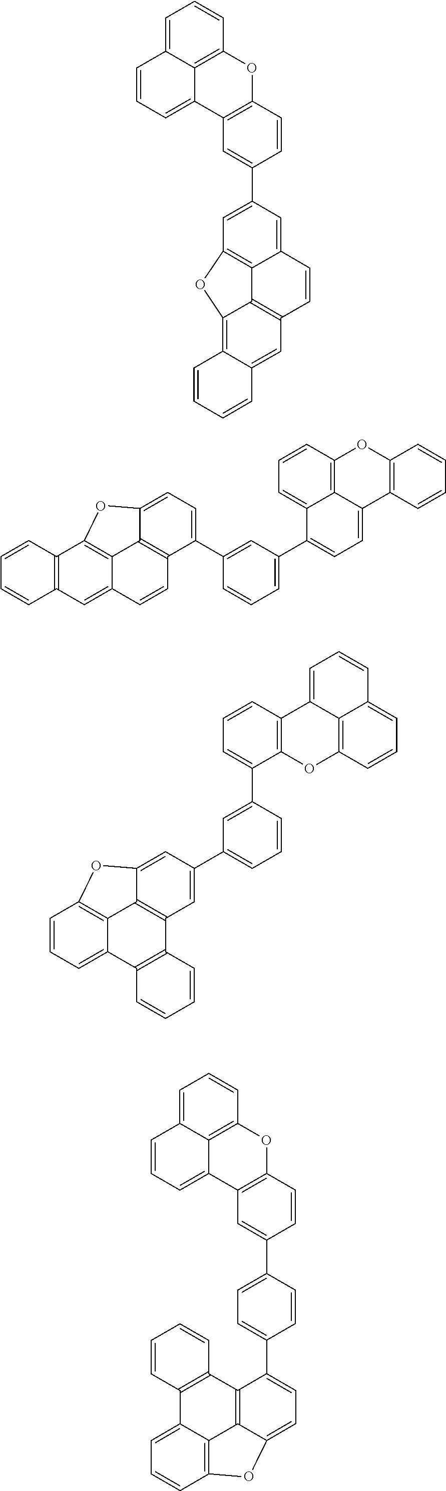 [SMILES: c1cc2c3c(cccc3c1)-c1cc(-c3cc4ccc5cc6ccccc6c6oc(c3)c4c56)ccc1O2 ; c1cc(-c2ccc3c4c(cccc24)Oc2ccccc2-3)cc(-c2ccc3oc4c5ccccc5cc5ccc2c3c54)c1 ; c1cc(-c2cc3oc4cccc5c6ccccc6c(c2)c3c45)cc(-c2cccc3c2Oc2cccc4cccc-3c24)c1 ; c1cc2c3c(cccc3c1)-c1cc(-c3ccc(-c4ccc5oc6cccc7c8ccccc8c4c5c67)cc3)ccc1O2]

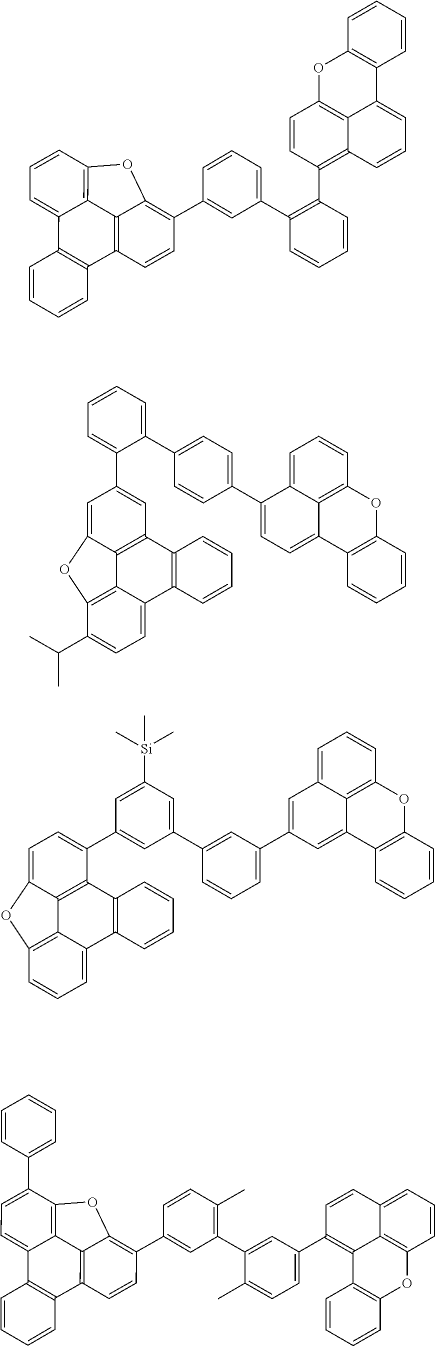 [SMILES: c1cc(-c2ccccc2-c2ccc3c4c(cccc24)-c2ccccc2O3)cc(-c2ccc3c4ccccc4c4cccc5oc2c3c54)c1 ; CC(C)c1ccc2c3ccccc3c3cc(-c4ccccc4-c4ccc(-c5ccc6c7c(cccc57)Oc5ccccc5-6)cc4)cc4oc1c2c43 ; C[Si](C)(C)c1cc(-c2cccc(-c3cc4c5c(cccc5c3)Oc3ccccc3-4)c2)cc(-c2ccc3oc4cccc5c6ccccc6c2c3c45)c1 ; Cc1ccc(-c2ccc3cccc4c3c2-c2ccccc2O4)cc1-c1cc(-c2ccc3c4ccccc4c4ccc(-c5ccccc5)c5oc2c3c54)ccc1C]

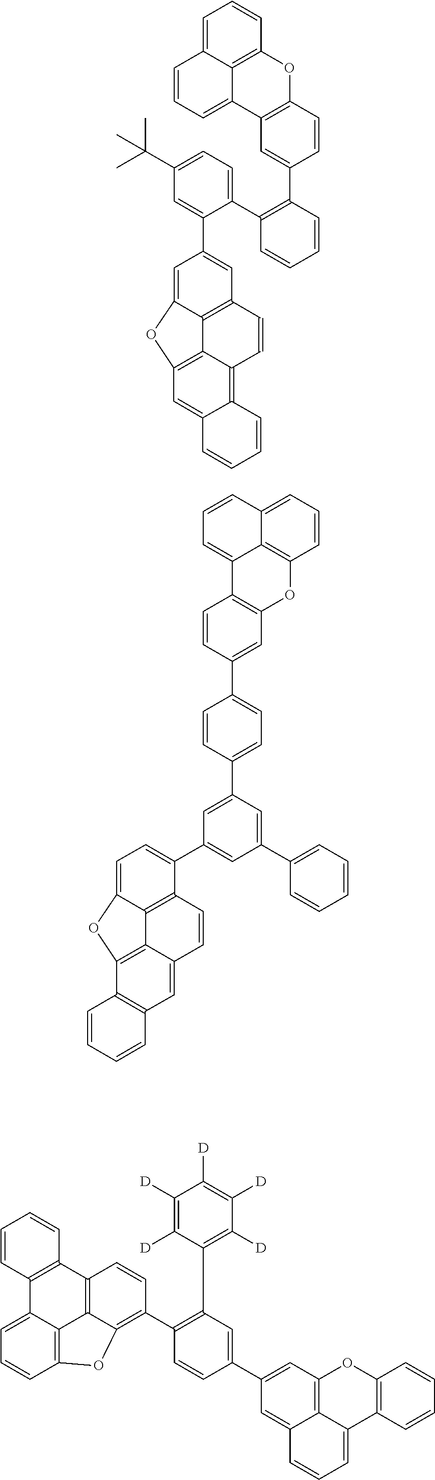

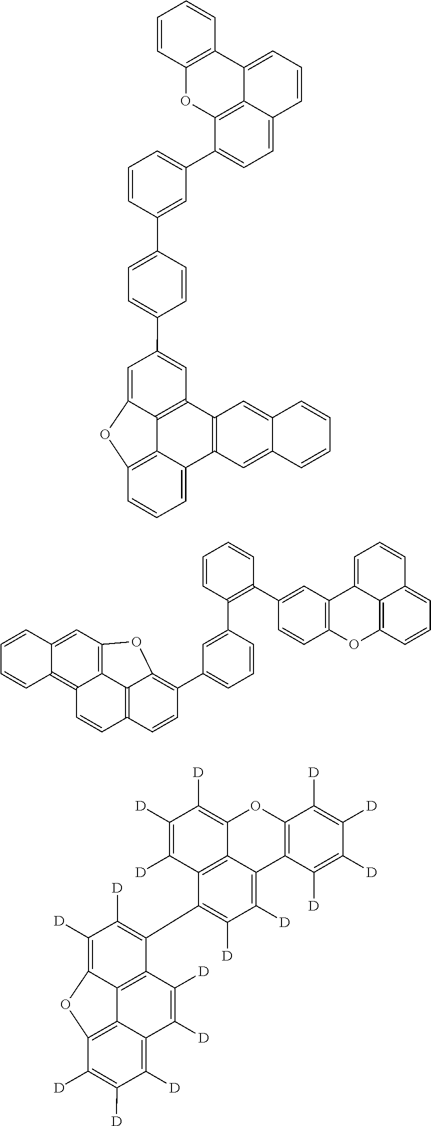 [SMILES: c1cc(-c2ccc(-c3cc4oc5cccc6c7cc8ccccc8cc7c(c3)c4c56)cc2)cc(-c2ccc3cccc4c3c2Oc2ccccc2-4)c1 ; c1cc(-c2ccccc2-c2ccc3c(c2)-c2cccc4cccc(c24)O3)cc(-c2ccc3ccc4c5ccccc5cc5oc2c3c54)c1 ; [2H]c1c([2H])c([2H])c2c(c1[2H])Oc1c([2H])c([2H])c([2H])c3c(-c4c([2H])c([2H])c5oc6c([2H])c([2H])c([2H])c7c([2H])c([2H])c4c5c67)c([2H])c([2H])c-2c13]

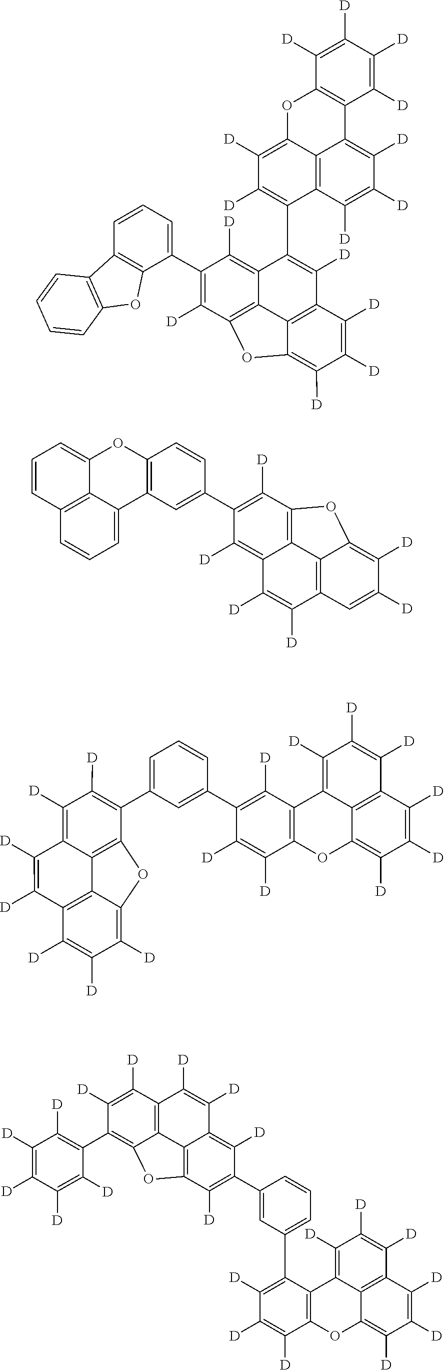

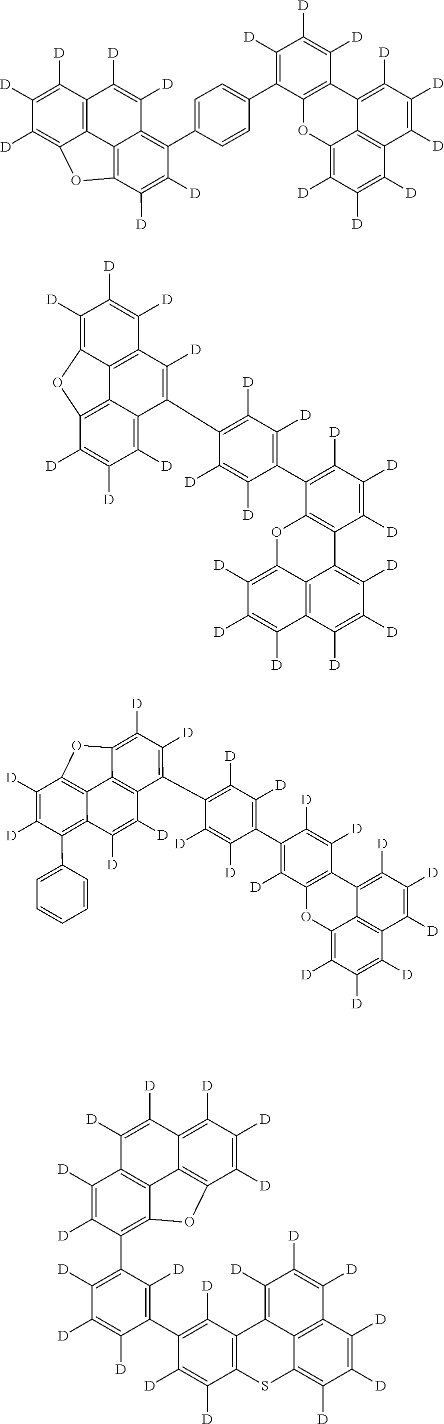

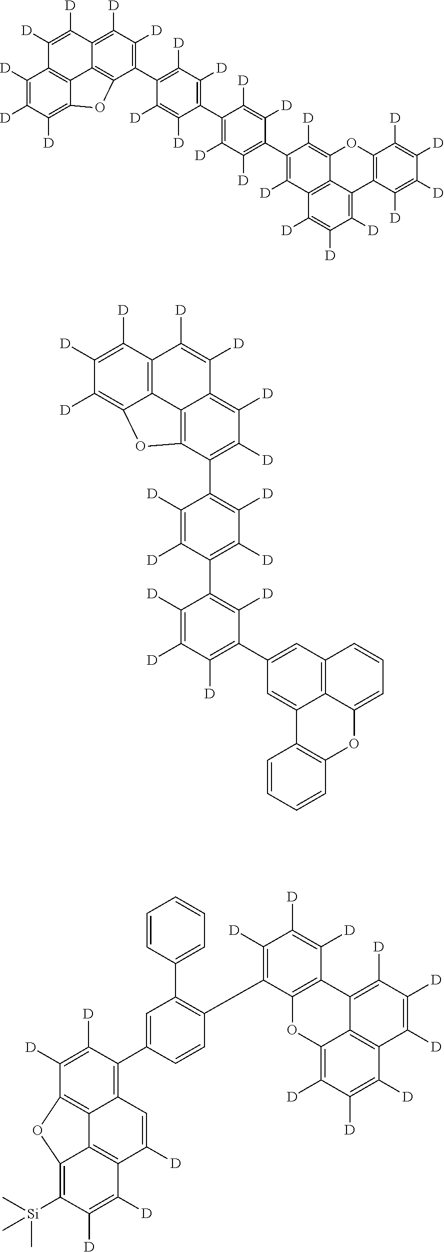

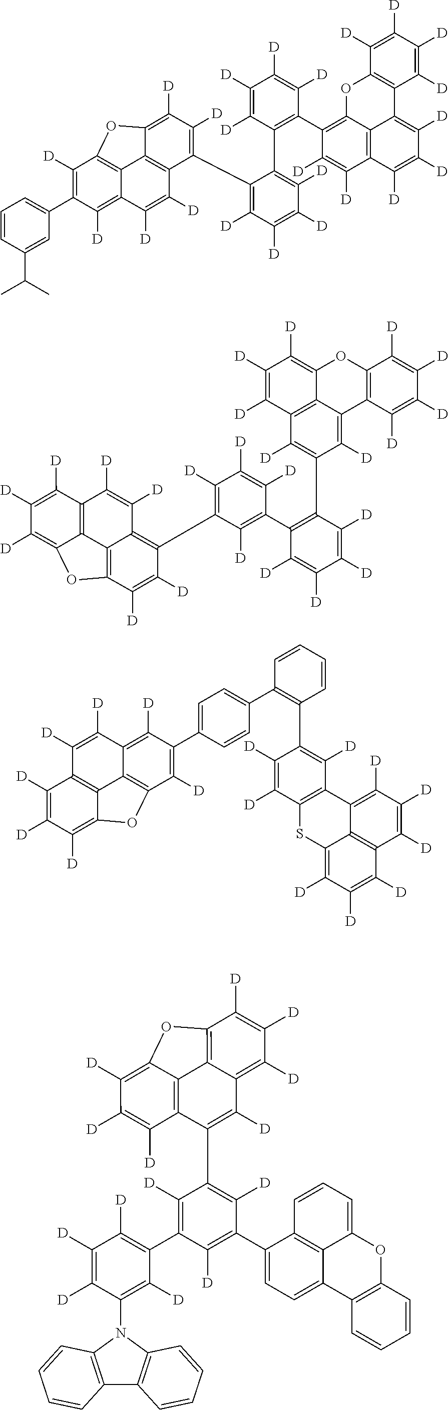

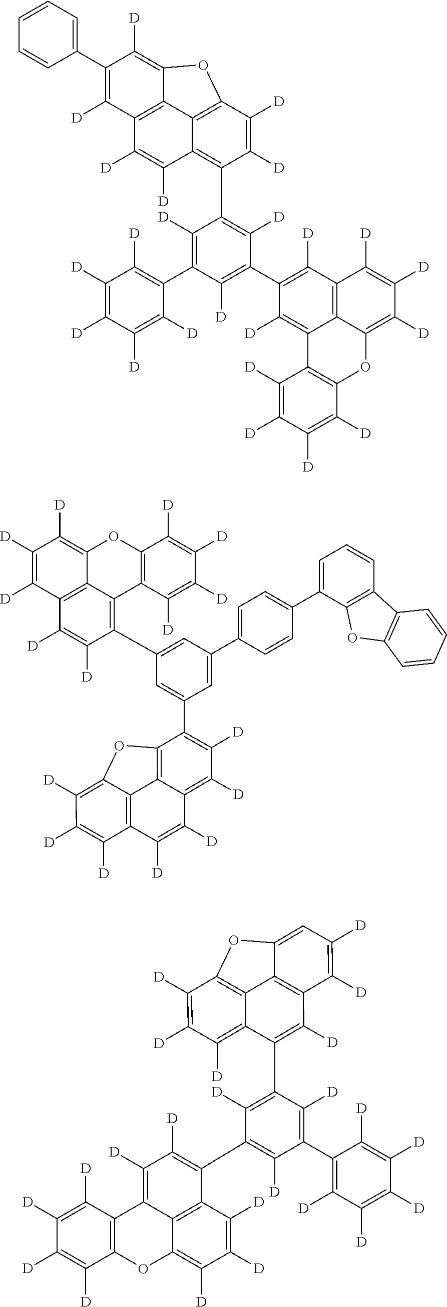

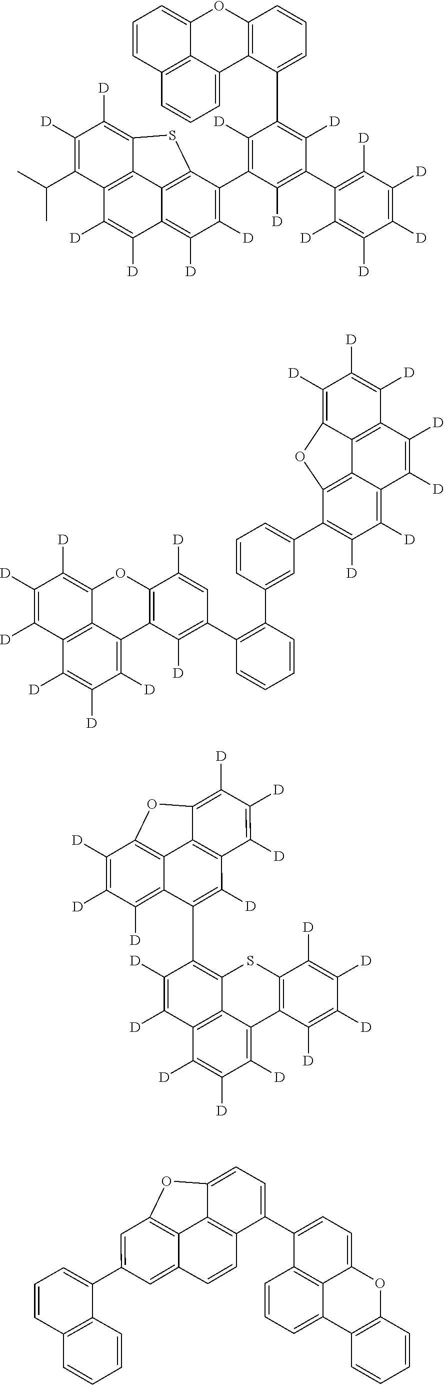

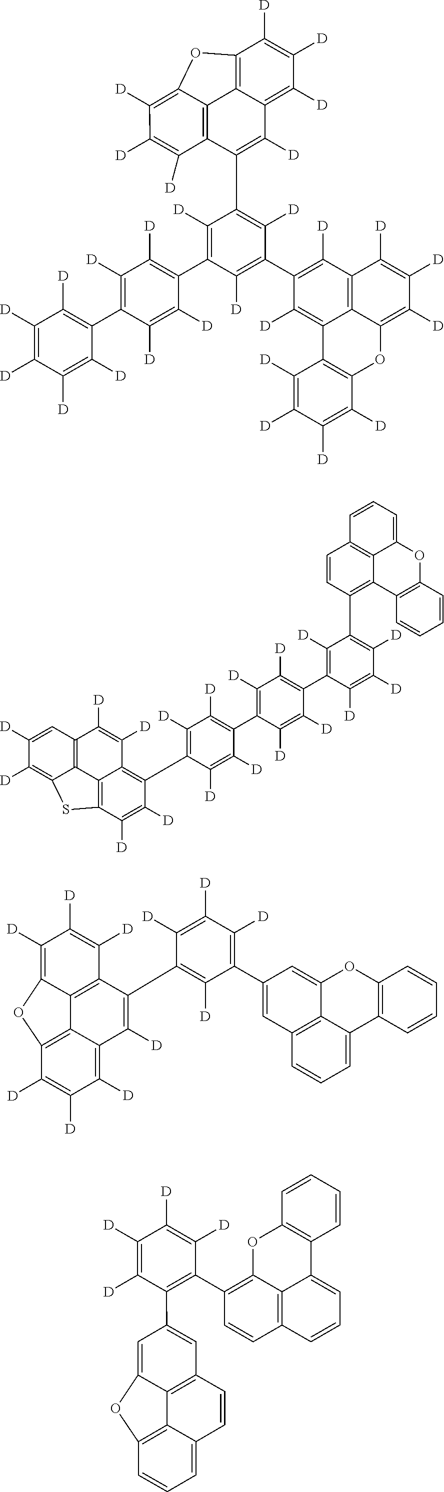

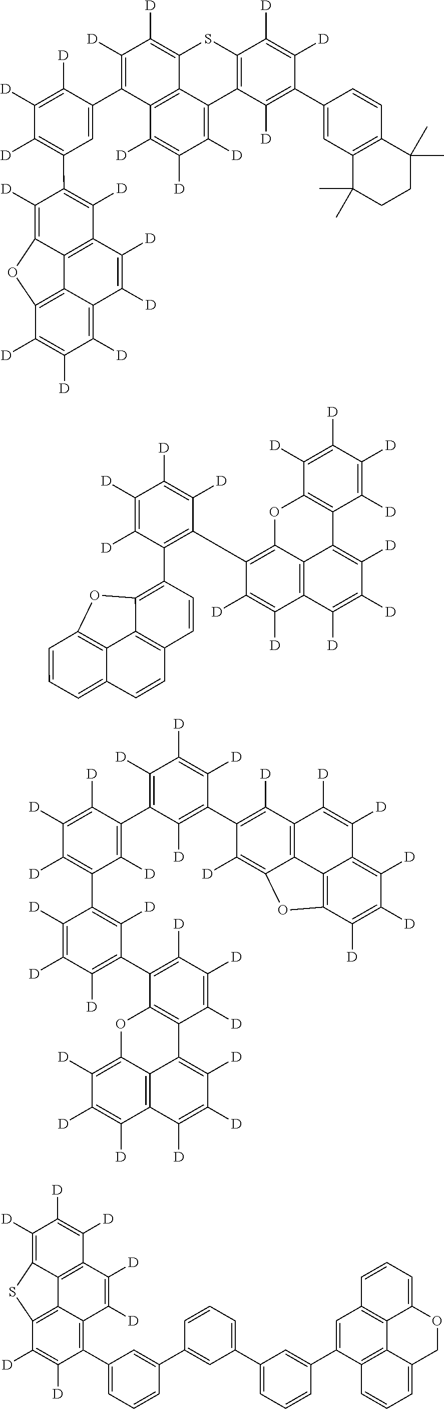

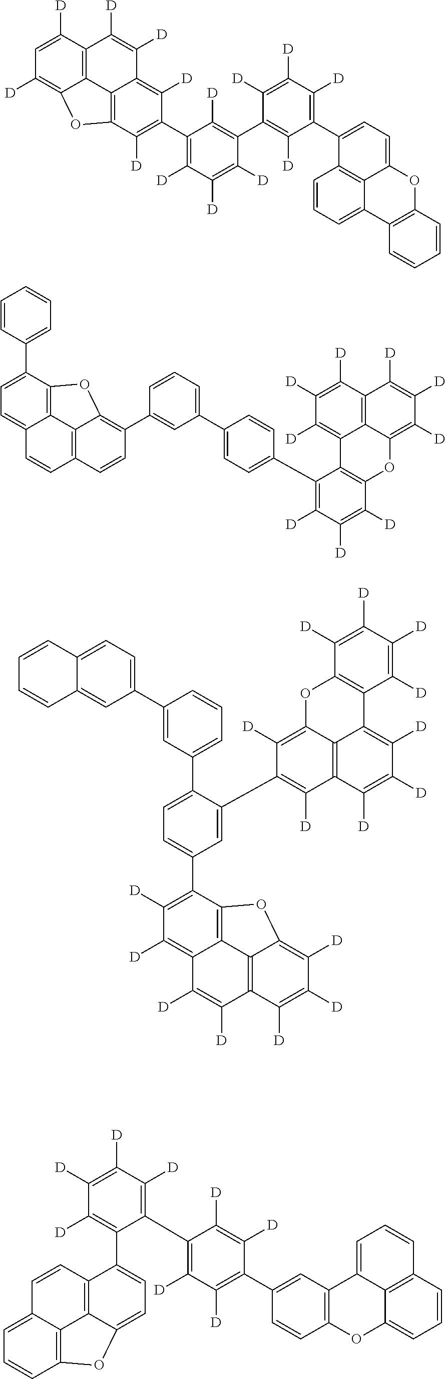

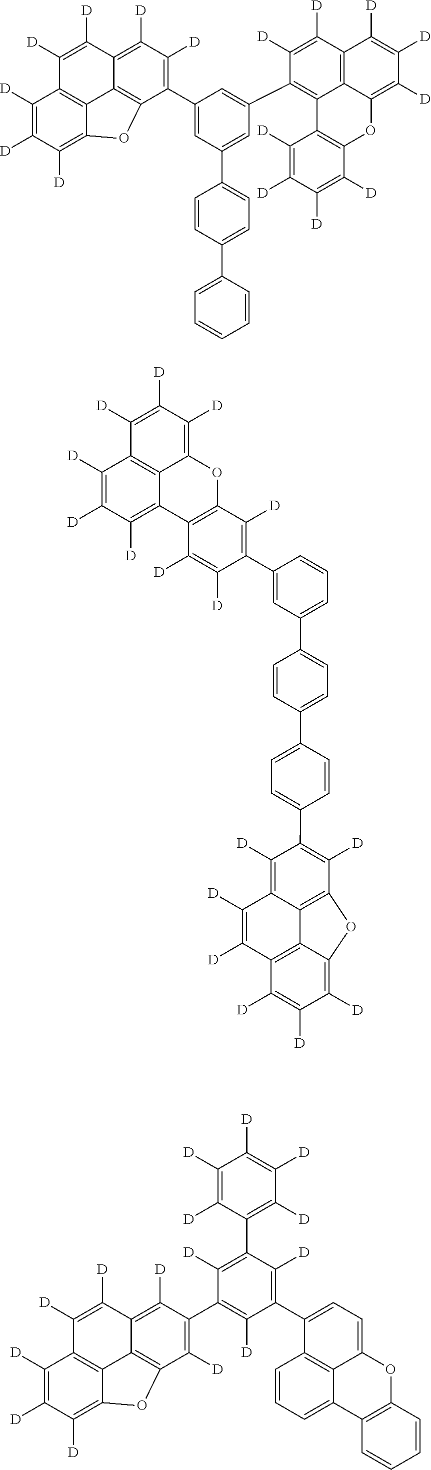

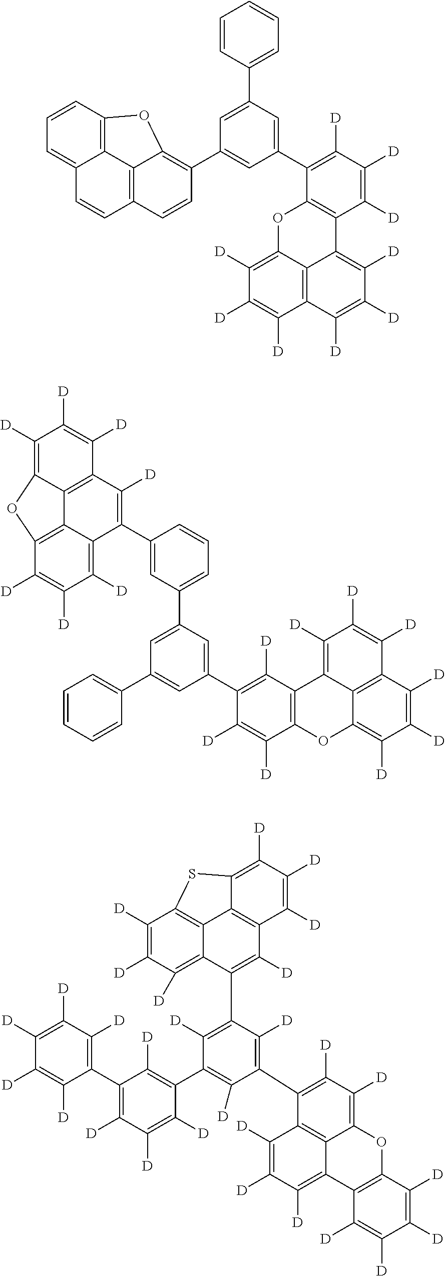

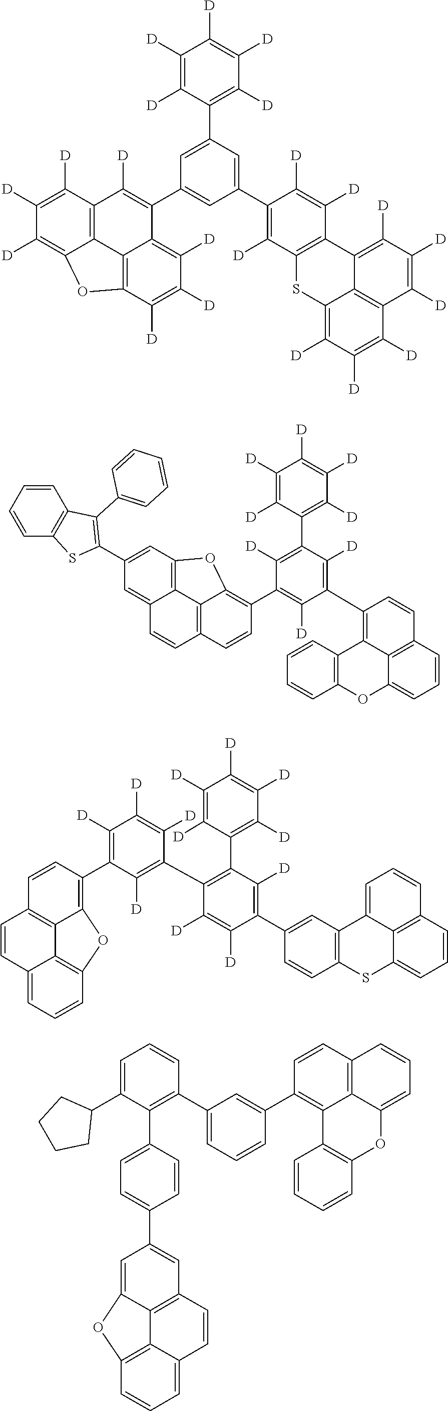

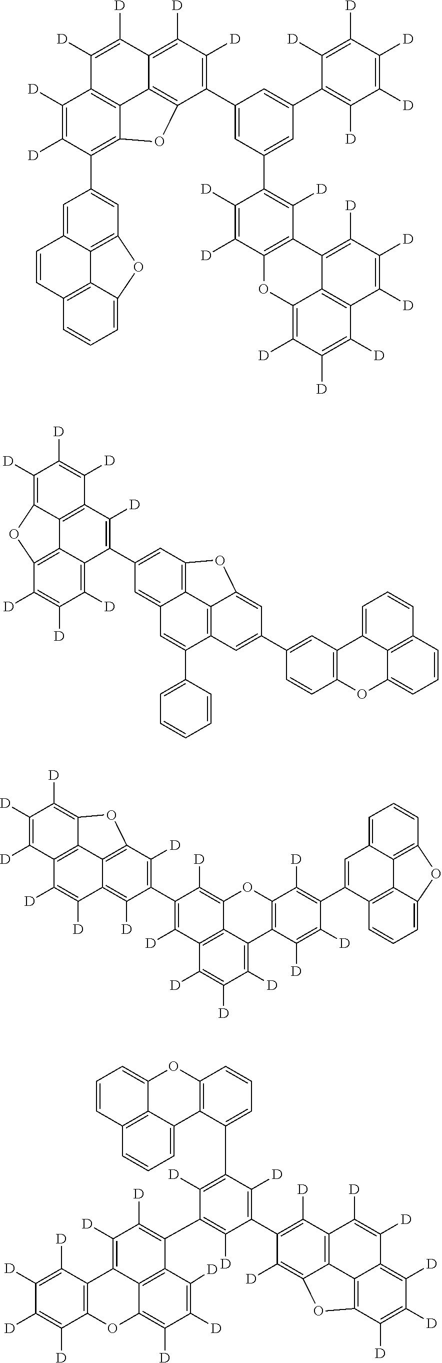

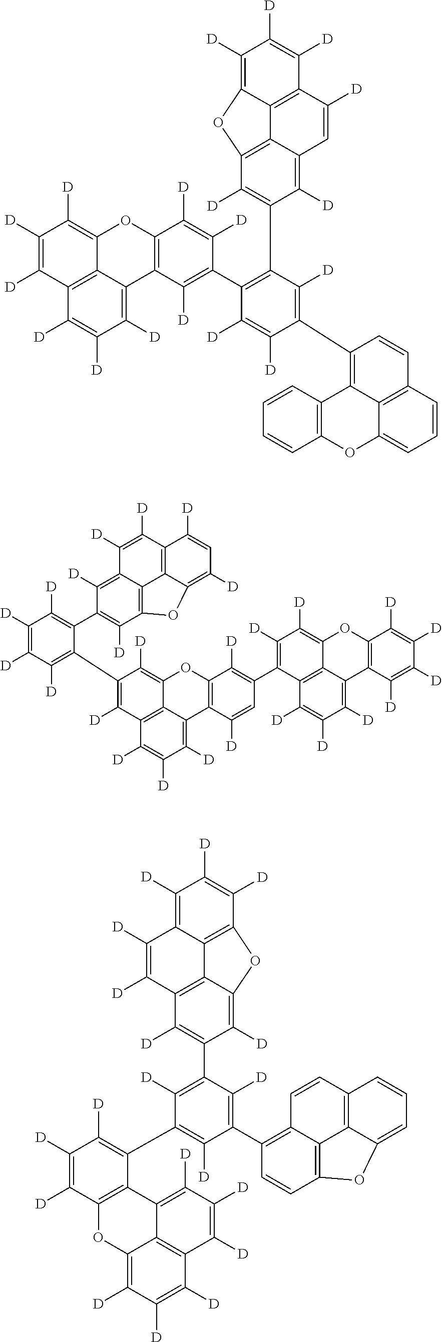

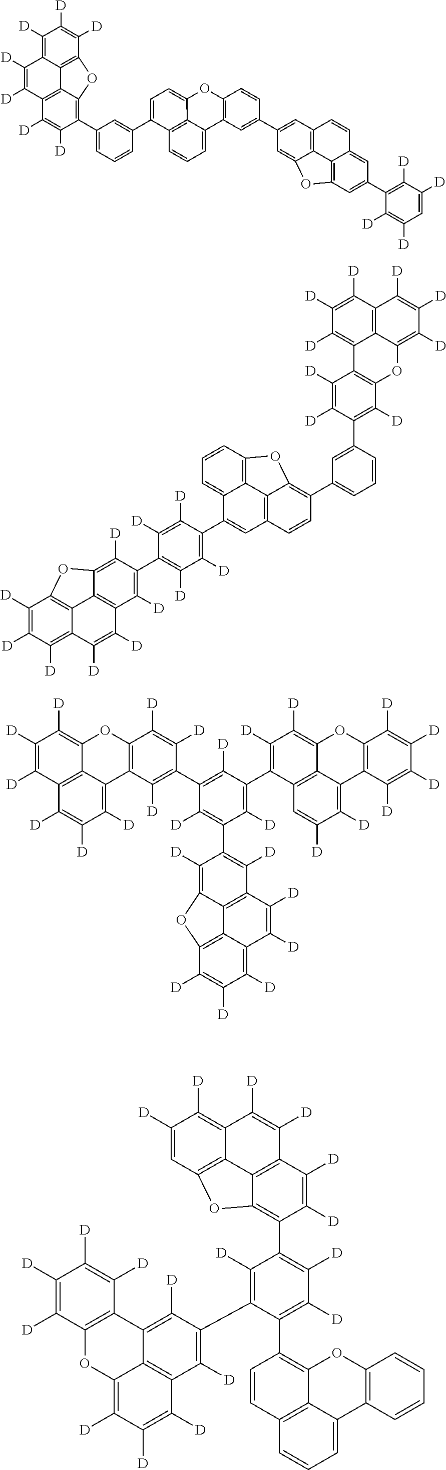

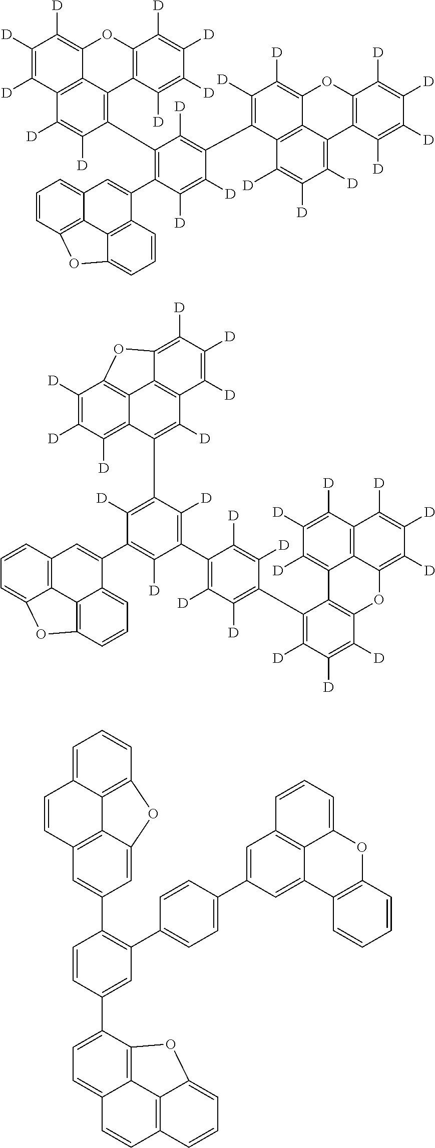

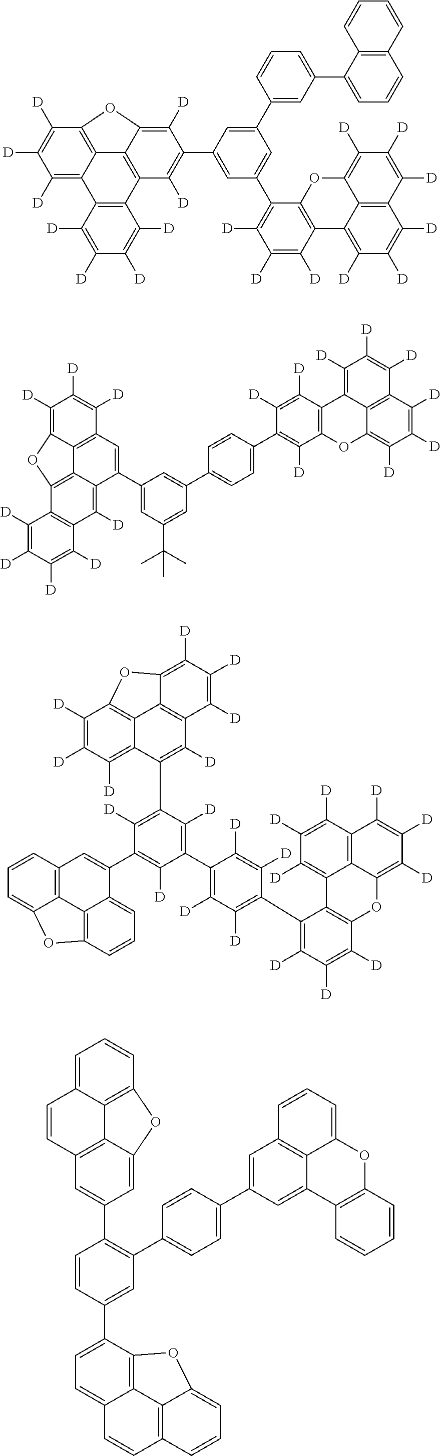

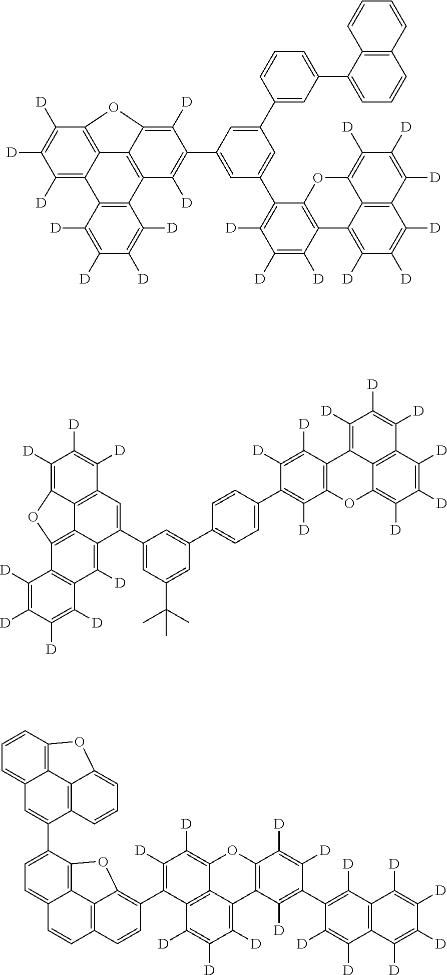

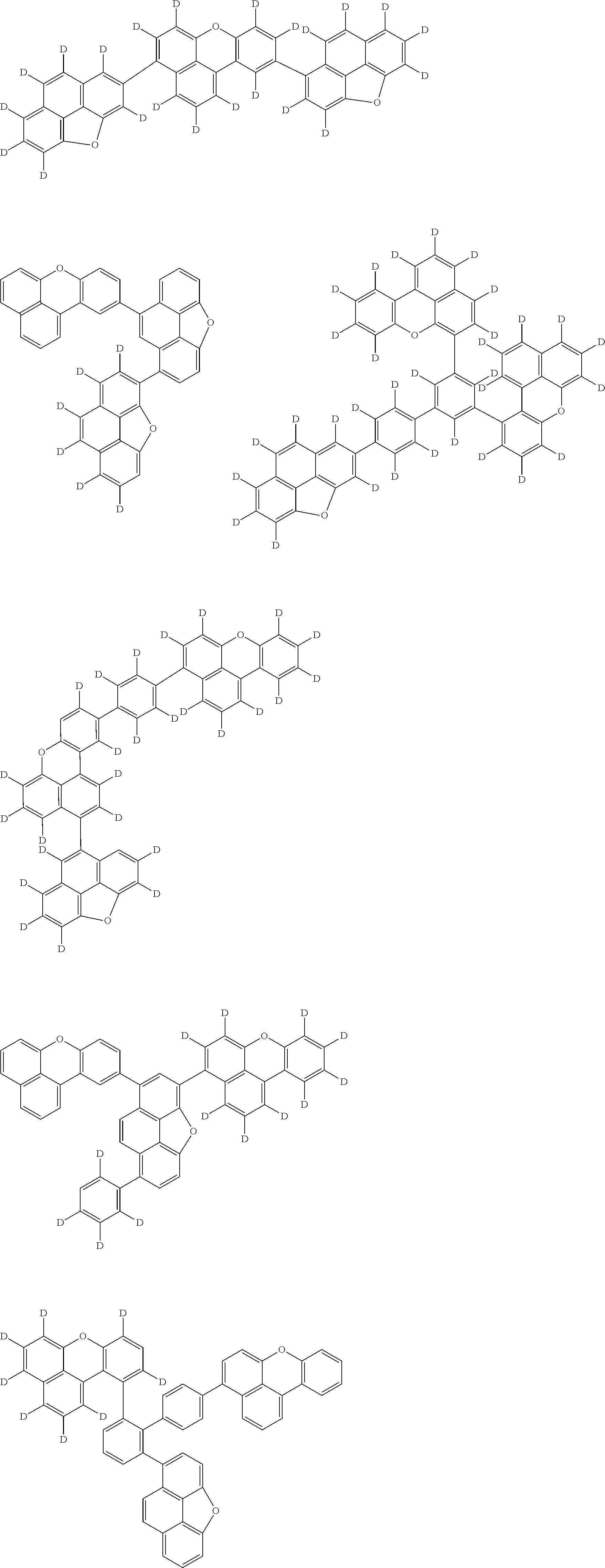

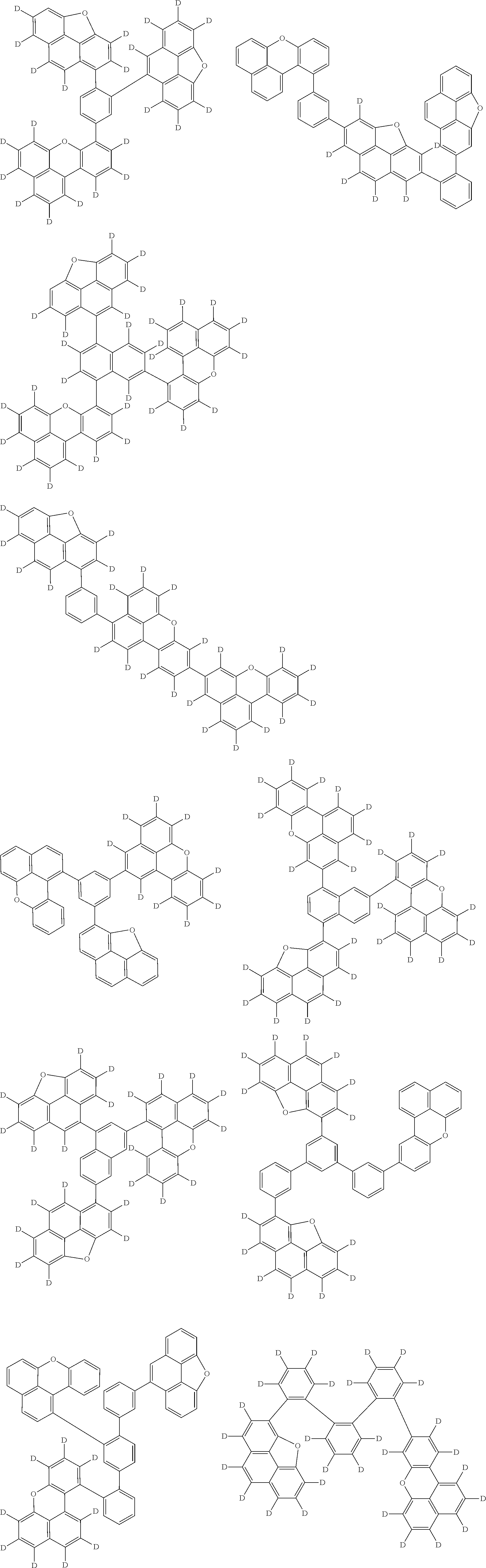

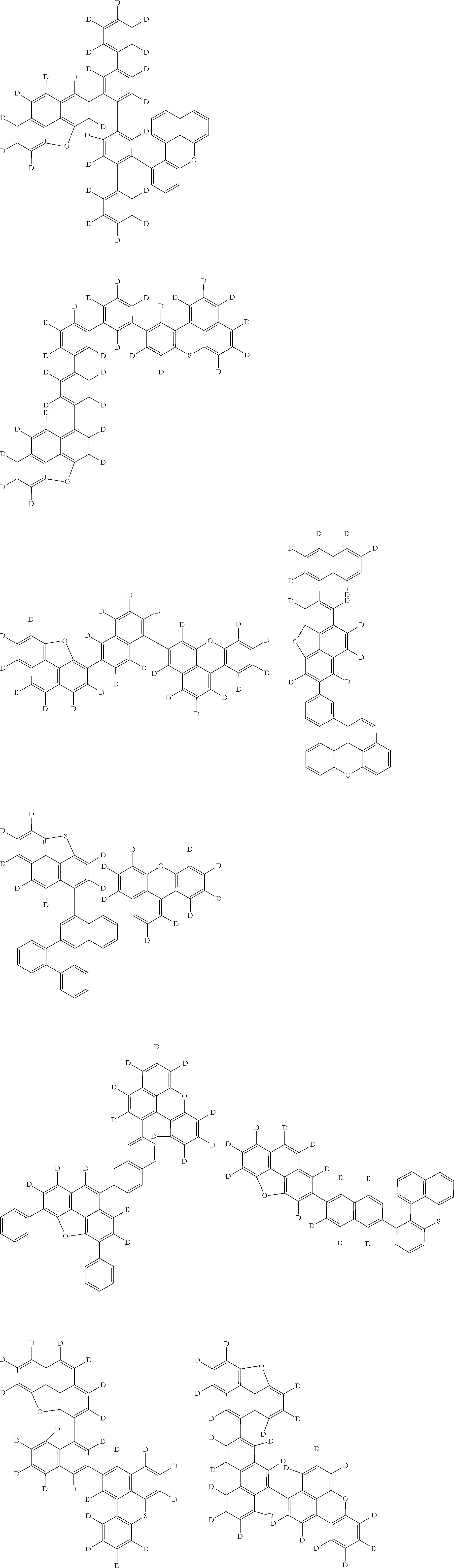

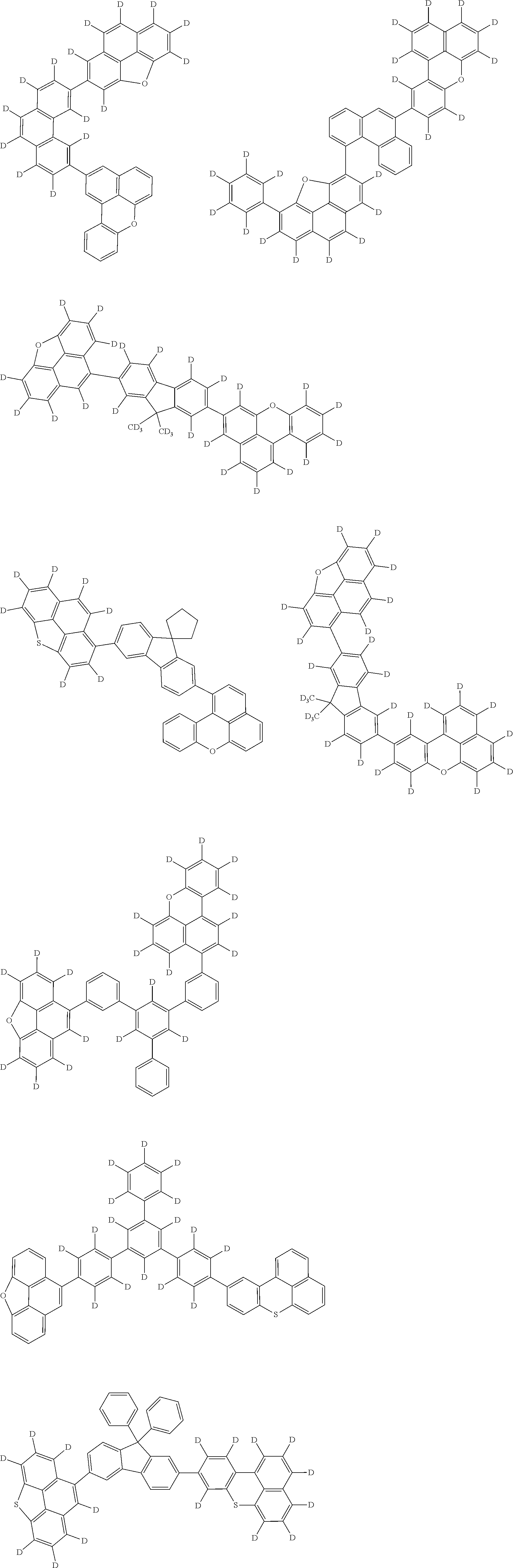

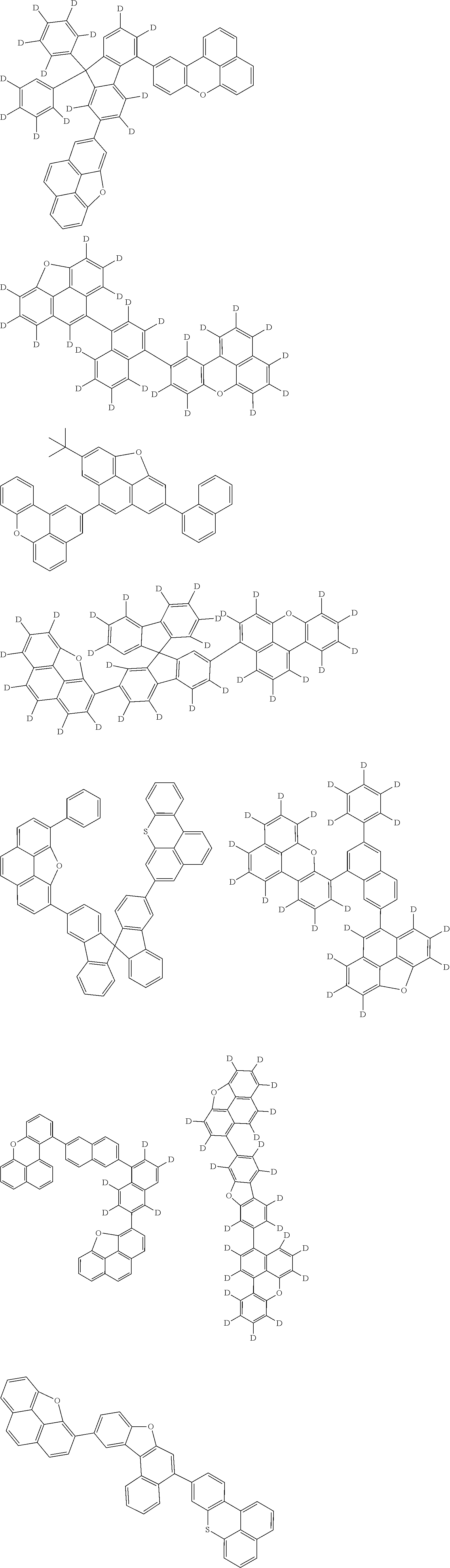

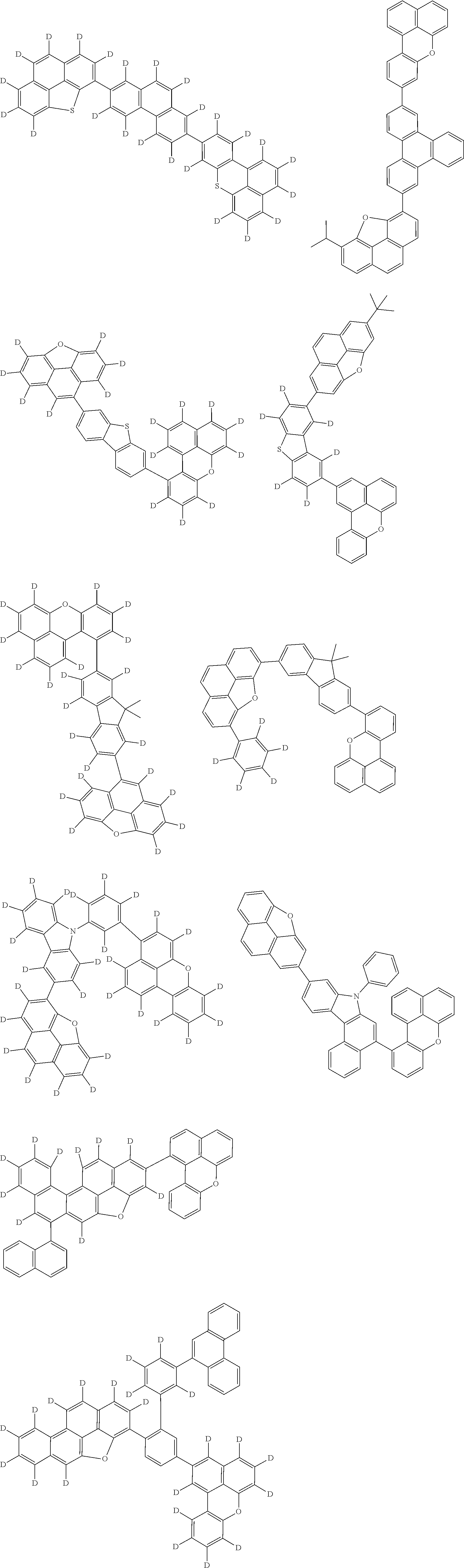

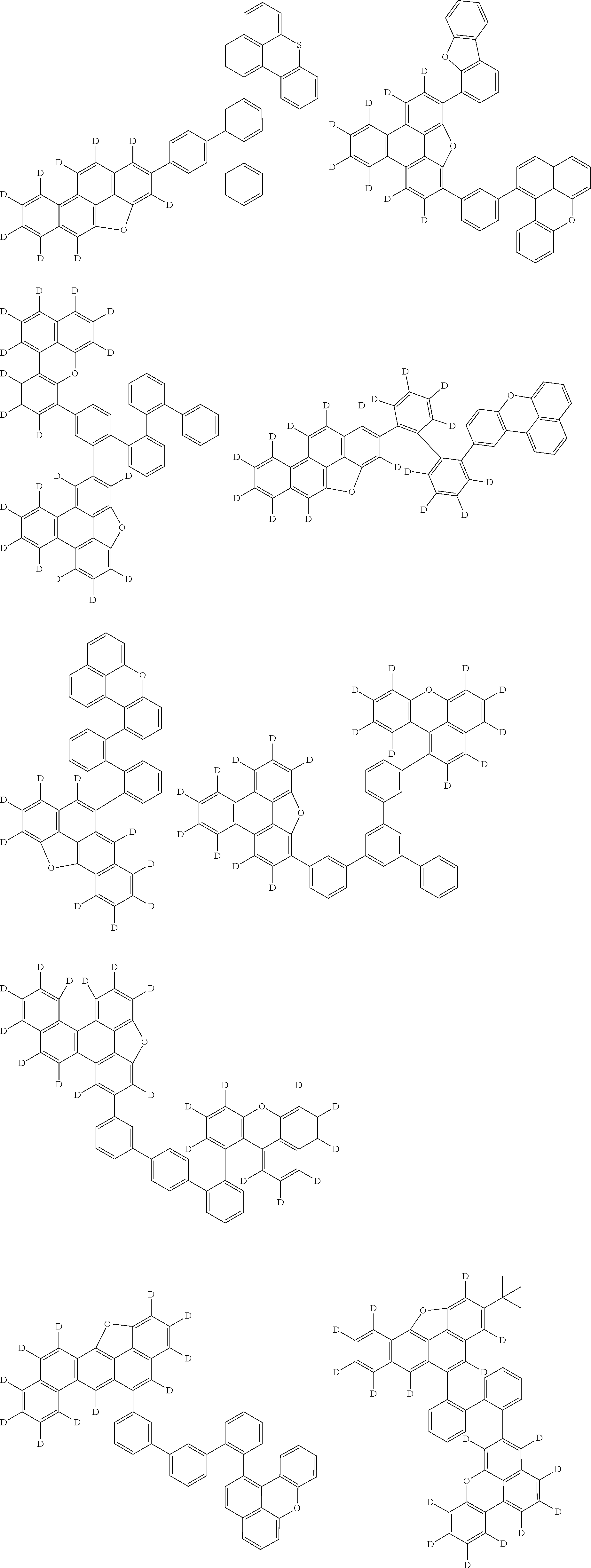

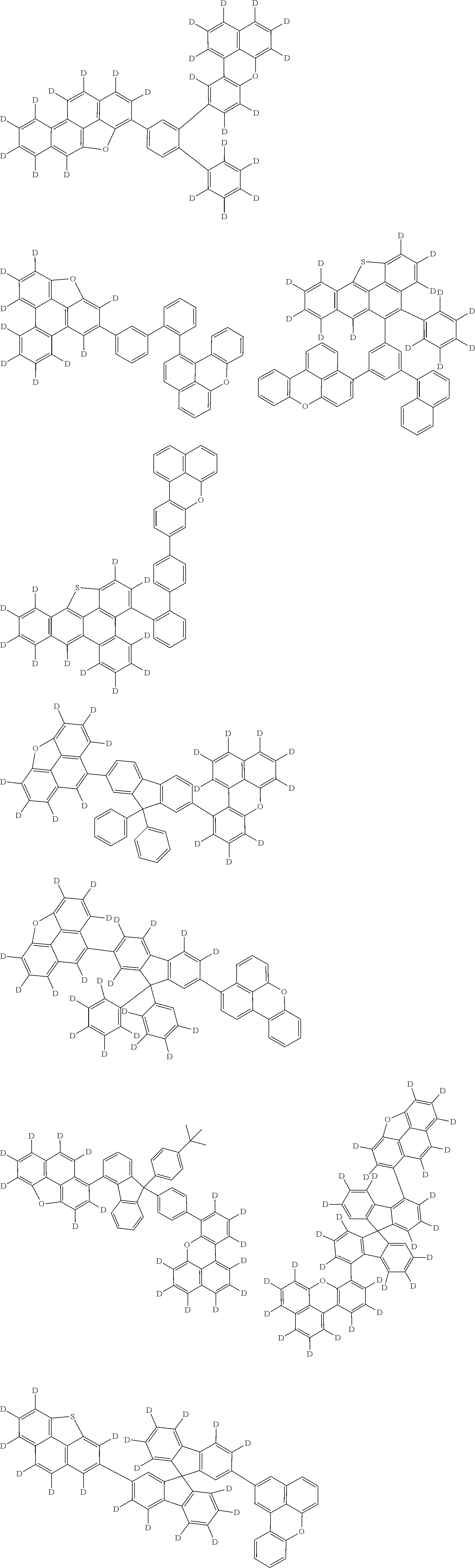

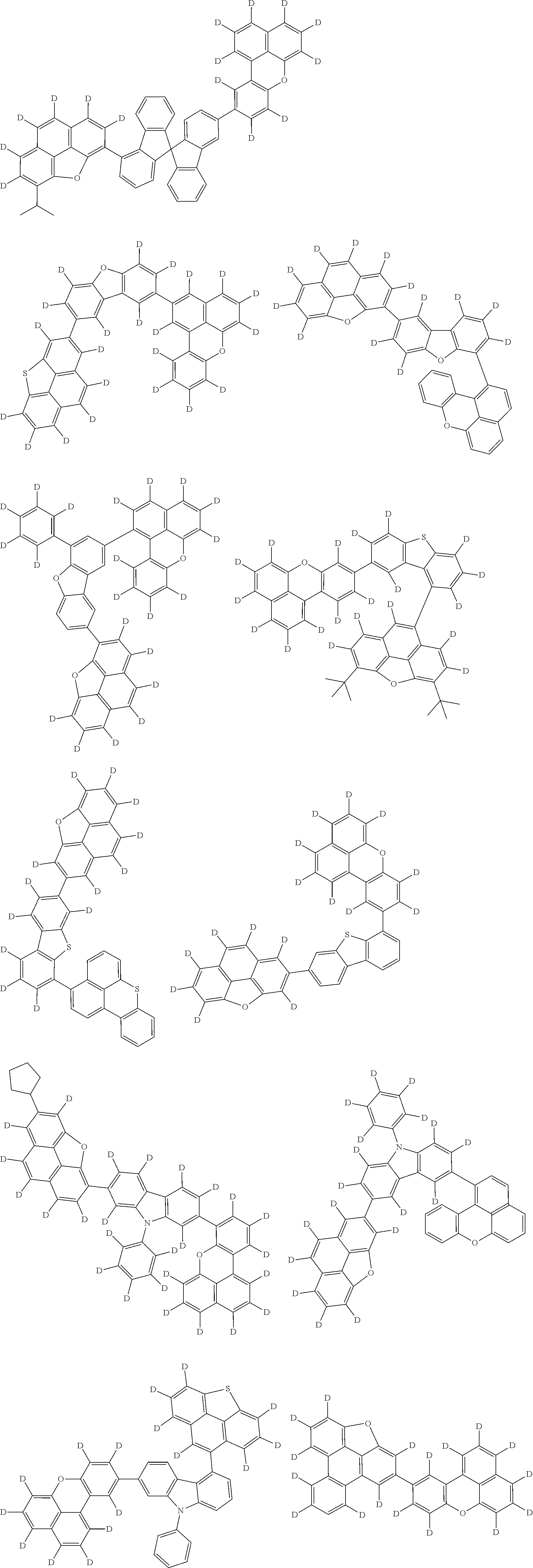

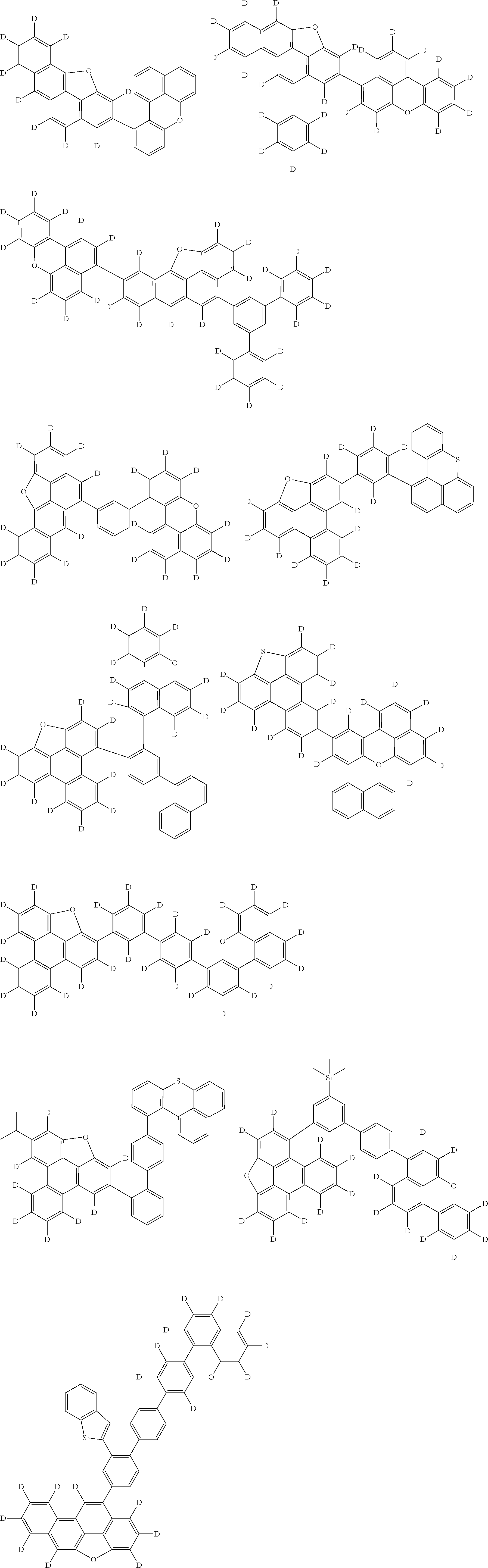

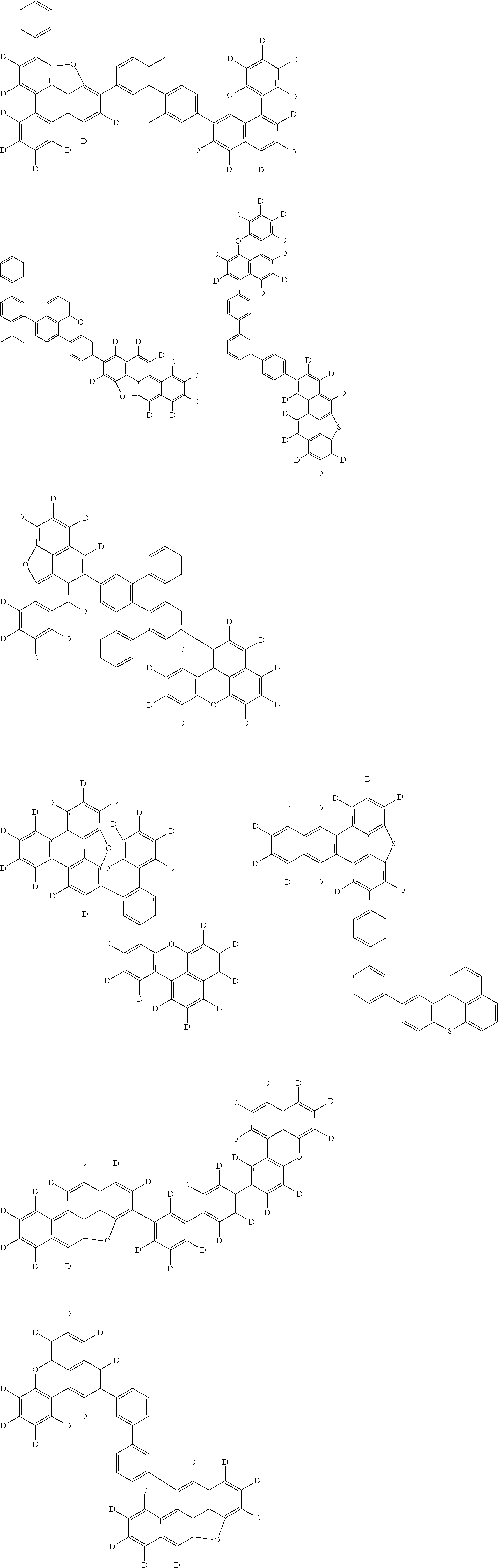

In the structures, hydrogen may be replaced with deuterium.

Further, the present specification provides an organic light emitting device including the above-described compound.

In an exemplary embodiment of the present specification, provided is an organic light emitting device including an anode; a cathode; and an organic material layer having one or more layers provided between the anode and the cathode, in which one or more layers of the organic material layer include the compound.

The organic material layer of the organic light emitting device of the present specification may have a single-layered structure, but may also have a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic layers.

In an exemplary embodiment of the present specification, the organic material layer includes a hole injection layer or a hole transport layer, and the hole injection layer or the hole transport layer includes the compound.

In an exemplary embodiment of the present specification, the organic material layer includes a light emitting layer, and the light emitting layer includes the compound. Specifically, the light emitting layer may include a host and a dopant including the compound.

In an exemplary embodiment of the present specification, the organic material layer includes an electron transport layer or an electron injection layer, and the electron transport layer or the electron injection layer includes the compound.

In an exemplary embodiment of the present specification, the organic material layer includes a light emitting layer, and further includes one or two or more layers selected from the group consisting of a hole injection layer, a hole transport layer, an electron transport layer, an electron injection layer, an electron blocking layer, and a hole blocking layer.

In an exemplary embodiment of the present specification, the organic light emitting device includes: an anode; a cathode; and an organic material layer having one or more layers provided between the anode and the cathode, the organic material layer includes: a light emitting layer; a hole transport region provided between the light emitting layer and the anode; and an electron transport region provided between the light emitting layer and the cathode, and the light emitting layer includes the compound.

In an exemplary embodiment of the present specification, as the organic material layer in the hole transport region, one or more may be selected from the group consisting of a hole transport layer, a hole injection layer, a layer which simultaneously transports and injects holes, and an electron blocking layer.

In an exemplary embodiment of the present specification, as the organic material layer in the electron transport region, one or more may be selected from the group consisting of an electron transport layer, an electron injection layer, a layer which simultaneously transports and injects electrons, and a hole blocking layer.

In another exemplary embodiment, the organic light emitting device may be a normal type organic light emitting device in which an anode, an organic material layer having one or more layers, and a cathode are sequentially stacked on a substrate.

In still another exemplary embodiment, the organic light emitting device may be an inverted type organic light emitting device in which a cathode, an organic material layer having one or more layers, and an anode are sequentially stacked on a substrate.

The organic light emitting device may have, for example, the stacking structure described below, but the stacking structure is not limited thereto.
(1) Anode/Hole transport layer/Light emitting layer/Cathode
(2) Anode/Hole injection layer/Hole transport layer/Light emitting layer/Cathode
(3) Anode/Hole injection layer/Hole buffer layer/Hole transport layer/Light emitting layer/Cathode
(4) Anode/Hole transport layer/Light emitting layer/Electron transport layer/Cathode
(5) Anode/Hole transport layer/Light emitting layer/Electron transport layer/Electron injection layer/Cathode
(6) Anode/Hole injection layer/Hole transport layer/Light emitting layer/Electron transport layer/Cathode
(7) Anode/Hole injection layer/Hole transport layer/Light emitting layer/Electron transport layer/Electron injection layer/Cathode
(8) Anode/Hole injection layer/Hole transport layer/Hole adjusting layer/Light emitting layer/Electron transport layer/Cathode
(9) Anode/Hole injection layer/Hole transport layer/Hole adjusting layer/Light emitting layer/Electron transport layer/Electron injection layer/Cathode
(10) Anode/Hole injection layer/Hole transport layer/Light emitting layer/Electron adjusting layer/Electron transport layer/Electron injection layer/Cathode
(11) Anode/Hole injection layer/Hole transport layer/Hole adjusting layer/Light emitting layer/Electron adjusting layer/Electron transport layer/Electron injection layer/Cathode
(12) Anode/Hole transport layer/First light emitting layer/Second light emitting layer/Cathode
(13) Anode/Hole transport layer/First light emitting layer/Second light emitting layer/Electron transport layer/Electron injection layer/Cathode
(14) Anode/Hole injection layer/Hole transport layer/First light emitting layer/Second light emitting layer/Electron transport layer/Cathode
(15) Anode/Hole injection layer/Hole transport layer/First light emitting layer/Second light emitting layer/Electron transport layer/Electron injection layer/Cathode
(16) Anode/Hole injection layer/Hole transport layer/Hole adjusting layer/First light emitting layer/Second light emitting layer/Electron transport layer/Cathode
(17) Anode/Hole injection layer/Hole transport layer/Hole adjusting layer/First light emitting layer/Second light emitting layer/Electron transport layer/Electron injection layer/Cathode
(18) Anode/Hole injection layer/Hole transport layer/First light emitting layer/Second light emitting layer/Electron adjusting layer/Electron transport layer/Electron injection layer/Cathode
(19) Anode/Hole injection layer/Hole transport layer/Hole adjusting layer/First light emitting layer/Second light emitting layer/Electron adjusting layer/Electron transport layer/Electron injection layer/Cathode

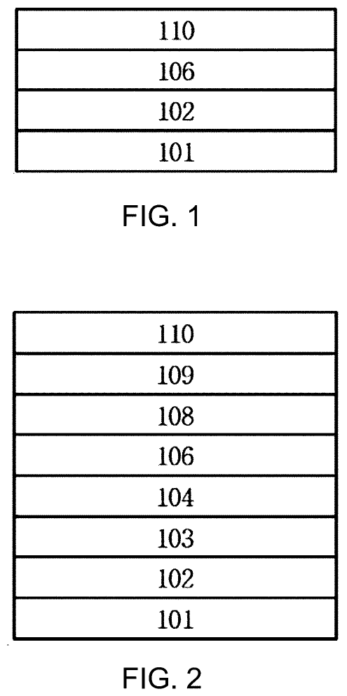

The structure of the organic light emitting device of the present specification may have structures as illustrated in FIGS. 1 to 4, but is not limited thereto.

FIG. 1 illustrates an example of an organic light emitting device according to an exemplary embodiment of the present invention, and exemplifies the structure of an organic light emitting device in which a substrate 101, an anode 102, a light emitting layer 106, and a cathode 110 are sequentially stacked. In the structure as described above, the compound of Chemical Formula 1 and the second compound, which is an anthracene derivative, may be each included in a single light emitting layer as a mixture or composition, or may be included in a light emitting layer having two layers.

FIG. 2 illustrates an example of an organic light emitting device according to an exemplary embodiment of the present invention, and exemplifies the structure of an organic light emitting device in which a substrate 101, an anode 102, a hole injection layer 103, a hole transport layer 104, a light emitting layer 106, an electron transport layer 108, an electron injection layer 109, and a cathode 110 are sequentially stacked. In the structure as described above, the compound of Chemical Formula 1 and the second compound, which is an anthracene derivative, may be each included in a single light emitting layer 106, or may be included in a light emitting layer having two layers 106.

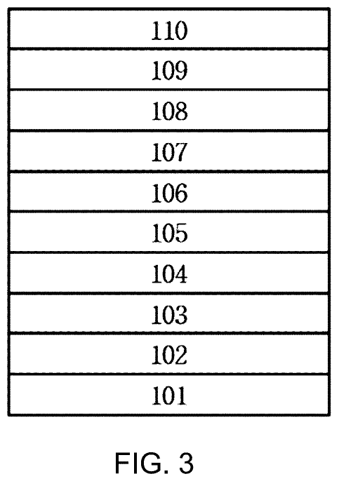

FIG. 3 illustrates an example of an organic light emitting device according to an exemplary embodiment of the present invention, and exemplifies the structure of an organic light emitting device in which a substrate 101, an anode 102, a hole injection layer 103, a hole transport layer 104, a hole adjusting layer 105, a light emitting layer 106, an electron adjusting layer 107, an electron transport layer 108, an electron injection layer 109, and a cathode 110 are sequentially stacked. In the structure as described above, the compound of Chemical Formula 1 and the second compound, which is an anthracene derivative, may be each included in a single light emitting layer, or may be included in a light emitting layer having two or more layers.

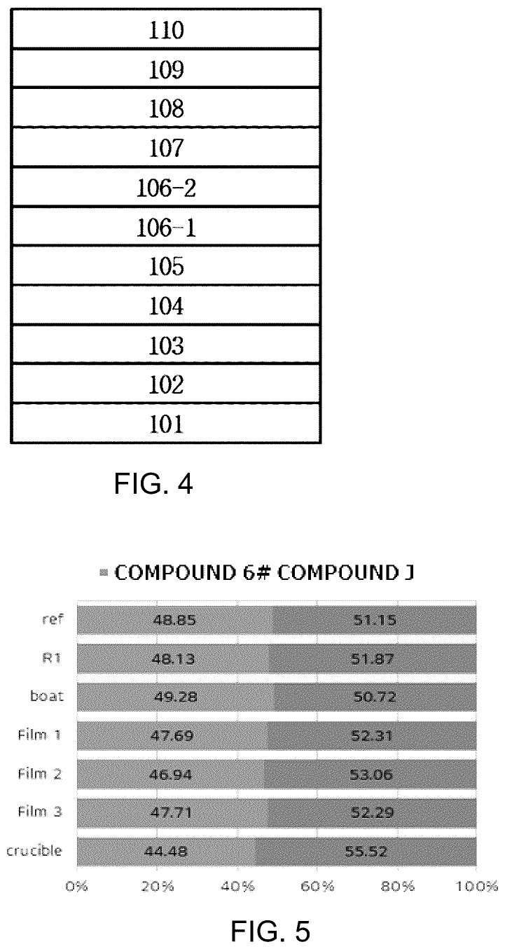

FIG. 4 illustrates an example of an organic light emitting device according to an exemplary embodiment of the present invention, and exemplifies the structure of an organic light emitting device in which a substrate 101, an anode 102, a hole injection layer 103, a hole transport layer 104, a hole adjusting layer 105, a first light emitting layer 106-1, a second light emitting layer 106-2, an electron adjusting layer 107, an electron transport layer 108, an electron injection layer 109, and a cathode 110 are sequentially stacked. In the structure described above, the compound of Chemical Formula 1 may be included in the first light emitting layer 106-1. Alternatively, the second compound, which is an anthracene derivative, may be each included in the second light emitting layer 106-2, or vice versa.

The organic light emitting device of the present specification may be manufactured by the materials and methods known in the art, except that one or more layers of the organic material layer include the compound of the present specification, that is, the compound.

When the organic light emitting device includes a plurality of organic material layers, the organic material layers may be formed of the same material or different materials.

The organic light emitting device of the present specification may be manufactured by the materials and methods known in the art, except that one or more layers of the organic material layer include the compound, that is, the compound of Chemical Formula 1.

For example, the organic light emitting device of the present specification may be manufactured by sequentially stacking an anode, an organic material layer, and a cathode on a substrate. In this case, the organic light emitting device may be manufactured by depositing a metal or a metal oxide having conductivity, or an alloy thereof on a substrate to form an anode, forming an organic material layer including a hole injection layer, a hole transport layer, a light emitting layer, and an electron transport layer thereon, and then depositing a material, which may be used as a cathode, thereon, by using a physical vapor deposition (PVD) method such as sputtering or e-beam evaporation. In addition to the method described above, an organic light emitting device may be made by sequentially depositing a cathode material, an organic material layer, and an anode material on a substrate.

Further, the compound of Chemical Formula 1 may be formed as an organic material layer by not only a vacuum deposition method, but also a solution application method when an organic light emitting device is manufactured. Here, the solution application method means spin coating, dip coating, doctor blading, inkjet printing, screen printing, a spray method, roll coating, and the like, but is not limited thereto.

The anode is an electrode which injects holes, and as an anode material, materials having a high work function are usually preferred so as to facilitate the injection of holes into an organic material layer. Specific examples of the anode material which may be used in the present invention include: a metal, such as vanadium, chromium, copper, zinc, and gold, or an alloy thereof; a metal oxide, such as zinc oxide, indium oxide, indium tin oxide (ITO), and indium zinc oxide (IZO); a combination of a metal and an oxide, such as ZnO:Al or SnO₂:Sb; a conductive polymer, such as poly(3-methylthiophene), poly[3,4-(ethylene-1,2-dioxy)thiophene] (PEDOT), polypyrrole, and polyaniline; and the like, but are not limited thereto.

The cathode is an electrode which injects electrons, and as a cathode material, materials having a low work function are usually preferred so as to facilitate the injection of electrons into an organic material layer. Specific examples of the cathode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

The hole injection layer is a layer which injects holes from an electrode, and a hole injection material is preferably a compound which has a capability of transporting holes and thus has an effect of injecting holes at an anode and an excellent effect of injecting holes for a light emitting layer or a light emitting material, prevents excitons produced from the light emitting layer from moving to an electron injection layer or an electron injection material, and is also excellent in the ability to form a thin film. The highest occupied molecular orbital (HOMO) of the hole injection material is preferably a value between the work function of the anode material and the HOMO of the neighboring organic material layer. Specific examples of the hole injection material include metal porphyrin, oligothiophene, arylamine-based organic materials, hexanitrile hexaazatriphenylene-based organic materials, quinacridone-based organic materials, perylene-based organic materials, anthraquinone, polyaniline-based and polythiophene-based conductive polymers, and the like, but are not limited thereto.

According to an exemplary embodiment of the present specification, the hole injection layer includes a compound of the following Chemical Formula HI-1, but is not limited thereto.

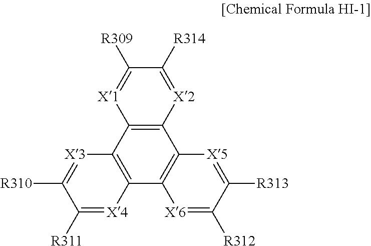

In Chemical Formula HI-1,
at least one of X′1 to X′6 is N, and the others are CH, and
R309 to R314 are the same as or different from each other, and are each independently hydrogen; deuterium; a cyano group; a substituted or unsubstituted alkyl group; a substituted or unsubstituted amine group; a substituted or unsubstituted aryl group; or a substituted or unsubstituted heteroaryl group, or are bonded to an adjacent group to form a substituted or unsubstituted ring.

According to an exemplary embodiment of the present specification, X′1 to X′6 are N.

According to an exemplary embodiment of the present specification, R309 to R314 are a cyano group.

According to an exemplary embodiment of the present specification, Chemical Formula HI-1 is the following compound.

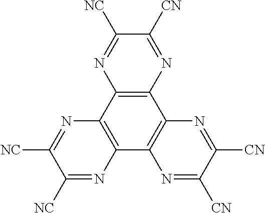 [SMILES: N#Cc1nc2c3nc(C#N)c(C#N)nc3c3nc(C#N)c(C#N)nc3c2nc1C#N]

The hole transport layer is a layer which accepts holes from a hole injection layer and transports the holes to a light emitting layer, and a hole transport material is suitably a material having high hole mobility which may accept holes from an anode or a hole injection layer and transfer the holes to a light emitting layer. Specific examples thereof include arylamine-based organic materials, conductive polymers, block copolymers having conjugated parts and non-conjugated parts together, and the like, but are not limited thereto.

The hole adjusting layer is a layer which may improve the service life and efficiency of a device by adjusting holes transported from a hole transport layer such that the holes are smoothly injected into a light emitting layer, and preventing electrons injected from an electron injection layer from passing through a light emitting layer and entering a hole injection layer. As the hole adjusting layer, the publicly-known material can be used without limitation, and the hole adjusting layer may be formed between a light emitting layer and a hole injection layer, between a light emitting layer and a hole transport layer, or between a light emitting layer and a layer which simultaneously injects and transports holes.

According to an exemplary embodiment of the present specification, the hole transport layer or hole adjusting layer includes a compound of following Chemical Formula HT-1, but is not limited thereto.

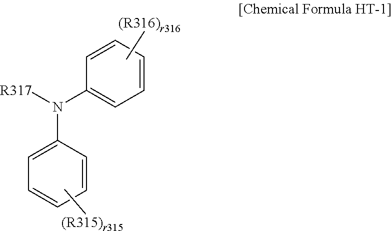

In Chemical Formula HT-1,
R315 to R317 are the same as or different from each other, and are each independently any one selected from the group consisting of hydrogen; deuterium; a substituted or unsubstituted alkyl group; a substituted or unsubstituted aryl group; a substituted or unsubstituted heteroaryl group; and a combination thereof, or are bonded to an adjacent group to form a substituted or unsubstituted ring,
r315 is an integer from 1 to 5, and when r315 is 2 or higher, two or more R315's are the same as or different from each other, and
r316 is an integer from 1 to 5, and when r316 is 2 or higher, two or more R316's are the same as or different from each other.

According to an exemplary embodiment of the present specification, R317 is any one selected from the group consisting of a substituted or unsubstituted aryl group; a substituted or unsubstituted heteroaryl group; and a combination thereof.

According to an exemplary embodiment of the present specification, R317 is any one selected from the group consisting of a carbazole group; a phenyl group; a biphenyl group; and a combination thereof.

According to an exemplary embodiment of the present specification, R315 and R316 are the same as or different from each other, and are each independently a substituted or unsubstituted aryl group, or are bonded to an adjacent group to form an aromatic hydrocarbon ring substituted with an alkyl group.

According to an exemplary embodiment of the present specification, R315 and R316 are the same as or different from each other, and are each independently a phenyl group or a phenanthrene group, or are bonded to an adjacent group to form indene substituted with a methyl group.

According to an exemplary embodiment of the present specification, Chemical Formula HT-1 is any one of the following compounds.

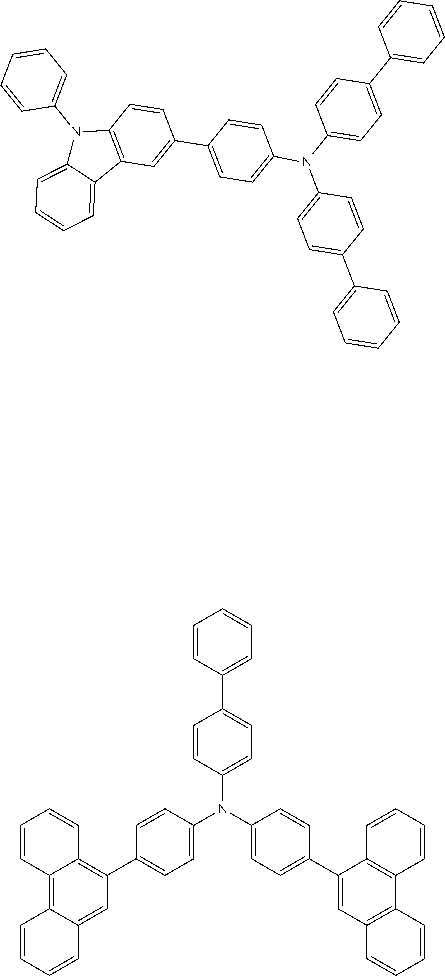 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(N(c3ccc(-c4cc5ccccc5c5ccccc45)cc3)c3ccc(-c4cc5ccccc5c5ccccc45)cc3)cc2)cc1]

The electron adjusting layer is a layer that adjusts electrons transferred from the electron transport layer to be smoothly injected into the light emitting layer, and known materials can be used without limitation.

According to an exemplary embodiment of the present specification, the electron adjusting layer includes a compound of the following Chemical Formula EG-1, but is not limited thereto.

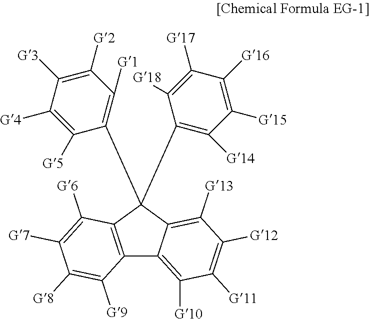

In Chemical Formula EG-1,
at least one of G′1 to G′18 is -L5-Ar5, the others are hydrogen, or G′1 and G′18 are linked through -L51- to form a substituted or unsubstituted ring,
L5 is a direct bond; or a substituted or unsubstituted arylene group,
Ar5 is a substituted or unsubstituted aryl group; or a substituted or unsubstituted heteroaryl group, and
L51 is 0; or S.

According to an exemplary embodiment of the present specification, L51 is 0.

According to an exemplary embodiment of the present specification, L51 is S.

According to an exemplary embodiment of the present specification, G′1 and G′18 are linked through -L51- to form a substituted or unsubstituted hetero ring.

According to an exemplary embodiment of the present specification, G′1 and G′18 are linked through -L51- to form a substituted or unsubstituted xanthene ring; or a substituted or unsubstituted thioxanthene ring.

According to an exemplary embodiment of the present specification, G′1 and G′18 are linked through —O— to form a substituted or unsubstituted xanthene ring.

According to an exemplary embodiment of the present specification, G′1 and G′18 are linked through —S— to form a substituted or unsubstituted thioxanthene ring.

According to an exemplary embodiment of the present specification, G′1 and G′18 are linked through —O— to form a xanthene ring.

According to an exemplary embodiment of the present specification, G′1 and G′18 are linked through —S— to form a thioxanthene ring.

According to an exemplary embodiment of the present specification, L5 is a direct bond; or a substituted or unsubstituted monocyclic or polycyclic arylene group having 6 to 30 carbon atoms.

According to an exemplary embodiment of the present specification, L5 is a direct bond; or a substituted or unsubstituted monocyclic or polycyclic arylene group having 6 to 20 carbon atoms.

According to an exemplary embodiment of the present specification, L5 is a direct bond; or a monocyclic or polycyclic arylene group having 6 to 30 carbon atoms.

According to an exemplary embodiment of the present specification, L5 is a direct bond; or a monocyclic or polycyclic arylene group having 6 to 20 carbon atoms.

According to an exemplary embodiment of the present specification, L5 is a direct bond; or a phenylene group.

According to an exemplary embodiment of the present specification, Ar5 is a substituted or unsubstituted triazine group.

According to an exemplary embodiment of the present specification, Ar5 is a triazine group which is unsubstituted or substituted with a monocyclic or polycyclic aryl group having 6 to 30 carbon atoms.

According to an exemplary embodiment of the present specification, Ar5 is a triazine group substituted with a phenyl group.

According to an exemplary embodiment of the present specification, Chemical Formula EG-1 is following compound.

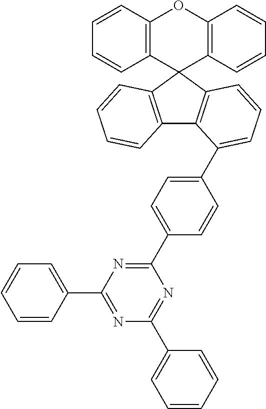 [SMILES: c1ccc(-c2nc(-c3ccccc3)nc(-c3ccc(-c4cccc5c4-c4ccccc4C54c5ccccc5Oc5ccccc54)cc3)n2)cc1]

In an exemplary embodiment of the present specification, the light emitting layer includes a host and a dopant. In this case, the host and the dopant are included at a weight ratio of 0.1:99.9 to 20:80.

Examples of a host material for the light emitting layer include fused aromatic ring derivatives, or hetero ring-containing compounds, and the like. Specifically, examples of the fused aromatic ring derivative include anthracene derivatives, pyrene derivatives, naphthalene derivatives, pentacene derivatives, phenanthrene compounds, fluoranthene compounds, and the like, and examples of the hetero ring-containing compound include carbazole derivatives, dibenzofuran, dibenzofuran derivatives, dibenzothiophene, dibenzothiophene derivatives, ladder-type furan compounds, pyrimidine derivatives, and the like, but the examples thereof are not limited thereto.

For the dopant for the light emitting layer, when the light emitting layer emits red light, it is possible to use a phosphorescent material such as bis(1-phenylisoquinoline)acetylacetonate iridium (PIQIr(acac)), bis(1-phenylquinoline)acetylacetonate iridium (PQIr(acac)), tris(1-phenylquinoline)iridium (PQIr), or octaethylporphyrin platinum (PtOEP), or a fluorescent material such as tris(8-hydroxyquinolino)aluminum (Alq₃) as a light emitting dopant, but the light emitting dopant is not limited thereto. When the light emitting layer emits green light, it is possible to use a phosphorescent material such as fac tris(2-phenylpyridine)iridium (Ir(ppy)₃), or a fluorescent material such as tris(8-hydroxyquinolino)aluminum (Alq3), as the light emitting dopant, but the light emitting dopant is not limited thereto. When the light emitting layer emits blue light, phosphorescent materials such as (4,6-F2ppy)₂Irpic, or fluorescent materials such as spiro-DPVBi, spiro-6P, distyrylbenzene (DSB), distyrylarylene (DSA), PFO-based polymers or PPV-based polymers may be used as the light emitting dopant, however, the light emitting dopant is not limited thereto.

In an exemplary embodiment of the present specification, the light emitting layer includes one or more first compounds of Chemical Formula 1 of the present application and the second compound which is an anthracene derivative.

In an exemplary embodiment of the present specification, the light emitting layer may be composed of a single layer, but may also be a plurality of layers having two or more layers.

When the light emitting layer is a single layer, the first compound of Chemical Formula 1 and the second compound which is an anthracene derivative are included in one layer. Such a single-layered light emitting layer may be formed by co-depositing the first compound of Chemical Formula 1 and the second compound which is an anthracene derivative.

When the light emitting layer has two or more layers, the first compound of Chemical Formula 1 and the second compound which is an anthracene derivative may be included in one layer, and the first compound of Chemical Formula 1 and the second compound which is an anthracene derivative may be included in different light emitting layers. For example, the light emitting layer may include a first light emitting layer including the first compound of Chemical Formula 1 and a second light emitting layer including the second compound which is an anthracene derivative. In this case, the second light emitting layer may be provided between the first light emitting layer and the cathode. The first light emitting layer and the second light emitting layer may be provided in contact with each other. When the first light emitting layer and the second light emitting layer each include the compound of Chemical Formula 1 and the second compound which is an anthracene derivative as hosts, the first light emitting layer and the second light emitting layer each further include a dopant compound. In this case, the first light emitting layer and the second light emitting layer may include the same type of dopant material and may include different types of dopant materials, but preferably include the same type of dopant material.

In an exemplary embodiment of the present specification, the light emitting layer having one or more layers includes the compound of Chemical Formula 1 and the second compound which is an anthracene derivative as hosts.

In an exemplary embodiment of the present specification, the second compound, which is an anthracene derivative, may include at least one compound of a compound of the following Chemical Formula 2 and a compound of the following Chemical Formula 3. Specifically, the second compound, which is an anthracene derivative, is a mixed host including two different types of anthracene derivatives, and may include a compound of the following Chemical Formula 2 and a compound of the following Chemical Formula 3.

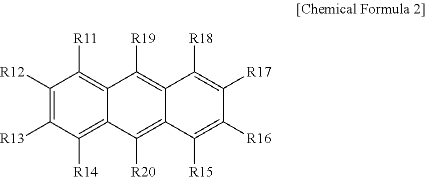

In Chemical Formula 2,
one or more of R11 to R20 are bonded to the * moiety of the following Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group,

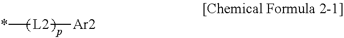

in Chemical Formula 2-1,
L2 is a direct bond; a substituted or unsubstituted arylene group; or a substituted or unsubstituted heteroarylene group,
Ar2 is a substituted or unsubstituted aryl group, and
p is an integer from 1 to 3, and when p is 2 or higher, the L2s in the parentheses are the same as or different from each other,

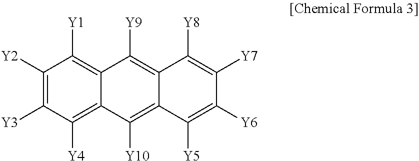

in Chemical Formula 3,
one or more of Y1 to Y10 are bonded to the * moiety of the following Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group,

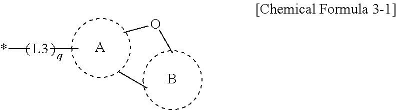

in Chemical Formula 3-1,
A and B are the same as or different from each other, and are each independently a substituted or unsubstituted aromatic hydrocarbon ring; or a substituted or unsubstituted aromatic hetero ring,
L3 is a direct bond; a substituted or unsubstituted arylene group; or a substituted or unsubstituted heteroarylene group, and
q is an integer from 1 to 3, and when q is 2 or higher, the L3s in the parentheses are the same as or different from each other.

The present inventors have discovered suitable conditions to be applied to a phenantrofuran- or phenanthrothiophene-type host (hereinafter, referred to as a compound of Chemical Formula 1) and an anthracene-type host (hereinafter, referred to as compounds of Chemical Formulae 2 and 3), which may be useful as blue fluorescent host materials and may simultaneously provide a stable co-evaporation mixture after premixing.

The light emitting layer of an organic light emitting device that exhibits excellent service life and efficiency requires more than two components (for example, three or four components). For this purpose, three or four raw materials are required to prepare such a light emitting layer, which is very complex and expensive compared to a standard two-component light emitting layer with a single host and a dopant, which require only two raw materials. Premixing two or more materials and evaporating the two or more materials from one raw material may reduce the complexity of the preparation process. A selected hole transport host and another type of host, such as an electron transport host, may be mixed and co-evaporated from one crucible to achieve stable evaporation.

However, the co-evaporation needs to be stable, that is, the composition of the evaporated film needs to be kept constant during the manufacturing process. Any compositional changes may adversely affect device performance. It may be assumed that in order to obtain stable co-evaporation from a mixture of compounds under vacuum, the materials need to have the same evaporation temperature under the same conditions. However, this will not be the only parameter to consider. When two compounds are mixed together, the compounds may interact with each other and their evaporation properties may differ from their individual properties. On the other hand, materials with slightly different evaporation temperatures may produce a stable co-evaporation mixture. Therefore, it is extremely difficult to achieve a stable co-evaporation mixture. To date, there have been tens of thousands of usable host materials in the literature, but very few examples of the stable co-evaporation mixture. The “evaporation temperature” of a material is measured in a sublimation crucible of a vacuum deposition tool, such as a VTE tool, at a deposition rate of 2 A/see on a surface located apart at a set distance from the evaporation source of the material to be evaporated, and under a constant pressure of 1×10⁻⁶ Torr to 1×10⁻⁹ Torr. As will be understood by those skilled in the art, the various measured values disclosed in the present specification, such as temperature, pressure, and deposition rate, are expected to have a nominal variation due to an expected permissible error in the measurements from which this quantitative value is calculated.

The disclosed content of the present specification describes a new class of two different types of anthracene hosts (Chemical Formulae 2 and 3) that may be premixed to provide a stable co-evaporation mixture useful as a blue fluorescent host material. A plurality of factors other than temperature may contribute to evaporation, for example, miscibility of different materials, and different phase transitions. The present inventors discovered that two materials may persistently co-evaporate when the materials have similar evaporation temperatures and similar rates of mass loss or similar vapor pressures. The rate of mass loss is defined as the percentage of mass loss over time (minutes) and is determined by measuring the time taken for the mass to be reduced by first 10% as measured by thermogravimetric analysis (TGA) under identical experimental conditions at the same constant temperature given for each compound after reaching the steady evaporation state. The given constant temperature is a temperature point selected such that the value of the mass loss rate is about 0.05%/min to 0.50%/min. Those skilled in the art will understand that in order to compare two parameters, the experimental conditions need to be constant. Methods of measuring the rate of mass loss and vapor pressure are well known in the art, and may be found, for example, in the literature (Bull. et al., Mater. Sci. 2011, 34, 7).

Among the two-component mixture of blue fluorescent hosts in the current state of the art, the prior document of the present invention is disclosed in JP 6328890 B2 presented. To prepare a blue fluorescent light emitting layer with such a mixed host material, three evaporation sources are required: two types of hosts and a blue fluorescent dopant. The concentrations of a co-host and a dopant are important for device performance, and the deposition rate of each component is typically measured individually during deposition. This complicates the manufacturing process and requires a lot of costs. Therefore, it is desirable to reduce the number of raw materials by mixing two or more components.

When two types of blue fluorescent hosts are premixed and the difference in composition is within a certain range during the preparation of a mixture through thermal sublimation, it means that the two blue fluorescent hosts may be co-deposited from one raw material. The uniform co-evaporation of the two hosts is important for the sustainability of the performance of a device manufactured from this mixture.

Hereinafter, a compound of Chemical Formula 2 will be described in detail.

According to an exemplary embodiment of the present specification, one to three of R11 to R20 are bonded to the * moiety of Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

According to another exemplary embodiment, one to three of R11 to R20 are bonded to the * moiety of Chemical Formula 2-1, and the others are the same as or different from each other, and are each independently hydrogen; deuterium; a halogen group; a cyano group; a nitro group; a hydroxyl group; a substituted or unsubstituted alkyl group having 1 to 20 carbon atoms; a substituted or unsubstituted aryl group having 6 to 60 carbon atoms; a substituted or unsubstituted cycloalkyl group having 3 to 60 carbon atoms; a substituted or unsubstituted aryl group having 2 to 60 carbon atoms; or a substituted or unsubstituted silyl group.

In another exemplary embodiment, R19 and R20 are bonded to the * moiety of Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

According to still another exemplary embodiment, R19, R20, and R18 are bonded to the * moiety of Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

In still another exemplary embodiment, R19, R20, and R17 are bonded to the * moiety of Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

According to an exemplary embodiment of the present specification, among R11 to R20, the substituents which are not bonded to Chemical Formula 2-1 are the same as or different from each other, and are each independently hydrogen, deuterium, or a dibenzofuranyl group.

According to an exemplary embodiment of the present specification, p is an integer of 3, and three L2s are the same as or different from each other.

In yet another exemplary embodiment, p is an integer of 2, and two L2s are the same as or different from each other.

According to an exemplary embodiment of the present specification, L2 is a direct bond; a substituted or unsubstituted arylene group having 6 to 60 carbon atoms; or a substituted or unsubstituted heteroarylene group having 2 to 60 carbon atoms. L2 may be substituted with deuterium.

In still yet another exemplary embodiment, L2 is a direct bond; a substituted or unsubstituted arylene group having 6 to carbon atoms; or a substituted or unsubstituted heteroarylene group having 2 to 30 carbon atoms.

According to another exemplary embodiment, L2 is a direct bond; a substituted or unsubstituted phenylene group; a substituted or unsubstituted biphenylylene group; a substituted or unsubstituted naphthylene group; a substituted or unsubstituted phenanthrenylene group; or a substituted or unsubstituted triphenylenylene group.

In a further exemplary embodiment, L2 is a direct bond; a phenylene group that is unsubstituted or substituted with deuterium; a biphenylylene group that is unsubstituted or substituted with deuterium; a naphthylene group that is unsubstituted or substituted with deuterium; a phenanthrenylene group that is unsubstituted or substituted with deuterium; or a triphenylenylene group that is unsubstituted or substituted with deuterium.

According to an exemplary embodiment of the present specification, Ar2 is a substituted or unsubstituted aryl group having 6 to 60 carbon atoms. Ar2 may be substituted with deuterium.

In another further exemplary embodiment, Ar2 is a substituted or unsubstituted aryl group having 6 to 30 carbon atoms.

According to another exemplary embodiment, Ar2 is a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group, or a substituted or unsubstituted triphenylenyl group.

In another further exemplary embodiment, Ar2 is a phenyl group that is unsubstituted or substituted with deuterium; a biphenyl group that is unsubstituted or substituted with deuterium; a terphenyl group that is unsubstituted or substituted with deuterium; a naphthyl group that is unsubstituted or substituted with deuterium; a phenanthrenyl group that is unsubstituted or substituted with deuterium; or a triphenylenyl group that is unsubstituted or substituted with deuterium.

As the compound of Chemical Formula 2, it is possible to use compounds described in JP 4070676 B2, KR 1477844 B1, U.S. Pat. No. 6,465,115 B2, JP 3148176 B2, JP 4025136 B2, JP 4188082 B2, JP 5015459 B2, KR 1979037 B1, KR 1550351 B1, KR 1503766 B1, KR 0826364 B1, KR 0749631 B1, KR 1115255 B1, KR 1538534 B1, and the like.

Hereinafter, a compound of Chemical Formula 3 will be described in detail.

According to an exemplary embodiment of the present specification, one or more of Y1 to Y10 are bonded to the * moiety of Chemical Formula 3-1, and the others are the same as or different from each other, and are each independently hydrogen; deuterium; or a substituted or unsubstituted aryl group having 6 to 60 carbon atoms.

According to an exemplary embodiment of the present specification, one to three of Y1 to Y10 are bonded to the * moiety of Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

In an exemplary embodiment of the present specification, one to three of Y1 to Y10 are bonded to the * moiety of Chemical Formula 3-1, and the others are the same as or different from each other, and are each independently hydrogen; deuterium; a halogen group; a cyano group; a nitro group; a hydroxyl group; a substituted or unsubstituted alkyl group having 1 to 20 carbon atoms; a substituted or unsubstituted aryl group having 6 to 60 carbon atoms; a substituted or unsubstituted cycloalkyl group having 3 to 60 carbon atoms; a substituted or unsubstituted heteroaryl group having 2 to 60 carbon atoms; or a substituted or unsubstituted silyl group.

In another exemplary embodiment, Y7, Y8, or Y9 is bonded to the * moiety of Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

According to another exemplary embodiment, Y7 and Y9 are bonded to the * moiety of Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

According to still another exemplary embodiment, Y9 and Y10 are bonded to the * moiety of Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group.

According to an exemplary embodiment of the present specification, among Y1 to Y10, the substituents which are not bonded to Chemical Formula 3-1 are the same as or different from each other, and are each independently hydrogen; deuterium; or a substituted or unsubstituted aryl group having 6 to 30 carbon atoms. Among Y1 to Y10, the substituents, which are not bonded to Chemical Formula 3-1, may be substituted with deuterium.

According to another exemplary embodiment, among Y1 to Y10, the substituents, which are not bonded to Chemical Formula 3-1, are the same as or different from each other, and are each independently hydrogen; deuterium; a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; or a substituted or unsubstituted triphenylenyl group, and the substituents may be further substituted with deuterium; a phenyl group that is unsubstituted or substituted with deuterium; a biphenyl group that is unsubstituted or substituted with deuterium; a terphenyl group that is unsubstituted or substituted with deuterium; a naphthyl group that is unsubstituted or substituted with deuterium; a phenanthrenyl group that is unsubstituted or substituted with deuterium; or a triphenylenyl group that is unsubstituted or substituted with deuterium.

In an exemplary embodiment of the present specification, q is an integer from 1 to 3, and when q is 2 or higher, two or more L3's are the same as or different from each other.

According to an exemplary embodiment of the present specification, L3 is a direct bond; a substituted or unsubstituted arylene group having 6 to 60 carbon atoms; or a substituted or unsubstituted heteroarylene group having 2 to 60 carbon atoms. L2 may be substituted with deuterium.

In another exemplary embodiment, L3 is a direct bond; a substituted or unsubstituted arylene group having 6 to 30 carbon atoms; or a substituted or unsubstituted heteroarylene group having 2 to 30 carbon atoms.

According to another exemplary embodiment, L3 is a direct bond; a substituted or unsubstituted phenylene group; a substituted or unsubstituted biphenylylene group; a substituted or unsubstituted naphthylene group; a substituted or unsubstituted phenanthrenylene group; or a substituted or unsubstituted triphenylenylene group.

In still another exemplary embodiment, L3 is a direct bond; a phenylene group that is unsubstituted or substituted with deuterium; a biphenylylene group that is unsubstituted or substituted with deuterium; a naphthylene group that is unsubstituted or substituted with deuterium; a phenanthrenylene group that is unsubstituted or substituted with deuterium; or a triphenylenylene group that is unsubstituted or substituted with deuterium.

According to an exemplary embodiment of the present specification, A and B are the same as or different from each other, and are each independently a substituted or unsubstituted aromatic hydrocarbon ring having 6 to 60 carbon atoms; or a substituted or unsubstituted aromatic hetero ring having 2 to 60 carbon atoms.

In an exemplary embodiment of the present specification, A and B are the same as or different from each other, and are each independently an aromatic hydrocarbon ring having 6 to 60 carbon atoms, which is unsubstituted or substituted with one or more substituents selected from the group consisting of deuterium and an aryl group that is unsubstituted or substituted with deuterium; or an aromatic hetero ring having 2 to 60 carbon atoms, which is unsubstituted or substituted with one or more substituents selected from the group consisting of deuterium and an aryl group that is unsubstituted or substituted with deuterium.

According to another exemplary embodiment, A and B are the same as or different from each other, and are each independently an aromatic hydrocarbon ring having 6 to 60 carbon atoms, which is unsubstituted or substituted with one or more substituents selected from the group consisting of deuterium and an aryl group having 6 to 30 carbon atoms, which is unsubstituted or substituted with deuterium; or an aromatic hetero ring having 2 to 60 carbon atoms, which is unsubstituted or substituted with one or more substituents selected from the group consisting of deuterium and an aryl group having 6 to 30 carbon atoms, which is unsubstituted or substituted with deuterium.

In another exemplary embodiment, A and B are the same as or different from each other, and are each independently substituted or unsubstituted benzene; substituted or unsubstituted naphthalene; substituted or unsubstituted phenanthrene; substituted or unsubstituted triphenylene; or substituted or unsubstituted dibenzofuran.

According to still another exemplary embodiment, A and B are the same as or different from each other, and are each independently benzene that is unsubstituted or substituted with one or more substituents selected from the group consisting of deuterium and an aryl group having 6 to 30 carbon atoms, which is unsubstituted or substituted with deuterium; naphthalene that is unsubstituted or substituted with one or more substituents selected from the group consisting of deuterium and an aryl group having 6 to 30 carbon atoms, which is unsubstituted or substituted with deuterium; phenanthrene that is unsubstituted or substituted with one or more substituents selected from the group consisting of deuterium and an aryl group having 6 to 30 carbon atoms, which is unsubstituted or substituted with deuterium; triphenylene that is unsubstituted or substituted with one or more substituents selected from the group consisting of deuterium and an aryl group having 6 to 30 carbon atoms, which is unsubstituted or substituted with deuterium; or dibenzofuran that is unsubstituted or substituted with one or more substituents selected from the group consisting of deuterium and an aryl group having 6 to 30 carbon atoms, which is unsubstituted or substituted with deuterium.

As the compound of Chemical Formula 3, it is possible to use compounds described in KR 1964435 B1, KR 1899728 B1, KR 1975945 B1, KR 2018-0098122 A, KR 2018-0102937 A, KR 2018-0103352 A, KR 1538534 B1, and the like.

In the organic light emitting device according to an exemplary embodiment of the present specification, the sublimation temperature (T_{sub1}) of the first compound of Chemical Formula 1 and the sublimation temperature (T_{sub2}) of the second compound satisfy the following Equation (1):

$$\vert {T}_{sub 1} - {T}_{sub 2} \vert \leq 20 °C . \qquad Equation ( 1 )$$

When the above-described configuration is satisfied, electrons are easily injected into the triplet energy level provided by the first compound of Chemical Formula 1 while reproducing the low voltage and high efficiency provided by the second compound, so that there is an advantage in that the light emitting efficiency is increased because the proportion of excitons formed is increased.

In the organic light emitting device according to an exemplary embodiment of the present specification, the light emitting layer includes a first light emitting layer and a second light emitting layer,
in which the first light emitting layer includes the compound of Chemical Formula 1 as a first host material, and
the second light emitting layer includes at least one of the compounds of Chemical Formulae 2 and 3 as a second host material. When such a configuration is satisfied, the efficiency of the organic light emitting device may be additionally improved.

In the organic light emitting device according to an exemplary embodiment of the present specification, the second light emitting layer is provided between the first light emitting layer and the cathode.

The organic light emitting device according to an exemplary embodiment of the present specification is provided such that the first light emitting layer and the second light emitting layer are brought into direct contact with each other. When such a configuration is satisfied, the efficiency of the organic light emitting device may be additionally improved.

In the organic light emitting device according to an exemplary embodiment of the present specification, the first light emitting layer includes a combination of the two or more compounds of Chemical Formula 1 as a first host material.

In the organic light emitting device according to an exemplary embodiment of the present specification, the light emitting layer further includes a dopant material.

As described above, the light emitting layer may include a light emitting layer having a single layer or two or more layers. When the light emitting layer is a single layer, the light emitting layer may further include a single dopant material or a combination of a plurality of dopant materials for the compound of Chemical Formula 1 and the second compound which is an anthracene derivative. Furthermore, when the light emitting layer has two layers, and includes a first light emitting layer including a compound of Chemical Formula 1 and a second light emitting layer including a second compound which is an anthracene derivative, each light emitting layer may include the same type of dopant material as each other, or different types of dopant materials. Preferably, two or more light emitting layers include the same type of dopant material.

In the organic light emitting device according to an exemplary embodiment of the present specification, the dopant material is a fluorescent dopant.

In the organic light emitting device according to an exemplary embodiment of the present specification, the fluorescent dopant is a blue fluorescent dopant.

In the organic light emitting device according to an exemplary embodiment of the present specification, the blue fluorescent dopant is a pyrene-based compound or a non-pyrene-based compound.

In the organic light emitting device according to an exemplary embodiment of the present specification, the non-pyrene-based compound is an arylamine-based compound or a boron-based compound.

Compared to the pyrene-based compound, when a non-pyrene-based compound is used as a fluorescent dopant, the efficiency may be improved and the service life may be increased due to the characteristic of the narrow full width at half maximum.

The pyrene-based compound may be a pyrene-based compound in which diarylamine is substituted, which has a smaller band gap than a host material, such as BD02 (Ni, Ni, N6, N6-tetraphenylpyrene-1,6-diamine).

The non-pyrene-based compound may include a metal complex (an iridium complex, a platinum complex, and the like), a boron-based compound (DABNA-1, and the like), an arylamine-based compound (Coumarin 6, and the like) as a compound which is not pyrene or a pyrene derivative, but is not limited thereto.

In an exemplary embodiment of the present specification, the pyrene-based compound may be a compound represented by the following Chemical Formula Z1, and the non-pyrene-based compound may be compounds represented by the following Chemical Formulae Z2 (an arylamine-based compound) and Z3 (a boron-based compound)

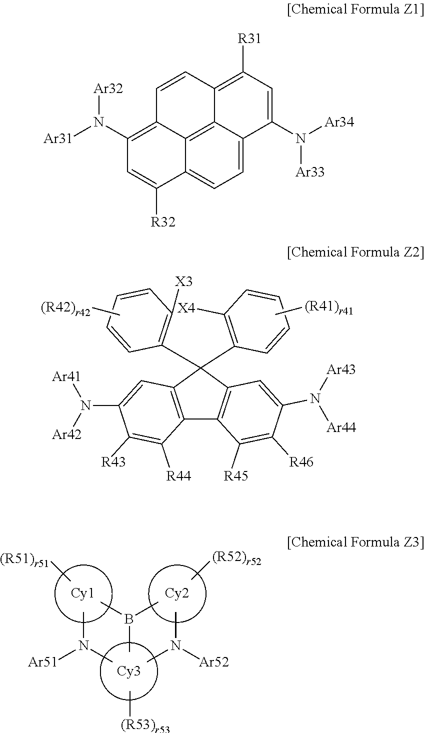

In Chemical Formulae Z1 to Z3,
X₃ and X₄ are each hydrogen or deuterium, or are directly bonded to each other to form a ring,
R41 and R42 are the same as or different from each other, and are each independently hydrogen, deuterium, or a substituted or unsubstituted alkyl group,
Ar31 to Ar34, Ar41 to Ar44, Ar51, and Ar52 are the same as or different from each other, and are each independently a substituted or unsubstituted aryl group, or substituted or unsubstituted heterocyclic group,
Cy1 to Cy3 are the same as or different from each other, and are each independently a monocyclic to polycyclic aromatic hydrocarbon ring, or monocyclic to polycyclic aromatic hetero ring,
R31, R32, R43 to R46, and R51 to R53 are the same as or different from each other, and are each independently hydrogen, deuterium, a halogen group, a cyano group, a substituted or unsubstituted alkyl group, a substituted or unsubstituted silyl group, a substituted or unsubstituted cycloalkyl group, a substituted or unsubstituted amine group, a substituted or unsubstituted aryl group, or a substituted or unsubstituted heterocyclic group, or are bonded to an adjacent substituent to form a substituted or unsubstituted ring, and
r41, r42, and r51 to r53 are each an integer from 0 to 4, and when r41, r42, and r51 to r53 are 2 or higher, substituents in the parenthesis are the same as or different from each other.

In an exemplary embodiment of the present specification, Ar3l to Ar34, Ar4l to Ar44, Ar5l, and Ar52 are the same as or different from each other, and are each independently a substituted or unsubstituted C6-C30 aryl group; or a substituted or unsubstituted C2-C30 heterocyclic group.

In an exemplary embodiment of the present specification, Ar31 to Ar34, Ar4l to Ar44, Ar5l, and Ar52 are the same as or different from each other, and are each independently a C6-C20 aryl group that is unsubstituted or substituted with one or more substituents selected from the group consisting of deuterium, a halogen group, and a C1-C6 alkyl group, or a substituent in which two or more groups selected from the above group are linked together; or a C2-C20 heterocyclic group that is unsubstituted or substituted with one or more substituents selected from the group consisting of deuterium, a halogen group, and a C1-C6 alkyl group, or a substituent in which two or more groups selected from the above group are linked together.

In an exemplary embodiment of the present specification, Cy1 to Cy3 are the same as or different from each other, and are each independently a monocyclic to tricyclic aromatic hydrocarbon ring; or a monocyclic to tricyclic aromatic hetero ring containing N, O, or S.

In an exemplary embodiment of the present specification, Cy1 to Cy3 are the same as or different from each other, and are each independently a benzene ring; a naphthalene ring; a furan ring; a thiophene ring; a benzofuran ring; a benzothiophene ring; a dibenzofuran ring; or a dibenzothiophene ring, and a C5-C10 aliphatic hydrocarbon ring may be further fused.

In an exemplary embodiment of the present specification, R31, R32, R43 to R46, and R51 to R53 are the same as or different from each other, and are each independently hydrogen; deuterium; a halogen group; a cyano group; a substituted or unsubstituted C1-C10 alkyl group; a substituted or unsubstituted C1-C30 alkylsilyl group; a substituted or unsubstituted C6-C90 arylsilyl group; a substituted or unsubstituted C3-C30 cycloalkyl group; a substituted or unsubstituted C1-C30 alkylamine group; a substituted or unsubstituted C6-C60 arylamine group; a substituted or unsubstituted C2-C60 heteroarylamine group; a substituted or unsubstituted C6-C30 aryl group; or a substituted or unsubstituted C2-C30 heterocyclic group, or are bonded to an adjacent substituent to form a C3-C30 hydrocarbon ring; or a substituted or unsubstituted C2-C30 hetero ring.

In an exemplary embodiment of the present specification, R31 and R32 are hydrogen or deuterium.

In an exemplary embodiment of the present specification, Ar31 to Ar34 are the same as or different from each other, and are each independently a substituted or unsubstituted C6-C30 aryl group; or a substituted or unsubstituted C2-C30 heterocyclic group.

In an exemplary embodiment of the present specification, Ar31 to Ar34 are the same as or different from each other, and are each independently a C6-C30 aryl group that is unsubstituted or substituted with an alkyl group; or a substituted or unsubstituted C2-C30 heterocyclic group containing 0.

In an exemplary embodiment of the present specification, Ar31 to Ar34 are the same as or different from each other, and are each independently a phenyl group that is unsubstituted or substituted with an alkyl group; or a substituted or unsubstituted dibenzofuranyl group.

In an exemplary embodiment of the present specification, Chemical Formula Z3, which is the boron-based compound, is any one of the following Chemical Formulae Z3-1 to Z3-3.

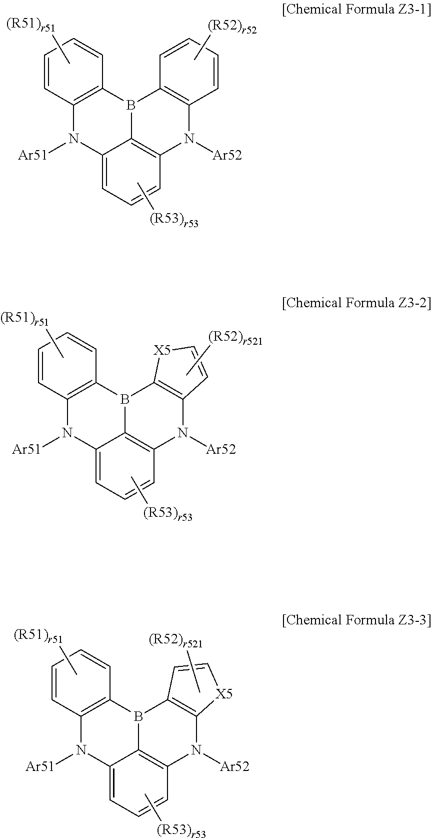

In Chemical Formulae Z3-1 to Z3-3,
R51 to R53, r52, Ar51, and Ar52 are the same as those defined in Chemical Formula Z3,
X₅ is 0; or S, and
r521 is an integer from 0 to 2, and when r521 is 2 or higher, R52's are the same as or different from each other.

In an exemplary embodiment of the present specification, Ar51 and Ar52 are the same as or different from each other, and are each independently a C6-C30 aryl group in which an aliphatic hydrocarbon ring is fused or not fused; or a C2-C30 heterocyclic group, and the aryl group or heterocyclic group is unsubstituted or substituted with one or more substituents selected from the group consisting of deuterium, a halogen group, and a C1-C10 alkyl group or a substituent in which two or more groups selected from the above group are linked together.

In an exemplary embodiment of the present specification, the aryl group or heterocyclic group of Ar51 includes a substituent instead of hydrogen at a position para-oriented with respect to the N to which the aryl group or heterocyclic group is linked.

In an exemplary embodiment of the present specification, the aryl group or heterocyclic group of Ar52 includes a substituent instead of hydrogen at a position para-oriented with respect to the N to which the aryl group or heterocyclic group is linked.

In an exemplary embodiment of the present specification, R51 to R53 are the same as or different from each other, and are each independently hydrogen; deuterium; a halogen group; a cyano group; a substituted or unsubstituted C1-C10 alkyl group; a substituted or unsubstituted C1-C30 alkylsilyl group; a substituted or unsubstituted C6-C90 arylsilyl group; a substituted or unsubstituted C3-C30 cycloalkyl group; a substituted or unsubstituted C1-C30 alkylamine group; a substituted or unsubstituted C6-C60 arylamine group; a substituted or unsubstituted C2-C60 heteroarylamine group; a substituted or unsubstituted C6-C30 aryl group; or a substituted or unsubstituted C2-C30 heterocyclic group, or are bonded to an adjacent substituent to form a C3-C30 aliphatic hydrocarbon ring; or a substituted or unsubstituted C6-C30 aromatic hydrocarbon ring.

In an exemplary embodiment of the present specification, R51 to R53 are the same as or different from each other, and are each independently hydrogen; deuterium; a methyl group; a tert-butyl group; a phenyl group; a phenyl group substituted with a tert-butyl group; a diphenylamine group; or a bis((tert-butyl)phenyl)amine group.

In an exemplary embodiment of the present specification, two adjacent R51's, two adjacent R52's, or two adjacent R53's are bonded to each other to form a substituted or unsubstituted ring. In this case, the ring is a C3-C30 aliphatic hydrocarbon ring; or a C6-C30 aromatic hydrocarbon ring, and is specifically cyclopentene, cyclohexene, a tetrahydronaphthalene ring, a benzene ring, or a naphthalene ring.

In an exemplary embodiment of the present specification, the dopant may be selected from the following structures, and is not limited thereto.

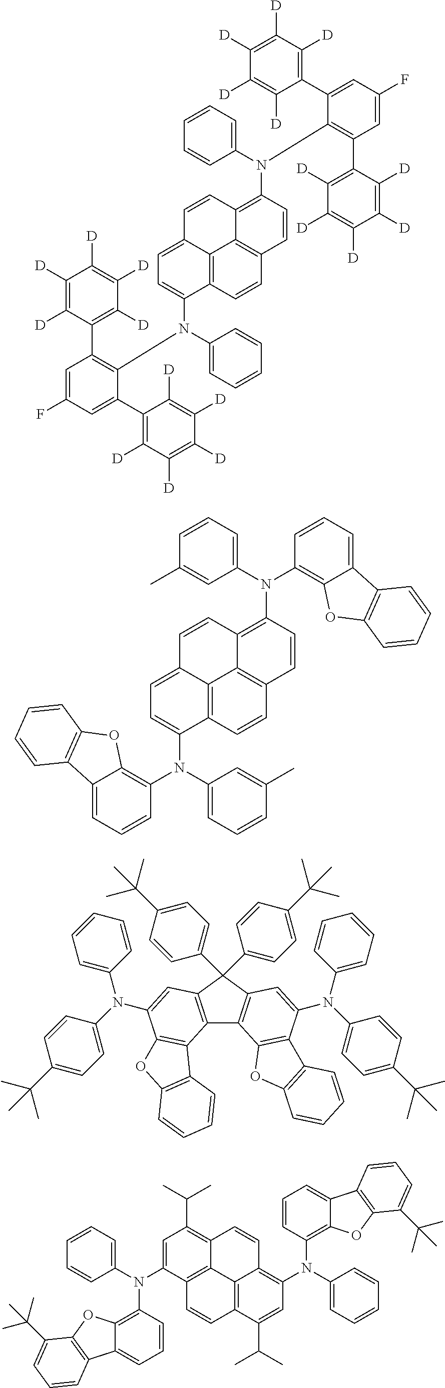

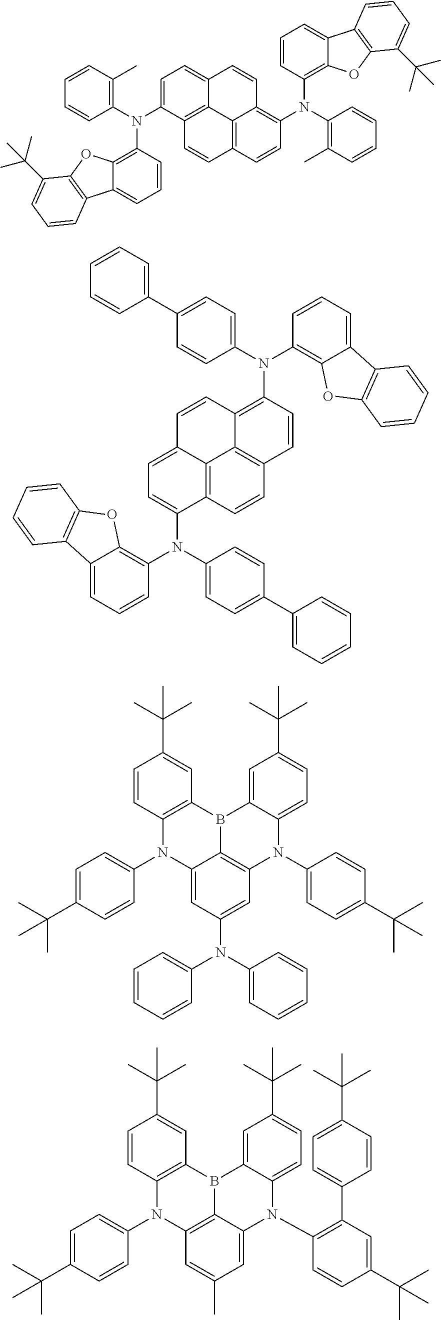

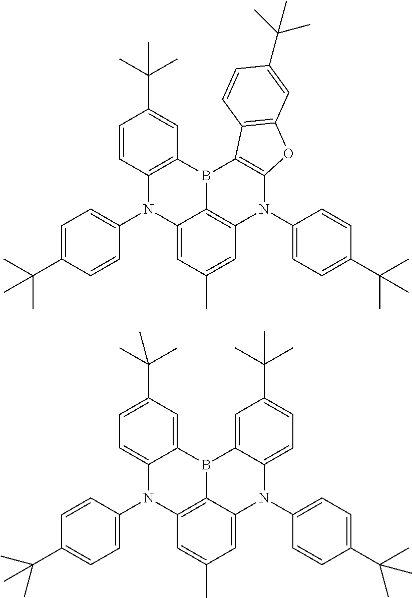

In an exemplary embodiment of the present specification, the dopant material may be included in the light emitting layer in an amount of 0.01 parts by weight to 50 parts by weight, 0.1 parts by weight to 30 parts by weight, 1 part by weight to 10 parts by weight, or 2 parts by weight, based on 100 parts by weight of the host material. Within the above range, energy transfer from the host to the dopant occurs efficiently. In this case, 100 parts by weight of the host material is based on the total weight of the compound of Chemical Formula 1 and/or the second compound.

When the above configuration is satisfied, the efficiency of the organic light emitting device may be additionally improved.

In the organic light emitting device according to an exemplary embodiment of the present specification, the deuterium substitution rate for at least one of the first compound of Chemical Formula 1 and the second compound is each independently 1% or more and 100% or less.

In an exemplary embodiment of the present specification, the deuterium substitution rate for at least one of the first compound of Chemical Formula 1 and the second compound may be each independently 1% or more, 10% or more, 20% or more, 30% or more, 40% or more, or 50% or more.

In an exemplary embodiment of the present specification, the deuterium substitution rate for at least one of the first compound of Chemical Formula 1 and the second compound may be each independently 100% or less, 99% or less, 98% or less, 97% or less, 96% or less, or 95% or less.

According to an exemplary embodiment of the present specification, the deuterium substitution rates for both the first compound of Chemical Formula 1 and the second compound are all 100%.

The deuterium substitution rate may be calculated by the above-described method. According to an additional example, the second compound is directly substituted with at least one deuterium. The light emitting layer of an organic light emitting device is a region that emits light, and is an interval where loss of molecules due to energy is large. Since the bond of carbon-deuterium is stronger than the bond of carbon-hydrogen and deuterium has a higher mass value than hydrogen, the zero point energy with carbon is lowered and the bond energy is high, so that by replacing the carbon-hydrogen bond included in the molecule of the compound of Chemical Formula 2 with a carbon-deuterium bond to increase the bonding energy of the molecule, a device having an excellent service life may be obtained.

$$Zero point energy = \sqrt{\frac{{m}_{C} + {m}_{H}}{{m}_{C} {m}_{H}}}$$

The organic light emitting device including Chemical Formula 1 and/or the second compound having a deuterium substitution rate according to an exemplary embodiment of the present specification has improved heat resistance and has an effect of improving the service life.

In the organic light emitting device according to an exemplary embodiment of the present specification, the triplet energy level (Tₕ₁) of the compound of Chemical Formula 1 is 2.30 eV or more.

In an exemplary embodiment of the present specification, the triplet energy level (Tₕ₁) of the compound of Chemical Formula 1 may be 2.30 eV or more, 2.40 eV or more, or 2.50 eV or more.

In the organic light emitting device according to an exemplary embodiment of the present specification, the triplet energy level (Tₕ₁) of the compound of Chemical Formula 1 is higher than the triplet energy level (Tₕ₂) of the second compound.

In the organic light emitting device according to an exemplary embodiment of the present specification, the triplet energy level (Tₕ₁) of the compound of Chemical Formula 1 may satisfy 2.30 eV≤Tₕ₁≤2.90 eV.

In an exemplary embodiment of the present specification, the triplet energy level (Tₕ₁) of the compound of Chemical Formula 1 may be more than 2.30 eV, more than 2.40 eV, or more than 2.50 eV.

In an exemplary embodiment of the present specification, the triplet energy level (Tₕ₁) of the compound of Chemical Formula 1 may be less than 2.90 eV, less than 2.80 eV, or less than 2.70 eV.

In the organic light emitting device according to an exemplary embodiment of the present specification, the triplet energy level (Tₕ₂) of the second compound may satisfy 1.40 eV≤Tₕ₂≤1.80 eV.

In an exemplary embodiment of the present specification, the triplet energy level (Tₕ₂) of the second compound may be more than 1.40 eV, more than 1.45 eV, more than 1.50 eV, or more than 1.55 eV.

In an exemplary embodiment of the present specification, the triplet energy level (Tₕ₂) of the second compound may be less than 1.80 eV, less than 1.75 eV, less than 1.70 eV, or less than 1.65 eV.

In the organic light emitting device according to an exemplary embodiment of the present specification, the triplet energy level (Tₕ₁) of the compound of Chemical Formula 1 and the triplet energy level (Tₕ₂) of the second compound may satisfy the following Equation (2).

$$Δ ( {T}_{h 1} - {T}_{h 2} ) \leq 1.5 eV \qquad Equation ( 2 )$$

In an exemplary embodiment of the present specification, the difference (Δ(Tₕ₁−Tₕ₂)) between the triplet energy level (Tₕ₁) of the compound of Chemical Formula 1 and the triplet energy level (Tₕ₂) of the second compound may be 1.50 eV or less, 1.45 eV or less, 1.40 eV or less, 1.35 eV or less, or 1.30 eV or less.

In the organic light emitting device according to an exemplary embodiment of the present specification, the triplet energy level (Tₕ₁) of the compound of Chemical Formula 1, the triplet energy level (Tₕ₂) of the second compound, and the triplet energy level (T_{BD}) of the blue fluorescent dopant may satisfy at least one of the following Equations (3) and (4).

$$Δ ( {T}_{BD} - {T}_{h 1} ) \leq 0.4 eV and \qquad Equation ( 3 )$$

$$Δ ( {T}_{BD} - {T}_{h 2} ) \geq 0.4 eV \qquad Equation ( 4 )$$

In an exemplary embodiment of the present specification, the difference (Δ(T_{BD}−Tₕ₁)) between the triplet energy level (Tₕ₁) of the compound of Chemical Formula 1 and the triplet energy level (T_{BD}) of the blue fluorescent dopant may be 0.40 eV or less, 0.35 eV or less, or 0.30 eV or less.

In an exemplary embodiment of the present specification, the difference (Δ(T_{BD}−Tₕ₂)) between the triplet energy level (Tₕ₂) of the second compound and the triplet energy level (T_{BD}) of the blue fluorescent dopant may be 0.40 eV or more, 0.45 eV or more, or 0.50 eV or more.

When any one of the configurations is satisfied, electrons are easily injected into the triplet energy level of the dopant, so that the efficiency is increased because the proportion of excitons formed is increased.

In the present specification, the triplet energy level (Tₕ₁) can be measured using spectroscopic instruments capable of measuring fluorescence and phosphorescence, and the measurement conditions are confirmed by preparing a solution at a concentration of 10⁻⁶ M using toluene or tetrahydrofuran (THF) as a solvent in an extremely low temperature state using liquefied nitrogen, irradiating the solution with a light source in the absorption wavelength band of a material to exclude a singlet light emission from the light emitting spectrum, and analyzing the light emitting spectrum in the triplet energy level. When electrons are excited from the light source, two components can be separated from each other in the extremely low temperature state because the time for electrons to stay at the triplet energy level is much longer than the time for the electrons to stay at the singlet energy level.

In the present specification, the triplet energy (E_{T1}) may be calculated by the following method. After a sample is cooled to 77 K, the sample for the measurement of phosphorescence is irradiated with excitation light (360 nm), the phosphorescence intensity is measured using a streak camera, and then a tangent to an ascending point of the phosphorescence spectrum is drawn, a wavelength value λedge [nm] at the intersection of the tangent and the x-axis is obtained, and an E_{T1} value converted into an energy value may be calculated by substituting this wavelength value for the following Equation (6). In this case, the light emitting spectrum was measured using a nitrogen laser (MNL200, manufactured by Lasertechnik Berlin GmbH) and a streak camera (C4334, manufactured by Hamamatsu Photonics K.K.).

$${E}_{T 1} [ eV ] = 1239.85 / λedge \qquad Equation ( 6 )$$

In an exemplary embodiment of the present specification, the triplet energy level (E_{T1}) may be calculated using Gaussian 03, a quantum chemical calculation program manufactured by Gaussian, Inc., USA. Specifically, a calculated value of the triplet energy may be obtained by the time-dependent-density functional theory (TD-DFT) with respect to a structure optimized using B3LYP (Becke, three-parameter, Lee-Yang-Parr) as a functional and 6-31G* as a basis function by using the density functional theory (DFT).

In the organic light emitting device according to an exemplary embodiment of the present specification, in the light emitting layer, the maximum light emitting wavelength (Amax) of the compound of Chemical Formula 1 and the second compound may each be in a range of 400 nm or more and 500 nm or less.

Preferably, the maximum light emitting wavelength (Amax) of the compound of Chemical Formula 1 and the second compound may each be in a range of 400 nm or more and 485 nm or less, or 400 nm or more and 470 nm or less. When such a configuration is satisfied, the light emitting layer may emit blue light.

The electron transport layer is a layer which accepts electrons from an electron injection layer and transports the electrons to a light emitting layer, and an electron transport material is suitably a material having high electron mobility which may proficiently accept electrons from a cathode and transfer the electrons to a light emitting layer. Specific examples thereof include: Al complexes of 8-hydroxyquinoline; complexes including Alq3; organic radical compounds; hydroxyflavone-metal complexes, and the like, but are not limited thereto. The electron transport layer may be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

According to an exemplary embodiment of the present specification, the electron transport layer includes a compound of the following Chemical Formula ET-1, but is not limited thereto.

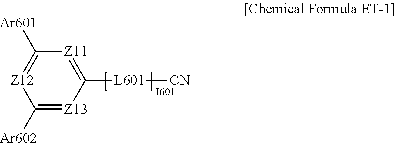

In Chemical Formula ET-1,
at least one of Z1l to Z13 is N, and the others are CH,
L601 is a direct bond; a substituted or unsubstituted arylene group; or a substituted or unsubstituted heteroarylene group,
Ar601 and Ar602 are the same as or different from each other, and are each independently a substituted or unsubstituted aryl group; or a substituted or unsubstituted heteroaryl group, and
1601 is an integer from 1 to 5, and when 1601 is 2 or higher, two or more L601's are the same as or different from each other.

According to an exemplary embodiment of the present specification, L601 is a substituted or unsubstituted monocyclic or polycyclic arylene group having 6 to 30 carbon atoms.

According to an exemplary embodiment of the present specification, L601 is a phenylene group; a biphenylylene group; or a naphthylene group.

According to an exemplary embodiment of the present specification, Ar601 and Ar602 are the same as or different from each other, and are each independently a substituted or unsubstituted monocyclic or polycyclic aryl group having 6 to 30 carbon atoms.

According to an exemplary embodiment of the present specification, Ar601 and Ar602 are a phenyl group.

According to an exemplary embodiment of the present specification, Chemical Formula ET-1 is the following compound.

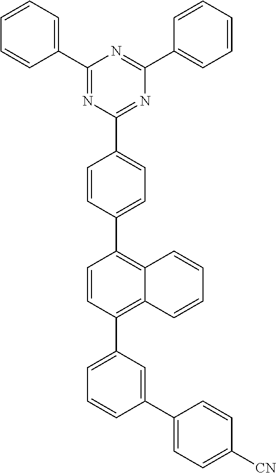 [SMILES: N#Cc1ccc(-c2cccc(-c3ccc(-c4ccc(-c5nc(-c6ccccc6)nc(-c6ccccc6)n5)cc4)c4ccccc34)c2)cc1]

The electron injection layer is a layer which injects electrons from an electrode, and an electron injection material is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The metal complex compound includes 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)-aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato) (o-cresolato)gallium, bis(2-methyl-8-quinolinato) (1-naphtholato)aluminum, bis(2-methyl-8-quinolinato) (2-naphtholato)gallium and the like, but is not limited thereto.

The capping layer is formed to prevent a considerable amount of light from being lost through total reflection of light in the organic light emitting device, and the capping layer has the performance capable of sufficiently protecting the underlying negative electrode and light emitting layer from external moisture penetration or contamination, and has a high refractive index, and thus may prevent light loss caused by total reflection, and materials in the related art can be used without limitation.

According to an exemplary embodiment of the present specification, the capping layer includes a compound of the following Chemical Formula CP-1, but is not limited thereto.

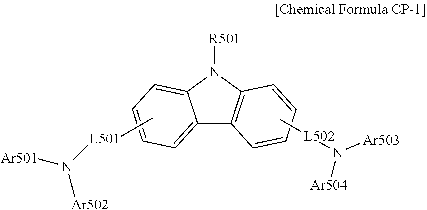

In Chemical Formula CP-1,
L501 and L502 are the same as or different from each other, and are each independently a direct bond; a substituted or unsubstituted arylene group; or a substituted or unsubstituted heteroarylene group, and
R501 and Ar501 to Ar504 are the same as or different from each other, and are each independently a substituted or unsubstituted aryl group; or a substituted or unsubstituted heteroaryl group, or adjacent groups are bonded to each other to form a substituted or unsubstituted ring.

According to an exemplary embodiment of the present specification, L501 and L502 are the same as or different from each other, and are each independently a substituted or unsubstituted monocyclic or polycyclic arylene group having 6 to 30 carbon atoms.

According to an exemplary embodiment of the present specification, L501 and L502 are a phenylene group.

According to an exemplary embodiment of the present specification, R501 and Ar501 to Ar504 are the same as or different from each other, and are each independently a substituted or unsubstituted monocyclic or polycyclic aryl group having 6 to 30 carbon atoms, or are bonded to an adjacent group to form a substituted or unsubstituted monocyclic or polycyclic hetero ring having 2 to 30 carbon atoms.

According to an exemplary embodiment of the present specification, R501 and Ar501 to Ar504 are a phenyl group, or are bonded to an adjacent group to form a carbazole which is unsubstituted or substituted with a phenyl group.

According to an exemplary embodiment of the present specification, Ar501 and L501 are bonded to form a carbazole substituted with a phenyl group.

According to an exemplary embodiment of the present specification, Ar503 is bonded to L502 to form a carbazole substituted with a phenyl group.

According to an exemplary embodiment of the present specification, Chemical Formula CP-1 is the following compound.

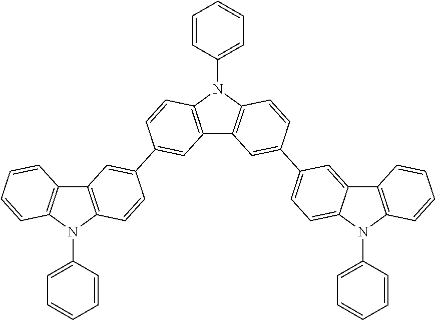 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

The organic light emitting device according to the present specification may be a top-emission type, a bottom-emission type or a dual-emission type depending on the materials used.

The organic light emitting device according to the present specification may be included and used in various electronic devices. For example, the electronic device may be a display panel, a touch panel, a solar module, a lighting device, and the like, and is not limited thereto.

### EXAMPLES

Hereinafter, the present application will be described in detail with reference to Examples for specifically describing the present application. However, the Examples according to the present application may be modified in various forms, and it is not interpreted that the scope of the present application is limited to the Examples described in detail below. The Examples of the present application are provided for more completely explaining the present application to the person with ordinary skill in the art.

### [Preparation Example 1] Synthesis of Formula 1

### 1-1) Synthesis of Chemical Formula A1

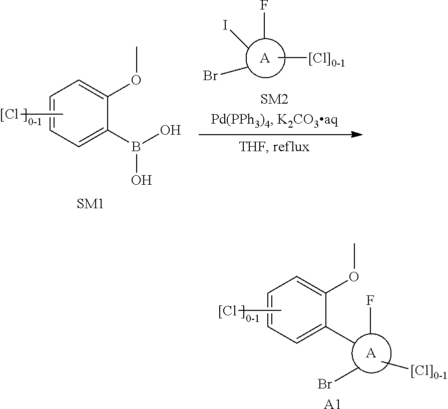

Here, Ring A is a monocyclic or polycyclic hydrocarbon ring.

After SM1 (1 eq) and SM2 (1.1 eq) were added to tetrahydrofuran (THF) (excess), a 2 M aqueous potassium carbonate solution (30 volume ratio compared to THF) was added thereto, and tetrakis triphenyl-phosphino palladium (Pd(PPh₃)₄, 2 mol %) was added thereto, and then the resulting mixture was stirred under heating at 85° C. for 10 hours. After the temperature was lowered to room temperature and the reaction was terminated, layers were separated by removing the aqueous potassium carbonate solution, and the residue was purified using a column with hexane and ethyl acetate to prepare Chemical Formula A1 (A1-1 to A1-17).

A1-1 to A1-17 in [Table A1] were synthesized in the same manner as in the method of synthesizing Chemical Formula A1, except that SM41 and SM42 were changed.

TABLE A1

| | | | | | MS |
|---|---|---|---|---|---|
| | SM1 | SM2 | A1 | Yield | [M + H]+ |
| A1-1 | 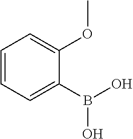 | 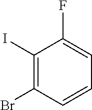 | 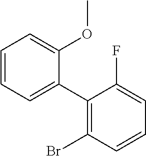 | 68% | 282.12 |
| A1-2 | 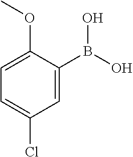 | 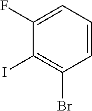 | 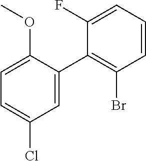 | 67% | 316.57 |
| A1-3 | 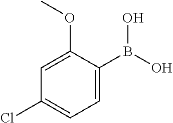 | 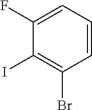 | 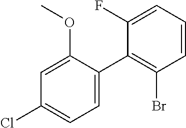 | 65% | 316.57 |
| A1-4 | 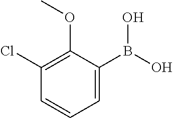 | 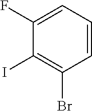 | 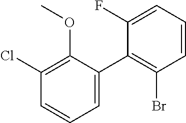 | 66% | 316.57 |
| A1-5 | 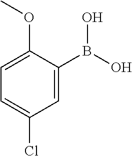 | 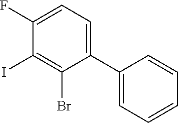 | 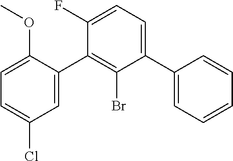 | 64% | 392.66 |
| A1-6 | 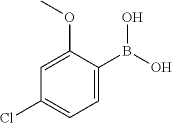 | 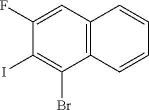 | 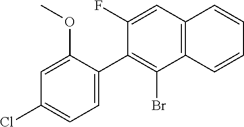 | 68% | 366.63 |
| A1-7 | 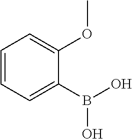 | 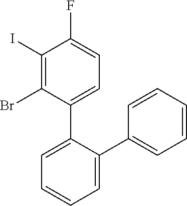 | 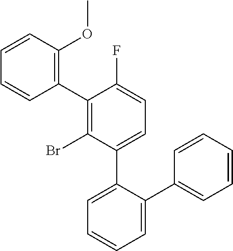 | 58% | 434.32 |
| A1-8 | 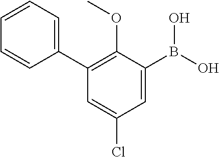 | 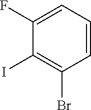 | 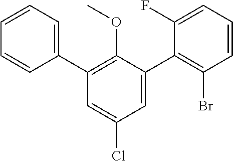 | 55% | 392.66 |
| A1-9 | 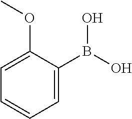 | 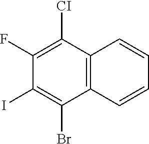 | 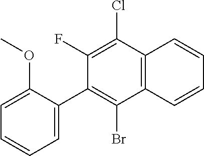 | 40% | 366.63 |
| A1-10 | 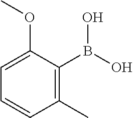 | 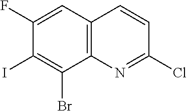 | 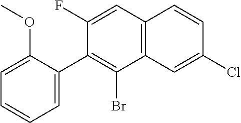 | 38% | 366.63 |
| A1-11 | 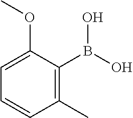 | 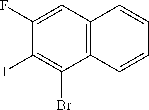 | 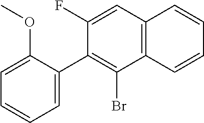 | 55% | 332.18 |
| A1-12 | 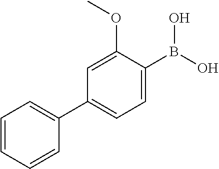 | 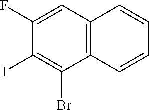 | 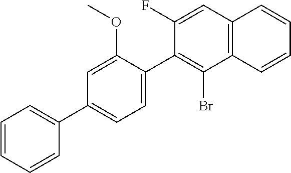 | 51% | 408.28 |
| A1-13 | 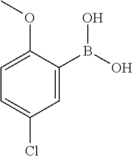 | 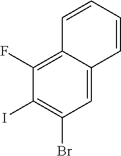 | 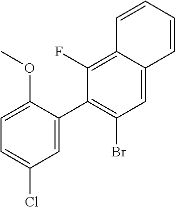 | 37% | 366.63 |
| A1-14 | 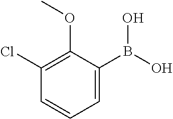 | 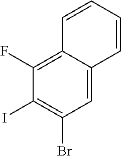 | 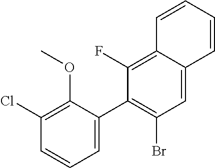 | 40% | 366.63 |
| A1-15 | 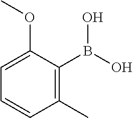 | 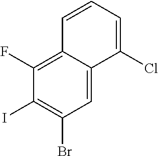 | 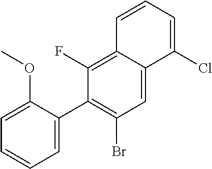 | 38% | 366.63 |
| A1-16 | 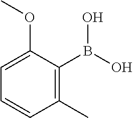 | 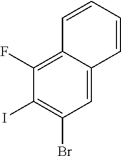 | 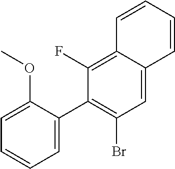 | 56% | 332.18 |
| A1-17 | 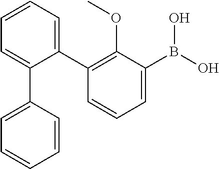 | 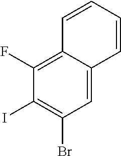 | 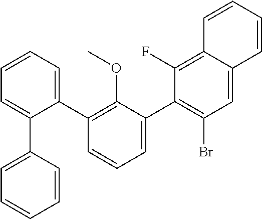 | 41% | 484.38 |

### 1-2) Synthesis of Chemical Formula A2

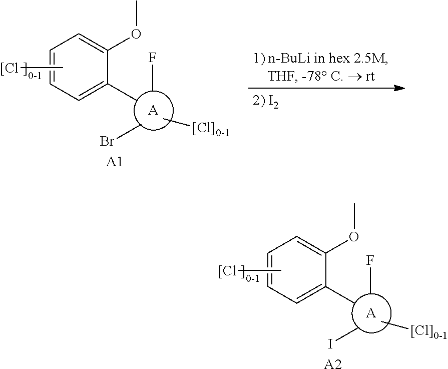

Here, Ring A is a monocyclic or polycyclic hydrocarbon ring.

Compound A1 dissolved in tetrahydrofuran (excess) was dissolved under nitrogen conditions, the compound was cooled to −78° C. 2.5 M n-BuLi (1.2 eq) was added dropwise thereto, the resulting mixture was stirred for 1 hour, and then iodine (1.1 eq) was added thereto, and the resulting mixture was stirred for 10 hours. After the temperature was increased to room temperature (rt), a 1 N hydrogen chloride solution was added dropwise thereto, and the organic layer was extracted, followed by further extraction twice with water. After the solvent was removed, the residue was columned with hexane and ethyl acetate to prepare Chemical Formula A2 (A2-1 to A2-17).

A2-1 to A2-17 in [Table A2] were synthesized in the same manner as in the method of synthesizing Chemical Formula A2, except that A1 was changed.

TABLE A2

| | | | | MS |
|---|---|---|---|---|
| | A1 | A2 | Yield | [M + H]+ |
| A2-1 | A1-1 | 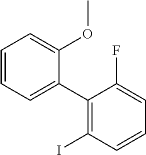 | 50% | 329.12 |
| A2-2 | A1-2 | 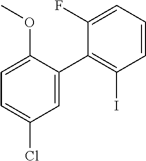 | 52% | 363.57 |
| A2-3 | A1-3 | 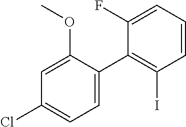 | 50% | 363.57 |
| A2-4 | A1-4 | 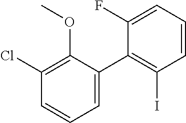 | 51% | 363.57 |
| A2-5 | A1-5 | 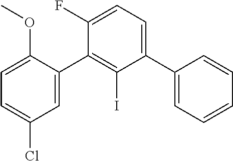 | 48% | 439.66 |
| A2-6 | A1-6 | 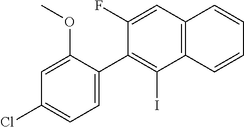 | 45% | 413.63 |
| A2-7 | A1-7 | 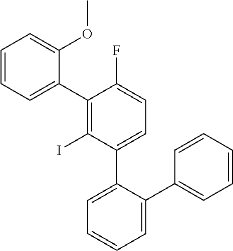 | 41% | 481.32 |
| A2-8 | A1-8 | 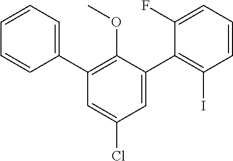 | 40% | 439.66 |
| A2-9 | A1-9 | 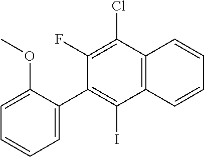 | 39% | 413.63 |
| A2-10 | A1-10 | 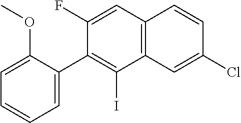 | 35% | 413.63 |
| A2-11 | A1-11 | 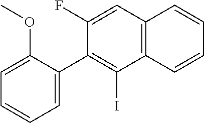 | 40% | 379.18 |
| A2-12 | A1-12 | 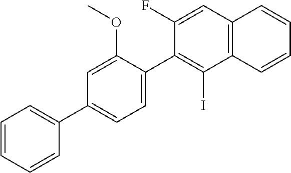 | 38% | 455.28 |
| A2-13 | A1-13 | 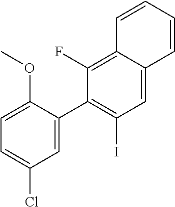 | 36% | 413.63 |
| A2-14 | A1-14 | 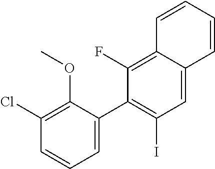 | 35% | 413.63 |
| A2-15 | A1-15 | 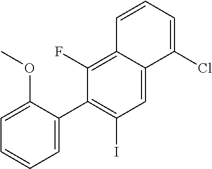 | 39% | 413.63 |
| A2-16 | A1-16 | 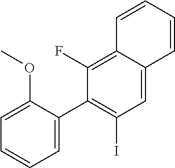 | 40% | 379.18 |
| A2-17 | A1-17 | 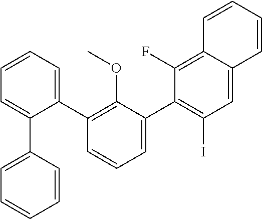 | 39% | 531.38 |

### 1-3) Synthesis of Chemical Formula A3

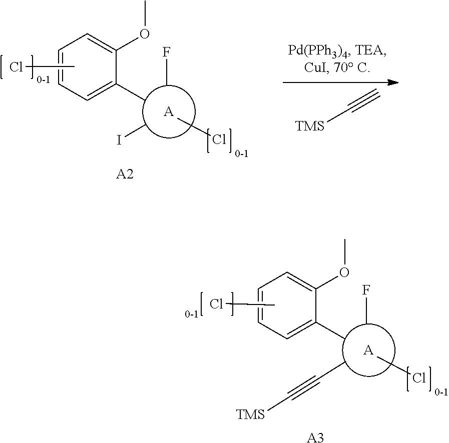

Here, Ring A is a monocyclic or polycyclic hydrocarbon ring.

Compound A2 (1 eq), trimethylsilylacetylene (TMS-≡, 5 eq), tetrakistriphenyl-phosphinopalladium (Pd(PPh₃)₄, 0.1 eq), copper iodide (CuI, 1 eq) and triethylamine (TEA, excess) were put into a container, and the resulting mixture was stirred under heating at 70° C. for 5 hours. After the reaction was terminated, the resulting product was cooled to room temperature, allowed to pass through a filter filled with celite/silica gel, and washed with chloroform, the solvent was removed, and the residue was columned with hexane and ethyl acetate to prepare Chemical Formula A3 (A3-1 to A3-17).

A3-1 to A3-17 in [Table A3] were synthesized in the same manner as in the method of synthesizing Chemical Formula A3, except that A2 was changed.

TABLE A3

| | | | | MS |
|---|---|---|---|---|
| | A2 | A3 | yield | [M + H]+ |
| A3-1 | A2-1 | 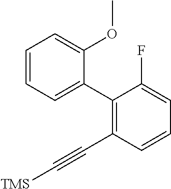 | 65% | 299.43 |
| A3-2 | A2-2 | 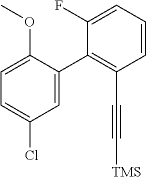 | 64% | 333.87 |
| A3-3 | A2-3 | 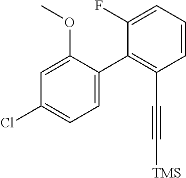 | 63% | 333.87 |
| A3-4 | A2-4 | 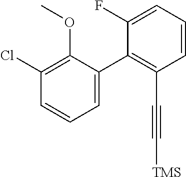 | 64% | 333.87 |
| A3-5 | A2-5 | 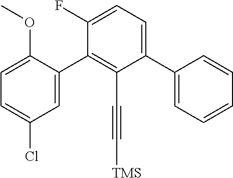 | 58% | 409.97 |
| A3-6 | A2-6 | 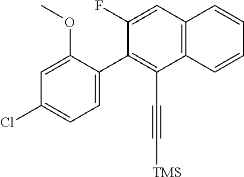 | 52% | 382.93 |
| A3-7 | A2-7 | 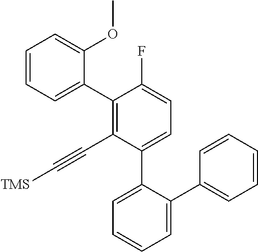 | 48% | 451.63 |
| A3-8 | A2-8 | 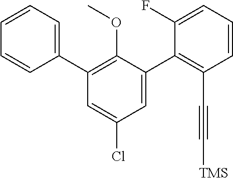 | 50% | 409.97 |
| A3-9 | A2-9 | 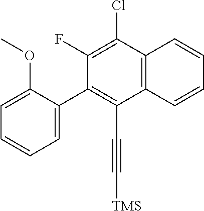 | 51% | 383.93 |
| A3-10 | A2-10 | 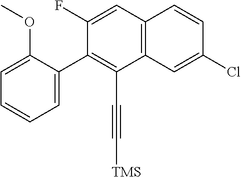 | 48% | 383.93 |
| A3-11 | A2-11 | 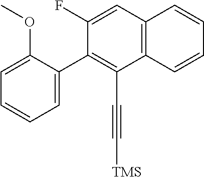 | 49% | 349.49 |
| A3-12 | A2-12 | 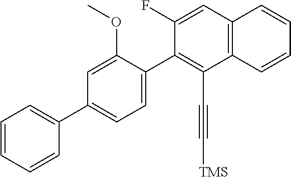 | 46% | 425.59 |
| A3-13 | A2-13 | 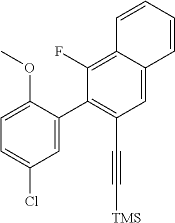 | 50% | 383.93 |
| A3-14 | A2-14 | 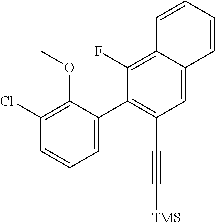 | 45% | 383.93 |
| A3-15 | A2-15 | 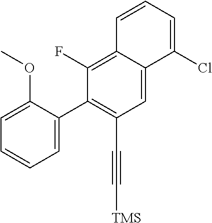 | 49% | 383.93 |
| A3-16 | A2-16 | 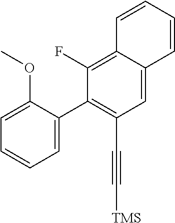 | 47% | 349.49 |
| A3-17 | A2-17 | 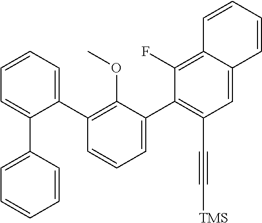 | 45% | 501.69 |

### 1-4) Synthesis of Chemical Formula A4

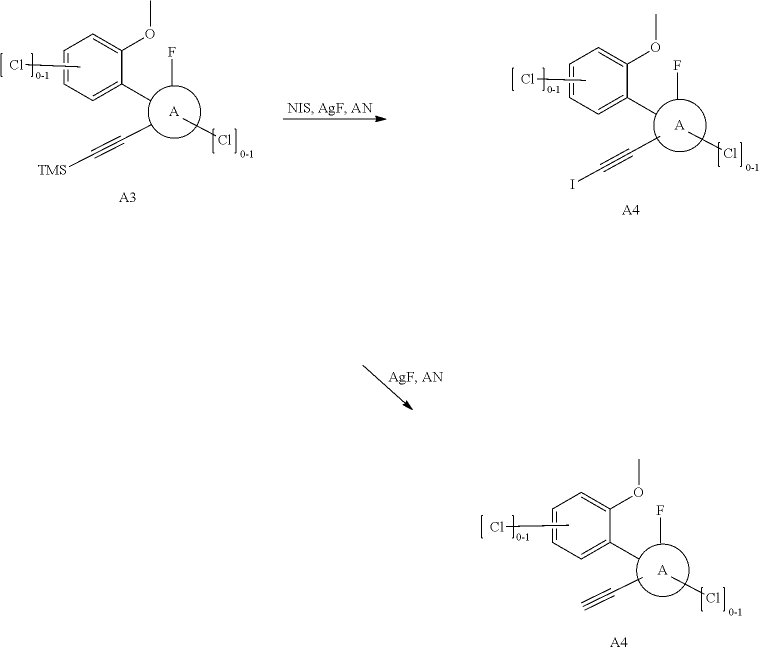

Here, Ring A is a monocyclic or polycyclic hydrocarbon ring.

Compound A3 (1 eq) was dissolved in acetonitrile (AN, excess), silver fluoride (1.4 eq) was added thereto, the resulting mixture was stirred for 30 minutes, and then N-iodosuccinimide (NIS, 1.4 eq) was added thereto. After the mixture was stirred at room temperature for 2 hours, it was confirmed that the reaction was terminated, and then the resulting product was filtered and washed with acetonitrile. The solvent was removed by distillation under reduced pressure, and the residue was columned with hexane and ethyl acetate to prepare the Chemical Formula A4 (A4-1 to A4-17).

A4-1 to A4-17 in [Table A4] were synthesized in the same manner as in the method of synthesizing Chemical Formula A4, except that A3 was changed.

TABLE A4

| | | | | | MS |
|---|---|---|---|---|---|
| | A3 | Reactant | A4 | yield | [M + H]+ |
| A4-1 | A3-1 | NIS, AgF, AN | 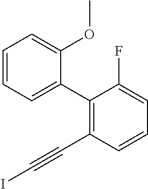 | 69% | 353.15 |
| A4-2 | A3-2 | AgF, AN | 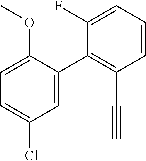 | 68% | 387.59 |
| A4-3 | A3-3 | AgF, AN | 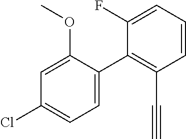 | 66% | 387.59 |
| A4-4 | A3-4 | AgF, AN | 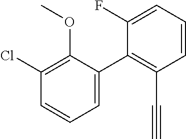 | 70% | 387.59 |
| A4-5 | A3-5 | AgF, AN | 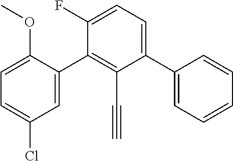 | 68% | 463.69 |
| A4-6 | A3-6 | AgF, AN | 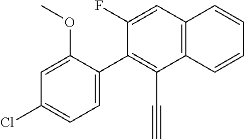 | 63% | 437.65 |
| A4-7 | A3-7 | NIS, AgF, AN | 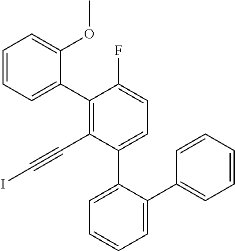 | 60% | 505.34 |
| A4-8 | A3-8 | AgF, AN | 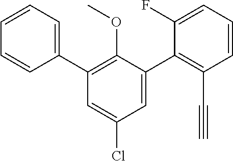 | 61% | 463.69 |
| A4-9 | A3-9 | AgF, AN | 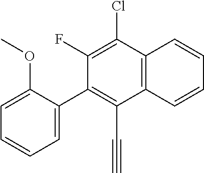 | 58% | 311.75 |
| A4-10 | A3-10 | AgF, AN | 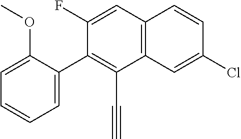 | 55% | 311.75 |
| A4-11 | A3-11 | AgF, AN | 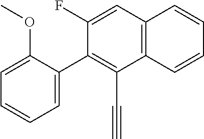 | 63% | 277.31 |
| A4-12 | A3-12 | AgF, AN | 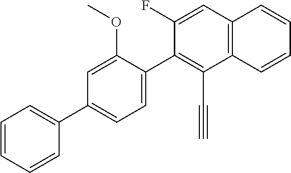 | 53% | 353.41 |
| A4-13 | A3-13 | AgF, AN | 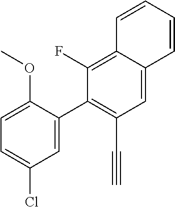 | 57% | 311.75 |
| A4-14 | A3-14 | AgF, AN | 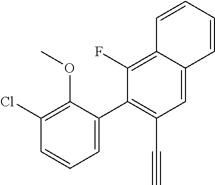 | 56% | 311.75 |
| A4-15 | A3-15 | AgF, AN | 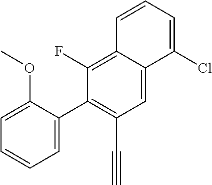 | 55% | 311.75 |
| A4-16 | A3-16 | AgF, AN | 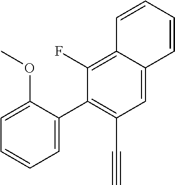 | 59% | 277.31 |
| A4-17 | A3-17 | AgF, AN | 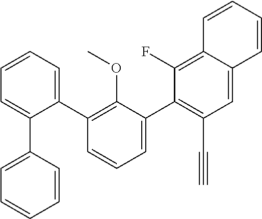 | 46% | 429.51 |

### 1-5) Synthesis of Chemical Formula AS

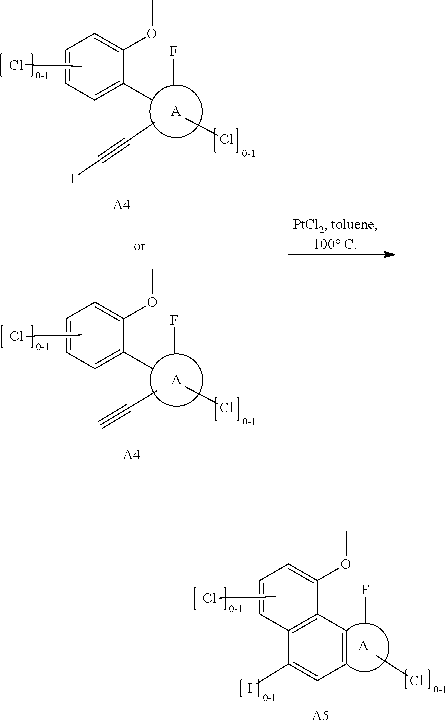

Here, Ring A is a monocyclic or polycyclic hydrocarbon ring.

Compound A4 (1 eq), platinum chloride (0.05 eq), and toluene (excess) were put into a container, the resulting mixture was stirred at 100° C. for 3 hours, it was confirmed that the reaction was terminated, and then the resulting product was cooled to room temperature. The product was filtered with a celite/silica gel pad, the solvent was removed by distillation under reduced pressure, and then the residue was columned with hexane and ethyl acetate to prepare Chemical Formula AS (A5-1 to AS-17).

A5-1 to A5-17 in [Table A5] were synthesized in the same manner as in the method of synthesizing Chemical Formula AS, except that A4 was changed.

TABLE A5

| | | | | MS |
|---|---|---|---|---|
| | A4 | A5 | yield | [M + H]+ |
| A5-1 | A4-1 | 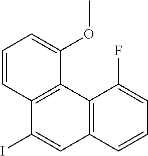 | 68% | 353.15 |
| A5-2 | A4-2 | 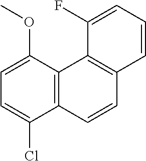 | 65% | 261.69 |
| A5-3 | A4-3 | 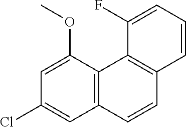 | 66% | 261.69 |
| A5-4 | A4-4 | 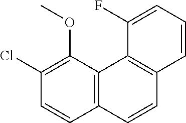 | 63% | 261.69 |
| A5-5 | A4-5 | 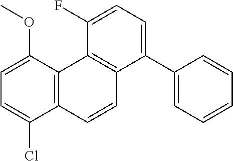 | 64% | 337.79 |
| A5-6 | A4-6 | 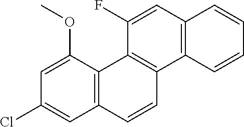 | 60% | 311.75 |
| A5-7 | A4-7 | 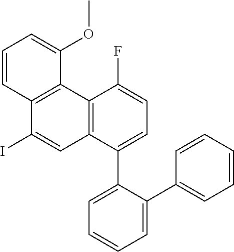 | 56% | 505.34 |
| A5-8 | A4-8 | 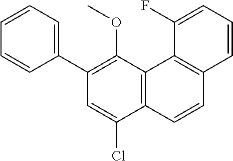 | 57% | 337.79 |
| A5-9 | A4-9 | 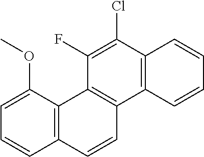 | 49% | 311.75 |
| A5-10 | A4-10 | 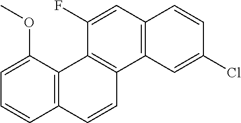 | 53% | 311.75 |
| A5-11 | A4-11 | 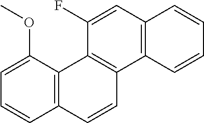 | 56% | 277.31 |
| A5-12 | A4-12 | 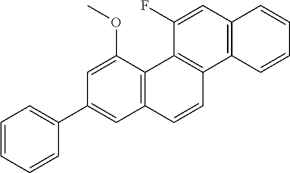 | 51% | 353.41 |
| A5-13 | A4-13 | 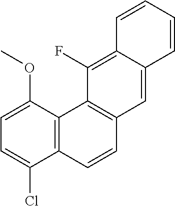 | 55% | 311.75 |
| A5-14 | A4-14 | 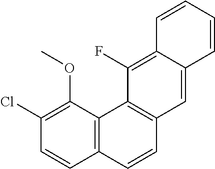 | 49% | 311.75 |
| A5-15 | A4-15 | 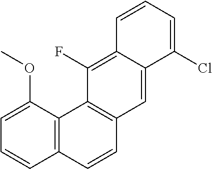 | 48% | 311.75 |
| A5-16 | A4-16 | 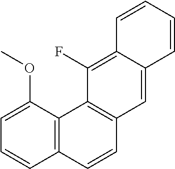 | 56% | 277.31 |
| A5-17 | A4-17 | 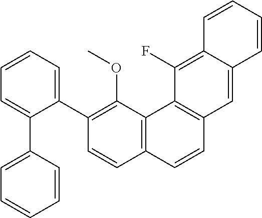 | 50% | 429.51 |

### 1-6) Synthesis of Chemical Formula A6

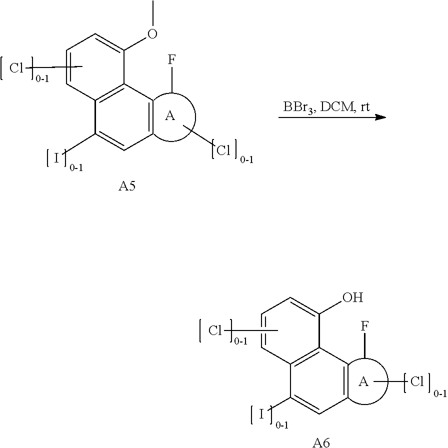

Here, Ring A is a monocyclic or polycyclic hydrocarbon ring.

After Compound A5 (1 eq) was dissolved in methylene chloride (excess), boron tribromide (BBr3, 1.4 eq) was added dropwise thereto, and the resulting mixture was stirred for 2 hours. After the reaction was completed, the reactant was added to water, extraction was performed, and then the extract was additionally washed with water, the solvent was removed by distillation under reduced pressure, hexane was added thereto, and the resulting mixture was stirred for 3 hours and then filtered to prepare Chemical Formula A6 (A6-1 to A6-17).

A6-1 to A6-17 in [Table A6] were synthesized in the same manner as in the method of synthesizing Chemical Formula A6, except that AS was changed.

TABLE A6

| | | | | MS |
|---|---|---|---|---|
| | A5 | A6 | Yield | [M + H]+ |
| A6-1 | A5-1 | 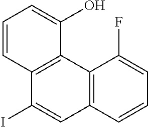 | 75% | 397.29 |
| A6-2 | A5-2 | 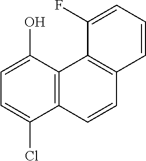 | 74% | 247.67 |
| A6-3 | A5-3 | 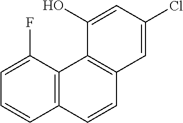 | 73% | 373.56 |
| A6-4 | A5-4 | 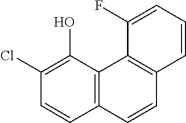 | 70% | 408.00 |
| A6-5 | A5-5 | 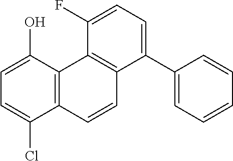 | 74% | 323.76 |
| A6-6 | A5-6 | 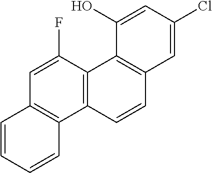 | 71% | 297.73 |
| A6-7 | A5-7 | 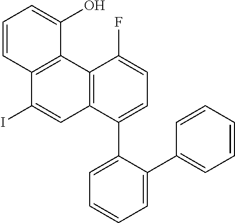 | 67% | 491.32 |
| A6-8 | A5-8 | 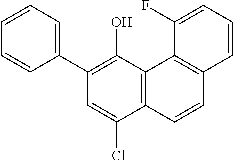 | 69% | 323.76 |
| A6-9 | A5-9 | 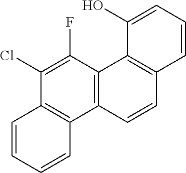 | 65% | 297.73 |
| A6-10 | A5-10 | 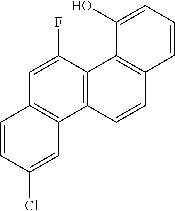 | 60% | 297.73 |
| A6-11 | A5-11 | 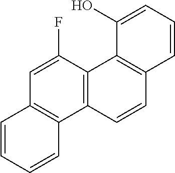 | 61% | 263.28 |
| A6-12 | A5-12 | 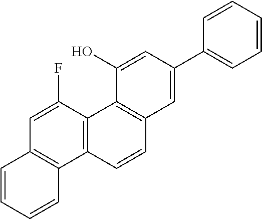 | 53% | 339.38 |
| A6-13 | A5-13 | 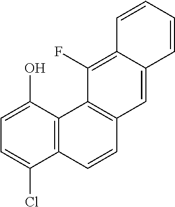 | 63% | 297.73 |
| A6-14 | A5-14 | 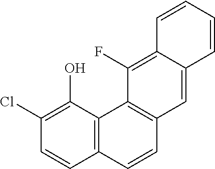 | 59% | 297.73 |
| A6-15 | A5-15 | 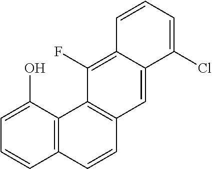 | 60% | 297.73 |
| A6-16 | A5-16 | 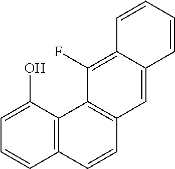 | 58% | 263.28 |
| A6-17 | A5-17 | 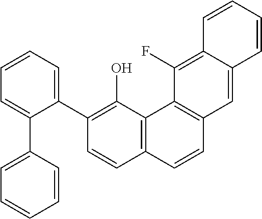 | 43% | 415.48 |

### 1-7) Synthesis of Chemical Formula A7

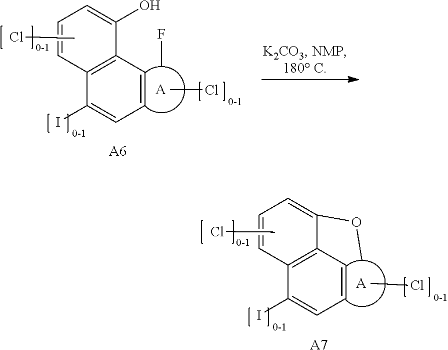

Here, Ring A is a monocyclic or polycyclic hydrocarbon ring.

After Compound A6 (1 eq) and potassium carbonate (2.5 eq) were added to N-methylpyrrolidone (NMP, excess), the mixture was stirred under heating at 180° C. for 3 hours. After it was confirmed that the reaction was terminated, the resulting product was cooled to room temperature, water (excess, two-fold compared to the amount of the input solvent) was added thereto, and then the resulting mixture was filtered, and the filtered solid was washed with water. Thereafter, the washed solid was recrystallized with ethyl acetate and ethanol to prepare Chemical Formula A7 (A7-1 to A7-17).

A7-1 to A7-17 in [Table A7] were synthesized in the same manner as in the method of synthesizing Chemical Formula A7, except that A6 was changed.

TABLE A7

| | | | | MS |
|---|---|---|---|---|
| | A6 | A7 | yield | [M + H]+ |
| A7-1 | A6-1 | 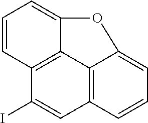 | 75% | 319.11 |
| A7-2 | A6-2 | 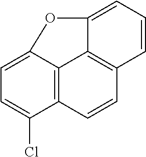 | 73% | 227.66 |
| A7-3 | A6-3 | 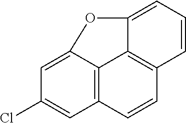 | 70% | 227.66 |
| A7-4 | A6-4 | 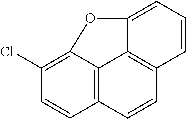 | 74% | 227.66 |
| A7-5 | A6-5 | 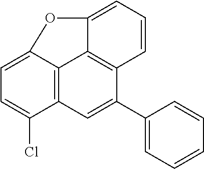 | 75% | 303.76 |
| A7-6 | A6-6 | 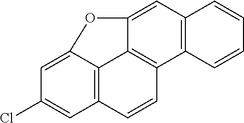 | 70% | 277.72 |
| A7-7 | A6-7 | 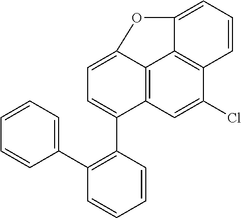 | 68% | 379.86 |
| A7-8 | A6-8 | 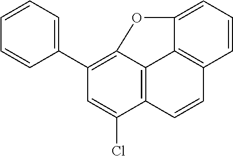 | 65% | 303.76 |
| A7-9 | A6-9 | 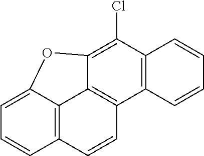 | 54% | 277.72 |
| A7-10 | A6-10 | 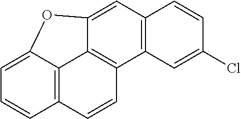 | 55% | 277.72 |
| A7-11 | A6-11 | 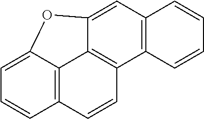 | 50% | 243.28 |
| A7-12 | A6-12 | 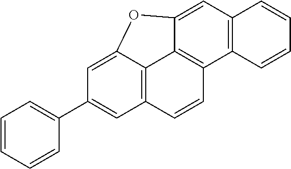 | 52% | 319.38 |
| A7-13 | A6-13 | 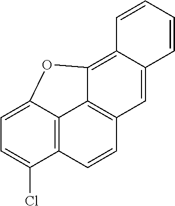 | 56% | 277.72 |
| A7-14 | A6-14 | 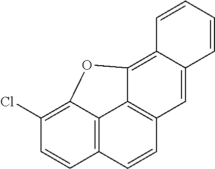 | 59% | 277.72 |
| A7-15 | A6-15 | 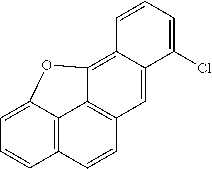 | 53% | 277.72 |
| A7-16 | A6-16 | 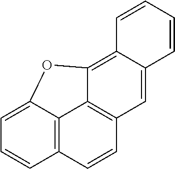 | 55% | 243.28 |
| A7-17 | A6-17 | 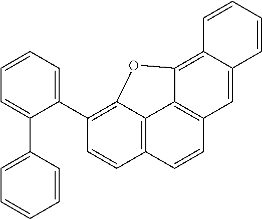 | 52% | 395.48 |

### 1-7B) Synthesis of Chemical Formula A7B

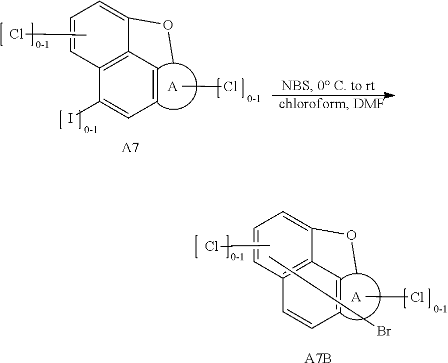

Here, Ring A is a monocyclic or polycyclic hydrocarbon ring.

Compound A7 (1 eq.) was dissolved in chloroform (excess) and stirred, after which N-bromosuccinimide (1.05 eq.), dissolved in dimethylformamide (3 times relative to N-bromosuccinimide), was added dropwise at 0° C. The temperature was then allowed to stabilize to room temperature, and the mixture was stirred for 5 hours. After confirming completion of the reaction, a saturated aqueous solution of sodium sulfate was added, and the mixture was extracted three times. The mixture was extracted three times with water, and the product was recrystallized from ethyl acetate and hexane to obtain the compound of Formula A7B (A7B-1 to A7B-4).

A7B-1 to A7B-4 in [Table A7B] were synthesized in the same manner as in the method of synthesizing Chemical Formula A7B, except that A7 was changed.

TABLE A7B

| | | | | MS |
|---|---|---|---|---|
| | A6 | A7B | yield | [M + H]+ |
| A7B-1 | A7-11 | 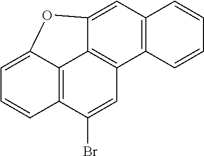 | 35% | 322.17 |
| A7B-2 | A7-12 | 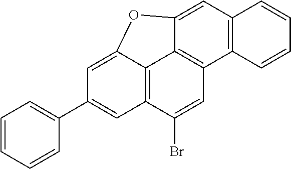 | 34% | 398.27 |
| A7B-3 | A7-16 | 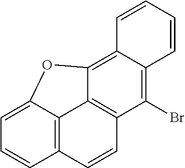 | 58% | 322.17 |
| A7B-4 | A7-17 | 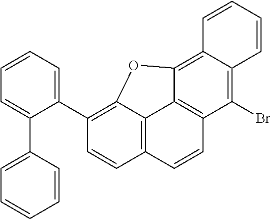 | 55% | 474.37 |

### 1-8) Synthesis of Chemical Formula A8

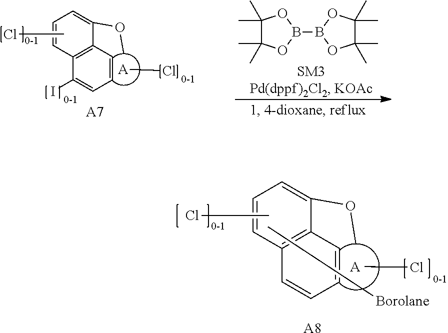

Here, Ring A is a monocyclic or polycyclic hydrocarbon ring.

Compound A7 (1 eq) and 4,4,4′, 4′, 5,5,5′, 5′-octamethyl-2,2′-bi(1,3,2-dioxaborolane) (SM3, 1.3 eq) were put into 1,4-dioxane (12-fold compared to A7 <mass ratio>), potassium acetate (KOAc, 3 eq) was added thereto, and the resulting mixture was stirred and refluxed. Bis(diphenylphosphino)-ferrocene dichloropalladium (Pd(dppf)₂Cl₂, 0.05 eq) was stirred in 1,4-dioxane for 5 minutes, and then added thereto, and after 2 hours, it was confirmed that the reaction was terminated, and then resulting product was cooled to room temperature. After ethanol and water were added thereto, the resulting mixture was filtered and purified by recrystallization with ethyl acetate and ethanol to prepare Chemical Formulae A8 (A8-1 to A8-8).

A8-1 to A8-8 in [Table A8] were synthesized in the same manner as in the method of synthesizing Chemical Formula A8, except that A7 was changed.

TABLE A8

| | | | | MS |
|---|---|---|---|---|
| | A7 | A8 | yield | [M+H] + |
| A8-1 | A7-1 | 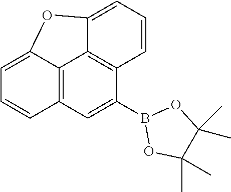 | 85% | 319.18 |
| A8-2 | A7-2 | 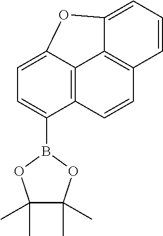 | 83% | 353.62 |
| A8-3 | A7-3 | 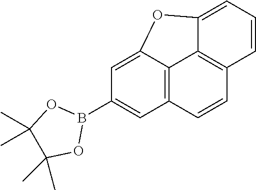 | 80% | 353.62 |
| A8-4 | A7-4 | 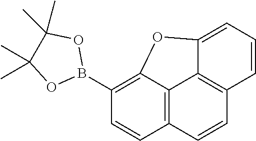 | 83% | 353.62 |
| A8-5 | A7-5 | 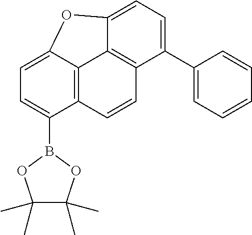 | 84% | 395.28 |
| A8-6 | A7-6 | 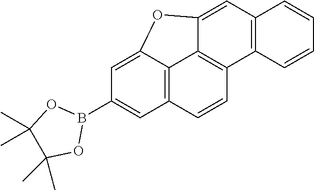 | 80% | 369.24 |
| A8-7 | A7-7 | 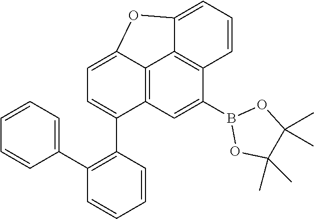 | 73% | 471.38 |
| A8-8 | A7-8 | 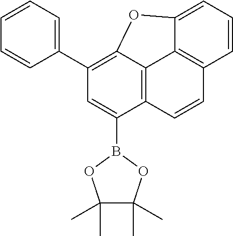 | 75% | 395.28 |

### 2-1) Synthesis of B1a

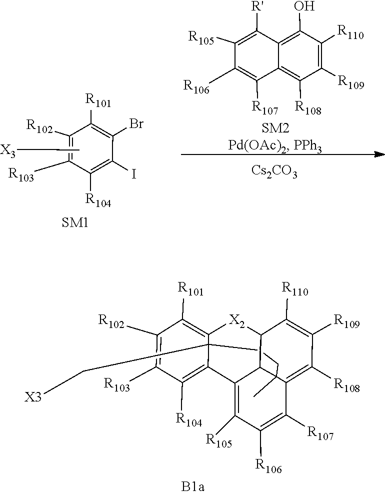

Here, R₁₀₁ to R₁₁₀ and X₂ are the same as those in Chemical Formula 1, X₃ in SM1 is a halogen group that is one of R₁₀₁ to R₁₀₄, X₃ in B1a is a halogen group that is one of R₁₀₁ to R₁₁₀, and R′ is hydrogen or a leaving group.

SM1 (1 eq), SM2 (1.1 eq), palladium acetate (Pd(OAc)₂, 0.05 eq), triphenylphosphine (PPh₃, 0.2 eq), and cesium carbonate (Cs₂CO₃, 4 eq) were added to dimethylformamide (excess) and stirred at 140° C. for 48 hours. After confirming completion of the reaction, the mixture was cooled to room temperature and extracted three times with ethyl acetate and water. The crude product was purified by silica gel column chromatography using ethyl acetate and hexane to afford the compound of Formula B1a (B1a-1 to B1a-7).

Except for substituting SM41 and SM42 in the synthesis of Formula B1a, the same procedure was applied to synthesize B1a-1 to B1a-7 as shown in [Table B1a].

TABLE Bla

| | | | | | MS |
|---|---|---|---|---|---|
| | SM1 | SM2 | Bla | yield | [M + H]+ |
| B1a-1 | 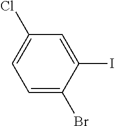 | 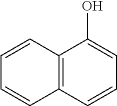 | 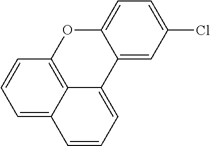 [SMILES: Clc1ccc2c(c1)-c1cccc3cccc(c13)O2] | 48% | 253.70 |
| B1a-2 | 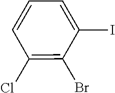 | 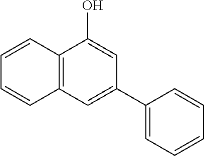 | 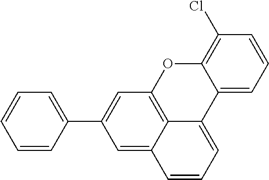 [SMILES: Clc1cccc2c1Oc1cc(-c3ccccc3)cc3cccc-2c13] | 50% | 329.80 |
| B1a-3 | 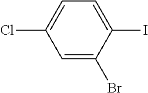 | 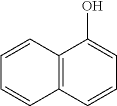 | 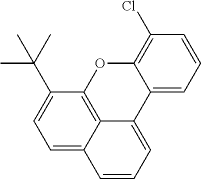 [SMILES: CC(C)(C)c1ccc2cccc3c2c1Oc1c(Cl)cccc1-3] | 52% | 253.70 |
| B1a-4 | 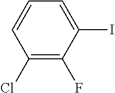 | 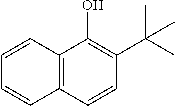 | 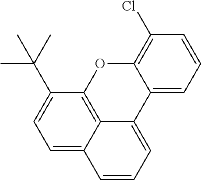 [SMILES: CC(C)(C)c1ccc2cccc3c2c1Oc1c(Cl)cccc1-3] | 50% | 309.81 |
| B1a-5 | 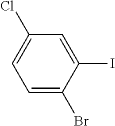 | 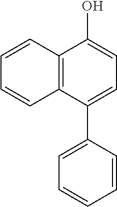 | 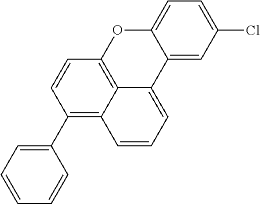 [SMILES: Clc1ccc2c(c1)-c1cccc3c(-c4ccccc4)ccc(c13)O2] | 43% | 329.80 |
| B1a-6 | 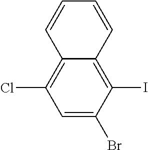 | 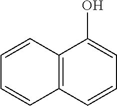 | 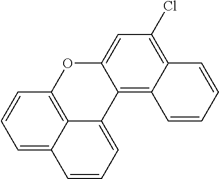 [SMILES: Clc1cc2c(c3ccccc13)-c1cccc3cccc(c13)O2] | 46% | 303.76 |
| B1a-7 | 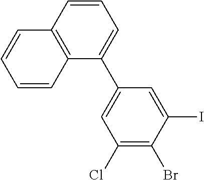 | 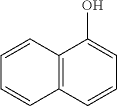 | 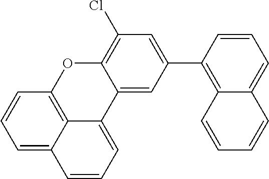 [SMILES: Clc1cc(-c2cccc3ccccc23)cc2c1Oc1cccc3cccc-2c13] | 44% | 379.86 |

### 2-2) Synthesis of B1b

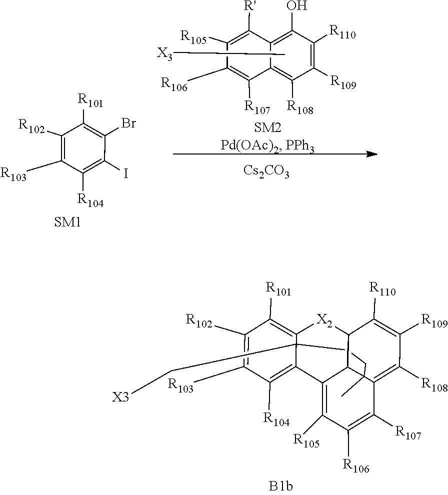

Here, R₁₀₁ to R₁₁₀ and X₂ are the same as those in Chemical Formula 1, R′ is hydrogen or a leaving group, X₃ in SM2 is a halogen group that is one of R′ and R₁₀₅ to R₁₁₀, and X₃ in B1b is a halogen group that is one of R₁₀₁ to R₁₁₀.

Except for substituting SM1 and SM2 in the synthesis of Formula B1b, the same procedure was applied to synthesize B1b-to B1b-5 as shown in [Table B1b].

TABLE B1b

| | | | | | MS |
|---|---|---|---|---|---|
| | SM1 | SM2 | B1b | yield | [M + H]+ |
| B1b-1 | 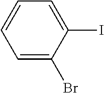 | 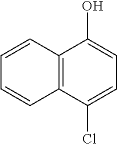 | 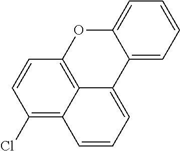 [SMILES: Clc1ccc2c3c(cccc13)-c1ccccc1O2] | 48% | 253.70 |
| B1b-2 | 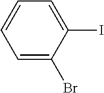 | 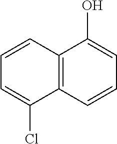 | 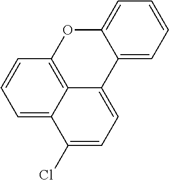 [SMILES: Clc1ccc2c3c(cccc13)Oc1ccccc1-2] | 46% | 253.70 |
| B1b-3 | 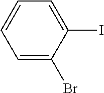 | 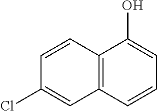 | 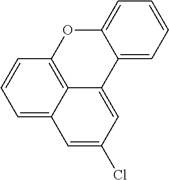 [SMILES: Clc1cc2c3c(cccc3c1)Oc1ccccc1-2] | 50% | 253.70 |
| B1b-4 | 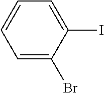 | 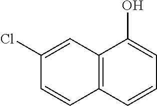 | 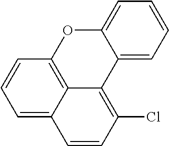 [SMILES: Clc1ccc2cccc3c2c1-c1ccccc1O3] | 48% | 253.70 |
| B1b-5 | 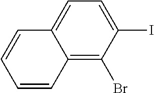 | 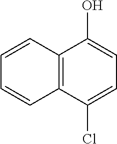 | 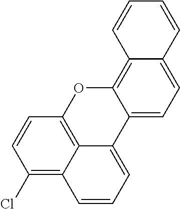 [SMILES: Clc1ccc2c3c(cccc13)-c1ccc3ccccc3c1O2] | 47% | 303.76 |

### 2-3) Synthesis of B2

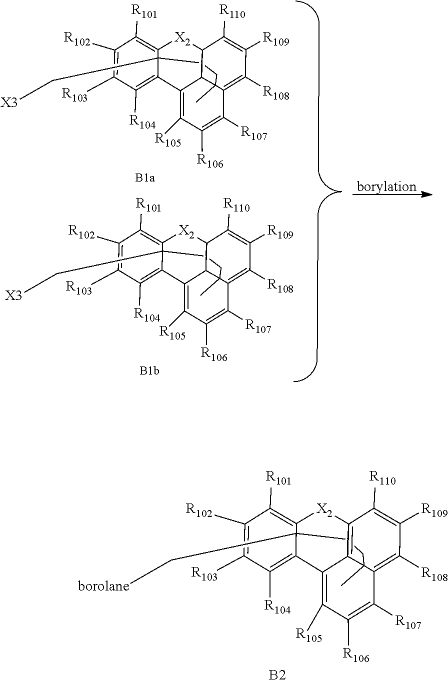

SM1 (one of Chemical Formulae B1a or Bib, 1 eq.) and pinacolato diboron (1.3 eq.) were added to 1,4-dioxane (12 times by weight relative to SM1), and potassium acetate (3 eq.) was further added, followed by stirring and refluxing. Bis(diphenylphosphino)ferrocene dichloropalladium (0.05 eq.) was stirred in 1,4-dioxane for 5 minutes and then added to the reaction mixture. After 2 hours, completion of the reaction was confirmed, and the mixture was cooled to room temperature. Ethanol and water were added, the mixture was filtered, and the crude product was purified by recrystallization from ethyl acetate and ethanol to afford the compound of Formula B2 (B2-1 to B2-11).

Except for substituting SM1 in the synthesis of Formula B2, the same procedure was applied to synthesize B2-1 to B2-11 as shown in [Table B2].

TABLE B2

| | | | | MS |
|---|---|---|---|---|
| | SM1 | B2 | yield | [M + H]+ |
| B2-1 | B1a-1 | 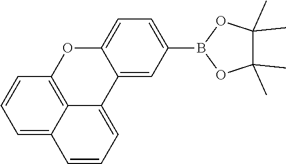 | 75% | 345.22 |
| B2-2 | B1b-1 | 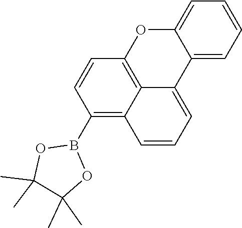 | 80% | 345.22 |
| B2-3 | B1a-2 | 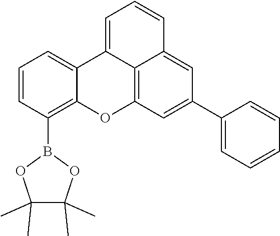 | 74% | 421.32 |
| B2-4 | B1a-3 | 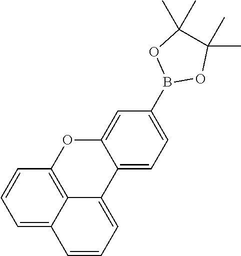 | 76% | 345.22 |
| B2-5 | B1a-4 | 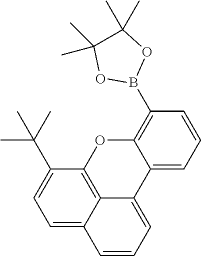 | 79% | 401.33 |
| B2-6 | B1b-2 | 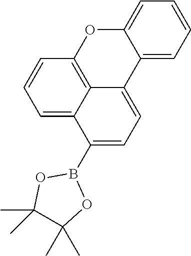 | 80% | 345.22 |
| B2-7 | B1b-3 | 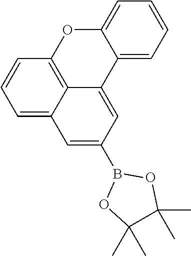 | 71% | 345.22 |
| B2-8 | B1a-5 | 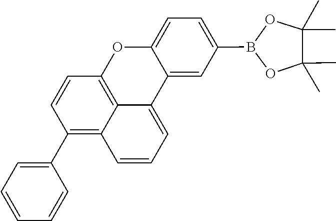 | 76% | 421.32 |
| B2-9 | B1b-4 | 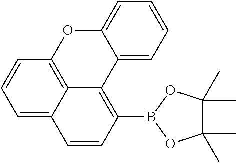 | 77% | 345.22 |
| B2-10 | B1b-5 | 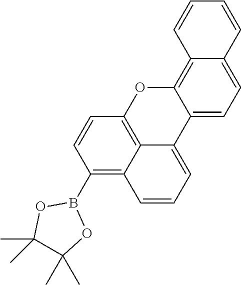 | 76% | 395.28 |
| B2-11 | B1a-7 | 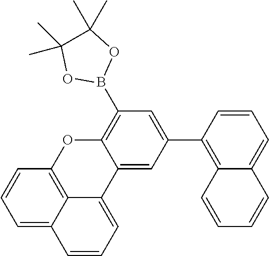 | 78% | 471.38 |

### 3-1) Synthesis of Int.

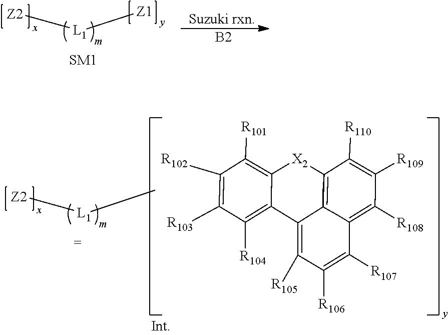

Here, R₁₀₁ to R₁₁₀, X₂, L₁, and m are the same as those in Chemical Formula 1, Z1 and Z2 are each a halogen group, x is 1 or 2, and y is 1 or 2.

SM1 (1 eq.) and B2 (1.1 eq. or 2.1 eq., depending on the number of Z1, i.e., y) were added to tetrahydrofuran (THF, excess), followed by the addition of 2 M aqueous potassium carbonate solution (30 volumes relative to THF). Tetrakis(triphenylphosphine)palladium (2 mol %) was then added, and the mixture was stirred at 85° C. for 10 hours. After cooling to room temperature and confirming completion of the reaction, the aqueous potassium carbonate solution was removed, the layers were separated, and the crude product was purified by column chromatography using hexane and ethyl acetate to afford the compound of Formula int. (int. 1 to int. 12).

Except for substituting SM1 and B2 in the synthesis of Formula int., the same procedure was applied to synthesize int. 1 to int. 12 as shown in [Table int.].

TABLE int.

| | | | | | MS |
|---|---|---|---|---|---|
| | SM1 | B2 | int. | yield | [M + H]+ |
| Int. 1 | 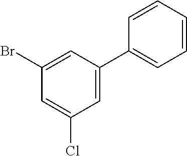 | B2-1 | 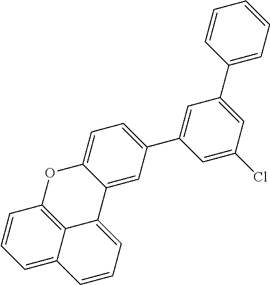 [SMILES: Clc1cc(-c2ccccc2)cc(-c2ccc3c(c2)-c2cccc4cccc(c24)O3)c1] | 65% | 405.89 |
| Int. 2 | 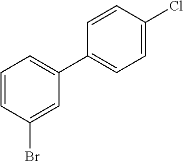 | B2-2 | 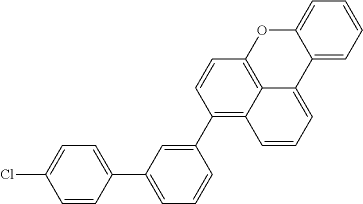 [SMILES: Clc1ccc(-c2cccc(-c3ccc4c5c(cccc35)-c3ccccc3O4)c2)cc1] | 68% | 455.95 |
| int. 3 | 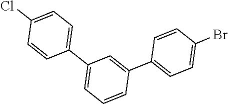 | B2-3 | 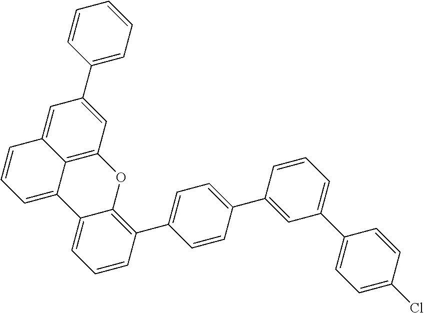 [SMILES: Clc1ccc(-c2cccc(-c3ccc(-c4cccc5c4Oc4cc(-c6ccccc6)cc6cccc-5c46)cc3)c2)cc1] | 63% | 558.09 |
| int. 4 | 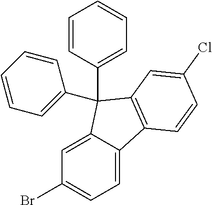 | B2-4 | 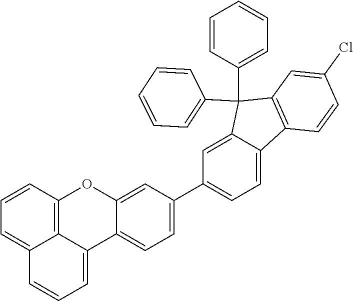 [SMILES: Clc1ccc2c(c1)C(c1ccccc1)(c1ccccc1)c1cc(-c3ccc4c(c3)Oc3cccc5cccc-4c35)ccc1-2] | 68% | 570.10 |
| int. 5 | 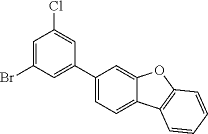 | B2-5 | 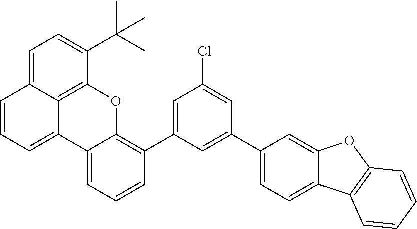 [SMILES: CC(C)(C)c1ccc2cccc3c2c1Oc1c(-c2cc(Cl)cc(-c4ccc5c(c4)oc4ccccc45)c2)cccc1-3] | 70% | 552.08 |
| int. 6 | 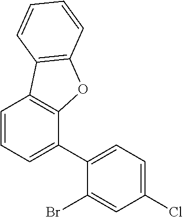 | B2-6 | 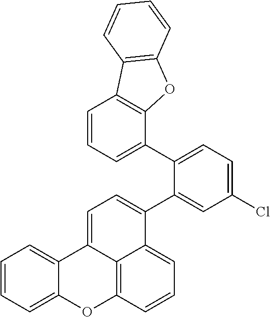 [SMILES: Clc1ccc(-c2cccc3c2oc2ccccc23)c(-c2ccc3c4c(cccc24)Oc2ccccc2-3)c1] | 63% | 495.97 |
| int. 7 | 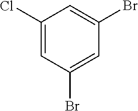 | B2-7 | 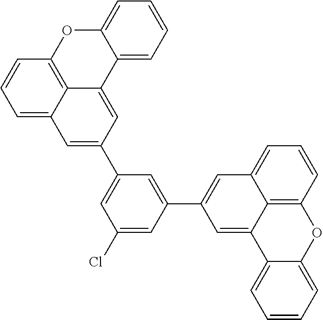 [SMILES: Clc1cc(-c2cc3c4c(cccc4c2)Oc2ccccc2-3)cc(-c2cc3c4c(cccc4c2)Oc2ccccc2-3)c1] | 64% | 546.03 |
| int. 8 | 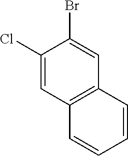 | B2-8 | 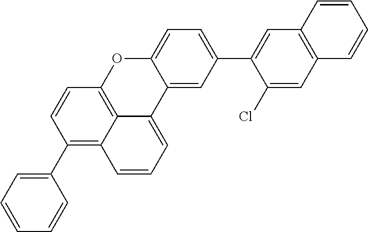 [SMILES: Clc1cc2ccccc2cc1-c1ccc2c(c1)-c1cccc3c(-c4ccccc4)ccc(c13)O2] | 71% | 455.95 |
| int. 9 | 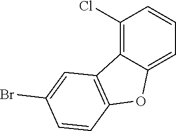 | B2-9 | 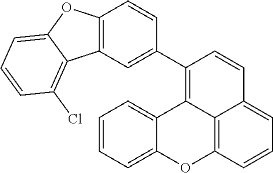 [SMILES: Clc1cccc2oc3ccc(-c4ccc5cccc6c5c4-c4ccccc4O6)cc3c12] | 68% | 419.88 |
| int. 10 | 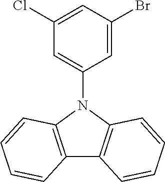 | B2-10 | 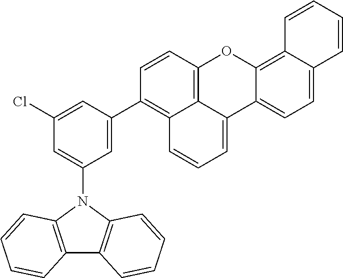 [SMILES: Clc1cc(-c2ccc3c4c(cccc24)-c2ccc4ccccc4c2O3)cc(-n2c3ccccc3c3ccccc32)c1] | 69% | 545.05 |
| int. 11 | 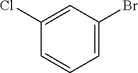 | B2-11 | 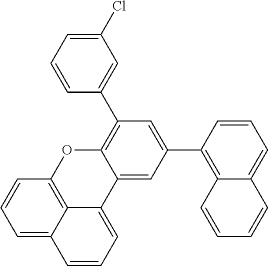 [SMILES: Clc1cccc(-c2cc(-c3cccc4ccccc34)cc3c2Oc2cccc4cccc-3c24)c1] | 63% | 455.95 |
| int. 12 | 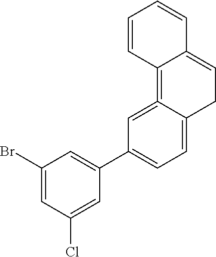 | B2-1 | 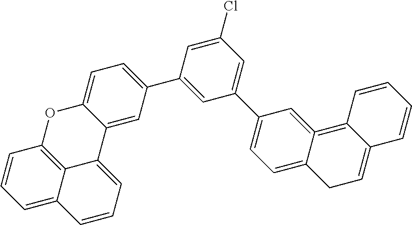 | 68% | 507.02 |

### 3-2) Synthesis of Chemical Formula 1

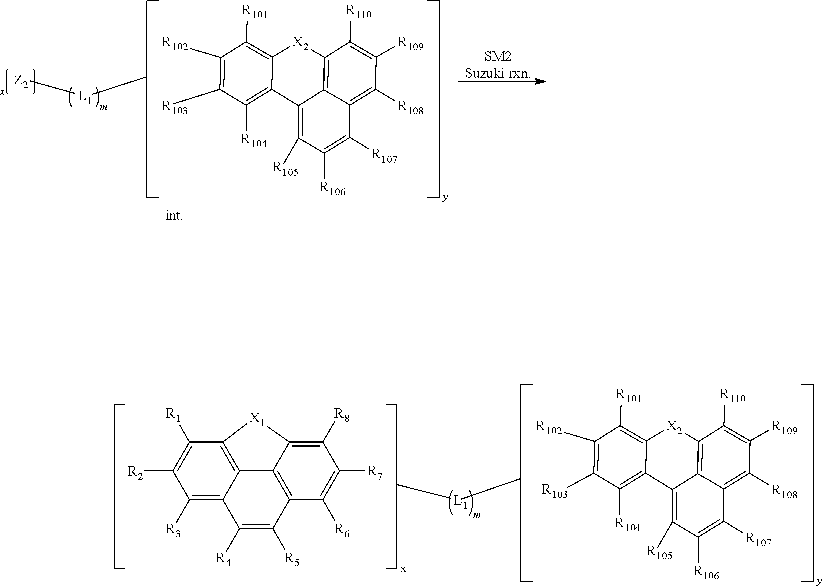

Here, R₁ to Rs, R₁₀₁ to R₁₁₀, X₁, X₂, L₁, and m are the same as those in Chemical Formula 1, Z2 is a halogen group, x is 1 or 2, and y is 1 or 2.

Int. (one of Int. 1 to Int. 12, 1 eq.) and SM2 (1.1 eq. or 2.1 eq., depending on the number of Z2, i.e., x) were added to tetrahydrofuran (THF, excess), followed by the addition of 2 M aqueous potassium carbonate solution (30 volumes relative to THF). Tetrakis(triphenylphosphine)palladium (2 mol %) was then added, and the mixture was stirred at 85° C. for 10 hours. After cooling to room temperature and confirming completion of the reaction, the aqueous potassium carbonate solution was removed, the layers were separated, and the crude product was purified by column chromatography using hexane and ethyl acetate to afford the compound of Formula 1 (Compounds 1-1 to 1-13 and Compounds I-1 to I-11).

Except for substituting SM41 and B2 in the synthesis of Formula int. with Int. and SM42 of [Table 1H] or with A7 or A7B, and SM42 of [Table 2H], respectively, the same procedure was applied to synthesize Compounds 1-1 to 1-13 and Compounds I-1 to I-11.

TABLE 1H

| | | | | | MS |
|---|---|---|---|---|---|
| Compound | Int. | SM2 | Product | yield | [M + H]+ |
| 1-1 | int. 1 | A8-1 | 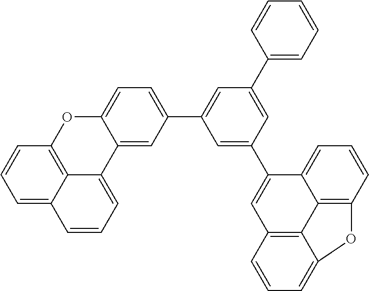 [SMILES: c1ccc(-c2cc(-c3ccc4c(c3)-c3cccc5cccc(c35)O4)cc(-c3cc4cccc5oc6cccc3c6c45)c2)cc1] | 72% | 561.65 |
| 1-2 | int. 2 | A8-7 | 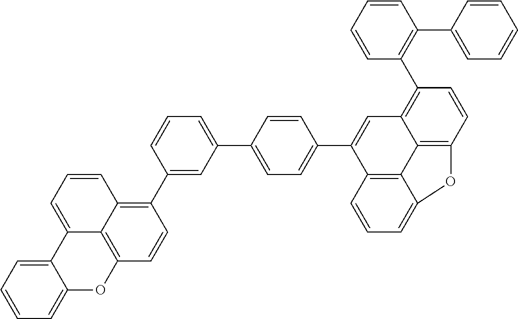 [SMILES: c1ccc(-c2ccccc2-c2ccc3oc4cccc5c(-c6ccc(-c7cccc(-c8ccc9c%10c(cccc8%10)-c8ccccc8O9)c7)cc6)cc2c3c45)cc1] | 73% | 713.85 |
| 1-3 | int. 3 | A8-1 | 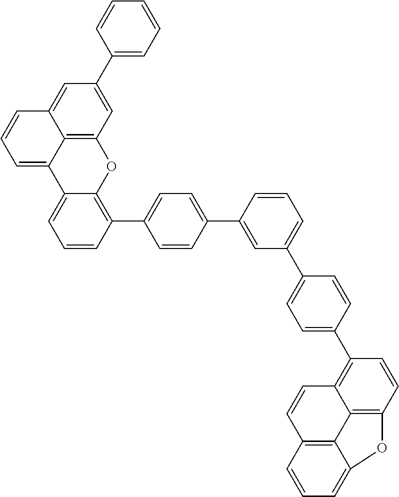 [SMILES: c1ccc(-c2cc3c4c(cccc4c2)-c2cccc(-c4ccc(-c5cccc(-c6ccc(-c7ccc8oc9cccc%10ccc7c8c%109)cc6)c5)cc4)c2O3)cc1] | 70% | 713.85 |
| 1-4 | int. 4 | A8-2 | 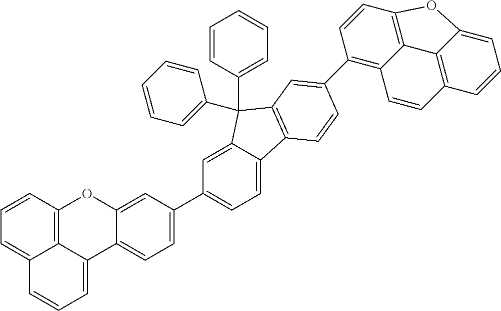 [SMILES: c1ccc(C2(c3ccccc3)c3cc(-c4ccc5c(c4)Oc4cccc6cccc-5c46)ccc3-c3ccc(-c4ccc5oc6cccc7ccc4c5c76)cc32)cc1] | 68% | 725.86 |
| 1-5 | int. 5 | A8-1 | 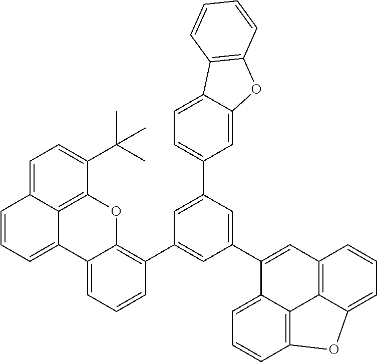 [SMILES: CC(C)(C)c1ccc2cccc3c2c1Oc1c(-c2cc(-c4ccc5c(c4)oc4ccccc45)cc(-c4cc5cccc6oc7cccc4c7c56)c2)cccc1-3] | 73% | 707.84 |
| 1-6 | int. 6 | A8-3 | 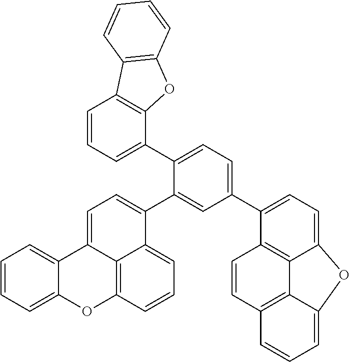 [SMILES: c1ccc2c(c1)Oc1cccc3c(-c4cc(-c5ccc6oc7cccc8ccc5c6c87)ccc4-c4cccc5c4oc4ccccc45)ccc-2c13] | 75% | 651.73 |
| 1-7 | int. 7 | A8-4 | 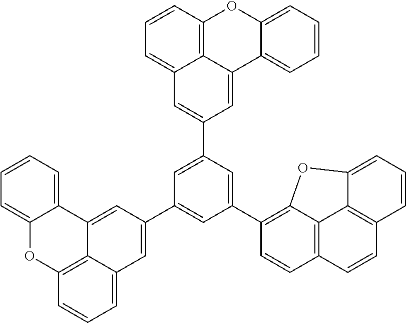 [SMILES: c1ccc2c(c1)Oc1cccc3cc(-c4cc(-c5cc6c7c(cccc7c5)Oc5ccccc5-6)cc(-c5ccc6ccc7cccc8oc5c6c78)c4)cc-2c13] | 73% | 701.79 |
| 1-8 | int. 8 | A8-5 | 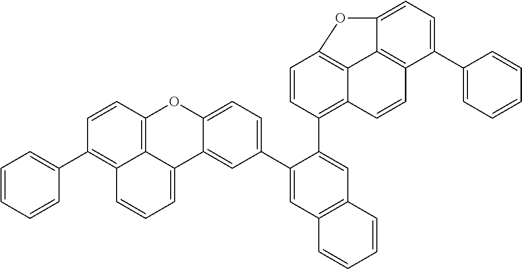 [SMILES: c1ccc(-c2ccc3c4c(cccc24)-c2cc(-c4cc5ccccc5cc4-c4ccc5oc6ccc(-c7ccccc7)c7ccc4c5c67)ccc2O3)cc1] | 70% | 687.81 |
| 1-9 | int. 9 | A8-3 | 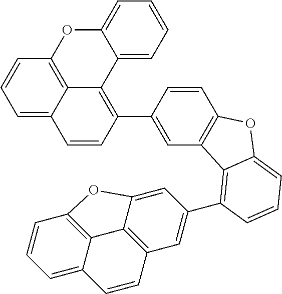 [SMILES: c1ccc2c(c1)Oc1cccc3ccc(-c4ccc5oc6cccc(-c7cc8ccc9cccc%10oc(c7)c8c9%10)c6c5c4)c-2c13] | 69% | 575.64 |
| 1-10 | int. 10 | A8-2 | 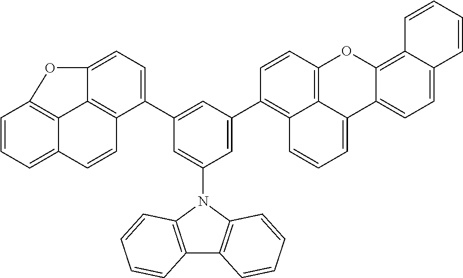 [SMILES: c1ccc2c3c(ccc2c1)-c1cccc2c(-c4cc(-c5ccc6oc7cccc8ccc5c6c87)cc(-n5c6ccccc6c6ccccc65)c4)ccc(c12)O3] | 64% | 700.81 |
| 1-11 | B1a-6 | A8-6 | 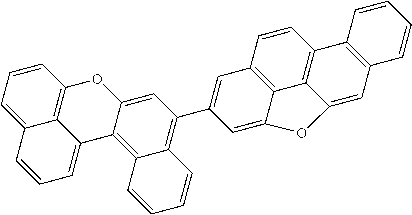 [SMILES: c1cc2c3c(cccc3c1)-c1c(cc(-c3cc4ccc5c6ccccc6cc6oc(c3)c4c65)c3ccccc13)O2] | 65% | 509.58 |
| 1-12 | Int. 11 | A8-2 | 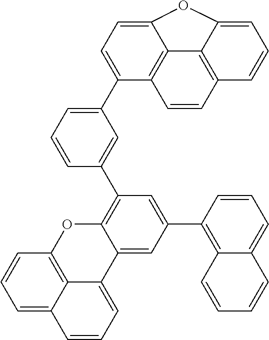 [SMILES: c1cc(-c2cc(-c3cccc4ccccc34)cc3c2Oc2cccc4cccc-3c24)cc(-c2ccc3oc4cccc5ccc2c3c54)c1] | 68% | 611.71 |
| 1-13 | Int. 12 | A8-8 | 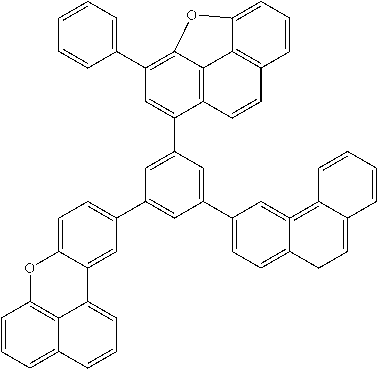 | 71% | 738.88 |

TABLE 2H

| | | | | | MS |
|---|---|---|---|---|---|
| Compound | A7 or A7B | SM2 | Product | Yield | [M + H]+ |
| I-1 | A7-9 | B2-3 | 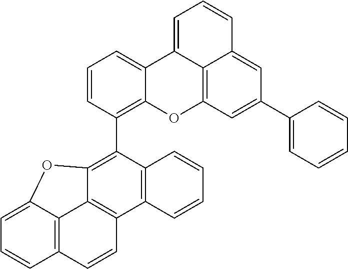 [SMILES: c1ccc(-c2cc3c4c(cccc4c2)-c2cccc(-c4c5ccccc5c5ccc6cccc7oc4c5c67)c2O3)cc1] | 68% | 535.61 |
| I-2 | A7-10 | B2-2 | 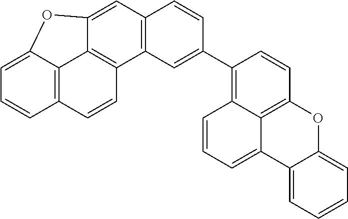 [SMILES: c1ccc2c(c1)Oc1ccc(-c3ccc4cc5oc6cccc7ccc(c4c3)c5c76)c3cccc-2c13] | 70% | 459.52 |
| I-3 | A7B-1 | B2-1 | 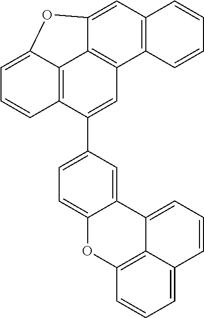 [SMILES: c1cc2c3c(cccc3c1)-c1cc(-c3cc4c5ccccc5cc5oc6cccc3c6c54)ccc1O2] | 71% | 459.52 |
| I-4 | A7B-1 | B2-5 | 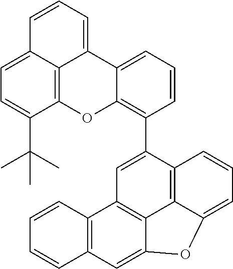 [SMILES: CC(C)(C)c1ccc2cccc3c2c1Oc1c(-c2cc4c5ccccc5cc5oc6cccc2c6c54)cccc1-3] | 67% | 515.62 |
| I-5 | A7B-2 | B2-7 | 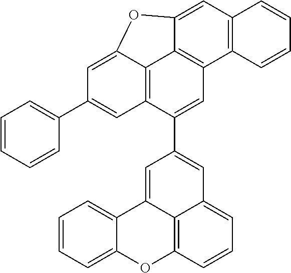 [SMILES: c1ccc(-c2cc3oc4cc5ccccc5c5cc(-c6cc7c8c(cccc8c6)Oc6ccccc6-7)c(c2)c3c45)cc1] | 69% | 535.61 |
| I-6 | A7-13 | B2-1 | 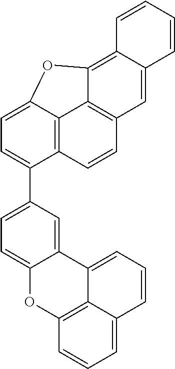 [SMILES: c1cc2c3c(cccc3c1)-c1cc(-c3ccc4oc5c6ccccc6cc6ccc3c4c65)ccc1O2] | 72% | 459.52 |
| I-7 | A7-14 | B2-2 | 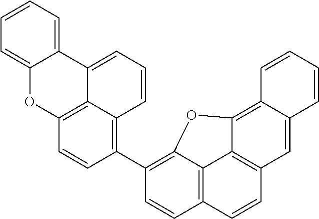 [SMILES: c1ccc2c(c1)Oc1ccc(-c3ccc4ccc5cc6ccccc6c6oc3c4c56)c3cccc-2c13] | 68% | 459.52 |
| I-8 | A7-15 | B2-8 | 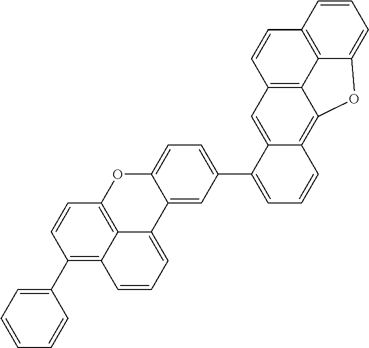 [SMILES: c1ccc(-c2ccc3c4c(cccc24)-c2cc(-c4cccc5c4cc4ccc6cccc7oc5c4c67)ccc2O3)cc1] | 69% | 535.61 |
| I-9 | A7B-3 | B2-4 | 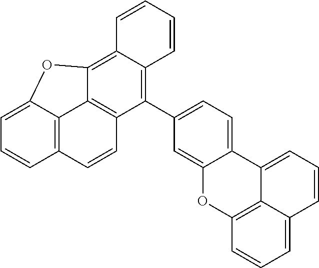 [SMILES: c1cc2c3c(cccc3c1)-c1ccc(-c3c4ccccc4c4oc5cccc6ccc3c4c65)cc1O2] | 66% | 459.52 |
| I-10 | A7B-3 | B2-10 | 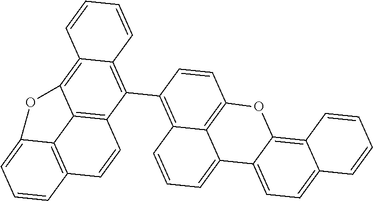 [SMILES: c1ccc2c3c(ccc2c1)-c1cccc2c(-c4c5ccccc5c5oc6cccc7ccc4c5c76)ccc(c12)O3] | 60% | 509.58 |
| I-11 | A7B-4 | B2-6 | 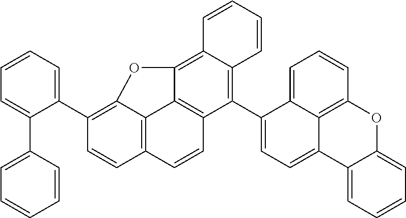 [SMILES: c1ccc(-c2ccccc2-c2ccc3ccc4c(-c5ccc6c7c(cccc57)Oc5ccccc5-6)c5ccccc5c5oc2c3c45)cc1] | 63% | 611.71 |

### 3-3) Synthesis of Chemical Formula 1D

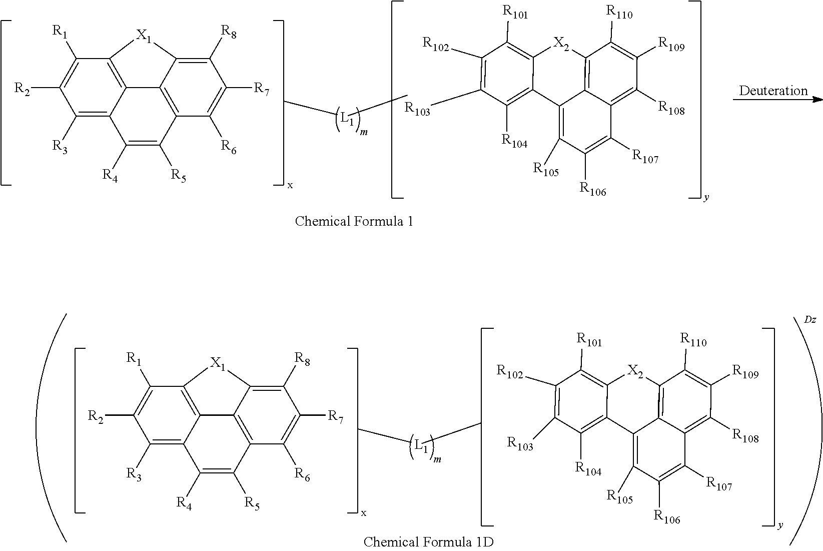

Here, R₁ to R₈, R₁₀₁ to R₁₁₀, X₁, X₂, L₁, x, y and m are the same as those in Chemical Formula 1, and Dz is the number of deuterium substitutions.

Chemical Formula 1 (1 eq) and trifluoromethanesulfonic acid (cat.) were put into C₆D₆(10- to 50-fold mass ratio compared to the reactant), and the resulting mixture was stirred at 70° C. for 10 minutes to 100 minutes. After the reaction was completed, D₂O (excess) was added thereto, the resulting solution was stirred for 30 minutes, and then trimethylamine (excess) was added dropwise thereto. The reaction solution was transferred to a separatory funnel, and an extraction with water and chloroform was performed. The extract was dried over MgSO₄, and then recrystallized by heating with toluene to obtain Compound 1D (Compounds 1-14 to 1-17 and I-12 to I-15) of the following Table 1D.

| | | | | | | | The | | D | |
|---|---|---|---|---|---|---|---|---|---|---|
| | | | | Product | Reaction | Product | number of | The number | substitution | Synthesis |
| | Chemical | | Reactant | theoretical | time | actual | total | of D | rate | yield |
| Compound | Formula 1 | Chemical Formula 1D | m/z | max m/z | (min) | max m/z | hydrogens | substitutions | (%) | (%) |
| 1-14 | 1-1 | 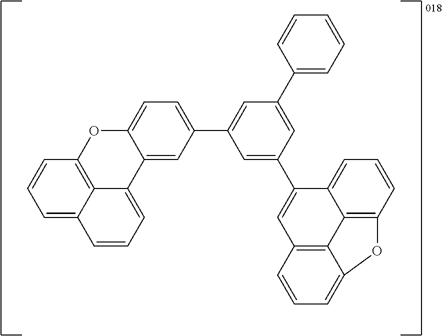 | 560 | 584 | 50 | 578 | 24 | 18 | 75% | 73% |
| 1-15 | 1-6 | 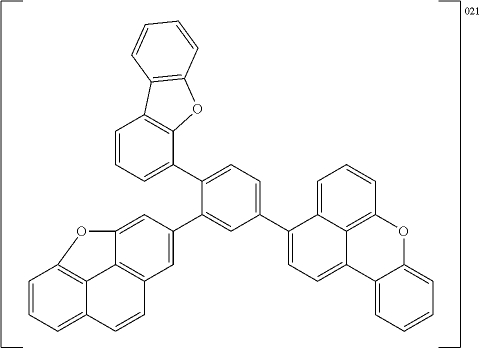 | 650 | 676 | 50 | 671 | 26 | 21 | 81% | 70% |
| 1-16 | 1-11 | 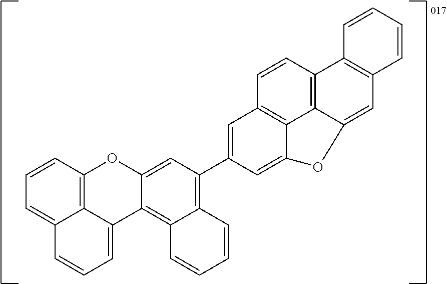 | 508 | 528 | 50 | 525 | 20 | 17 | 85% | 72% |
| 1-17 | 1-12 | 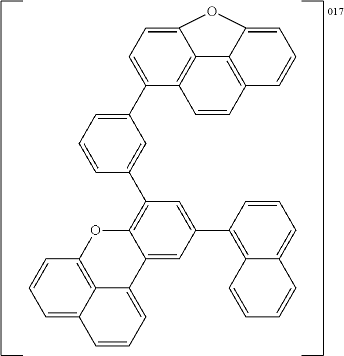 | 610 | 631 | 50 | 627 | 21 | 17 | 81% | 73% |
| I-12 | I-1 | 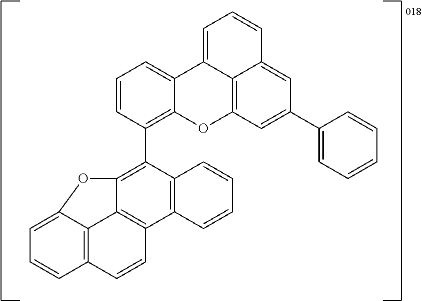 | 534 | 556 | 50 | 552 | 22 | 18 | 81% | 64% |
| I-13 | I-3 | 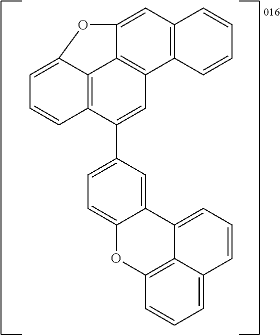 | 458 | 476 | 50 | 474 | 18 | 16 | 88% | 60% |
| I-14 | I-9 | 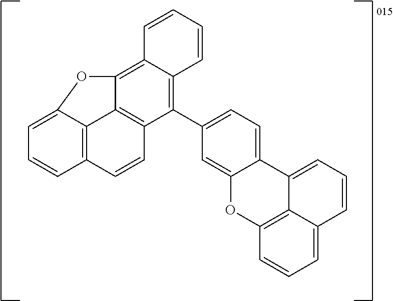 | 458 | 476 | 50 | 474 | 18 | 16 | 88% | 63% |
| I-15 | I-10 | 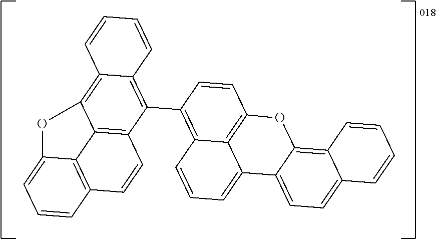 | 508 | 528 | 50 | 526 | 20 | 18 | 90% | 67% |

Each product has a different degree of deuterium substitution depending on the reaction time, and the substitution rate was determined according to the maximum m/z (M+) value.

The Compounds 1-14 to 1-17 and I-12 to I-15, which are the products were synthesized with reference to in-house prior literature such as KR 1790854 B1.

### [Preparation Example 2] Synthesis of Formula 2

### 1) Synthesis of Aryl-Based Chemical Formula 2

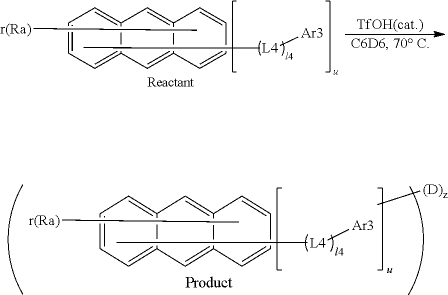

Here, Ar3 is a substituted or unsubstituted aryl group.

The reactant (1 eq) and trifluoromethanesulfonic acid (TfOH, cat.) were put into C₆D₆ (10- to 50-fold mass ratio compared to the reactant), and the resulting mixture was stirred at 70° C. for 10 minutes to 100 minutes. After the reaction was completed, D₂O (excess) was added thereto, the resulting solution was stirred for 30 minutes, and then trimethylamine (excess) was added dropwise thereto. The reaction solution was transferred to a separatory funnel, and an extraction with water and chloroform was performed. The extract was dried over MgSO₄, and then recrystallized by heating with toluene to obtain the products of the following [Table 2-1].

| Exem- | | | | | | | | | | |
|---|---|---|---|---|---|---|---|---|---|---|
| plary | | | | | | Pro- | The | The | | |
| deute- | | | | | Reac- | duct | number | number | D | Syn- |
| rium | | | Re- | Product | tion | actual | of total | of D | sub- | thesis |
| com- | | | actant | theoretical | time | max | hydro- | sub- | stitution | yield |
| pound | Reactant | Product | m/z | max m/z | (min) | m/z | gens | stitutions | rate (%) | (%) |
| Com- pound 1-A | Compound A 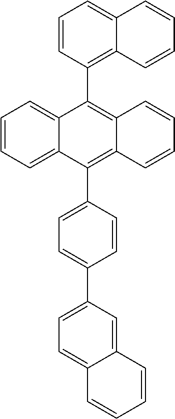 [SMILES: c1ccc2cc(-c3ccc(-c4c5ccccc5c(-c5cccc6ccccc56)c5ccccc45)cc3)ccc2c1] | 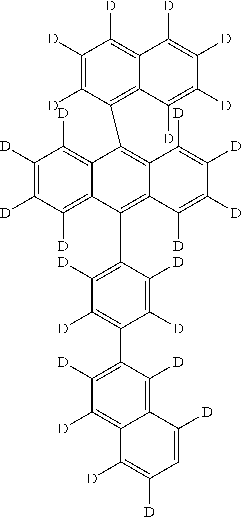 [SMILES: [2H]c1cc([2H])c2c([2H])c(-c3c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c3[2H])c([2H])c([2H])c2c1[2H]] | 506 | 531 | 50 | 530 | 25 | 24 | 96% | 70% |
| Com- pound 1-B | Compound B 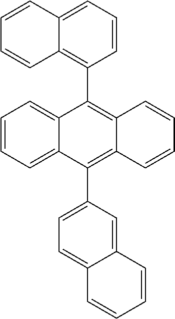 [SMILES: c1ccc2cc(-c3c4ccccc4c(-c4cccc5ccccc45)c4ccccc34)ccc2c1] | 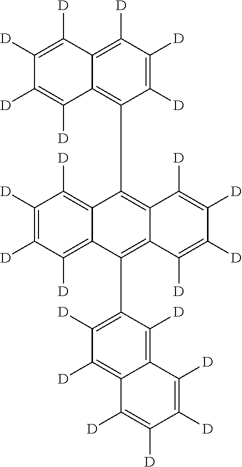 [SMILES: [2H]c1c([2H])c([2H])c2c([2H])c(-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)c([2H])c([2H])c2c1[2H]] | 430 | 452 | 50 | 452 | 22 | 22 | 100% | 73% |
| Com- pound 1-C | Compound C 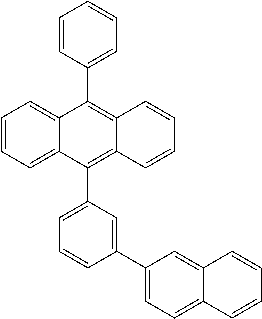 [SMILES: c1ccc(-c2c3ccccc3c(-c3cccc(-c4ccc5ccccc5c4)c3)c3ccccc23)cc1] | 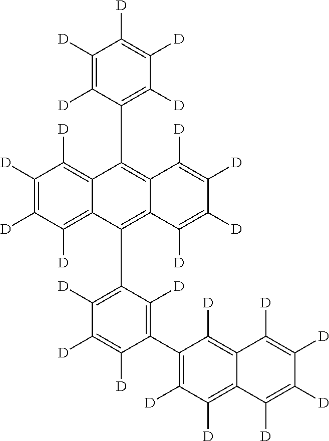 [SMILES: [2H]c1c([2H])c([2H])c(-c2c3c([2H])c([2H])c([2H])c([2H])c3c(-c3c([2H])c([2H])c([2H])c(-c4c([2H])c([2H])c5c([2H])c([2H])c([2H])c([2H])c5c4[2H])c3[2H])c3c([2H])c([2H])c([2H])c([2H])c23)c([2H])c1[2H]] | 456 | 480 | 45 | 473 | 24 | 17 | 71% | 74% |
| Com- pound 1-D | Compound D 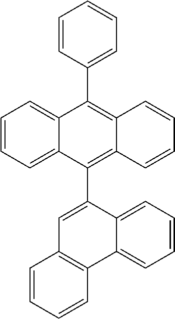 [SMILES: c1ccc(-c2c3ccccc3c(-c3cc4ccccc4c4ccccc34)c3ccccc23)cc1] | 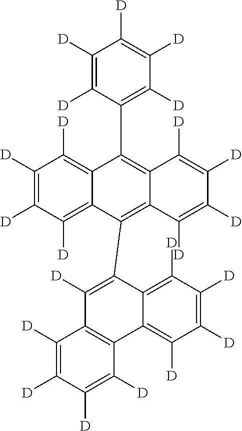 [SMILES: [2H]c1c([2H])c([2H])c(-c2c3c([2H])c([2H])c([2H])c([2H])c3c(-c3c([2H])c4c([2H])c([2H])c([2H])c([2H])c4c4c([2H])c([2H])c([2H])c([2H])c34)c3c([2H])c([2H])c([2H])c([2H])c23)c([2H])c1[2H]] | 430 | 452 | 30 | 443 | 22 | 13 | 59% | 69% |
| Com- pound 1-E | Compound E 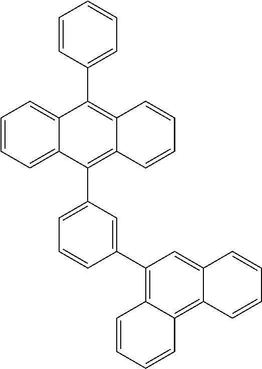 [SMILES: c1ccc(-c2c3ccccc3c(-c3cccc(-c4cc5ccccc5c5ccccc45)c3)c3ccccc23)cc1] | 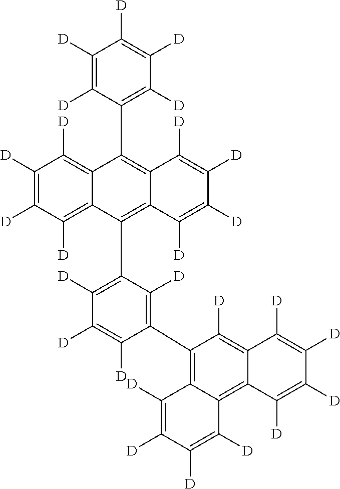 [SMILES: [2H]c1c([2H])c([2H])c(-c2c3c([2H])c([2H])c([2H])c([2H])c3c(-c3c([2H])c([2H])c([2H])c(-c4c([2H])c5c([2H])c([2H])c([2H])c([2H])c5c5c([2H])c([2H])c([2H])c([2H])c45)c3[2H])c3c([2H])c([2H])c([2H])c([2H])c23)c([2H])c1[2H]] | 506 | 532 | 40 | 522 | 26 | 16 | 62% | 65% |
| Com- pound 1-F | Compound F 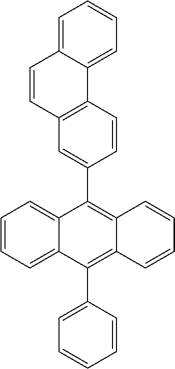 [SMILES: c1ccc(-c2c3ccccc3c(-c3ccc4c(ccc5ccccc54)c3)c3ccccc23)cc1] | 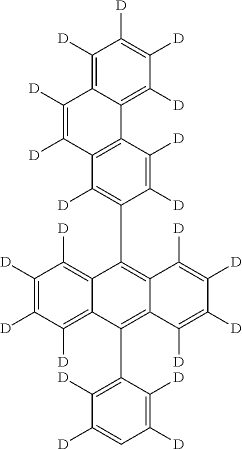 [SMILES: [2H]c1cc([2H])c([2H])c(-c2c3c([2H])c([2H])c([2H])c([2H])c3c(-c3c([2H])c([2H])c4c(c3[2H])c([2H])c([2H])c3c([2H])c([2H])c([2H])c([2H])c34)c3c([2H])c([2H])c([2H])c([2H])c23)c1[2H]] | 430 | 452 | 40 | 448 | 22 | 18 | 82% | 70% |
| Com- pound 1-C | Compound G 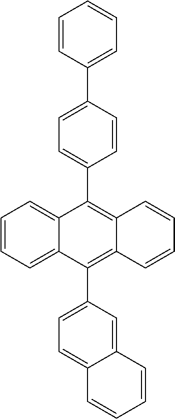 [SMILES: c1ccc(-c2ccc(-c3c4ccccc4c(-c4ccc5ccccc5c4)c4ccccc34)cc2)cc1] | 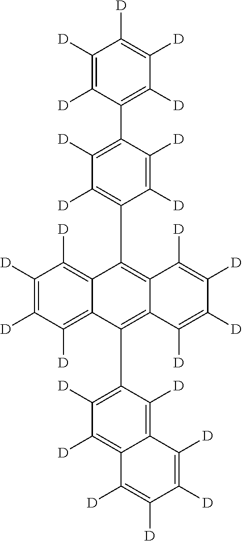 [SMILES: [2H]c1c([2H])c([2H])c(-c2c([2H])c([2H])c(-c3c4c([2H])c([2H])c([2H])c([2H])c4c(-c4c([2H])c([2H])c5c([2H])c([2H])c([2H])c([2H])c5c4[2H])c4c([2H])c([2H])c([2H])c([2H])c34)c([2H])c2[2H])c([2H])c1[2H]] | 456 | 480 | 50 | 478 | 24 | 22 | 92% | 73% |
| Com- pound 1-H | Compound H 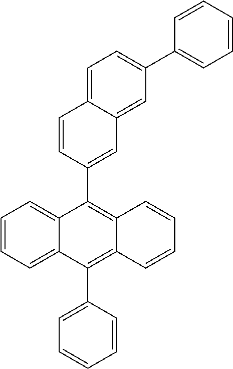 [SMILES: c1ccc(-c2ccc3ccc(-c4c5ccccc5c(-c5ccccc5)c5ccccc45)cc3c2)cc1] | 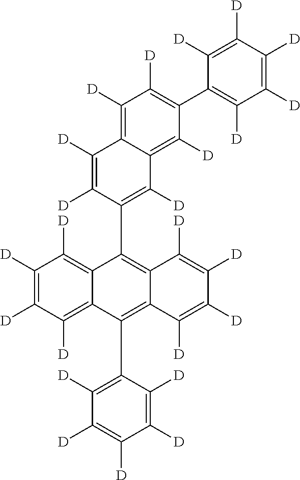 [SMILES: [2H]c1c([2H])c([2H])c(-c2c([2H])c([2H])c3c([2H])c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c([2H])c3c2[2H])c([2H])c1[2H]] | 456 | 480 | 25 | 468 | 24 | 12 | 50% | 71% |
| Com- pound 1-I | Compound I 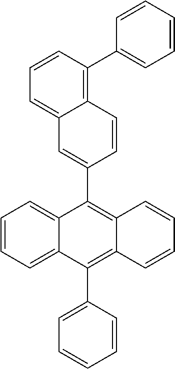 [SMILES: c1ccc(-c2cccc3cc(-c4c5ccccc5c(-c5ccccc5)c5ccccc45)ccc23)cc1] | 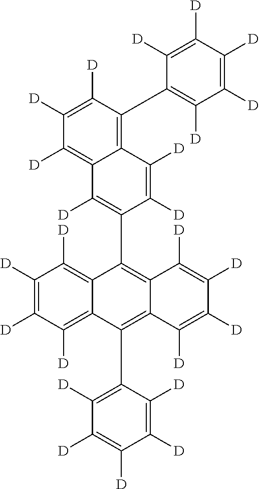 [SMILES: [2H]c1c([2H])c([2H])c(-c2c([2H])c([2H])c([2H])c3c([2H])c(-c4c5c([2H])c([2H])c([2H])c([2H])c5c(-c5c([2H])c([2H])c([2H])c([2H])c5[2H])c5c([2H])c([2H])c([2H])c([2H])c45)c([2H])c([2H])c23)c([2H])c1[2H]] | 456 | 480 | 40 | 477 | 24 | 21 | 88% | 68% |
| Com- pound 1-J | Compound J 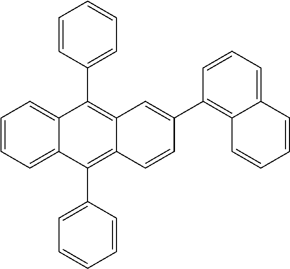 [SMILES: c1ccc(-c2c3ccccc3c(-c3ccccc3)c3cc(-c4cccc5ccccc45)ccc23)cc1] | 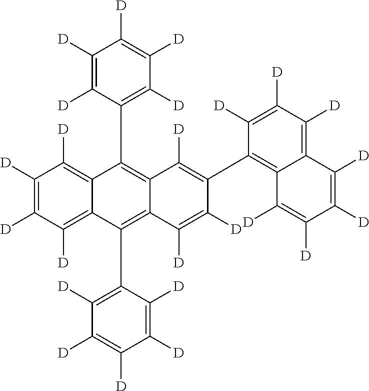 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4c([2H])c([2H])c([2H])c5c([2H])c([2H])c([2H])c([2H])c45)c([2H])c([2H])c23)c([2H])c1[2H]] | 456 | 480 | 50 | 477 | 24 | 21 | 88% | 65% |
| Com- pound 1-K | Compound K 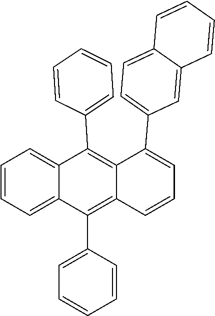 [SMILES: c1ccc(-c2c3ccccc3c(-c3ccccc3)c3c(-c4ccc5ccccc5c4)cccc23)cc1] | 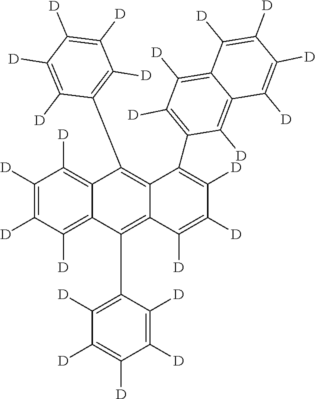 [SMILES: [2H]c1c([2H])c([2H])c(-c2c3c([2H])c([2H])c([2H])c([2H])c3c(-c3c([2H])c([2H])c([2H])c([2H])c3[2H])c3c(-c4c([2H])c([2H])c5c([2H])c([2H])c([2H])c([2H])c5c4[2H])c([2H])c([2H])c([2H])c23)c([2H])c1[2H]] | 456 | 480 | 50 | 476 | 24 | 20 | 83% | 70% |
| Com- pound 1-L | Compound L 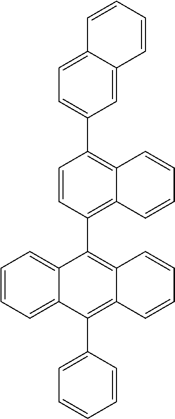 [SMILES: c1ccc(-c2c3ccccc3c(-c3ccc(-c4ccc5ccccc5c4)c4ccccc34)c3ccccc23)cc1] | 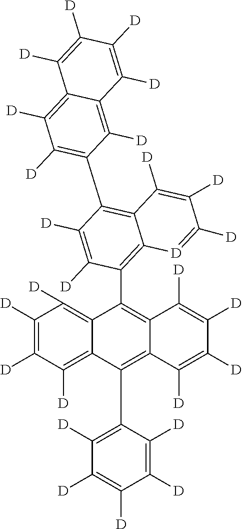 | 506 | 532 | 50 | 529 | 26 | 23 | 88% | 66% |
| Com- pound 1-M | Compound M 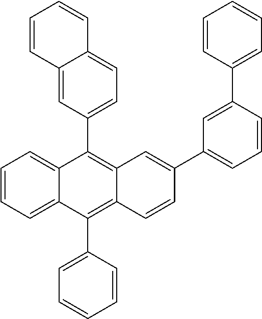 [SMILES: c1ccc(-c2cccc(-c3ccc4c(-c5ccccc5)c5ccccc5c(-c5ccc6ccccc6c5)c4c3)c2)cc1] | 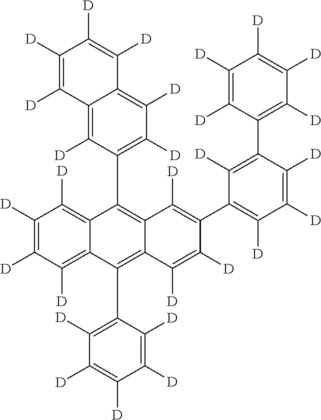 [SMILES: [2H]c1c([2H])c([2H])c(-c2c([2H])c([2H])c([2H])c(-c3c([2H])c([2H])c4c(-c5c([2H])c([2H])c([2H])c([2H])c5[2H])c5c([2H])c([2H])c([2H])c([2H])c5c(-c5c([2H])c([2H])c6c([2H])c([2H])c([2H])c([2H])c6c5[2H])c4c3[2H])c2[2H])c([2H])c1[2H]] | 532 | 560 | 30 | 551 | 28 | 19 | 68% | 72% |
| Com- pound I-N | Compound N 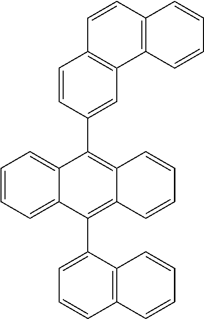 [SMILES: c1ccc2c(-c3c4ccccc4c(-c4ccc5ccc6ccccc6c5c4)c4ccccc34)cccc2c1] | 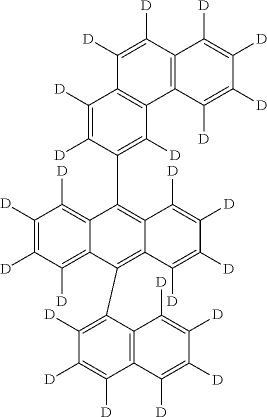 [SMILES: [2H]c1c([2H])c([2H])c2c(-c3c4c([2H])c([2H])c([2H])c([2H])c4c(-c4c([2H])c([2H])c5c([2H])c([2H])c6c([2H])c([2H])c([2H])c([2H])c6c5c4[2H])c4c([2H])c([2H])c([2H])c([2H])c34)c([2H])c([2H])c([2H])c2c1[2H]] | 480 | 504 | 50 | 500 | 24 | 20 | 83% | 73% |
| Com- pound 1-O | Compound 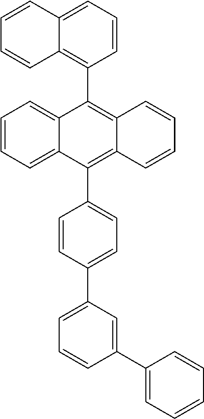 [SMILES: c1ccc(-c2cccc(-c3ccc(-c4c5ccccc5c(-c5cccc6ccccc56)c5ccccc45)cc3)c2)cc1] | 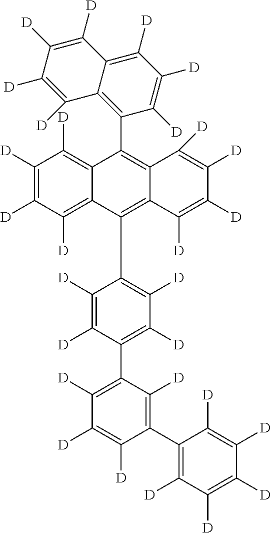 [SMILES: [2H]c1c([2H])c([2H])c(-c2c([2H])c([2H])c([2H])c(-c3c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c3[2H])c2[2H])c([2H])c1[2H]] | 532 | 560 | 40 | 556 | 28 | 24 | 86% | 71% |
| Com- pound 1-P | Compound P 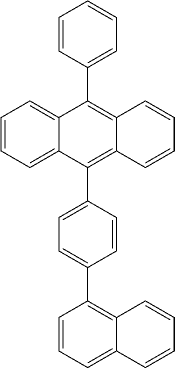 [SMILES: c1ccc(-c2c3ccccc3c(-c3ccc(-c4cccc5ccccc45)cc3)c3ccccc23)cc1] | 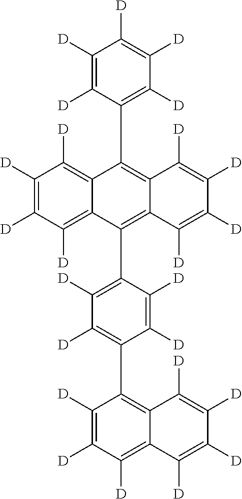 [SMILES: [2H]c1c([2H])c([2H])c(-c2c3c([2H])c([2H])c([2H])c([2H])c3c(-c3c([2H])c([2H])c(-c4c([2H])c([2H])c([2H])c5c([2H])c([2H])c([2H])c([2H])c45)c([2H])c3[2H])c3c([2H])c([2H])c([2H])c([2H])c23)c([2H])c1[2H]] | 456 | 480 | 60 | 480 | 24 | 24 | 100% | 73% |
| Com- pound 1-Q | Compound Q 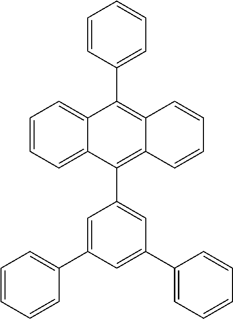 [SMILES: c1ccc(-c2cc(-c3ccccc3)cc(-c3c4ccccc4c(-c4ccccc4)c4ccccc34)c2)cc1] | 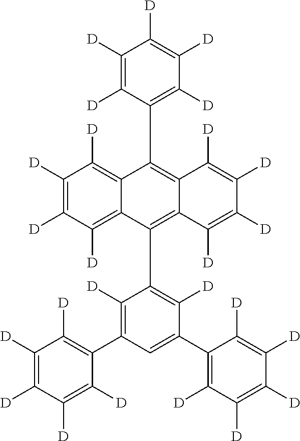 [SMILES: [2H]c1c([2H])c([2H])c(-c2cc(-c3c([2H])c([2H])c([2H])c([2H])c3[2H])c([2H])c(-c3c4c([2H])c([2H])c([2H])c([2H])c4c(-c4c([2H])c([2H])c([2H])c([2H])c4[2H])c4c([2H])c([2H])c([2H])c([2H])c34)c2[2H])c([2H])c1[2H]] | 482 | 507 | 35 | 500 | 25 | 18 | 72% | 65% |
| Com- pound 1-R | Compound R 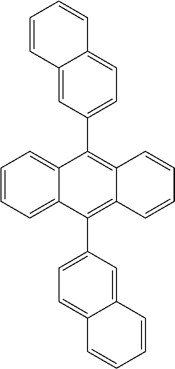 [SMILES: c1ccc2cc(-c3c4ccccc4c(-c4ccc5ccccc5c4)c4ccccc34)ccc2c1] | 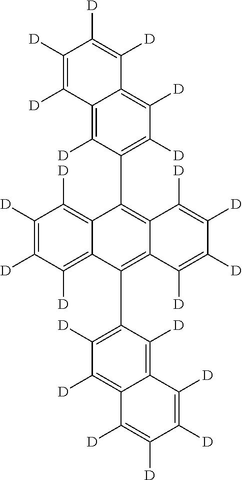 [SMILES: [2H]c1c([2H])c([2H])c2c([2H])c(-c3c4c([2H])c([2H])c([2H])c([2H])c4c(-c4c([2H])c([2H])c5c([2H])c([2H])c([2H])c([2H])c5c4[2H])c4c([2H])c([2H])c([2H])c([2H])c34)c([2H])c([2H])c2c1[2H]] | 430 | 452 | 50 | 447 | 22 | 17 | 77% | 66% |
| Com- pound 1-S | Compound S 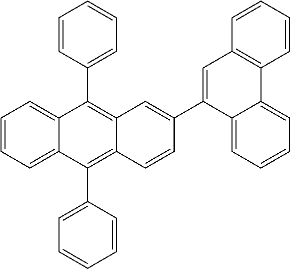 [SMILES: c1ccc(-c2c3ccccc3c(-c3ccccc3)c3cc(-c4cc5ccccc5c5ccccc45)ccc23)cc1] | 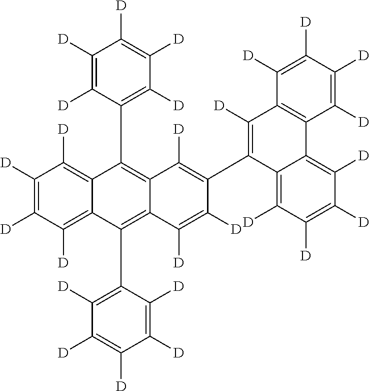 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4c([2H])c5c([2H])c([2H])c([2H])c([2H])c5c5c([2H])c([2H])c([2H])c([2H])c45)c([2H])c([2H])c23)c([2H])c1[2H]] | 506 | 532 | 60 | 530 | 26 | 24 | 92% | 71% |
| Com- pound 1-T | Compound T 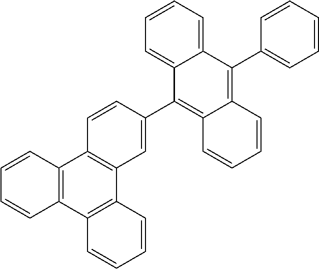 [SMILES: c1ccc(-c2c3ccccc3c(-c3ccc4c5ccccc5c5ccccc5c4c3)c3ccccc23)cc1] | 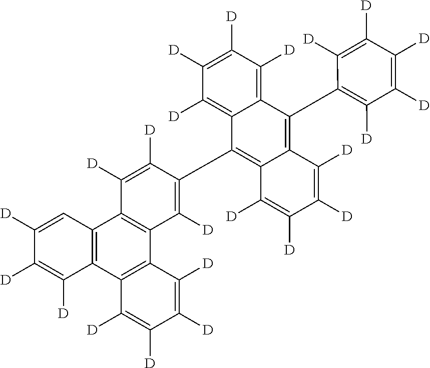 [SMILES: [2H]c1cc2c(c([2H])c1[2H])c1c([2H])c([2H])c([2H])c([2H])c1c1c([2H])c(-c3c4c([2H])c([2H])c([2H])c([2H])c4c(-c4c([2H])c([2H])c([2H])c([2H])c4[2H])c4c([2H])c([2H])c([2H])c([2H])c34)c([2H])c([2H])c21] | 480 | 503 | 45 | 498 | 23 | 18 | 78% | 70% |
| Com- pound 1-U | Compound U 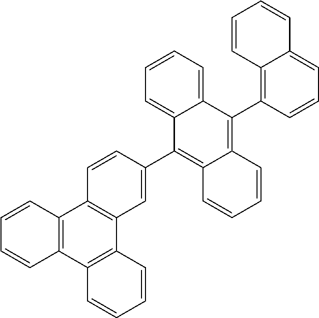 [SMILES: c1ccc2c(-c3c4ccccc4c(-c4ccc5c6ccccc6c6ccccc6c5c4)c4ccccc34)cccc2c1] | 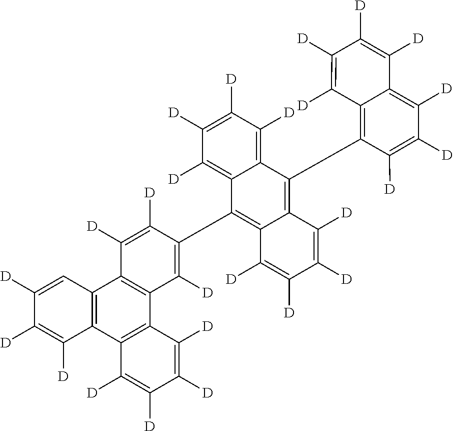 [SMILES: [2H]c1cc2c(c([2H])c1[2H])c1c([2H])c([2H])c([2H])c([2H])c1c1c([2H])c(-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)c([2H])c([2H])c21] | 530 | 555 | 45 | 549 | 25 | 19 | 76% | 68% |
| Com- pound 1-V | Compound V 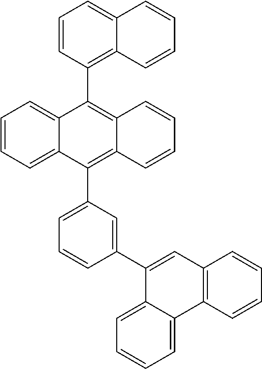 [SMILES: c1cc(-c2c3ccccc3c(-c3cccc4ccccc34)c3ccccc23)cc(-c2cc3ccccc3c3ccccc23)c1] | 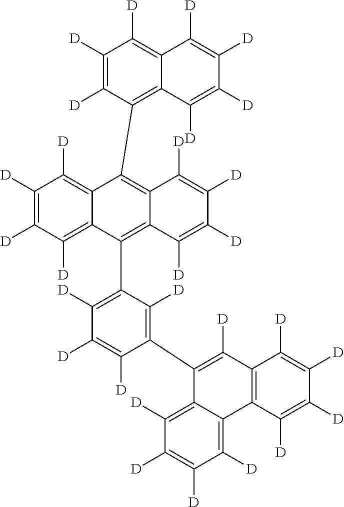 [SMILES: [2H]c1c([2H])c(-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c(-c2c([2H])c3c([2H])c([2H])c([2H])c([2H])c3c3c([2H])c([2H])c([2H])c([2H])c23)c1[2H]] | 556 | 584 | 50 | 580 | 28 | 24 | 86% | 64% |
| Com- pound 1-W | Compound W 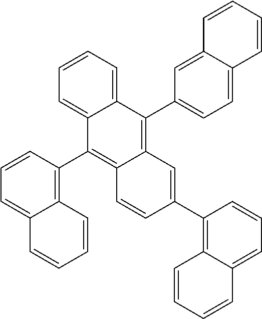 [SMILES: c1ccc2cc(-c3c4ccccc4c(-c4cccc5ccccc45)c4ccc(-c5cccc6ccccc56)cc34)ccc2c1] | 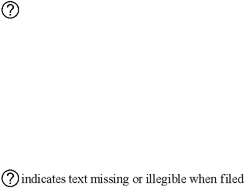 | 556 | 584 | 60 | 582 | 28 | 26 | 93% | 70% |

Each product has a different degree of deuterium substitution depending on the reaction time, and the substitution rate was determined according to the maximum m/z (M+) value.

Compounds A to W, which are the reactants, were synthesized with reference to the prior literature such as JP 4070676 B2, KR 1477844 B1, U.S. Pat. No. 6,465,115 B2, JP 3148176 B2, JP 4025136 B2, JP 4188082 B2, JP 5015459 B2, KR 1979037 B1, KR 1550351 B1, KR 1503766 B1, KR 0826364 B1, KR 0749631 B1, and KR 1115255 B1. Further, for Compounds 1-A to 1-W, which are deuterium-substituted products, the prior literature of KR 1538534 B1 was referenced.

### 2) Synthesis of Furan-Based Chemical Formula 2

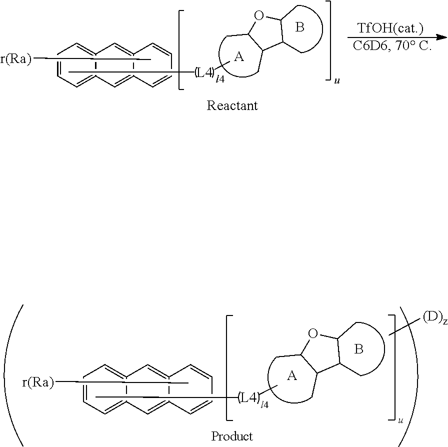

The reactant (1 eq) and trifluoromethanesulfonic acid (cat.) were put into C₆D₆(10- to 50-fold mass ratio compared to the reactant), and the resulting mixture was stirred at 70° C. for 10 minutes to 100 minutes. After the reaction was completed, D₂O (excess) was added thereto, the resulting solution was stirred for 30 minutes, and then trimethylamine (excess) was added dropwise thereto. The reaction solution was transferred to a separatory funnel, and an extraction with water and chloroform was performed. The extract was dried over MgSO₄, and then recrystallized by heating with toluene to obtain the products the following [Table 2-2] to [Table 2-3].

| | | | | | Re- | | The | | D | Syn- |
|---|---|---|---|---|---|---|---|---|---|---|
| Exemplary | | | Re- | Product | action | Product | number of | The number | sub- | thesis |
| deuterium | | | actant | theoretical | time | actual | total | of D | stitution | yield |
| compound | Reactant | Product | m/z | max m/z | (min) | max m/z | hydrogens | substitutions | rate (%) | (%) |
| Compound 2-1 | Compound 1# 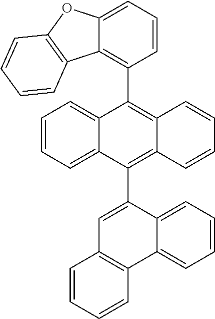 [SMILES: c1ccc2c(c1)cc(-c1c3ccccc3c(-c3cccc4oc5ccccc5c34)c3ccccc13)c1ccccc12] | 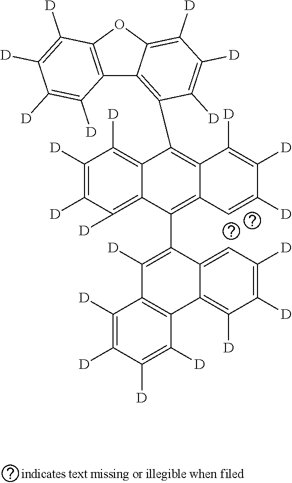 [SMILES: [2H]c1cc2c(-c3c([2H])c4c([2H])c([2H])c([2H])c([2H])c4c4c([2H])c([2H])c([2H])cc34)c3c([2H])c([2H])c([2H])c([2H])c3c(-c3c([2H])c([2H])c([2H])c4oc5c([2H])c([2H])c([2H])c([2H])c5c34)c2c([2H])c1[2H]] | 520 | 544 | 30 | 538 | 24 | 18 | 75% | 70% |
| Compound 2-2 | Compound 2# 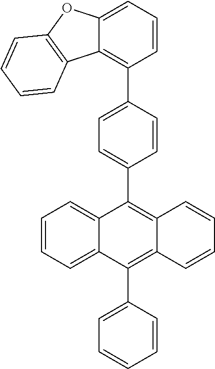 [SMILES: c1ccc(-c2c3ccccc3c(-c3ccc(-c4cccc5oc6ccccc6c45)cc3)c3ccccc23)cc1] | 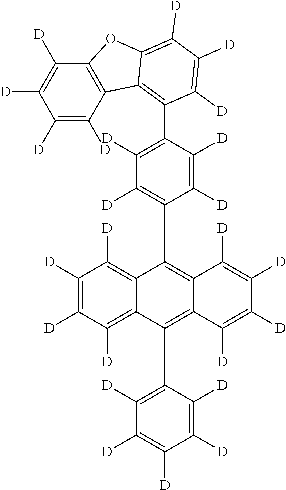 [SMILES: [2H]c1c([2H])c([2H])c(-c2c3c([2H])c([2H])c([2H])c([2H])c3c(-c3c([2H])c([2H])c(-c4c([2H])c([2H])c([2H])c5oc6c([2H])c([2H])c([2H])c([2H])c6c45)c([2H])c3[2H])c3c([2H])c([2H])c([2H])c([2H])c23)c([2H])c1[2H]] | 496 | 520 | 30 | 513 | 24 | 17 | 71% | 73% |
| Compound 2-3 | Compound 3# 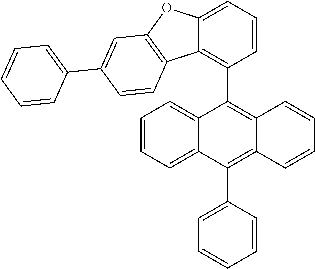 [SMILES: c1ccc(-c2ccc3c(c2)oc2cccc(-c4c5ccccc5c(-c5ccccc5)c5ccccc45)c23)cc1] | 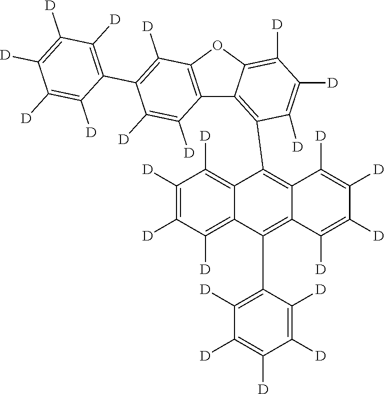 [SMILES: [2H]c1c([2H])c([2H])c(-c2c([2H])c([2H])c3c(oc4c([2H])c([2H])c([2H])c(-c5c6c([2H])c([2H])c([2H])c([2H])c6c(-c6c([2H])c([2H])c([2H])c([2H])c6[2H])c6c([2H])c([2H])c([2H])c([2H])c56)c43)c2[2H])c([2H])c1[2H]] | 496 | 520 | 30 | 520 | 24 | 24 | 100% | 74% |
| Compound 2-4 | Compound 4# 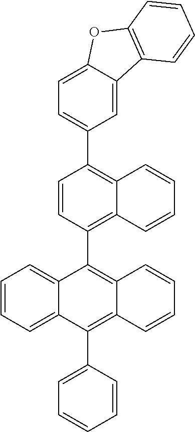 [SMILES: c1ccc(-c2c3ccccc3c(-c3ccc(-c4ccc5oc6ccccc6c5c4)c4ccccc34)c3ccccc23)cc1] | 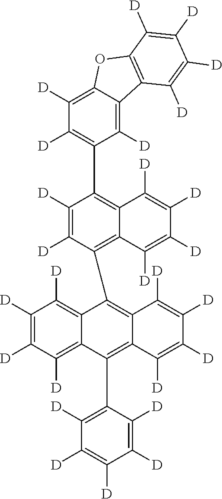 [SMILES: [2H]c1c([2H])c([2H])c(-c2c3c([2H])c([2H])c([2H])c([2H])c3c(-c3c([2H])c([2H])c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c23)c([2H])c1[2H]] | 546 | 572 | 15 | 560 | 26 | 14 | 54% | 69% |
| Compound 2-5 | Compound 5# 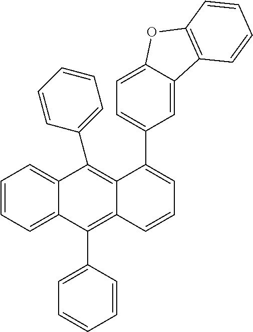 [SMILES: c1ccc(-c2c3ccccc3c(-c3ccccc3)c3c(-c4ccc5oc6ccccc6c5c4)cccc23)cc1] | 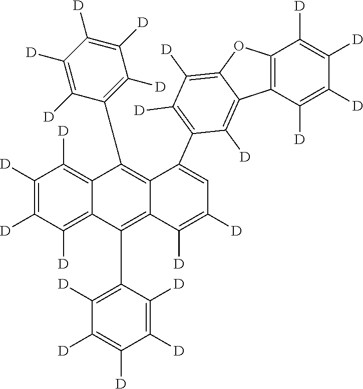 [SMILES: [2H]c1cc(-c2c([2H])c([2H])c3oc4c([2H])c([2H])c([2H])c([2H])c4c3c2[2H])c2c(-c3c([2H])c([2H])c([2H])c([2H])c3[2H])c3c([2H])c([2H])c([2H])c([2H])c3c(-c3c([2H])c([2H])c([2H])c([2H])c3[2H])c2c1[2H]] | 496 | 519 | 25 | 510 | 23 | 14 | 61% | 70% |
| Compound 2-6 | Compound 6# 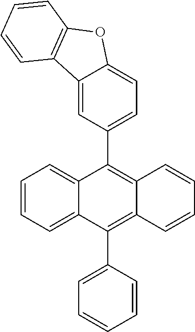 [SMILES: c1ccc(-c2c3ccccc3c(-c3ccc4oc5ccccc5c4c3)c3ccccc23)cc1] | 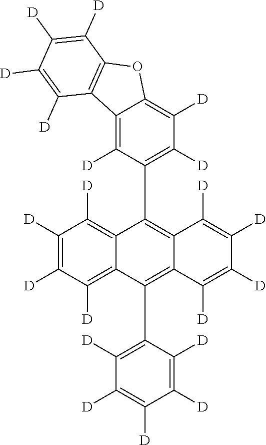 [SMILES: [2H]c1c([2H])c([2H])c(-c2c3c([2H])c([2H])c([2H])c([2H])c3c(-c3c([2H])c([2H])c4oc5c([2H])c([2H])c([2H])c([2H])c5c4c3[2H])c3c([2H])c([2H])c([2H])c([2H])c23)c([2H])c1[2H]] | 420 | 440 | 35 | 438 | 20 | 18 | 90% | 73% |
| Compound 2-7 | Compound 7# 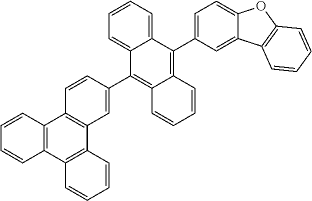 [SMILES: c1ccc2c(c1)oc1ccc(-c3c4ccccc4c(-c4ccc5c6ccccc6c6ccccc6c5c4)c4ccccc34)cc12] | 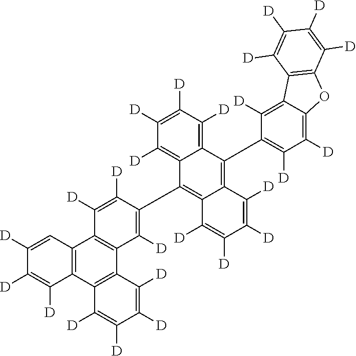 [SMILES: [2H]c1cc2c(c([2H])c1[2H])c1c([2H])c([2H])c([2H])c([2H])c1c1c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c([2H])c([2H])c21] | 570 | 595 | 40 | 591 | 25 | 21 | 84% | 71% |
| Compound 2-8 | Compound 8# 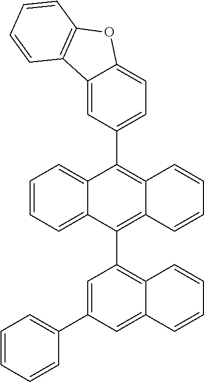 [SMILES: c1ccc(-c2cc(-c3c4ccccc4c(-c4ccc5oc6ccccc6c5c4)c4ccccc34)c3ccccc3c2)cc1] | 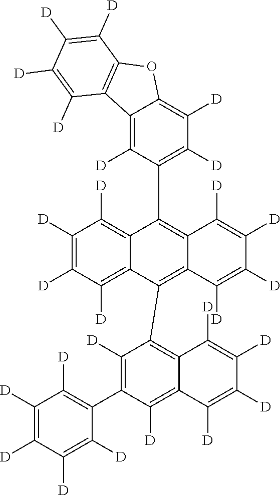 [SMILES: [2H]c1c([2H])c([2H])c(-c2c([2H])c(-c3c4c([2H])c([2H])c([2H])c([2H])c4c(-c4c([2H])c([2H])c5oc6c([2H])c([2H])c([2H])c([2H])c6c5c4[2H])c4c([2H])c([2H])c([2H])c([2H])c34)c3c([2H])c([2H])c([2H])c([2H])c3c2[2H])c([2H])c1[2H]] | 546 | 572 | 10 | 558 | 26 | 12 | 46% | 68% |
| Compound 2-9 | Compound 9# 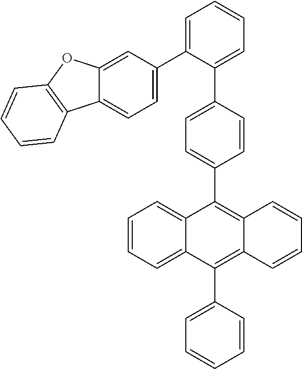 [SMILES: c1ccc(-c2c3ccccc3c(-c3ccc(-c4ccccc4-c4ccc5c(c4)oc4ccccc45)cc3)c3ccccc23)cc1] | 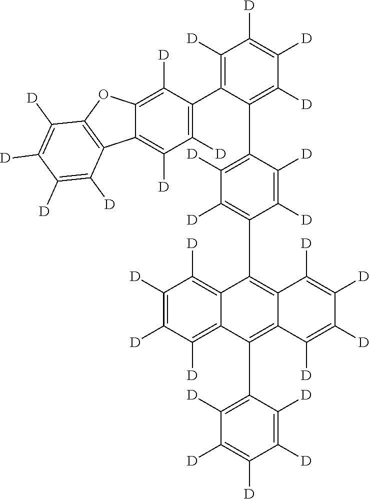 [SMILES: [2H]c1c([2H])c([2H])c(-c2c3c([2H])c([2H])c([2H])c([2H])c3c(-c3c([2H])c([2H])c(-c4c([2H])c([2H])c([2H])c([2H])c4-c4c([2H])c([2H])c5c(oc6c([2H])c([2H])c([2H])c([2H])c65)c4[2H])c([2H])c3[2H])c3c([2H])c([2H])c([2H])c([2H])c23)c([2H])c1[2H]] | 572 | 600 | 60 | 596 | 28 | 24 | 86% | 65% |
| Compound 2-10 | Compound 10# 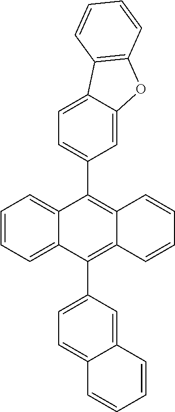 [SMILES: c1ccc2cc(-c3c4ccccc4c(-c4ccc5c(c4)oc4ccccc45)c4ccccc34)ccc2c1] | 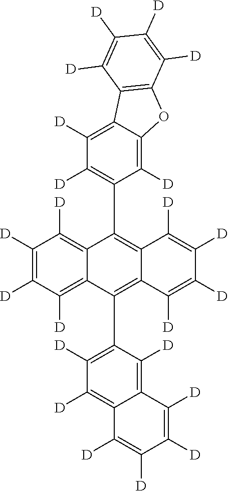 [SMILES: [2H]c1c([2H])c([2H])c2c(oc3c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c([2H])c([2H])c32)c1[2H]] | 470 | 492 | 50 | 492 | 22 | 22 | 100% | 66% |
| Compound 2-11 | Compound 11# 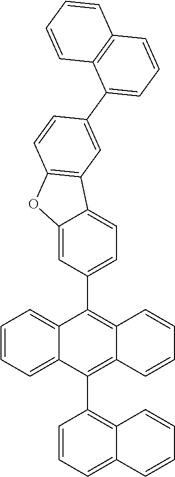 [SMILES: c1ccc2c(-c3ccc4oc5cc(-c6c7ccccc7c(-c7cccc8ccccc78)c7ccccc67)ccc5c4c3)cccc2c1] | 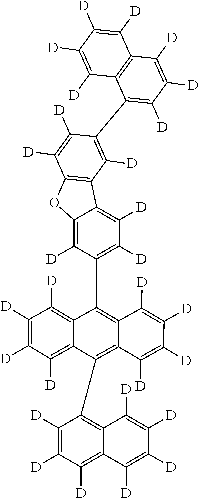 [SMILES: [2H]c1c(-c2c([2H])c([2H])c([2H])c3c([2H])c([2H])c([2H])c([2H])c23)c([2H])c2c(oc3c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c([2H])c32)c1[2H]] | 596 | 624 | 55 | 620 | 28 | 24 | 86% | 72% |
| Compound 2-12 | Compound 12# 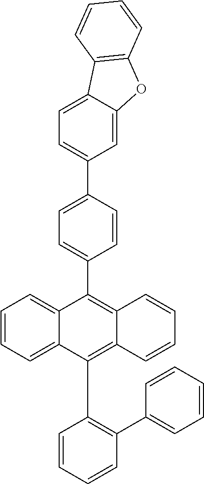 [SMILES: c1ccc(-c2ccccc2-c2c3ccccc3c(-c3ccc(-c4ccc5c(c4)oc4ccccc45)cc3)c3ccccc23)cc1] | 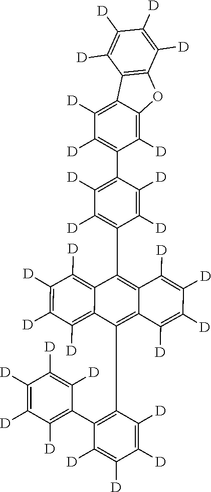 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c(-c4c([2H])c([2H])c5c(oc6c([2H])c([2H])c([2H])c([2H])c65)c4[2H])c([2H])c3[2H])c3c([2H])c([2H])c([2H])c([2H])c23)c([2H])c1[2H]] | 572 | 600 | 40 | 593 | 28 | 21 | 75% | 71% |
| Compound 2-13 | Compound 13# 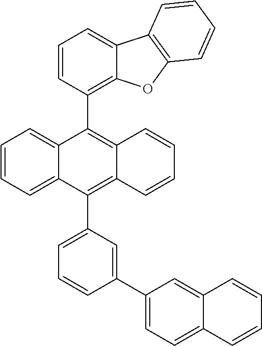 [SMILES: c1cc(-c2ccc3ccccc3c2)cc(-c2c3ccccc3c(-c3cccc4c3oc3ccccc34)c3ccccc23)c1] | 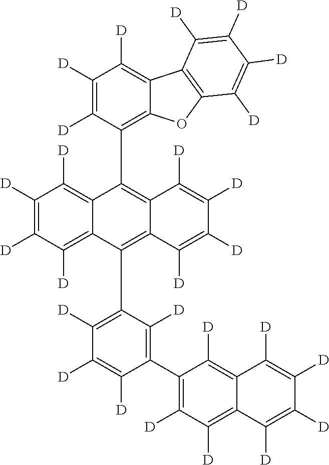 [SMILES: [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3c([2H])c([2H])c([2H])c4c3oc3c([2H])c([2H])c([2H])c([2H])c34)c3c([2H])c([2H])c([2H])c([2H])c23)c1[2H]] | 546 | 572 | 75 | 572 | 26 | 26 | 100% | 73% |
| Compound 2-14 | Compound 14# 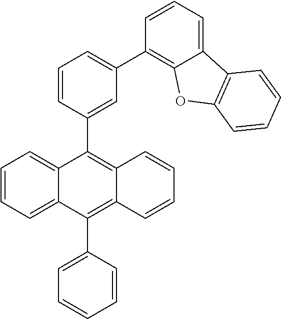 [SMILES: c1ccc(-c2c3ccccc3c(-c3cccc(-c4cccc5c4oc4ccccc45)c3)c3ccccc23)cc1] | 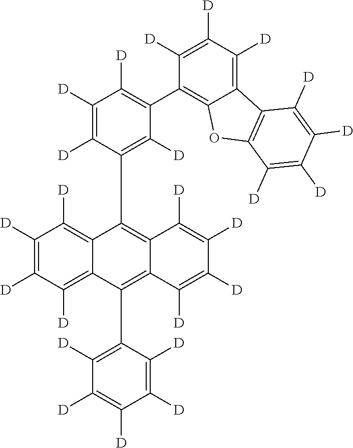 [SMILES: [2H]c1c([2H])c([2H])c(-c2c3c([2H])c([2H])c([2H])c([2H])c3c(-c3c([2H])c([2H])c([2H])c(-c4c([2H])c([2H])c([2H])c5c4oc4c([2H])c([2H])c([2H])c([2H])c45)c3[2H])c3c([2H])c([2H])c([2H])c([2H])c23)c([2H])c1[2H]] | 496 | 520 | 60 | 519 | 24 | 23 | 96% | 65% |
| Compound 2-15 | Compound 15# 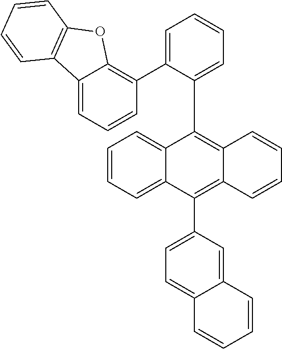 [SMILES: c1ccc(-c2cccc3c2oc2ccccc23)c(-c2c3ccccc3c(-c3ccc4ccccc4c3)c3ccccc23)c1] | 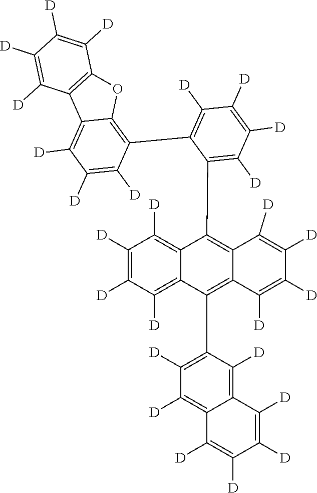 [SMILES: [2H]c1c([2H])c([2H])c(-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c(-c2c([2H])c([2H])c([2H])c3c2oc2c([2H])c([2H])c([2H])c([2H])c23)c1[2H]] | 546 | 572 | 60 | 568 | 26 | 22 | 85% | 66% |
| Compound 2-16 | Compound 16# 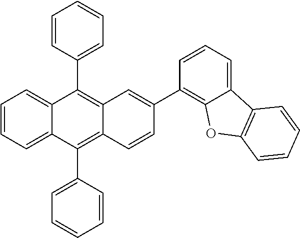 [SMILES: c1ccc(-c2c3ccccc3c(-c3ccccc3)c3cc(-c4cccc5c4oc4ccccc45)ccc23)cc1] | 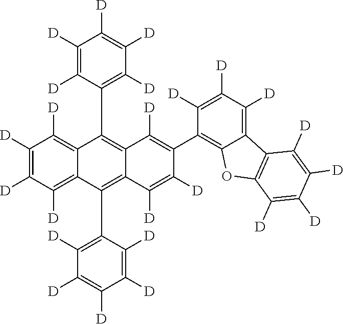 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4c([2H])c([2H])c([2H])c5c4oc4c([2H])c([2H])c([2H])c([2H])c45)c([2H])c([2H])c23)c([2H])c1[2H]] | 496 | 520 | 50 | 518 | 24 | 22 | 92% | 70% |
| Compound 2-17 | Compound 17# 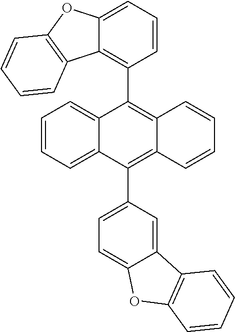 [SMILES: c1ccc2c(c1)oc1ccc(-c3c4ccccc4c(-c4cccc5oc6ccccc6c45)c4ccccc34)cc12] | 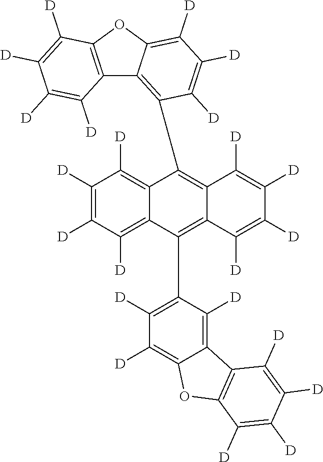 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6oc7c([2H])c([2H])c([2H])c([2H])c7c56)c5c([2H])c([2H])c([2H])c([2H])c45)c([2H])c32)c1[2H]] | 510 | 532 | 20 | 525 | 22 | 15 | 68% | 68% |
| Compound 2-18 | Compound 18# 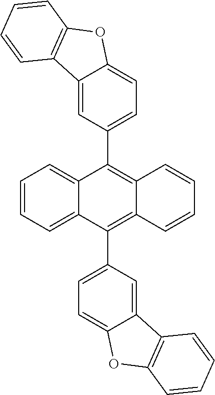 [SMILES: c1ccc2c(c1)oc1ccc(-c3c4ccccc4c(-c4ccc5oc6ccccc6c5c4)c4ccccc34)cc12] | 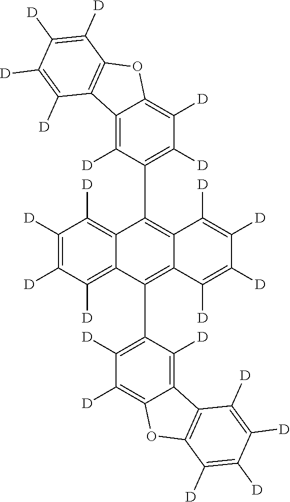 [SMILES: [2H]c1c([2H])c([2H])c2c(oc3c([2H])c([2H])c(-c4c5c([2H])c([2H])c([2H])c([2H])c5c(-c5c([2H])c([2H])c6oc7c([2H])c([2H])c([2H])c([2H])c7c6c5[2H])c5c([2H])c([2H])c([2H])c([2H])c45)c([2H])c32)c1[2H]] | 510 | 532 | 30 | 528 | 22 | 18 | 82% | 64% |
| Compound 2-19 | Compound 19# 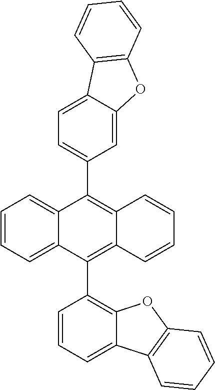 [SMILES: c1ccc2c(c1)oc1cc(-c3c4ccccc4c(-c4cccc5c4oc4ccccc45)c4ccccc34)ccc12] | 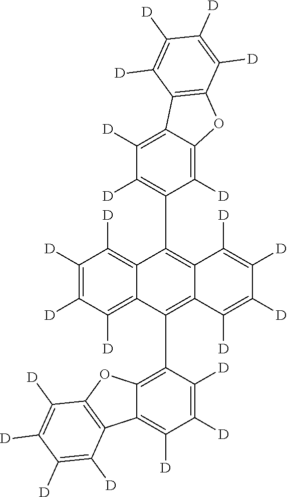 [SMILES: [2H]c1c([2H])c([2H])c2c(oc3c([2H])c(-c4c5c([2H])c([2H])c([2H])c([2H])c5c(-c5c([2H])c([2H])c([2H])c6c5oc5c([2H])c([2H])c([2H])c([2H])c56)c5c([2H])c([2H])c([2H])c([2H])c45)c([2H])c([2H])c32)c1[2H]] | 510 | 532 | 40 | 531 | 22 | 21 | 95% | 68% |
| Compound 2-20 | Compound 20# 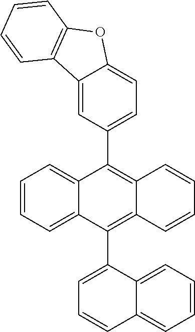 [SMILES: c1ccc2c(-c3c4ccccc4c(-c4ccc5oc6ccccc6c5c4)c4ccccc34)cccc2c1] | 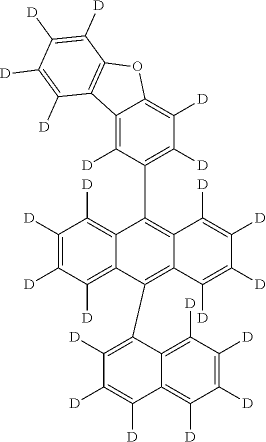 [SMILES: [2H]c1c([2H])c([2H])c2c(oc3c([2H])c([2H])c(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c([2H])c32)c1[2H]] | 470 | 492 | 60 | 492 | 22 | 22 | 100% | 70% |
| Compound 2-21 | Compound 21# 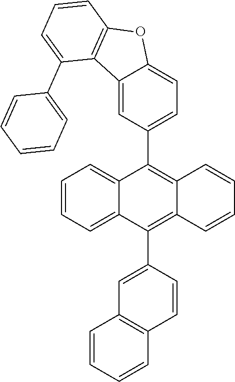 [SMILES: c1ccc(-c2cccc3oc4ccc(-c5c6ccccc6c(-c6ccc7ccccc7c6)c6ccccc56)cc4c23)cc1] | 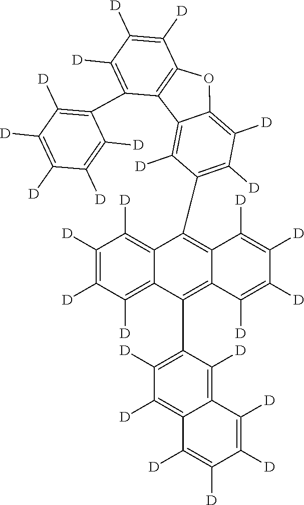 [SMILES: [2H]c1c([2H])c([2H])c(-c2c([2H])c([2H])c([2H])c3oc4c([2H])c([2H])c(-c5c6c([2H])c([2H])c([2H])c([2H])c6c(-c6c([2H])c([2H])c7c([2H])c([2H])c([2H])c([2H])c7c6[2H])c6c([2H])c([2H])c([2H])c([2H])c56)c([2H])c4c23)c([2H])c1[2H]] | 546 | 572 | 30 | 569 | 26 | 23 | 88% | 69% |
| Compound 2-22 | Compound 22# 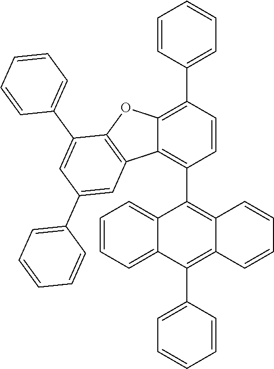 [SMILES: c1ccc(-c2cc(-c3ccccc3)c3oc4c(-c5ccccc5)ccc(-c5c6ccccc6c(-c6ccccc6)c6ccccc56)c4c3c2)cc1] | 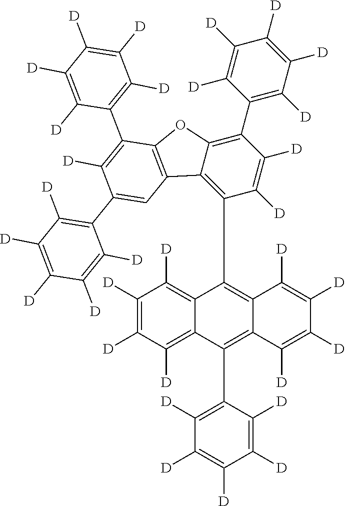 [SMILES: [2H]c1c([2H])c([2H])c(-c2cc3c(oc4c(-c5c([2H])c([2H])c([2H])c([2H])c5[2H])c([2H])c([2H])c(-c5c6c([2H])c([2H])c([2H])c([2H])c6c(-c6c([2H])c([2H])c([2H])c([2H])c6[2H])c6c([2H])c([2H])c([2H])c([2H])c56)c43)c(-c3c([2H])c([2H])c([2H])c([2H])c3[2H])c2[2H])c([2H])c1[2H]] | 648 | 680 | 50 | 677 | 32 | 29 | 91% | 72% |
| Compound 2-23 | Compound 23# 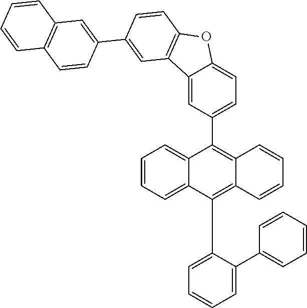 [SMILES: c1ccc(-c2ccccc2-c2c3ccccc3c(-c3ccc4oc5ccc(-c6ccc7ccccc7c6)cc5c4c3)c3ccccc23)cc1] | 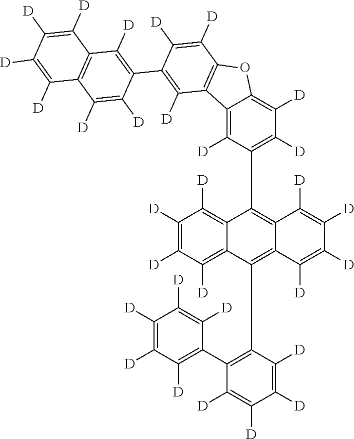 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c([2H])c([2H])c(-c6c([2H])c([2H])c7c([2H])c([2H])c([2H])c([2H])c7c6[2H])c([2H])c5c4c3[2H])c3c([2H])c([2H])c([2H])c([2H])c23)c([2H])c1[2H]] | 622 | 652 | 50 | 648 | 30 | 26 | 87% | 73% |
| Compound 2-24 | Compound 24# 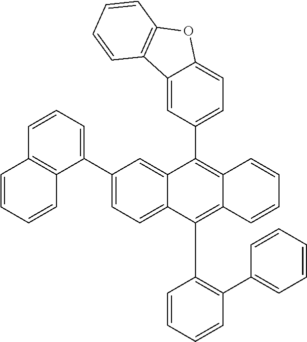 [SMILES: c1ccc(-c2ccccc2-c2c3ccccc3c(-c3ccc4oc5ccccc5c4c3)c3cc(-c4cccc5ccccc45)ccc23)cc1] | 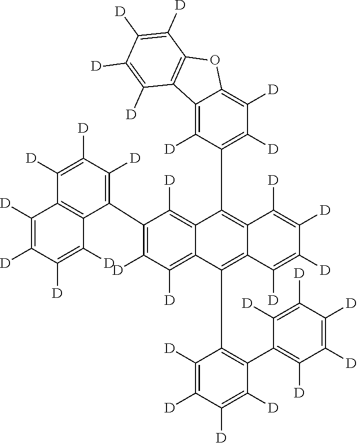 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c4oc5c([2H])c([2H])c([2H])c([2H])c5c4c3[2H])c3c([2H])c(-c4c([2H])c([2H])c([2H])c5c([2H])c([2H])c([2H])c([2H])c45)c([2H])c([2H])c23)c([2H])c1[2H]] | 622 | 652 | 60 | 648 | 30 | 26 | 87% | 80% |
| Compound 2-25 | Compound 25# 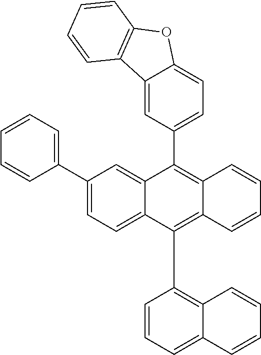 [SMILES: c1ccc(-c2ccc3c(-c4cccc5ccccc45)c4ccccc4c(-c4ccc5oc6ccccc6c5c4)c3c2)cc1] | 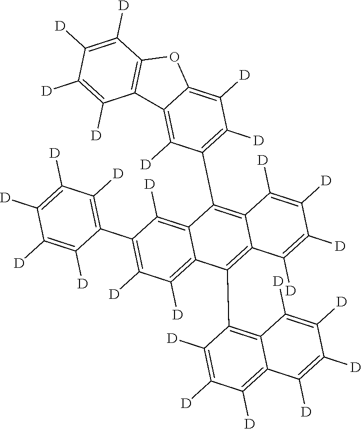 [SMILES: [2H]c1c([2H])c([2H])c(-c2c([2H])c([2H])c3c(-c4c([2H])c([2H])c([2H])c5c([2H])c([2H])c([2H])c([2H])c45)c4c([2H])c([2H])c([2H])c([2H])c4c(-c4c([2H])c([2H])c5oc6c([2H])c([2H])c([2H])c([2H])c6c5c4[2H])c3c2[2H])c([2H])c1[2H]] | 546 | 572 | 50 | 568 | 26 | 22 | 85% | 71% |
| Compound 2-26 | Compound 26# 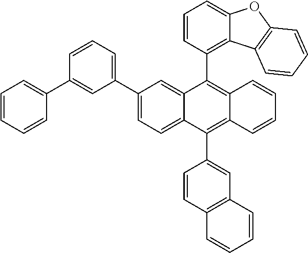 [SMILES: c1ccc(-c2cccc(-c3ccc4c(-c5ccc6ccccc6c5)c5ccccc5c(-c5cccc6oc7ccccc7c56)c4c3)c2)cc1] | 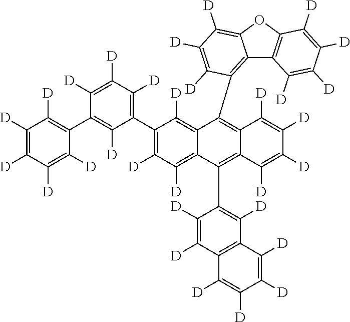 [SMILES: [2H]c1c([2H])c([2H])c(-c2c([2H])c([2H])c([2H])c(-c3c([2H])c([2H])c4c(-c5c([2H])c([2H])c6c([2H])c([2H])c([2H])c([2H])c6c5[2H])c5c([2H])c([2H])c([2H])c([2H])c5c(-c5c([2H])c([2H])c([2H])c6oc7c([2H])c([2H])c([2H])c([2H])c7c56)c4c3[2H])c2[2H])c([2H])c1[2H]] | 622 | 652 | 70 | 650 | 30 | 28 | 93% | 73% |
| Compound 2-27 | Compound 27# 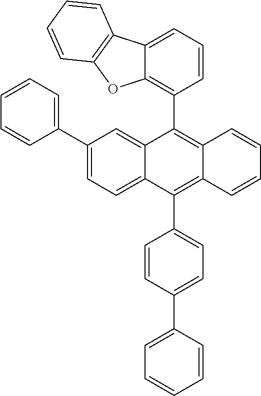 [SMILES: c1ccc(-c2ccc(-c3c4ccccc4c(-c4cccc5c4oc4ccccc45)c4cc(-c5ccccc5)ccc34)cc2)cc1] | 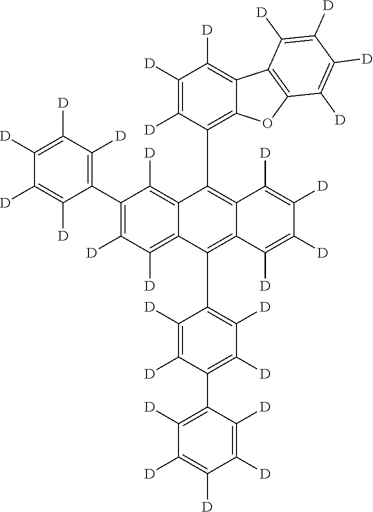 [SMILES: [2H]c1c([2H])c([2H])c(-c2c([2H])c([2H])c(-c3c4c([2H])c([2H])c([2H])c([2H])c4c(-c4c([2H])c([2H])c([2H])c5c4oc4c([2H])c([2H])c([2H])c([2H])c45)c4c([2H])c(-c5c([2H])c([2H])c([2H])c([2H])c5[2H])c([2H])c([2H])c34)c([2H])c2[2H])c([2H])c1[2H]] | 572 | 599 | 60 | 598 | 27 | 26 | 96% | 72% |

Each product has a different degree of deuterium substitution depending on the reaction time, and the substitution rate was determined according to the maximum m/z (M+) value.

Compounds 1 # to 27 #, which are the reactants, were synthesized with reference to the prior literature such as KR 1964435 B1, KR 1899728 B1, KR 1975945 B1, KR 2018-0098122 A, KR 2018-0102937 A, and KR 2018-0103352 A. Furthermore, for Compounds 2-1 to 2-27, which are deuterium-substituted products, the prior literature of KR 1538534 B1 was referenced.

| | | | | | | | The | The | D | |
|---|---|---|---|---|---|---|---|---|---|---|
| | | | | Product | Re- | Product | number | number | substi- | Syn- |
| Exemplary | | | Re- | theo- | action | actual | of total | of D | tution | thesis |
| deuterium | | | actant | retical | time | max | hydro- | sub- | rate | yield |
| compound | Reactant | Product | m/z | max m/z | (min) | m/z | gens | stitutions | (%) | (%) |
| Compound 2-28 | Compound 28# 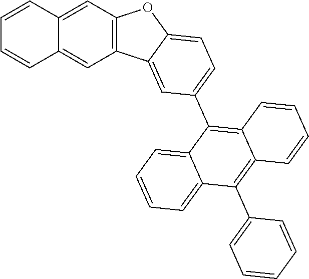 [SMILES: c1ccc(-c2c3ccccc3c(-c3ccc4oc5cc6ccccc6cc5c4c3)c3ccccc23)cc1] | 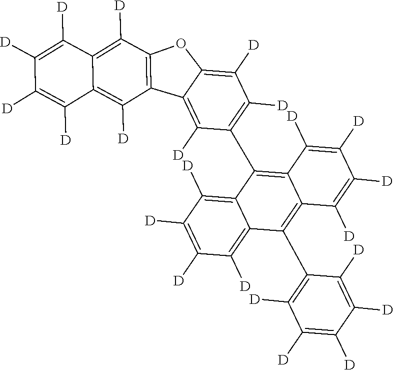 [SMILES: [2H]c1c([2H])c([2H])c(-c2c3c([2H])c([2H])c([2H])c([2H])c3c(-c3c([2H])c([2H])c4oc5c([2H])c6c([2H])c([2H])c([2H])c([2H])c6c([2H])c5c4c3[2H])c3c([2H])c([2H])c([2H])c([2H])c23)c([2H])c1[2H]] | 470 | 492 | 50 | 490 | 22 | 20 | 91% | 65% |
| Compound 2-29 | Compound 29# 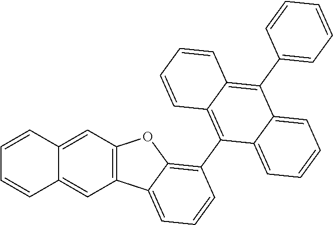 [SMILES: c1ccc(-c2c3ccccc3c(-c3cccc4c3oc3cc5ccccc5cc34)c3ccccc23)cc1] | 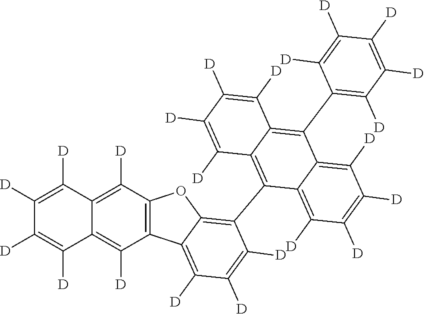 [SMILES: [2H]c1c([2H])c([2H])c(-c2c3c([2H])c([2H])c([2H])c([2H])c3c(-c3c([2H])c([2H])c([2H])c4c3oc3c([2H])c5c([2H])c([2H])c([2H])c([2H])c5c([2H])c34)c3c([2H])c([2H])c([2H])c([2H])c23)c([2H])c1[2H]] | 470 | 492 | 30 | 486 | 22 | 16 | 73% | 70% |
| Compound 2-30 | Compound 30# 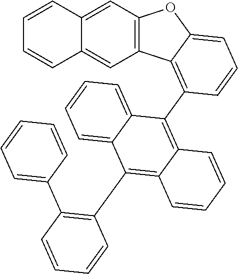 [SMILES: c1ccc(-c2ccccc2-c2c3ccccc3c(-c3cccc4oc5cc6ccccc6cc5c34)c3ccccc23)cc1] | 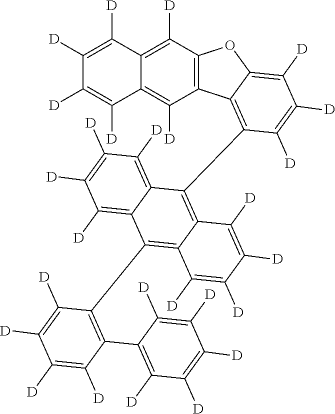 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c6c([2H])c([2H])c([2H])c([2H])c6c([2H])c5c34)c3c([2H])c([2H])c([2H])c([2H])c23)c([2H])c1[2H]] | 546 | 572 | 50 | 569 | 26 | 23 | 88% | 71% |
| Compound 2-31 | Compound 31# 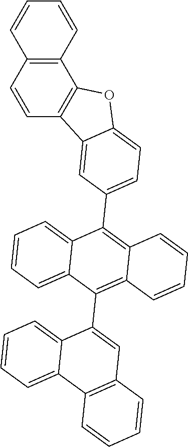 [SMILES: c1ccc2c(c1)cc(-c1c3ccccc3c(-c3ccc4oc5c6ccccc6ccc5c4c3)c3ccccc13)c1ccccc12] | 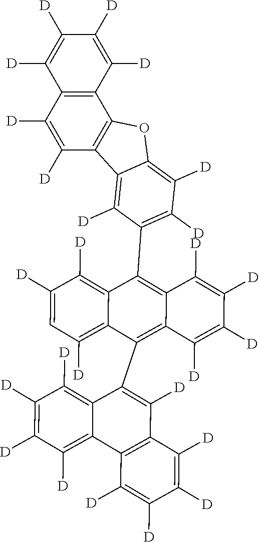 [SMILES: [2H]c1cc([2H])c2c(-c3c([2H])c4c([2H])c([2H])c([2H])c([2H])c4c4c([2H])c([2H])c([2H])c([2H])c34)c3c([2H])c([2H])c([2H])c([2H])c3c(-c3c([2H])c([2H])c4oc5c6c([2H])c([2H])c([2H])c([2H])c6c([2H])c([2H])c5c4c3[2H])c2c1[2H]] | 570 | 596 | 30 | 590 | 26 | 20 | 77% | 65% |
| Compound 2-32 | Compound 32# 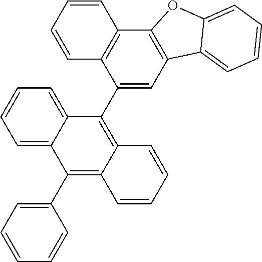 [SMILES: c1ccc(-c2c3ccccc3c(-c3cc4c5ccccc5oc4c4ccccc34)c3ccccc23)cc1] | 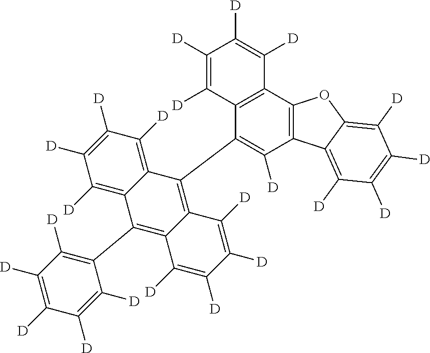 [SMILES: [2H]c1c([2H])c([2H])c(-c2c3c([2H])c([2H])c([2H])c([2H])c3c(-c3c([2H])c4c(oc5c([2H])c([2H])c([2H])c([2H])c54)c4c([2H])c([2H])c([2H])c([2H])c34)c3c([2H])c([2H])c([2H])c([2H])c23)c([2H])c1[2H]] | 470 | 492 | 40 | 489 | 22 | 19 | 86% | 66% |
| Compound 2-33 | Compound 33# 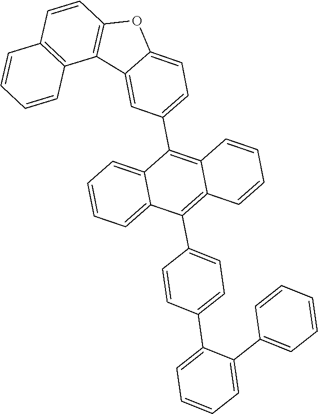 [SMILES: c1ccc(-c2ccccc2-c2ccc(-c3c4ccccc4c(-c4ccc5oc6ccc7ccccc7c6c5c4)c4ccccc34)cc2)cc1] | 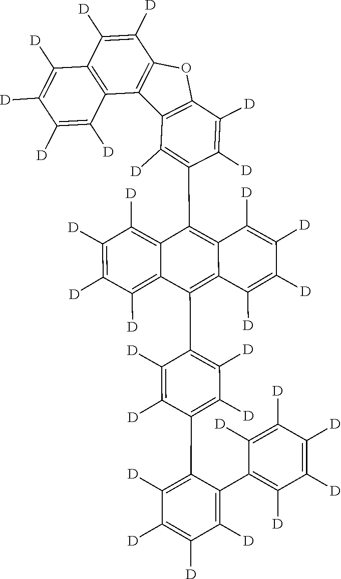 [SMILES: [2H]c1c([2H])c([2H])c(-c2c([2H])c([2H])c([2H])c([2H])c2-c2c([2H])c([2H])c(-c3c4c([2H])c([2H])c([2H])c([2H])c4c(-c4c([2H])c([2H])c5oc6c([2H])c([2H])c7c([2H])c([2H])c([2H])c([2H])c7c6c5c4[2H])c4c([2H])c([2H])c([2H])c([2H])c34)c([2H])c2[2H])c([2H])c1[2H]] | 622 | 652 | 50 | 649 | 30 | 27 | 90% | 71% |
| Compound 2-34 | Compound 34# 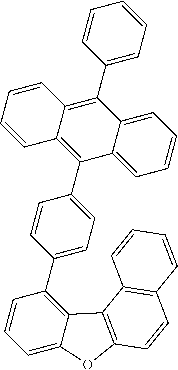 [SMILES: c1ccc(-c2c3ccccc3c(-c3ccc(-c4cccc5oc6ccc7ccccc7c6c45)cc3)c3ccccc23)cc1] | 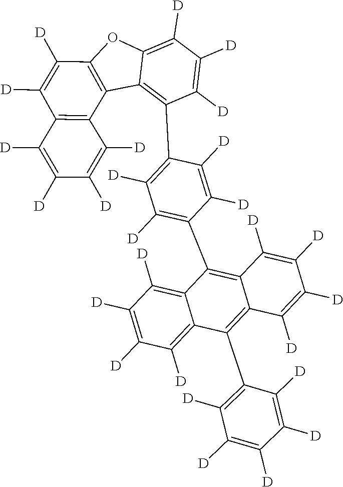 [SMILES: [2H]c1c([2H])c([2H])c(-c2c3c([2H])c([2H])c([2H])c([2H])c3c(-c3c([2H])c([2H])c(-c4c([2H])c([2H])c([2H])c5oc6c([2H])c([2H])c7c([2H])c([2H])c([2H])c([2H])c7c6c45)c([2H])c3[2H])c3c([2H])c([2H])c([2H])c([2H])c23)c([2H])c1[2H]] | 546 | 572 | 35 | 567 | 26 | 21 | 81% | 70% |
| Compound 2-35 | Compound 35# 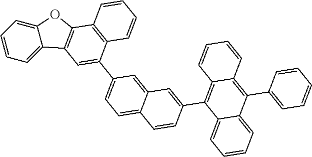 [SMILES: c1ccc(-c2c3ccccc3c(-c3ccc4ccc(-c5cc6c7ccccc7oc6c6ccccc56)cc4c3)c3ccccc23)cc1] | 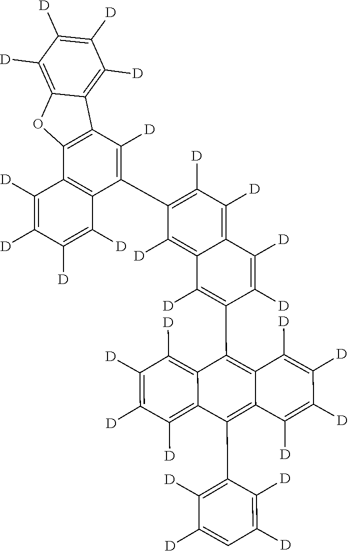 [SMILES: [2H]c1cc([2H])c([2H])c(-c2c3c([2H])c([2H])c([2H])c([2H])c3c(-c3c([2H])c([2H])c4c([2H])c([2H])c(-c5c([2H])c6c(oc7c([2H])c([2H])c([2H])c([2H])c76)c6c([2H])c([2H])c([2H])c([2H])c56)c([2H])c4c3[2H])c3c([2H])c([2H])c([2H])c([2H])c23)c1[2H]] | 596 | 624 | 40 | 619 | 28 | 23 | 82% | 75% |
| Compound 2-36 | Compound 36# 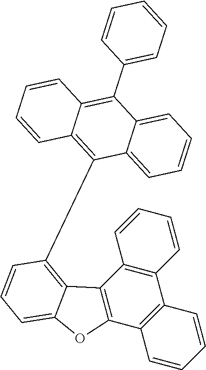 [SMILES: c1ccc(-c2c3ccccc3c(-c3cccc4oc5c6ccccc6c6ccccc6c5c34)c3ccccc23)cc1] | 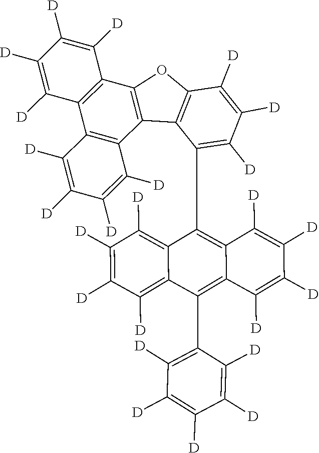 [SMILES: [2H]c1c([2H])c([2H])c(-c2c3c([2H])c([2H])c([2H])c([2H])c3c(-c3c([2H])c([2H])c([2H])c4oc5c6c([2H])c([2H])c([2H])c([2H])c6c6c([2H])c([2H])c([2H])c([2H])c6c5c34)c3c([2H])c([2H])c([2H])c([2H])c23)c([2H])c1[2H]] | 520 | 544 | 60 | 543 | 24 | 23 | 96% | 69% |
| Compound 2-37 | Compound 37# 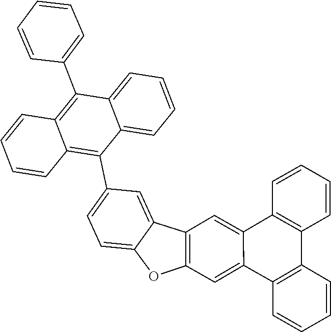 [SMILES: c1ccc(-c2c3ccccc3c(-c3ccc4oc5cc6c7ccccc7c7ccccc7c6cc5c4c3)c3ccccc23)cc1] | 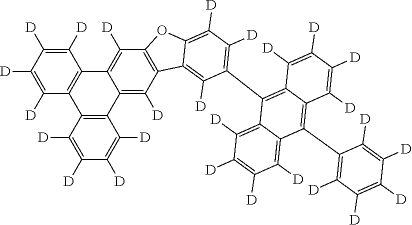 [SMILES: [2H]c1c([2H])c([2H])c(-c2c3c([2H])c([2H])c([2H])c([2H])c3c(-c3c([2H])c([2H])c4oc5c([2H])c6c7c([2H])c([2H])c([2H])c([2H])c7c7c([2H])c([2H])c([2H])c([2H])c7c6c([2H])c5c4c3[2H])c3c([2H])c([2H])c([2H])c([2H])c23)c([2H])c1[2H]] | 570 | 596 | 60 | 594 | 26 | 24 | 92% | 68% |
| Compound 2-38 | Compound 38# 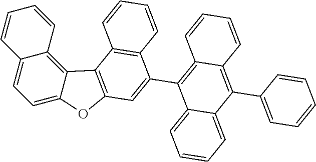 [SMILES: c1ccc(-c2c3ccccc3c(-c3cc4oc5ccc6ccccc6c5c4c4ccccc34)c3ccccc23)cc1] | 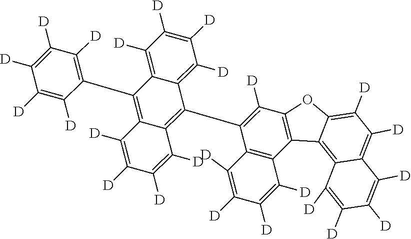 [SMILES: [2H]c1c([2H])c([2H])c(-c2c3c([2H])c([2H])c([2H])c([2H])c3c(-c3c([2H])c4oc5c([2H])c([2H])c6c([2H])c([2H])c([2H])c([2H])c6c5c4c4c([2H])c([2H])c([2H])c([2H])c34)c3c([2H])c([2H])c([2H])c([2H])c23)c([2H])c1[2H]] | 520 | 544 | 35 | 538 | 24 | 18 | 75% | 63% |
| Compound 2-39 | Compound 39# 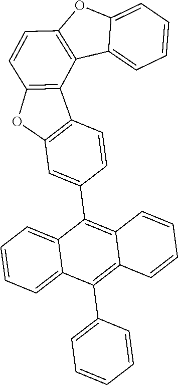 [SMILES: c1ccc(-c2c3ccccc3c(-c3ccc4c(c3)oc3ccc5oc6ccccc6c5c34)c3ccccc23)cc1] | 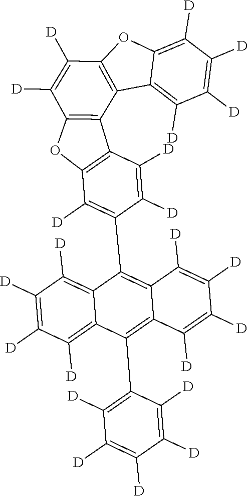 [SMILES: [2H]c1c([2H])c([2H])c(-c2c3c([2H])c([2H])c([2H])c([2H])c3c(-c3c([2H])c([2H])c4c(oc5c([2H])c([2H])c6oc7c([2H])c([2H])c([2H])c([2H])c7c6c54)c3[2H])c3c([2H])c([2H])c([2H])c([2H])c23)c([2H])c1[2H]] | 510 | 532 | 60 | 531 | 22 | 21 | 95% | 66% |
| Compound 2-40 | Compound 40# 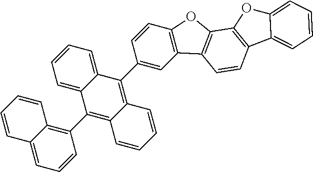 [SMILES: c1ccc2c(-c3c4ccccc4c(-c4ccc5oc6c(ccc7c8ccccc8oc76)c5c4)c4ccccc34)cccc2c1] | 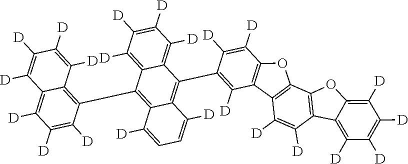 [SMILES: [2H]c1c([2H])c([2H])c2c(oc3c4oc5c([2H])c([2H])c(-c6c7c([2H])ccc([2H])c7c(-c7c([2H])c([2H])c([2H])c8c([2H])c([2H])c([2H])c([2H])c78)c7c([2H])c([2H])c([2H])c([2H])c67)c([2H])c5c4c([2H])c([2H])c32)c1[2H]] | 560 | 584 | 15 | 572 | 24 | 12 | 50% | 65% |
| Compound 2-41 | Compound 41# 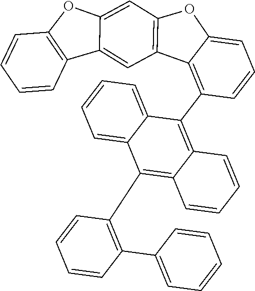 [SMILES: c1ccc(-c2ccccc2-c2c3ccccc3c(-c3cccc4oc5cc6oc7ccccc7c6cc5c34)c3ccccc23)cc1] | 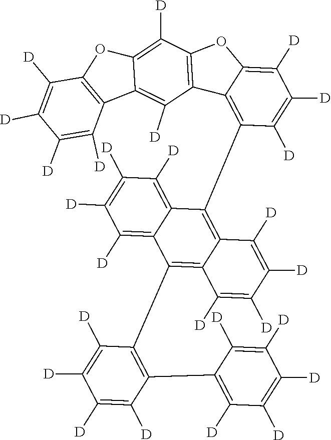 [SMILES: [2H]c1c([2H])c([2H])c(-c2c([2H])c([2H])c([2H])c([2H])c2-c2c3c([2H])c([2H])c([2H])c([2H])c3c(-c3c([2H])c([2H])c([2H])c4oc5c([2H])c6oc7c([2H])c([2H])c([2H])c([2H])c7c6c([2H])c5c34)c3c([2H])c([2H])c([2H])c([2H])c23)c([2H])c1[2H]] | 586 | 612 | 40 | 607 | 26 | 21 | 81% | 67% |
| Compound 2-42 | Compound 42# 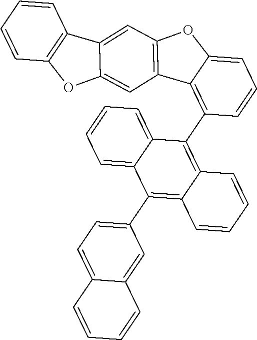 [SMILES: c1ccc2cc(-c3c4ccccc4c(-c4cccc5oc6cc7c(cc6c45)oc4ccccc47)c4ccccc34)ccc2c1] | 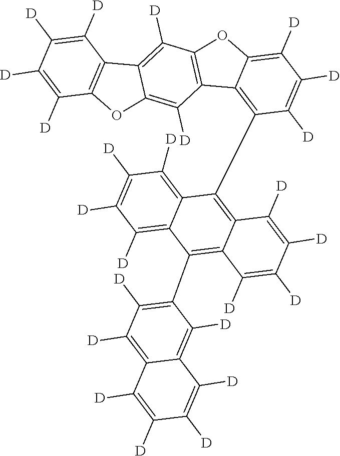 [SMILES: [2H]c1c([2H])c([2H])c2c(oc3c([2H])c4c(oc5c([2H])c([2H])c([2H])c(-c6c7c([2H])c([2H])c([2H])c([2H])c7c(-c7c([2H])c([2H])c8c([2H])c([2H])c([2H])c([2H])c8c7[2H])c7c([2H])c([2H])c([2H])c([2H])c67)c54)c([2H])c32)c1[2H]] | 560 | 584 | 75 | 584 | 24 | 24 | 100% | 70% |
| Compound 2-43 | Compound 43# 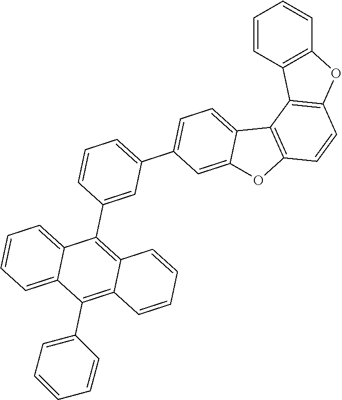 [SMILES: c1ccc(-c2c3ccccc3c(-c3cccc(-c4ccc5c(c4)oc4ccc6oc7ccccc7c6c45)c3)c3ccccc23)cc1] | 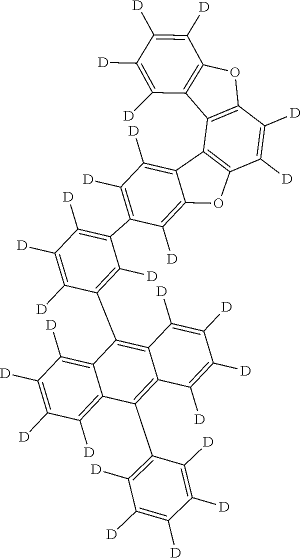 [SMILES: [2H]c1c([2H])c([2H])c(-c2c3c([2H])c([2H])c([2H])c([2H])c3c(-c3c([2H])c([2H])c([2H])c(-c4c([2H])c([2H])c5c(oc6c([2H])c([2H])c7oc8c([2H])c([2H])c([2H])c([2H])c8c7c65)c4[2H])c3[2H])c3c([2H])c([2H])c([2H])c([2H])c23)c([2H])c1[2H]] | 586 | 612 | 60 | 610 | 26 | 24 | 92% | 70% |
| Compound 2-44 | Compound 44# 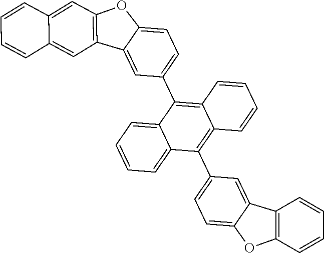 [SMILES: c1ccc2cc3c(cc2c1)oc1ccc(-c2c4ccccc4c(-c4ccc5oc6ccccc6c5c4)c4ccccc24)cc13] | 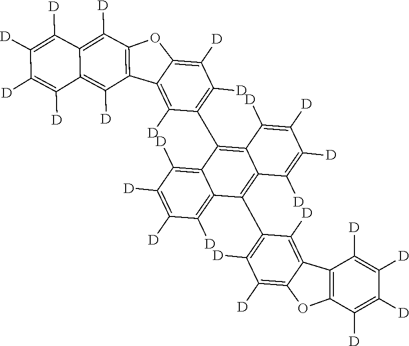 [SMILES: [2H]c1c([2H])c([2H])c2c(oc3c([2H])c([2H])c(-c4c5c([2H])c([2H])c([2H])c([2H])c5c(-c5c([2H])c([2H])c6oc7c([2H])c8c([2H])c([2H])c([2H])c([2H])c8c([2H])c7c6c5[2H])c5c([2H])c([2H])c([2H])c([2H])c45)c([2H])c32)c1[2H]] | 560 | 584 | 60 | 580 | 24 | 20 | 83% | 69% |
| Compound 2-45 | Compound 45# 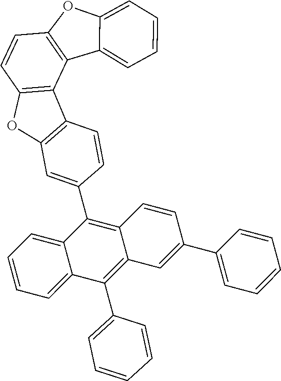 [SMILES: c1ccc(-c2ccc3c(-c4ccc5c(c4)oc4ccc6oc7ccccc7c6c45)c4ccccc4c(-c4ccccc4)c3c2)cc1] | 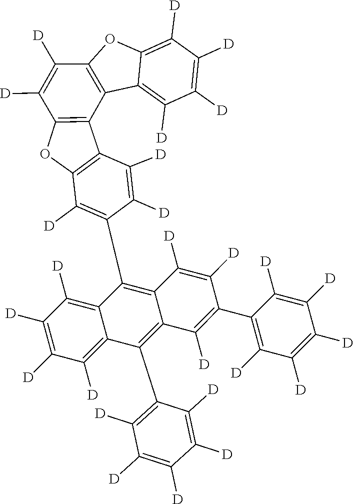 [SMILES: [2H]c1c([2H])c([2H])c(-c2c([2H])c([2H])c3c(-c4c([2H])c([2H])c5c(oc6c([2H])c([2H])c7oc8c([2H])c([2H])c([2H])c([2H])c8c7c65)c4[2H])c4c([2H])c([2H])c([2H])c([2H])c4c(-c4c([2H])c([2H])c([2H])c([2H])c4[2H])c3c2[2H])c([2H])c1[2H]] | 586 | 612 | 50 | 607 | 26 | 21 | 81% | 75% |
| Compound 2-46 | Compound 46# 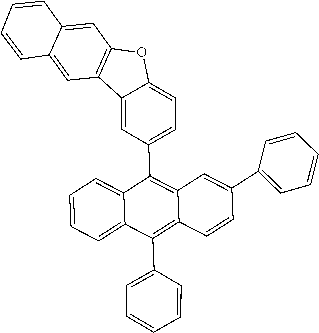 [SMILES: c1ccc(-c2ccc3c(-c4ccccc4)c4ccccc4c(-c4ccc5oc6cc7ccccc7cc6c5c4)c3c2)cc1] | 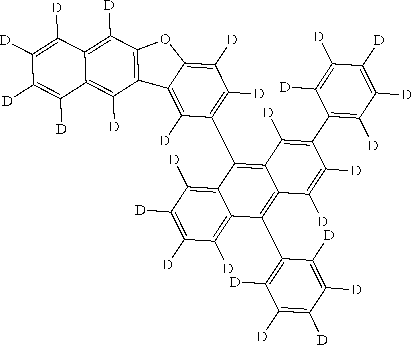 [SMILES: [2H]c1c([2H])c([2H])c(-c2c([2H])c([2H])c3c(-c4c([2H])c([2H])c([2H])c([2H])c4[2H])c4c([2H])c([2H])c([2H])c([2H])c4c(-c4c([2H])c([2H])c5oc6c([2H])c7c([2H])c([2H])c([2H])c([2H])c7c([2H])c6c5c4[2H])c3c2[2H])c([2H])c1[2H]] | 546 | 572 | 60 | 568 | 26 | 22 | 85% | 72% |
| Compound 2-47 | Compound 47# 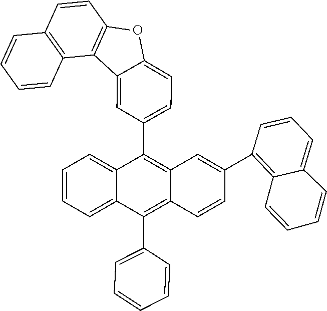 [SMILES: c1ccc(-c2c3ccccc3c(-c3ccc4oc5ccc6ccccc6c5c4c3)c3cc(-c4cccc5ccccc45)ccc23)cc1] | 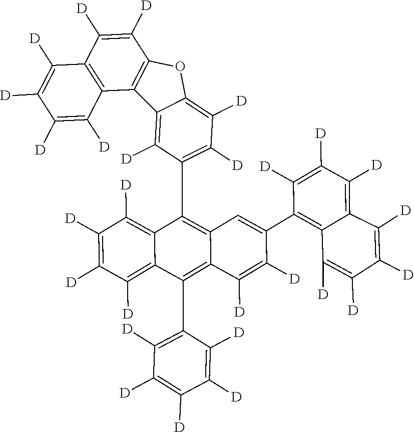 [SMILES: [2H]c1c([2H])c([2H])c(-c2c3c([2H])c([2H])c(-c4c([2H])c([2H])c([2H])c5c([2H])c([2H])c([2H])c([2H])c45)cc3c(-c3c([2H])c([2H])c4oc5c([2H])c([2H])c6c([2H])c([2H])c([2H])c([2H])c6c5c4c3[2H])c3c([2H])c([2H])c([2H])c([2H])c23)c([2H])c1[2H]] | 596 | 624 | 60 | 620 | 28 | 24 | 86% | 70% |

Each product has a different degree of deuterium substitution depending on the reaction time, and the substitution rate was determined according to the maximum m/z (M+) value.

Compounds 28 # to 47 #, which are the reactants, were synthesized with reference to the in-house prior literature such as KR 1994238 B1, KR 1670193 B1, KR 1754445 B1, and KR 1368164 B1. Furthermore, for Compounds 2-28 to 2-47, which are deuterium-substituted products, the prior literature of KR 1538534 BA was referenced.

### [Experimental Example 1] Dipole Moment (DM) and Sublimation Temperature of Preparation Example 2

TABLE 2-4

| | | Sublimation | | | Sublimation | | | Sublimation |
|---|---|---|---|---|---|---|---|---|
| | DM | temperature | | DM | temperature | | DM | temperature |
| Compound A | 0.29 | 270 | Compound 1# | 1.01 | 260 | Compound 23# | 0.94 | 290 |
| Compound B | 0.17 | 250 | Compound 2# | 1.21 | 270 | Compound 24# | 1.11 | 300 |
| Compound C | 0.21 | 260 | Compound 3# | 1.23 | 270 | Compound 25# | 1.05 | 270 |
| Compound D | 0.18 | 240 | Compound 4# | 0.81 | 290 | Compound 26# | 1.02 | 300 |
| Compound E | 0.12 | 260 | Compound 5# | 1.24 | 260 | Compound 27# | 1.07 | 270 |
| Compound F | 0.15 | 240 | Compound 6# | 0.82 | 240 | Compound 28# | 1.03 | 270 |
| Compound G | 0.06 | 250 | Compound 7# | 0.89 | 260 | Compound 29# | 1.02 | 260 |
| Compound H | 0.20 | 250 | Compound 8# | 0.70 | 260 | Compound 30# | 1.12 | 280 |
| Compound I | 0.23 | 250 | Compound 9# | 1.20 | 280 | Compound 31# | 0.94 | 290 |
| Compound J | 0.16 | 250 | Compound 10# | 0.69 | 260 | Compound 32# | 0.91 | 270 |
| Compound K | 0.29 | 240 | Compound 11# | 0.73 | 290 | Compound 33# | 1.01 | 290 |
| Compound L | 0.21 | 260 | Compound 12# | 1.01 | 270 | Compound 34# | 0.98 | 280 |
| Compound M | 0.24 | 270 | Compound 13# | 0.64 | 260 | Compound 35# | 1.15 | 300 |
| Compound N | 0.21 | 260 | Compound 14# | 0.60 | 250 | Compound 36# | 1.04 | 260 |
| Compound O | 0.18 | 270 | Compound 15# | 0.63 | 270 | Compound 37# | 1.10 | 270 |
| Compound P | 0.15 | 260 | Compound 16# | 1.01 | 260 | Compound 38# | 0.90 | 280 |
| Compound Q | 0.17 | 260 | Compound 17# | 0.74 | 260 | Compound 39# | 1.15 | 280 |
| Compound R | 0.02 | 270 | Compound 18# | 0.49 | 260 | Compound 40# | 1.17 | 290 |
| Compound S | 0.30 | 260 | Compound 19# | 0.64 | 260 | Compound 41# | 1.25 | 280 |
| Compound T | 0.08 | 260 | Compound 20# | 0.66 | 260 | Compound 42# | 1.22 | 290 |
| Compound U | 0.12 | 270 | Compound 21# | 0.95 | 270 | Compound 43# | 1.28 | 290 |
| Compound V | 0.17 | 290 | Compound 22# | 1.02 | 290 | Compound 44# | 1.01 | 290 |
| Compound W | 0.24 | 290 | | | | Compound 45# | 1.21 | 290 |
| | | | | | | Compound 46# | 1.20 | 280 |
| | | | | | | Compound 47# | 1.22 | 290 |

In this case, the unit of dipole moment (DM) is debye, and the unit of sublimation temperature is ° C.

The physical properties of the compounds synthesized in the Preparation Examples are shown in [Table 2-4]. For the physical properties of Compounds A to W, although Compounds 1-A to 1-W have a difference in chemical structure between carbon-hydrogen skeleton and carbon-deuterium skeleton, the basic chemical skeletons are the same and the difference in dipole moment is almost the same, so that the numerical values for the physical properties are commonly referred to as Compounds A to W. Further, Compounds 1 # to 47 # are the same as Compounds 2-1 to 2-47, and the numerical values thereof were commonly referred to as Compounds 1 # to 47 #.

Among the compounds synthesized in Preparation Example 2, the aryl-based compound of Chemical Formula 2 (host1) and the furan-based compound of Chemical Formula 2 (host2) are made to have a difference in dipole moment in their chemical structures. Compounds A to W that correspond to the aryl-based Chemical Formula 2 have a skeleton based only on carbon and hydrogen by having an aryl-based substituent, and the compartmentalization of the chemical structure with fewer and more electrons is limited, thereby showing the results that the dipole moment (DM) does not exceed a maximum of 0.3 debye. In contrast, it can be confirmed that furan-based compounds 1 # to 47 # of Chemical Formula 2 have a dipole moment (DM) relatively higher than those of Compounds A to W of the aryl-based chemical formulae by substituting an anthracene skeleton with furan including aryl or heteroaryl including relatively electron-rich oxygen to have the potential capable of deepening the compartmentalization of electrons in a chemical structure having a carbon-hydrogen skeleton. Therefore, the combination of the two structures presented in the present document has a range in which the DM difference between the two hosts exceeds at least 0.2.

$$\vert {DM}_{host 1} - {DM}_{host 2} \vert > 0.2$$

In addition, it could be confirmed that the sublimation temperature of the compound synthesized in the Preparation Example is less than 400° C., and when the compound has a sublimation temperature of 400° C. or more, the compound has many limitations when used as a material for an organic electroluminescent device.

Moreover, in order to pre-sublimate and prepare a mixture of one of the aryl-based compounds of Chemical Formula 2 and one of the furan-based compounds of Chemical Formula 2, it is preferred that the following formula be satisfied.

$$\vert {T}_{sub 1} - {T}_{sub 2} \vert \leq 20 °C .$$

### [Experimental Example 2] Preparation Example of Mixture

The premixability of two compounds (one each of Chemical Formulae 2 and 3) corresponding to the two hosts to be mixed was tested by high pressure liquid chromatography (HPLC) analysis of the evaporated film. In the preparation example of this mixture, Compound J (Chemical Formula 2) and Compound 6 # (Chemical Formula 3) were used.

For this purpose, 0.15 g of Compound J (or Compound 1-J) and 0.15 g of Compound 6 # (or Compound 2-6) were mixed at 1 1 and pulverized. 0.3 g of the mixture was loaded into the evaporation source of a VTE vacuum chamber. Pressure was reduced to a pressure of 10⁻⁷ Torr by pumping the chamber. The premixed components were deposited onto a glass substrate at a rate of 2 Å/sec. After a 600 Å-thick film was deposited without interrupting the deposition process to avoid cooling the feedstock and maintain the feedstock at a suitable temperature, the substrate was consecutively replaced further twice. The deposited film was analyzed by HPLC, and the results are shown in the following FIG. 5. These three substrate samples were taken and labeled with Film1 to Film3 as described below. The compositions of Compound J (Chemical Formula 2) and Compound 6 # (Chemical Formula 3) were not significantly changed from Film 1 to Film 3. A pre- and post-deposition concentration change within 10%, preferably 5% throughout the process is considered excellent and useful for commercially available OLED applications, and it appears that the above concentration change may be maintained when the difference in sublimation temperature between the two mixed compounds is 20° C. or less. The difference in sublimation temperature between Compound J and Compound 6 # was 10° C.

As shown below, the slight variation in concentration did not show any variation tendency in the device, and the sample collection and HPLC analysis could be described by the following Equation 2. More specifically, the first compound (Compound J) or the second compound (Compound 6 #) has a concentration C1 in the mixture, has a concentration C2 in a film formed by evaporating the mixture at a deposition rate of 1 Å/s to 10 Å/s on a surface located at a fixed distance from a point where the mixture is evaporated in a high vacuum evaporation system with a chamber base pressure of 1×10⁻⁴ Torr to 1×10⁻⁹ Torr, and satisfies the following equation.

$$\vert ( C 1 - C 2 ) / C 1 \vert < 10 \%$$

For the compound represented by Compound 6 #, when the concentration C1 in the composition is 48.85%, the change in concentration is 2.3% because the concentration of Film 1 is 47.69%, the change in concentration is 3.9% because the concentration of Film 2 is 46.94%, and the change in concentration is 2.3% because the concentration of Film 3 is 47.71%.

The change in concentration was not significantly changed in the produced Films 1 to 3, and it could be observed that the deviation between the films was small. Accordingly, the conditions described above are desirable.

### [Experimental Example 3] Comparative Preparation Example of Mixture

The following results are exemplarily shown in the results of mixtures out of the conditions presented in the present specification. In the comparative preparation example of the following mixture, Compound B (Chemical Formula 2) and Compound 24 # (Chemical Formula 3) were used.

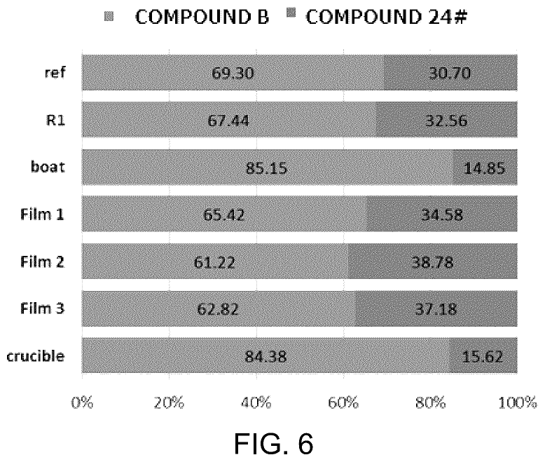

For mixing, 0.21 g of Compound B (or Compound 1-B) and 0.09 g of Compound 24 # (or Compound 2-24) were mixed at 7: 3 and pulverized. The conditions for manufacture were the same as those for the preparation example of the mixture in Preparation Example 3, and the results are shown in FIG. 6.

Based on the above Equation 2; for the compound represented by Compound B, when the concentration C1 in the composition is 69.30%, the change in concentration is 5.5% because the concentration of Film 1 is 65.42%, the change in concentration is 11.6% because the concentration of Film 2 is 61.22%, and the change in concentration is 9.3% because the concentration of Film 3 is 62.82%.

The change in concentration was significantly different changed in the produced Films 1 to 3, and in particular, a change of 10% or more was observed, and it could be observed that the deviation between the films was large. Accordingly, the conditions described above are not desirable.

### [Experimental Example 4] Manufacture of OLED

### Example 1

As a positive electrode, a substrate on which ITO/Ag/ITO were deposited to have a thickness of 70/1,000/70 A was cut into a size of 50 mm×50 mm×0.5 mm, put into distilled water in which a detergent was dissolved, and ultrasonically washed. A product manufactured by Fischer Co., was used as the detergent, and distilled water twice filtered using a filter manufactured by Millipore Co., was used as the distilled water. After the ITO was washed for 30 minutes, ultrasonic washing was repeated twice with distilled water for 10 minutes. After the washing using distilled water was completed, ultrasonic washing was conducted using isopropyl alcohol, acetone, and methanol solvents in this order, and drying was then conducted.

The following compound HI-1 was thermally vacuum-deposited to have a thickness of 50 Å on the positive electrode thus prepared, thereby forming a hole injection layer, and the following compound HT1 as a material which transports holes was vacuum-deposited to have a thickness of 1,150 A thereon, thereby forming a hole transport layer, Next, a hole adjusting layer was formed using the following compound EB1 (150 Å). Next, the host Compound 1-1 synthesized in Preparation Example 1 and Dopant BD1 (2 wt %) were vacuum-deposited to a thickness of 60 Å to form a light emitting layer as a first light emitting layer, and then the host Compound A of Preparation Example 2 and the following compound BD1 as dopant (2 wt %) were vacuum deposited to a thickness of 300 Å to form a light emitting layer as a second light emitting layer. Thereafter, the following compound HB1 was deposited to have a thickness of 50 Å, thereby forming an electron adjusting layer, and the following Compounds ET1 and Liq were mixed at 5:5 (mass ratio), thereby forming an electron transport layer having a thickness of 250 Å. Sequentially, magnesium and lithium fluoride (LiF) were deposited to have a thickness of 50 Å to form a film as an electron injection layer <EIL>, magnesium and silver (1:4) were used to form a negative electrode having a thickness of 200 Å, and then the following compound CP1 was deposited to have a thickness of 600 Å, thereby completing a device. In the aforementioned procedure, the deposition rates of the organic materials were each maintained at 1 Å/sec.

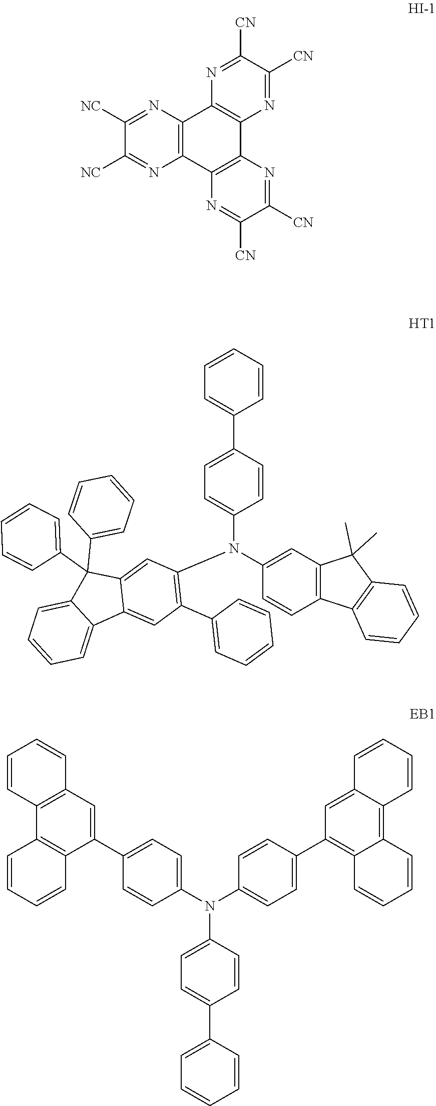 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3cc4c(cc3-c3ccccc3)-c3ccccc3C4(c3ccccc3)c3ccccc3)cc21 ; c1ccc(-c2ccc(N(c3ccc(-c4cc5ccccc5c5ccccc45)cc3)c3ccc(-c4cc5ccccc5c5ccccc45)cc3)cc2)cc1]

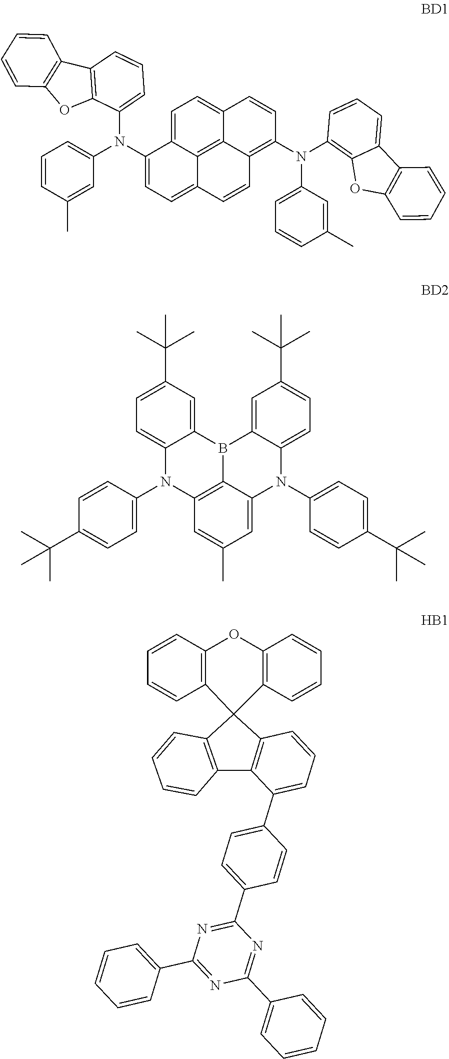 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2nc(-c3ccccc3)nc(-c3ccc(-c4cccc5c4-c4ccccc4C54c5ccccc5Oc5ccccc54)cc3)n2)cc1]

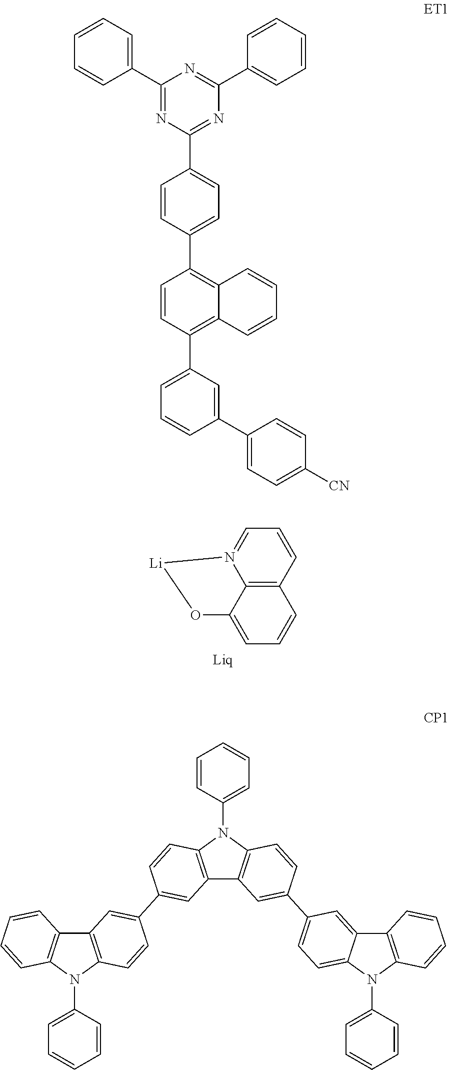

### Comparative Examples 1 to 8 and Examples 2 to 86

Devices were manufactured in the same manner as in Example 1, except that in Comparative Examples 1 to 8 and Examples 2 to 86, the materials shown in the following [Table 1A] were used as materials for the light emitting layer.

For the devices manufactured in Comparative Examples 1 to 8 and Examples 1 to 86, the driving voltage, light emitting efficiency, and a time (LT95) for reaching a 95% value compared to the initial luminance were measured at a current density of 20 mA/cm². The results are shown in the following [Table 1A].

TABLE 1A

| | | | | | Device results | | | |
|---|---|---|---|---|---|---|---|---|
| | First light | | Second light | | Voltage(V) | Cd/A | Color | Service life |
| Experimental | emitting layer | | emitting layer | | (@ 20 | (@ 20 | coordinate | (T95, h) (@ |
| Example | Host | Dopant | Host | Dopant | mA/cm²) | mA/cm²) | (x, y) | 20 mA/cm²) |
| Comparative | — | — | Compound A | BD1 | 3.90 | 5.34 | (0.140, 0.050) | 40.8 |
| Example 1 | | | | | | | | |
| Comparative | — | — | Compound 1-H | BD1 | 3.91 | 5.20 | (0.140, 0.049) | 42.4 |
| Example 2 | | | | | | | | |
| Comparative | — | — | Compound 1-M | BD1 | 3.86 | 5.28 | (0.140, 0.060) | 44.5 |
| Example 3 | | | | | | | | |
| Comparative | — | — | Compound 1-H | BD2 | 3.98 | 5.46 | (0.127, 0.095) | 38.1 |
| Example 4 | | | | | | | | |
| Comparative | — | — | Compound 2 | BD1 | 3.85 | 5.26 | (0.140, 0.051) | 40.1 |
| Example 5 | | | | | | | | |
| Comparative | — | — | Compound 37# | BD1 | 3.80 | 5.19 | (0.140, 0.058) | 36.1 |
| Example 6 | | | | | | | | |
| Comparative | — | — | Compound 2-3 | BD1 | 3.81 | 5.30 | (0.140, 0.052) | 44.5 |
| Example 7 | | | | | | | | |
| Comparative | — | — | Compound 2-3 | BD2 | 3.90 | 5.41 | (0.127, 0.097) | 37.4 |
| Example 8 | | | | | | | | |
| Example 1 | Compound 1-1 | BD1 | Compound A | BD1 | 3.53 | 5.92 | (0.140, 0.051) | 57.9 |
| Example 2 | Compound 1-2 | BD1 | Compound 1-B | BD1 | 3.54 | 5.70 | (0.141, 0.049) | 62.8 |
| Example 3 | Compound 1-12 | BD1 | Compound 1-C | BD1 | 3.56 | 5.70 | (0.140, 0.050) | 63.8 |
| Example 4 | Compound 1-6 | BD1 | Compound 1-D | BD1 | 3.54 | 5.73 | (0.141, 0.049) | 61.4 |
| Example 5 | Compound 1-1 | BD1 | Compound E | BD1 | 3.58 | 5.77 | (0.140, 0.050) | 56.1 |
| Example 6 | Compound 1-3 | BD1 | Compound 1-F | BD1 | 3.57 | 5.73 | (0.140, 0.050) | 60.0 |
| Example 7 | Compound 1-4 | BD1 | Compound 1-G | BD1 | 5.60 | 5.78 | (0.141, 0.049) | 63.2 |
| Example 8 | Compound 1-5 | BD1 | Compound H | BD1 | 3.56 | 5.81 | (0.140, 0.051) | 57.4 |
| Example 9 | Compound 1-6 | BD1 | Compound 1-I | BD1 | 3.54 | 5.76 | (0.141, 0.049) | 61.5 |
| Example 10 | Compound 1-7 | BD1 | Compound 1-J | BD1 | 3.50 | 5.85 | (0.140, 0.060) | 67.5 |
| Example 11 | Compound 1-8 | BD1 | Compound 1-K | BD1 | 3.53 | 5.89 | (0.140, 0.058) | 62.4 |
| Example 12 | Compound 1-9 | BD1 | Compound L | BD1 | 3.60 | 5.76 | (0.140, 0.050) | 58.1 |
| Example 13 | Compound 1-10 | BD1 | Compound 1-M | BD1 | 3.54 | 5.88 | (0.140, 0.058) | 60.8 |
| Example 14 | Compound 1-11 | BD1 | Compound 1-N | BD1 | 3.58 | 5.79 | (0.140, 0.052) | 60.8 |
| Example 15 | Compound 1-4 | BD1 | Compound 1-O | BD1 | 3.51 | 5.70 | (0.140, 0.052) | 62.4 |
| Example 16 | Compound 1-13 | BD1 | Compound P | BD1 | 3.57 | 5.68 | (0.141, 0.049) | 56.9 |
| Example 17 | Compound 1-5 | BD1 | Compound 1-Q | BD1 | 3.58 | 5.71 | (0.140, 0.051) | 65.2 |
| Example 18 | Compound 1-2 | BD1 | Compound 1-R | BD1 | 3.55 | 5.76 | (0.140, 0.058) | 64.6 |
| Example 19 | Compound 1-7 | BD1 | Compound S | BD1 | 3.49 | 5.88 | (0.140, 0.051) | 54.6 |
| Example 20 | Compound 1-6 | BD1 | Compound 1-T | BD1 | 3.61 | 5.85 | (0.140, 0.050) | 61.8 |
| Example 21 | Compound 1-10 | BD1 | Compound U | BD1 | 3.59 | 5.73 | (0.140, 0.051) | 56.8 |
| Example 22 | Compound 1-9 | BD1 | Compound 1-V | BD1 | 3.55 | 5.77 | (0.140, 0.050) | 66.5 |
| Example 23 | Compound 1-11 | BD1 | Compound 1-W | BD1 | 3.50 | 5.86 | (0.140, 0.060) | 61.8 |
| Example 24 | Compound 1-14 | BD1 | Compound A | BD1 | 3.52 | 5.93 | (0.140, 0.051) | 70.1 |
| Example 25 | Compound 1-15 | BD1 | Compound 1-D | BD1 | 3.54 | 5.73 | (0.141, 0.049) | 72.6 |
| Example 26 | Compound 1-16 | BD1 | Compound 1-N | BD1 | 3.59 | 5.79 | (0.140, 0.052) | 73.0 |
| Example 27 | Compound 1-17 | BD1 | Compound 1-C | BD1 | 3.56 | 5.69 | (0.140, 0.050) | 75.2 |
| Example 28 | Compound 1-14 | BD2 | Compound A | BD1 | 3.59 | 6.18 | (0.134, 0.068) | 65.4 |
| Example 29 | Compound 1-15 | BD1 | Compound 1-D | BD2 | 3.61 | 5.90 | (0.132, 0.073) | 67.0 |
| Example 30 | Compound 1-16 | BD2 | Compound 1-N | BD2 | 3.71 | 6.03 | (0.128, 0.095) | 67.4 |
| Example 31 | Compound 1-17 | BD2 | Compound 1-C | BD2 | 3.68 | 5.91 | (0.128, 0.096) | 68.9 |
| Example 32 | Compound 1-12 | BD1 | Compound 2-1 | BD1 | 3.39 | 5.75 | (0.140, 0.053) | 60.3 |
| Example 33 | Compound 1-2 | BD1 | Compound 2-2 | BD1 | 3.43 | 5.70 | (0.140, 0.056) | 58.4 |
| Example 34 | Compound 1-9 | BD1 | Compound 2-3 | BD1 | 3.43 | 5.83 | (0.141, 0.054) | 57.0 |
| Example 35 | Compound 1-2 | BD1 | Compound 4# | BD1 | 3.40 | 5.88 | (0.140, 0.056) | 53.4 |
| Example 36 | Compound 1-13 | BD1 | Compound 5# | BD1 | 3.48 | 5.67 | (0.140, 0.062) | 55.1 |
| Example 37 | Compound 1-10 | BD1 | Compound 2-6 | BD1 | 3.46 | 5.83 | (0.140, 0.053) | 56.4 |
| Example 38 | Compound 1-4 | BD1 | Compound 2-7 | BD1 | 3.52 | 5.71 | (0.140, 0.053) | 55.9 |
| Example 39 | Compound 1-11 | BD1 | Compound 2-8 | BD1 | 3.47 | 5.73 | (0.140, 0.053) | 58.6 |
| Example 40 | Compound 1-2 | BD1 | Compound 2-9 | BD1 | 3.56 | 5.70 | (0.140, 0.058) | 60.9 |
| Example 41 | Compound 1-3 | BD1 | Compound 2-10 | BD1 | 3.55 | 5.69 | (0.140, 0.053) | 60.7 |
| Example 42 | Compound 1-6 | BD1 | Compound 2-11 | BD1 | 3.49 | 5.65 | (0.140, 0.057) | 59.0 |
| Example 43 | Compound 1-9 | BD1 | Compound 12# | BD1 | 3.55 | 5.73 | (0.141, 0.058) | 53.0 |
| Example 44 | Compound 1-12 | BD1 | Compound 2-13 | BD1 | 3.56 | 5.71 | (0.140, 0.053) | 56.8 |
| Example 45 | Compound 1-6 | BD1 | Compound 14# | BD1 | 3.55 | 5.72 | (0.140, 0.056) | 52.1 |
| Example 46 | Compound 1-3 | BD1 | Compound 2-15 | BD1 | 3.52 | 5.7 | (0.140, 0.055) | 58.6 |
| Example 47 | Compound 1-12 | BD1 | Compound 2-16 | BD1 | 3.5 | 5.77 | (0.141, 0.061) | 60.8 |
| Example 48 | Compound 1-2 | BD1 | Compound 2-17 | BD1 | 3.45 | 5.68 | (0.141, 0.061) | 57.4 |
| Example 49 | Compound 1-9 | BD1 | Compound 2-18 | BD1 | 3.44 | 5.66 | (0.141, 0.061) | 59.2 |
| Example 50 | Compound 1-2 | BD1 | Compound 2-19 | BD1 | 3.46 | 5.63 | (0.141, 0.061) | 56.8 |
| Example 51 | Compound 1-13 | BD1 | Compound 2-20 | BD1 | 3.49 | 5.90 | (0.140, 0.053) | 59.0 |
| Example 52 | Compound 1-10 | BD1 | Compound 2-21 | BD1 | 3.53 | 5.90 | (0.140, 0.056) | 58.1 |
| Example 53 | Compound 1-4 | BD1 | Compound 22# | BD1 | 3.47 | 5.74 | (0.141, 0.057) | 53.5 |
| Example 54 | Compound 1-2 | BD1 | Compound 23# | BD1 | 3.52 | 5.79 | (0.141, 0.057) | 53.0 |
| Example 55 | Compound 1-2 | BD1 | Compound 2-24 | BD1 | 3.44 | 5.80 | (0.141, 0.065) | 58.9 |
| Example 56 | Compound 1-4 | BD1 | Compound 2-25 | BD1 | 3.49 | 5.83 | (0.140, 0.066) | 59.1 |
| Example 57 | Compound 1-2 | BD1 | Compound 2-26 | BD1 | 3.46 | 5.84 | (0.140, 0.066) | 58.6 |
| Example 58 | Compound 1-1 | BD1 | Compound 2-27 | BD1 | 3.48 | 5.81 | (0.140, 0.067) | 58.5 |
| Example 59 | Compound 1-3 | BD1 | Compound 28# | BD1 | 3.40 | 5.75 | (0.140, 0.057) | 51.6 |
| Example 60 | Compound 1-11 | BD1 | Compound 2-29 | BD1 | 3.39 | 5.83 | (0.140, 0.057) | 59.4 |
| Example 61 | Compound 1-1 | BD1 | Compound 2-30 | BD1 | 3.36 | 5.85 | (0.140, 0.056) | 56.8 |
| Example 62 | Compound 1-3 | BD1 | Compound 31# | BD1 | 3.40 | 5.79 | (0.140, 0.057) | 53.0 |
| Example 63 | Compound 1-6 | BD1 | Compound 2-32 | BD1 | 3.38 | 5.75 | (0.141, 0.057) | 61.2 |
| Example 64 | Compound 1-9 | BD1 | Compound 2-33 | BD1 | 3.40 | 5.83 | (0.140, 0.057) | 59.4 |
| Example 65 | Compound 1-2 | BD1 | Compound 34# | BD1 | 3.35 | 5.81 | (0.140, 0.056) | 55.0 |
| Example 66 | Compound 1-2 | BD1 | Compound 2-35 | BD1 | 3.38 | 5.73 | (0.141, 0.062) | 58.0 |
| Example 67 | Compound 1-7 | BD1 | Compound 2-36 | BD1 | 3.36 | 5.70 | (0.140, 0.059) | 56.4 |
| Example 68 | Compound 1-6 | BD1 | Compound 2-37 | BD1 | 3.41 | 5.68 | (0.140, 0.060) | 54.1 |
| Example 69 | Compound 1-8 | BD1 | Compound 38# | BD1 | 3.43 | 5.69 | (0.140, 0.059) | 52.3 |
| Example 70 | Compound 1-4 | BD1 | Compound 2-39 | BD1 | 3.38 | 5.70 | (0.141, 0.060) | 60.7 |
| Example 71 | Compound 1-12 | BD1 | Compound 2-40 | BD1 | 3.35 | 5.78 | (0.140, 0.061) | 58.4 |
| Example 72 | Compound 1-2 | BD1 | Compound 41# | BD1 | 3.39 | 5.79 | (0.141, 0.060) | 51.8 |
| Example 73 | Compound 1-2 | BD1 | Compound 2-42 | BD1 | 3.36 | 5.77 | (0.141, 0.060) | 61.1 |
| Example 74 | Compound 1-5 | BD1 | Compound 2-43 | BD1 | 3.34 | 5.70 | (0.140, 0.064) | 60.4 |
| Example 75 | Compound 1-7 | BD1 | Compound 2-44 | BD1 | 3.38 | 5.70 | (0.140, 0.062) | 56.1 |
| Example 76 | Compound 1-3 | BD1 | Compound 45# | BD1 | 3.33 | 5.80 | (0.140, 0.068) | 53.6 |
| Example 77 | Compound 1-13 | BD1 | Compound 2-46 | BD1 | 3.38 | 5.79 | (0.141, 0.064) | 57.4 |
| Example 78 | Compound 1-10 | BD1 | Compound 2-47 | BD1 | 3.36 | 5.77 | (0.141, 0.064) | 58.2 |
| Example 79 | Compound 1-14 | BD1 | Compound 2-30 | BD1 | 3.37 | 5.85 | (0.140, 0.056) | 68.0 |
| Example 80 | Compound 1-15 | BD1 | Compound 2-32 | BD1 | 3.38 | 5.76 | (0.141, 0.057) | 71.4 |
| Example 81 | Compound 1-16 | BD1 | Compound 2-29 | BD1 | 3.40 | 5.82 | (0.140, 0.057) | 69.6 |
| Example 82 | Compound 1-17 | BD1 | Compound 2-40 | BD1 | 3.35 | 5.78 | (0.140, 0.061) | 70.8 |
| Example 83 | Compound 1-14 | BD2 | Compound 2-30 | BD1 | 3.43 | 6.10 | (0.133, 0.069) | 61.0 |
| Example 84 | Compound 1-15 | BD1 | Compound 2-32 | BD2 | 3.42 | 6.05 | (0.133, 0.068) | 64.9 |
| Example 85 | Compound 1-16 | BD2 | Compound 2-29 | BD2 | 3.52 | 6.09 | (0.128, 0.099) | 59.8 |
| Example 86 | Compound 1-17 | BD2 | Compound 2-40 | BD2 | 3.48 | 6.01 | (0.128, 0.103) | 61.0 |

The results in [Table 1A] shows a case where the device has a structure in which the light emitting layer is formed of two layers, the host of the first light emitting layer and the host of the second light emitting layer are the single hosts of each layer, and the compound of Chemical Formula 1 is included as a host of the first light emitting layer. Comparative Examples 1 to 4 are the cases in which an aryl-based anthracene host was used as a host of the second light emitting layer, and exhibited lower efficiency and service life, particularly significantly higher voltage, than Examples 1 to 86. Among these, Comparative Examples 2 and 3 have a structure in which the host of the second light emitting layer includes deuterium, and it could be seen that even though there was an effect of improving the service life of a certain portion, the effect on the overall device performance, which was significantly lower than that of Examples, was not significant. Moreover, when Comparative Example 2 is compared with Comparative Example 4 in which compound BD2, which is a boron-based blue dopant, is also applied, the tendency is maintained.

Comparative Examples 5 to 8 are cases where a furan-based anthracene host was used as a host of the second light emitting layer, and also exhibit particularly low voltage compared to Comparative Examples 1 to 4, but also exhibit low efficiency and service life and high voltage compared to Examples 1 to 86. Among these, Comparative Example 7 has a structure in which the host of the second light emitting layer includes deuterium as in Comparative Examples 2 and 3, and it could be seen that even though there was an effect of improving the service life of a certain portion, the effect on the overall device performance, which was significantly lower than that of the Examples, was not significant. Moreover, when Comparative Example 7 is compared with Comparative Example 8 in which compound BD2, which is a boron-based blue dopant, is also applied, the tendency is also maintained.

In Examples 1 to 86 of the present application, it can be seen that the performance of the device has been secured while applying the compound corresponding to Chemical Formula 1 as a host in the first light emitting layer to grow a role of injecting holes and serving as a barrier of electrons, changing the light emitting region in the light emitting layer, and maintaining low voltage characteristics and efficiency which are considered to be important in a blue light emitting device using an anthracene host in which an existing aryl-based or furan-based material is substituted as a host of the second light emitting layer.

Examples 1 to 23 are the case where the compound of Chemical Formula 1 was used as the host of the first light emitting layer and the aryl-based anthracene host was used as the host of the second light emitting layer, the Examples were evaluated as a case where the H compound to which deuterium is not applied is introduced as a host of the second emitting layer (Example 1, 5, 8, 12, 16 and 21) and a case where the D compound to which deuterium is applied is introduced (the others), as a host of the second emitting layer, and the Examples tended to confirm the effect of the light emitting layer having two layers in each case. The present inventors have seen data in which the service life can be additionally improved through the deuterium substitution of the host of the second light emitting layer, and could also confirm with certainty that the device performance is improved when the compound of Chemical Formula 1 is used as the host of the first light emitting layer.

Thus, in Examples 24 to 27, the compound of Chemical Formula 1 was substituted with deuterium and applied as a host of the first light emitting layer compared to Examples 1, 4, 14, and 3 to compare the device results. In the case of the first light emitting layer formed adjacent to a hole adjusting layer, additional service life improvement could be observed through deuterium substitution.

In Examples 28 to 31, the tendency and performance of the devices were confirmed by only changing compound BD1 to compound BD2 as a blue dopant in the devices of Examples 24 to 27, and when compound BD2, a boron-based dopant, was used, a tendency to maintain the service life improvement while improving the additional efficiency was confirmed.

Examples 32 to 58 are the cases where anthracene derivatives to which various heteroaryls such as dibenzofuran, naphthobenzofuran, and triphenyldifuran are bonded were introduced as hosts of the second light emitting layer, unlike Examples 1 to 23, and exhibited characteristics of having relatively low voltage and low service life compared to Examples 1 to 23, but in the structure of a device manufactured by applying the compound of Chemical Formula 1 as a host of the first light emitting layer, it was possible to confirm an improvement in the device while maintaining the characteristics as in Examples 1 to 23.

In Examples 79 to 86, changes were introduced as in Examples 24 to 31, and the results were also verified as excellent device performance.

### Examples 1A to 38A

Devices were manufactured in the same manner as in Example 1, except that in Examples 1A to 38A, the materials shown in the following [Table 1B] were used as materials for the light emitting layer.

For the devices manufactured in Examples 1A to 38A, the driving voltage, light emitting efficiency, and a time (LT95) for reaching a 95% value compared to the initial luminance were measured at a current density of 20 mA/cm². The results are shown in the following [Table 1B].

TABLE 1B

| | | | | | Device results | | | |
|---|---|---|---|---|---|---|---|---|
| | | | | | | | | Service life |
| | First light | | Second light | | Voltage (V) | Cd/A | Color | (T95, h) |
| Experimental | emitting layer | | emitting layer | | (@ 20 mA/ | (@ 20 | coordinate | (@ 20 mA/ |
| Example | Host | Dopant | Host | Dopant | cm²) | mA/cm²) | (x, y) | cm²) |
| Example 1A | Compound I-1 | BD1 | Compound A | BD1 | 3.54 | 5.90 | (0.140, 0.051) | 63.1 |
| Example 2A | Compound I-3 | BD1 | Compound 1-B | BD1 | 3.52 | 5.83 | (0.141, 0.049) | 65.2 |
| Example 3A | Compound I-5 | BD1 | Compound 1-C | BD1 | 3.55 | 5.81 | (0.140, 0.050) | 60.5 |
| Example 4A | Compound I-11 | BD1 | Compound 1-D | BD1 | 3.50 | 5.80 | (0.141, 0.049) | 61.8 |
| Example 5A | Compound I-4 | BD1 | Compound E | BD1 | 3.49 | 5.79 | (0.140, 0.050) | 60.1 |
| Example 6A | Compound I-7 | BD1 | Compound 1-F | BD1 | 3.56 | 5.75 | (0.140, 0.050) | 63.2 |
| Example 7A | Compound I-8 | BD1 | Compound 1-J | BD1 | 3.35 | 5.91 | (0.140, 0.060) | 66.0 |
| Example 8A | Compound I-9 | BD1 | Compound 1-K | BD1 | 3.55 | 5.86 | (0.140, 0.058) | 64.2 |
| Example 9A | Compound I-2 | BD1 | Compound L | BD1 | 3.52 | 5.81 | (0.140, 0.050) | 60.2 |
| Example 10A | Compound I-6 | BD1 | Compound 1-T | BD1 | 3.56 | 5.80 | (0.140, 0.050) | 61.8 |
| Example 11A | Compound I-10 | BD1 | Compound U | BD1 | 3.51 | 5.81 | (0.140, 0.051) | 60.9 |
| Example 12A | Compound I-12 | BD1 | Compound A | BD1 | 3.53 | 5.90 | (0.140, 0.051) | 73.9 |
| Example 13A | Compound I-13 | BD1 | Compound 1-B | BD1 | 3.52 | 5.83 | (0.141, 0.049) | 77.0 |
| Example 14A | Compound I-14 | BD1 | Compound 1-K | BD1 | 3.55 | 5.85 | (0.140, 0.058) | 76.5 |
| Example 15A | Compound I-15 | BD1 | Compound U | BD1 | 3.52 | 5.81 | (0.140, 0.051) | 73.9 |
| Example 16A | Compound I-12 | BD2 | Compound A | BD1 | 3.60 | 6.01 | (0.137, 0.060) | 70.0 |
| Example 17A | Compound I-13 | BD1 | Compound 1-B | BD2 | 3.59 | 5.95 | (0.138, 0.058) | 73.0 |
| Example 18A | Compound I-14 | BD2 | Compound 1-K | BD2 | 3.66 | 6.06 | (0.133, 0.064) | 69.8 |
| Example 19A | Compound I-15 | BD2 | Compound U | BD2 | 3.65 | 6.08 | (0.133, 0.068) | 68.4 |
| Example 20A | Compound I-1 | BD1 | Compound 2-1 | BD1 | 3.41 | 6.08 | (0.140, 0.053) | 56.9 |
| Example 21A | Compound I-3 | BD1 | Compound 2-3 | BD1 | 3.43 | 6.01 | (0.141, 0.054) | 59.1 |
| Example 22A | Compound I-5 | BD1 | Compound 5# | BD1 | 3.48 | 5.77 | (0.140, 0.062) | 55.6 |
| Example 23A | Compound I-11 | BD1 | Compound 2-11 | BD1 | 3.46 | 5.73 | (0.140, 0.057) | 58.1 |
| Example 24A | Compound I-4 | BD1 | Compound 12# | BD1 | 3.53 | 5.79 | (0.141, 0.058) | 55.0 |
| Example 25A | Compound I-7 | BD1 | Compound 2-30 | BD1 | 3.42 | 5.81 | (0.140, 0.056) | 56.7 |
| Example 26A | Compound I-8 | BD1 | Compound 31# | BD1 | 3.40 | 5.82 | (0.140, 0.057) | 53.6 |
| Example 27A | Compound I-9 | BD1 | Compound 2-33 | BD1 | 3.41 | 5.80 | (0.140, 0.057) | 58.4 |
| Example 28A | Compound I-2 | BD1 | Compound 2-42 | BD1 | 3.37 | 5.71 | (0.141, 0.060) | 58.0 |
| Example 29A | Compound I-6 | BD1 | Compound 2-43 | BD1 | 3.38 | 5.79 | (0.140, 0.064) | 59.6 |
| Example 30A | Compound I-10 | BD1 | Compound 2-40 | BD1 | 3.39 | 5.77 | (0.140, 0.061) | 59.1 |
| Example 31A | Compound I-12 | BD1 | Compound 2-1 | BD1 | 3.40 | 6.08 | (0.140, 0.053) | 66.7 |
| Example 32A | Compound I-13 | BD1 | Compound 2-3 | BD1 | 3.43 | 6.01 | (0.141, 0.054) | 69.5 |
| Example 33A | Compound I-14 | BD1 | Compound 2-33 | BD1 | 3.40 | 5.81 | (0.140, 0.057) | 67.8 |
| Example 34A | Compound I-15 | BD1 | Compound 2-40 | BD1 | 3.39 | 5.77 | (0.140, 0.061) | 70.1 |
| Example 35A | Compound I-12 | BD2 | Compound 2-1 | BD1 | 3.45 | 6.15 | (0.137, 0.059) | 63.0 |
| Example 36A | Compound I-13 | BD1 | Compound 2-3 | BD2 | 3.46 | 6.09 | (0.138, 0.061) | 66.7 |
| Example 37A | Compound I-14 | BD2 | Compound 2-33 | BD2 | 3.51 | 6.05 | (0.133, 0.067) | 60.1 |
| Example 38A | Compound I-15 | BD2 | Compound 2-40 | BD2 | 3.50 | 5.90 | (0.133, 0.071) | 61.4 |

In Examples 1A to 38A, changes were introduced in the same manner as in Examples 1 to 86, and the results were verified to be excellent device performance in the same manner as in Examples 1 to 86.

### <Example 87> Manufacture of OLED

As a positive electrode, a substrate on which ITO/Ag/ITO were deposited to have a thickness of 70/1,000/70 Å was cut into a size of 50 mm×50 mm×0.5 mm, put into distilled water in which a detergent was dissolved, and ultrasonically washed. A product manufactured by Fischer Co., was used as the detergent, and distilled water twice filtered using a filter manufactured by Millipore Co., was used as the distilled water. After the ITO was washed for 30 minutes, ultrasonic washing was conducted twice repeatedly using distilled water for 10 minutes. After the washing using distilled water was completed, ultrasonic washing was conducted using isopropyl alcohol, acetone, and methanol solvents in this order, and drying was then conducted.

The following compound HI-1 was thermally vacuum-deposited to have a thickness of 50 Å on the positive electrode thus prepared, thereby forming a hole injection layer, and the following compound HT1 as a material which transports holes was vacuum-deposited to have a thickness of 1,150 Å thereon, thereby forming a hole transport layer. Next, a hole adjusting layer was formed using the following compound EB1 (150 Å). Next, the host Compound 1-12 synthesized in Preparation Example 1 and the following compound BD1 as dopant (2 wt %) were vacuum-deposited to a thickness of 60 Å to form a light emitting layer as a first light emitting layer, and then the host Compound B of Preparation Example 2, Compound 2-3 of Preparation Example 2, and the following compound BD1 as dopant (2 wt %) were vacuum deposited to a thickness of 300 Å to form a light emitting layer as a second light emitting layer. Thereafter, the following compound HB1 was deposited to have a thickness of 50 Å, thereby forming an electron adjusting layer, and the following Compounds ET1 and Liq were mixed at 5:5 (mass ratio), thereby forming an electron transport layer having a thickness of 250 A. Sequentially, magnesium and lithium fluoride (LiF) were deposited to have a thickness of 50 Å to form a film as an electron injection layer <EIL>, magnesium and silver (1:4) were used to form a negative electrode having a thickness of 200 Å, and then the following compound CP1 was deposited to have a thickness of 600 Å, thereby completing a device. In the aforementioned procedure, the deposition rates of the organic materials were each maintained at 1 Å/sec.

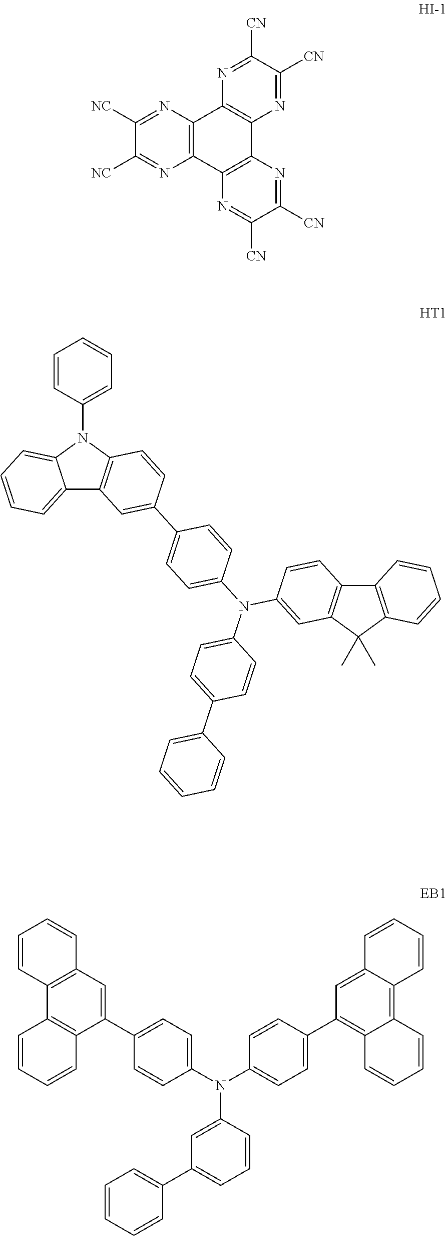 [SMILES: N#Cc1nc2c3nc(C#N)c(C#N)nc3c3nc(C#N)c(C#N)nc3c2nc1C#N ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2cccc(N(c3ccc(-c4cc5ccccc5c5ccccc45)cc3)c3ccc(-c4cc5ccccc5c5ccccc45)cc3)c2)cc1]

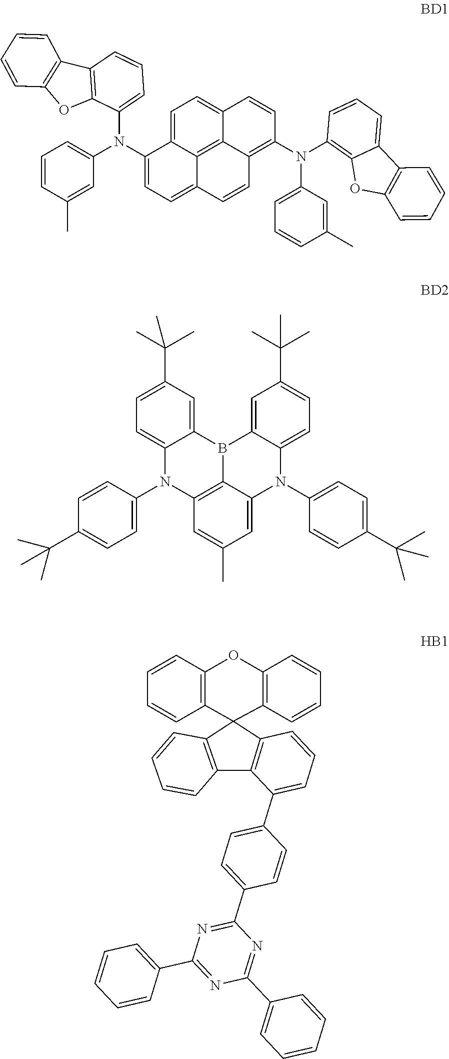 [SMILES: Cc1cccc(N(c2ccc3ccc4c(N(c5cccc(C)c5)c5cccc6c5oc5ccccc56)ccc5ccc2c3c54)c2cccc3c2oc2ccccc23)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(-c2nc(-c3ccccc3)nc(-c3ccc(-c4cccc5c4-c4ccccc4C54c5ccccc5Oc5ccccc54)cc3)n2)cc1]

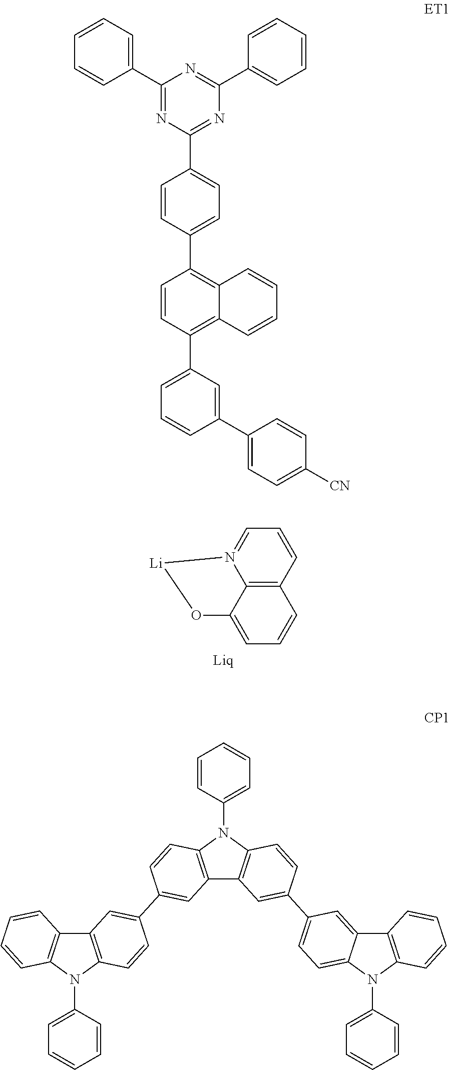

### Comparative Examples 9 to 18 and Examples 88 to 122

Devices were manufactured in the same manner as in Example 87, except that in Comparative Examples 9 to 18 and Examples 88 to 122, the materials shown in the following Table 2 were used as materials for the light emitting layer.

However, as shown in [Table 2A], the host of the second light emitting layer exhibited both an example in which the light emitting layer is formed by co-depositing two hosts through another evaporation source during the manufacture of a device and an example in which two host compounds were used for a pre-mixture prepared in advance.

In particular, the conditions for preparing the pre-mixture using the host of the second light emitting layer have been mentioned above, and [Table 3] shows that the two hosts of the second light emitting layer applied to Comparative Examples 9 to 18 and Examples 87 to 122 in [Table 2A] can be used to prepare the pre-mixture. For the devices manufactured in Comparative Examples 9 to 18 and Examples 87 to 122, the driving voltage, light emitting efficiency, and a time (LT95) for reaching a 95% value compared to the initial luminance were measured at a current density of 20 mA/cm². The results are shown in the following [Table 2A].

TABLE 2A

| | | | Second light | | | | | |
|---|---|---|---|---|---|---|---|---|
| | | | emitting layer | | Device results | | | |
| | | | First | | | | | Service life |
| | First light | | host:Second | | Voltage (V) | Cd/A | Color | (T95, h) |
| Experimental | emitting layer | | host (ratio, | | (@20 mA/ | (@20 mA/ | coordinate | (@20 mA/ |
| Example | Host | Dopant | mixing method) | Dopant | cm²) | cm²) | (x, y) | cm²) |
| Comparative | — | — | 1-A:B (1:1) | BD1 | 3.86 | 5.43 | (0.140, 0.050) | 43.1 |
| Example 9 | | | | | | | | |
| Comparative | — | — | 1-A:B (1:1, | BD1 | 3.86 | 5.44 | (0.140, 0.050) | 44.0 |
| Example 10 | | | pre-mixture) | | | | | |
| Comparative | — | — | J:28# (1:1) | BD1 | 3.79 | 5.30 | (0.141, 0.055) | 38.9 |
| Example 11 | | | | | | | | |
| Comparative | — | — | J:28# (1:1, | BD1 | 3.78 | 5.30 | (0.134, 0.071) | 39.1 |
| Example 12 | | | pre-mixture) | | | | | |
| Comparative | — | — | 1-J:28# (1:3) | BD1 | 3.75 | 5.26 | (0.141, 0.055) | 39.3 |
| Example 13 | | | | | | | | |
| Comparative | — | — | 28#:2-39 (1:1) | BD1 | 3.71 | 5.25 | (0.141, 0.059) | 35.1 |
| Example 14 | | | | | | | | |
| Comparative | — | — | 28#:2-39 (1:1, | BD1 | 3.71 | 5.26 | (0.141, 0.059) | 35.2 |
| Example 15 | | | pre-mixture) | | | | | |
| Comparative | — | — | 28#:2-39 (2:1) | BD1 | 3.73 | 5.28 | (0.135, 0.075) | 34.3 |
| Example 16 | | | | | | | | |
| Comparative | — | — | 1-A:B (1:1) | BD2 | 3.92 | 5.61 | (0.129, 0.099) | 35.3 |
| Example 17 | | | | | | | | |
| Comparative | — | — | J:28# (1:1) | BD2 | 3.84 | 5.52 | (0.129, 0.099) | 33.4 |
| Example 18 | | | | | | | | |
| Example 87 | 1-12 | BD1 | B:2-3 (1:1) | BD1 | 3.38 | 6.13 | (0.140, 0.052) | 66.1 |
| Example 88 | 1-6 | BD1 | A:2-6 (1:1) | BD1 | 3.40 | 6.10 | (0.140, 0.052) | 66.9 |
| Example 89 | 1-1 | BD1 | 1-L:32# (1:1) | BD1 | 3.38 | 5.99 | (0.141, 0.055) | 63.1 |
| Example 90 | 1-3 | BD1 | V:2-23 (1:1) | BD1 | 3.40 | 6.02 | (0.140, 0.054) | 63.4 |
| Example 91 | 1-8 | BD1 | 1-S:9# (1:1) | BD1 | 3.44 | 5.90 | (0.140, 0.054) | 67.8 |
| Example 92 | 1-1 | BD1 | 1-U:43# (1:1) | BD1 | 3.30 | 6.01 | (0.141, 0.058) | 60.1 |
| Example 93 | 1-2 | BD1 | G:2-15 (1:1) | BD1 | 3.35 | 6.00 | (0.140, 0.053) | 59.5 |
| Example 94 | 1-13 | BD1 | 1-J:10# (1:1) | BD1 | 3.42 | 6.10 | (0.140, 0.051) | 60.8 |
| Example 95 | 1-5 | BD1 | L:2-17 (1:1) | BD1 | 3.35 | 5.93 | (0.141, 0.057) | 59.7 |
| Example 96 | 1-4 | BD1 | U:2-25 (1:1) | BD1 | 3.36 | 6.08 | (0.140, 0.064) | 65.4 |
| Example 97 | 1-7 | BD1 | 1-W:24# (1:1) | BD1 | 3.32 | 6.05 | (0.140, 0.064) | 64.8 |
| Example 98 | 1-6 | BD1 | 1-I:12# (1:1) | BD1 | 3.40 | 5.93 | (0.140, 0.054) | 68.0 |
| Example 99 | 1-10 | BD1 | 1-V:35# (1:1) | BD1 | 3.33 | 6.10 | (0.141, 0.057) | 60.2 |
| Example 100 | 1-11 | BD1 | O:2-40 (1:1) | BD1 | 3.31 | 6.08 | (0.141, 0.059) | 60.6 |
| Example 101 | 1-10 | BD1 | 1-R:45# (1:1) | BD1 | 3.30 | 6.13 | (0.140, 0.067) | 59.7 |
| Example 102 | 1-9 | BD1 | 1-T:41# (1:1) | BD1 | 3.33 | 6.05 | (0.141, 0.059) | 63.1 |
| Example 103 | 1-1 | BD1 | 1-L:2-32 (1:1) | BD1 | 3.39 | 5.99 | (0.141, 0.055) | 73.5 |
| Example 104 | 1-6 | BD1 | 1-A:2-6 (1:1) | BD1 | 3.40 | 6.09 | (0.140, 0.052) | 78.4 |
| Example 105 | 1-11 | BD1 | 1-O:2-40 (1:1) | BD1 | 3.32 | 6.08 | (0.141, 0.059) | 72.8 |
| Example 106 | 1-12 | BD1 | 1-B:2-3 (1:1) | BD1 | 3.37 | 6.14 | (0.140, 0.052) | 78.4 |
| Example 107 | 1-14 | BD1 | 1-L:2-32 (1:1) | BD1 | 3.38 | 5.99 | (0.141, 0.055) | 82.4 |
| Example 108 | 1-15 | BD1 | 1-A:2-6 (1:1) | BD1 | 3.41 | 6.10 | (0.140, 0.052) | 88.6 |
| Example 109 | 1-16 | BD1 | 1-O:2-40 (1:1) | BD1 | 3.32 | 6.08 | (0.141, 0.059) | 82.3 |
| Example 110 | 1-17 | BD1 | 1-B:2-3 (1:1) | BD1 | 3.37 | 6.13 | (0.140, 0.052) | 87.1 |
| Example 111 | 1-14 | BD1 | 1-L:2-32 (1:1 | BD1 | 3.39 | 5.99 | (0.141, 0.055) | 83.9 |
| | | | pre-mixture) | | | | | |
| Example 112 | 1-15 | BD1 | 1-A:2-6 (1:1, | BD1 | 3.40 | 6.10 | (0.140, 0.052) | 89.8 |
| | | | pre-mixture) | | | | | |
| Example 113 | 1-16 | BD1 | 1-O:2-40 (1:1 | BD1 | 3.32 | 6.08 | (0.141, 0.059) | 84.5 |
| | | | pre-mixture) | | | | | |
| Example 114 | 1-17 | BD1 | 1-B:2-3 (1:1 | BD1 | 3.36 | 6.13 | (0.140, 0.052) | 89.5 |
| | | | pre-mixture) | | | | | |
| Example 115 | 1-14 | BD1 | 1-L:2-32 (1:1, | BD2 | 3.46 | 6.08 | (0.134, 0.069) | 74.5 |
| | | | pre-mixture) | | | | | |
| Example 116 | 1-15 | BD2 | 1-A:2-6 (1:1, | BD1 | 3.48 | 6.18 | (0.132, 0.071) | 80.1 |
| | | | pre-mixture) | | | | | |
| Example 117 | 1-16 | BD2 | 1-O:2-40 (1:1 | BD2 | 3.49 | 6.23 | (0.128, 0.097) | 73.5 |
| | | | pre-mixture) | | | | | |
| Example 118 | 1-17 | BD2 | 1-B:2-3 (1:1 | BD2 | 3.48 | 6.31 | (0.128, 0.098) | 77.6 |
| | | | pre-mixture) | | | | | |
| Example 119 | 1-14 | BD1 | 1-L:2-32 (2:1, | BD2 | 3.48 | 6.13 | (0.133, 0.065) | 75.0 |
| | | | pre-mixture) | | | | | |
| Example 120 | 1-15 | BD2 | 1-A:2-6 (1:2, | BD1 | 3.44 | 6.15 | (0.133, 0.073) | 78.5 |
| | | | pre-mixture) | | | | | |
| Example 121 | 1-16 | BD2 | 1-O:2-40 (3:1, | BD2 | 3.53 | 6.33 | (0.127, 0.093) | 73.9 |
| | | | pre-mixture) | | | | | |
| Example 122 | 1-17 | BD2 | 1-B:2-3 (1:3, | BD2 | 3.4 | 6.28 | (0.128, 0.101) | 70.6 |
| | | | pre-mixture) | | | | | |

TABLE 3

| Second light emitting | | | \|DMₕₒₛₜ₁ − | | | \|T_{sub1} − |
|---|---|---|---|---|---|---|
| layer host | DMₕₒₛₜ₁ | DMₕₒₛₜ₂ | DMₕₒₛₜ₂\| | T_{sub1} | T_{sub2} | T_{sub2}\| |
| Compound A:Compound B | 0.29 | 0.17 | 0.12 | 270 | 250 | 20 |
| Compound J:Compound 28# | 0.16 | 1.03 | 0.87 | 250 | 270 | 20 |
| Compound 28#:Compound 39# | 1.03 | 1.15 | 0.12 | 270 | 280 | 10 |
| Compound A:Compound 3# | 0.29 | 1.15 | 0.86 | 270 | 270 | 0 |
| Compound B:Compound 6# | 0.17 | 0.82 | 0.65 | 250 | 240 | 10 |
| Compound L:Compound 32# | 0.21 | 0.91 | 0.70 | 260 | 270 | 10 |
| Compound V:Compound 23# | 0.17 | 0.94 | 0.77 | 290 | 290 | 0 |
| Compound S:Compound 9# | 0.30 | 1.20 | 0.90 | 260 | 280 | 20 |
| Compound U:Compound 43# | 0.12 | 1.28 | 1.16 | 270 | 290 | 20 |
| Compound G:Compound 15# | 0.06 | 0.63 | 0.57 | 250 | 270 | 20 |
| Compound J:Compound 10# | 0.16 | 0.69 | 0.53 | 250 | 260 | 10 |
| Compound L:Compound 17# | 0.21 | 0.74 | 0.53 | 260 | 260 | 0 |
| Compound U:Compound 25# | 0.12 | 1.05 | 0.93 | 270 | 270 | 0 |
| Compound W:Compound 24# | 0.24 | 1.11 | 0.87 | 290 | 300 | 10 |
| Compound I:Compound 12# | 0.23 | 1.01 | 0.78 | 250 | 270 | 20 |
| Compound V:Compound 35# | 0.17 | 1.15 | 0.98 | 290 | 300 | 10 |
| Compound O:Compound 40# | 0.18 | 1.17 | 0.99 | 270 | 290 | 20 |
| Compound R:Compound 45# | 0.02 | 1.21 | 1.19 | 270 | 290 | 20 |
| Compound T:Compound 41# | 0.08 | 1.25 | 1.17 | 260 | 280 | 20 |

The results in [Table 2A] show a case where the light emitting layer is formed of two layers, the host of the first light emitting layer is a single host and is a compound of Chemical Formula 1, and the host of the second light emitting layer is a mixed host and is composed of a mixed host of two types of anthracenes co-deposited or pre-mixed. Comparative Examples 9 and 10 are examples in which among the widely used aryl-based anthracene compounds, a H compound (a skeleton in which only hydrogen is substituted) and a D compound (a compound in which deuterium is partially or completely substituted) are used to be co-deposited or to prepare and deposit a pre-mixture, thereby forming a host of the second light emitting layer. Additionally, Comparative Examples 11, 12, and 13 are examples in which an aryl-based anthracene compound and two types of heteroaryl-based anthracene compounds (one or more of the two types are the D compound) are used to be co-deposited or to prepare and deposit a pre-mixture, thereby forming a host of the second light emitting layer. Comparative Examples 14, 15, and 16 are examples in which two types of heteroaryl-based anthracene compounds (one or more of the two types are the D compound) are used to be co-deposited or to prepare and deposit a pre-mixture, thereby forming a host of the second light emitting layer.

It can be seen that when compared with the case of a single host in Comparative Examples 1 to 8 in [Table 1A] described above, forming a light emitting layer using a mixed host improves the performance of the device. Furthermore, in Comparative Examples 17 and 18, it can be confirmed that the characteristics are maintained and the improvement in device performance is maintained even when as in Comparative Example 4 in [Table 1A], a boron-based blue dopant is applied and a mixed host (co-deposited or prepared a pre-mixture) is used.

It can be observed that Examples 87 to 102 exhibit very excellent voltage and efficiency characteristics and may also increase the service life compared to Comparative Examples 9 to 18 by using the compound of Chemical Formula 1 as the host of the first light emitting layer and co-depositing various combinations of anthracene compounds (aryl-based or heteroaryl-based compounds, one of which is a deuterium-substituted D compound) (1:1 mass ratio) to form a host of the second light emitting layer.

In Examples 103 to 106, devices were manufactured by co-depositing both hosts with a deuterium-substituted D compound, unlike the hosts of the second light emitting layer in Examples 87 to 102, and an additional service life increase of 10% on average was shown, compared to Examples 89, 88, 100 and 87. In Examples 107 to 110, among the compounds of the claims in the first light emitting layer, Compounds 1-14 to 1-17, which are deuterated versions of Compounds 1-1, 1-6, 1-11 and 1-12, were applied to construct devices, unlike Examples 103 to 106. Further, in this regard, in the case of the first light emitting layer formed adjacent to a hole adjusting layer as mentioned in the results of [Table 1A], additional service life improvement could be observed through deuterium substitution.

Examples 111 to 114 are devices manufactured by changing the method of co-depositing the host of the second light emitting layer of Examples 107 to 110 to a method in which a pre-mixture of two types of hosts was formed and then evaporated from a single deposition source. The conditions for forming the pre-mixture were performed with reference to the above-described description, and the device performance of the co-deposited device was maintained or stably improved due to the uniform mixing effect of the molecular units.

Examples 115 to 118 are cases in which BD2 (a boron-based blue dopant) is applied to one or all of the dual light emitting layers and a pre-mixture host is applied, and it can be seen that while maintaining high efficiency compared to a pyrene dopant, the advantages of pre-mixed hosts are maintained.

Examples 119 to 122 are cases in which the pre-mixture of the second light emitting layer in Examples 115 to 118 was prepared by changing the ratio of the two compounds and deposited to form a host, and shows that the advantages and disadvantages of the two compounds may be applied through the ratio and reflected in the device.

### Examples 39A to 69A

Devices were manufactured in the same manner as in Example 87, except that in Examples 39A to 69A, the materials shown in the following [Table 2B] were used as materials for the light emitting layer.

For the devices manufactured in Examples 39A to 69A, the driving voltage, light emitting efficiency, and a time (LT95) for reaching a 95% value compared to the initial luminance were measured at a current density of 20 mA/cm². The results are shown in the following [Table 2B].

TABLE 2B

| | | | Second light | | | | | |
|---|---|---|---|---|---|---|---|---|
| | | | emitting layer | | Device results | | | |
| | | | First | | | | | Service life |
| | First light | | host:Second | | Voltage | Cd/A | Color | (T95, h) |
| Experimental | emitting layer | | host (ratio, | | (V) (@20 | (@20 | coordinate | (@20 |
| Example | Host | Dopant | mixing method) | Dopant | mA/cm²) | mA/cm²) | (x, y) | mA/cm²) |
| Example 39A | 2-1 | BD1 | B:2-3 (1:1) | BD1 | 3.42 | 6.16 | (0.140, 0.052) | 65.2 |
| Example 40A | 2-3 | BD1 | 1-L:32# (1:1) | BD1 | 3.40 | 6.05 | (0.141, 0.055) | 63.8 |
| Example 41A | 2-5 | BD1 | 1-U:43# (1:1) | BD1 | 3.31 | 6.03 | (0.141, 0.058) | 64.0 |
| Example 42A | 2-11 | BD1 | A:2-6 (1:1) | BD1 | 3.40 | 6.06 | (0.140, 0.052) | 63.2 |
| Example 43A | 2-4 | BD1 | G:2-15 (1:1) | BD1 | 3.35 | 6.11 | (0.140, 0.053) | 63.5 |
| Example 44A | 2-7 | BD1 | 1-J:10# (1:1) | BD1 | 3.39 | 6.10 | (0.140, 0.051) | 64.7 |
| Example 45A | 2-8 | BD1 | 1-I:12# (1:1) | BD1 | 3.40 | 6.02 | (0.140, 0.054) | 66.0 |
| Example 46A | 2-9 | BD1 | 1-V:35# (1:1) | BD1 | 3.41 | 6.15 | (0.141, 0.057) | 62.1 |
| Example 47A | 2-2 | BD1 | O:2-40 (1:1) | BD1 | 3.36 | 6.11 | (0.141, 0.059) | 63.4 |
| Example 48A | 2-6 | BD1 | 1-R:45# (1:1) | BD1 | 3.33 | 6.13 | (0.140, 0.067) | 63.0 |
| Example 49A | 2-10 | BD1 | 1-T:41# (1:1) | BD1 | 3.32 | 6.12 | (0.141, 0.059) | 64.7 |
| Example 50A | 2-12 | BD1 | B:2-3 (1:1) | BD1 | 3.41 | 6.16 | (0.140, 0.052) | 76.0 |
| Example 51A | 2-13 | BD1 | 1-L:32# (1:1) | BD1 | 3.40 | 6.05 | (0.141, 0.055) | 77.5 |
| Example 52A | 2-14 | BD1 | 1-V:35# (1:1) | BD1 | 3.41 | 6.14 | (0.141, 0.057) | 73.9 |
| Example 53A | 2-15 | BD1 | 1-T:41# (1:1) | BD1 | 3.32 | 6.12 | (0.141, 0.059) | 75.1 |
| Example 54A | 2-12 | BD1 | 1-B:C2-3 (1:1) | BD1 | 3.41 | 6.16 | (0.140, 0.052) | 82.6 |
| Example 55A | 2-13 | BD1 | 1-L:2-32 (1:1) | BD1 | 3.40 | 6.05 | (0.141, 0.055) | 81.8 |
| Example 56A | 2-14 | BD1 | 1-V:2-35 (1:1) | BD1 | 3.41 | 6.14 | (0.141, 0.057) | 80.2 |
| Example 57A | 2-15 | BD1 | 1-T:2-41 (1:1) | BD1 | 3.32 | 6.12 | (0.141, 0.059) | 81.9 |
| Example 58A | 2-12 | BD2 | 1-B:2-3 (1:1) | BD1 | 3.48 | 6.23 | (0.137, 0.060) | 77.9 |
| Example 59A | 2-13 | BD1 | 1-L:2-32 (1:1) | BD2 | 3.48 | 6.10 | (0.138, 0.063) | 76.2 |
| Example 60A | 2-14 | BD2 | 1-V:2-35 (1:1) | BD2 | 3.53 | 6.25 | (0.134, 0.068) | 70.2 |
| Example 61A | 2-15 | BD2 | 1-T:2-41 (1:1) | BD2 | 3.50 | 6.29 | (0.134, 0.069) | 71.6 |
| Example 62A | 2-12 | BD2 | 1-B:2-3 (1:1, | BD1 | 3.46 | 6.25 | (0.137, 0.060) | 78.3 |
| | | | pre-mixture) | | | | | |
| Example 63A | 2-13 | BD1 | 1-L:2-32 (1:1, | BD2 | 3.48 | 6.12 | (0.138, 0.063) | 77.1 |
| | | | pre-mixture) | | | | | |
| Example 64A | 2-14 | BD2 | 1-V:2-35 (1:1, | BD2 | 3.52 | 6.25 | (0.134, 0.068) | 71.5 |
| | | | pre-mixture) | | | | | |
| Example 65A | 2-15 | BD2 | 1-T:2-41 (1:1, | BD2 | 3.50 | 6.30 | (0.134, 0.069) | 73.0 |
| | | | pre-mixture) | | | | | |
| Example 66A | 2-12 | BD2 | 1-B:2-3 (2:1, | BD1 | 3.49 | 6.26 | (0.136, 0.058) | 80.0 |
| | | | pre-mixture) | | | | | |
| Example 67A | 2-13 | BD1 | 1-L:2-32 (1:2, | BD2 | 3.45 | 6.20 | (0.137, 0.067) | 75.9 |
| | | | pre-mixture) | | | | | |
| Example 68A | 2-14 | BD2 | 1-V:2-35 (3:1, | BD2 | 3.58 | 6.28 | (0.130, 0.060) | 71.0 |
| | | | pre-mixture) | | | | | |
| Example 69A | 2-15 | BD2 | 1-T:2-41 (1:4, | BD2 | 3.44 | 6.12 | (0.137, 0.073) | 73.0 |
| | | | pre-mixture) | | | | | |

In Examples 39A to 69A, changes were introduced in the same manner as in Examples 87 to 122, and the results were verified to be excellent device performance in the same manner as in Examples 1 to 86.

1. A compound of the following Chemical Formula 1: 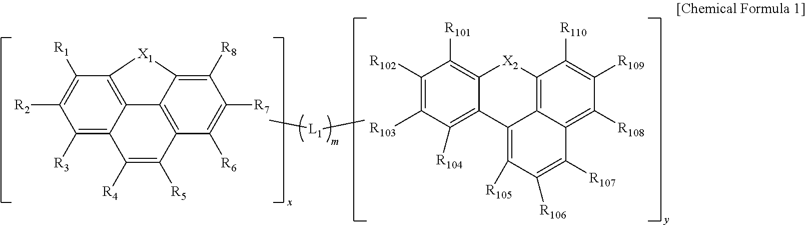
wherein, in Chemical Formula 1,
X₁ and X₂ are the same as or different from each other, and are each independently O or S,
L₁ is a direct bond, a substituted or unsubstituted arylene group or a substituted or unsubstituted divalent heterocyclic group,
here, L₁ is further unsubstituted or substituted with deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and optionally, at least one of the substituents of L₁ is optionally bonded to an adjacent substituent to form one or more substituted or unsubstituted rings,
one of R₁ to R₈ and one of R₁₀₁ to R₁₁₀ are each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring,
m is an integer from 1 to 4, and when m is 2 or higher, L₁'s are the same as or different from each other,
x is 1 or 2, and y is 1 or 2, and
when x and y are each 2, structures in the parenthesis are the same as or different from each other.

2. The compound of claim 1, wherein x is 1 and y is 1,
x is 1 and y is 2, or
x is 2 and y is 1.

3. The compound of claim 1, wherein x is 1, y is 1, and L₁ is the following Chemical Formula A or B: 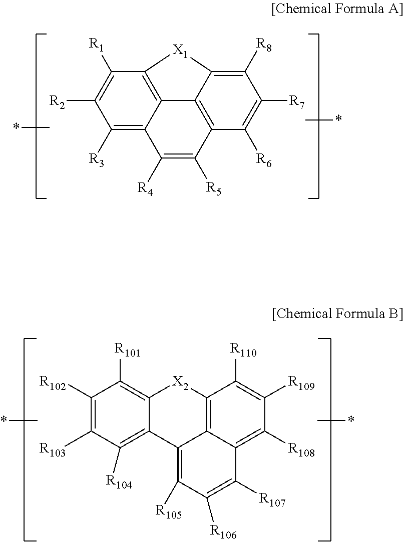
in Chemical Formulae A and B,
X₁ and X₂ are the same as or different from each other, and are each independently O or S,
two of R₁ to R₈ and two of R₁₀₁ to R₁₁₀ are each linked to an adjacent group, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring, and
* denotes a bonding position.

4. The compound of claim 1, wherein x is 1, y is 1, and one of R₁ to R₈ and R₁₀₁ to R₁₁₀ is the following Chemical Formula A-1 or B-1: 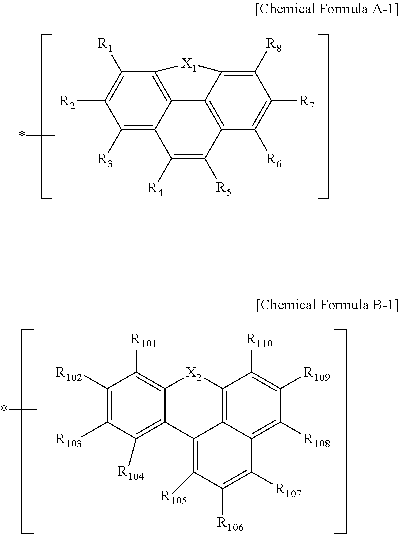
in Chemical Formulae A-1 and B-1,
X₁ and X₂ are the same as or different from each other, and are each independently O or S,
one of R₁ to R₈ and one of R₁₀₁ to R₁₁₀ are each linked to an adjacent group, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring, and
* denotes a bonding position.

5. The compound of claim 1, wherein at least one of R₁ and R₂, R₂ and R₃, R₄ and R₅, R₁₀₁ and R₁₀₂, R₁₀₂ and R₁₀₃, R₁₀₃ and R₁₀₄, R₁₀₅ and R₁₀₆, R₁₀₆ and R₁₀₇, R₁₀₈ and R₁₀₉, and R₁₀₉ and R₁₁₀ are bonded to form a substituted or unsubstituted hydrocarbon ring.

6. A compound of any one of the following Chemical Formulae 11 to 13: 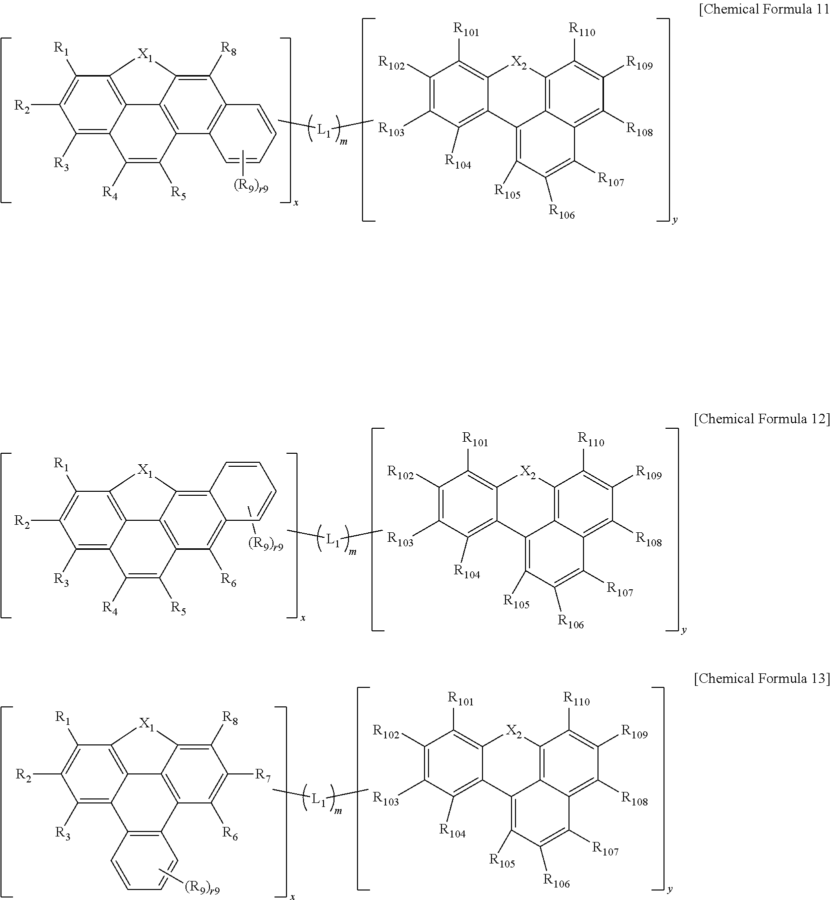
in Chemical Formulae 11 to 13,
X₁ and X₂ are the same as or different from each other, and are each independently O or S,
L₁ is a direct bond, a substituted or unsubstituted arylene group or a substituted or unsubstituted divalent heterocyclic group,
here, L₁ is further unsubstituted or substituted with deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and optionally, at least one of the substituents of L₁ is optionally bonded to an adjacent substituent to form one or more substituted or unsubstituted rings,
one of R₁ to R₉ and one of R₁₀₁ to R₁₁₀ are each linked to L₁, and the others are the same as or different from each other, and are each independently hydrogen, deuterium, a hydroxyl group, a cyano group, a halogen group, a substituted or unsubstituted alkyl group, a substituted or unsubstituted cycloalkyl group, a substituted or unsubstituted aryl group or a substituted or unsubstituted heterocyclic group, and are optionally bonded to an adjacent substituent to form a substituted or unsubstituted ring,
m is an integer from 1 to 4, and when m is 2 or higher, L₁'s are the same as or different from each other,
x is 1 or 2, and y is 1 or 2,
when x and y are each 2, structures in the parenthesis are the same as or different from each other, and
r9 is an integer from 1 to 4, and when r9 is 2 or higher, R₉'s are the same as or different from each other.

7. The compound of claim 1, wherein Chemical Formula 1 is any one of the following structures:

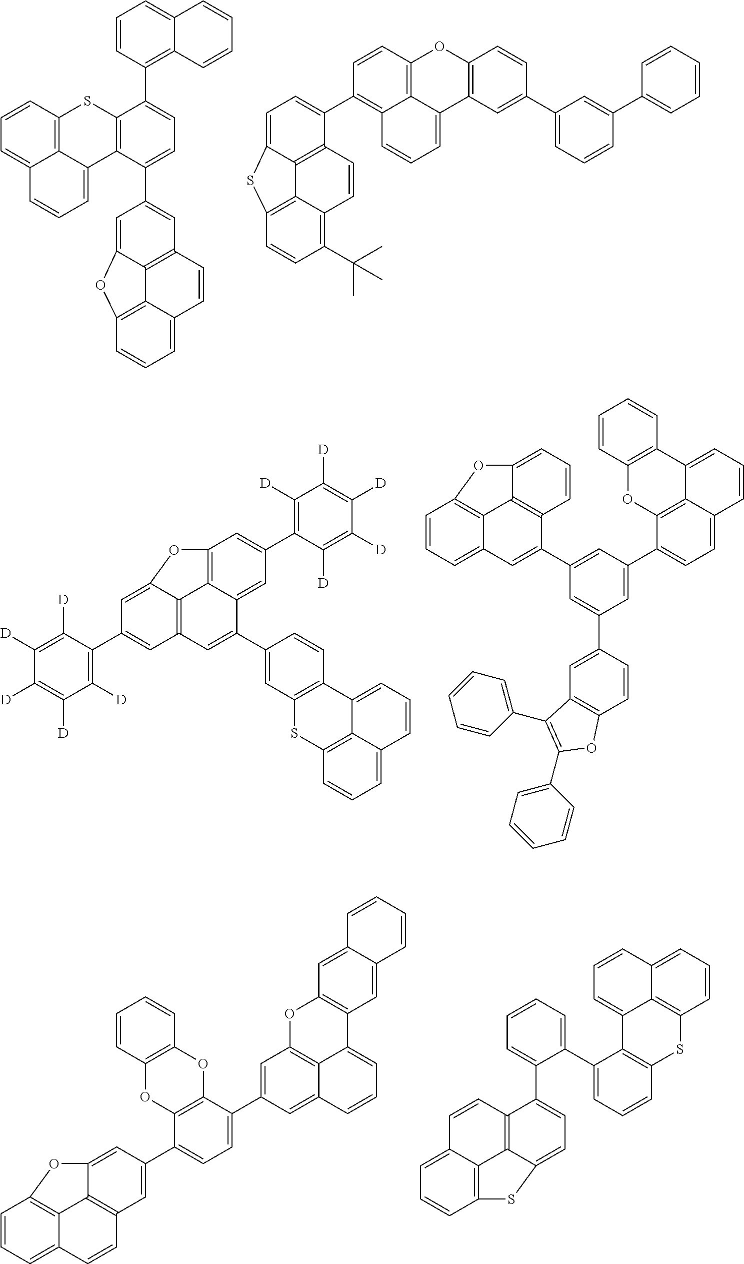

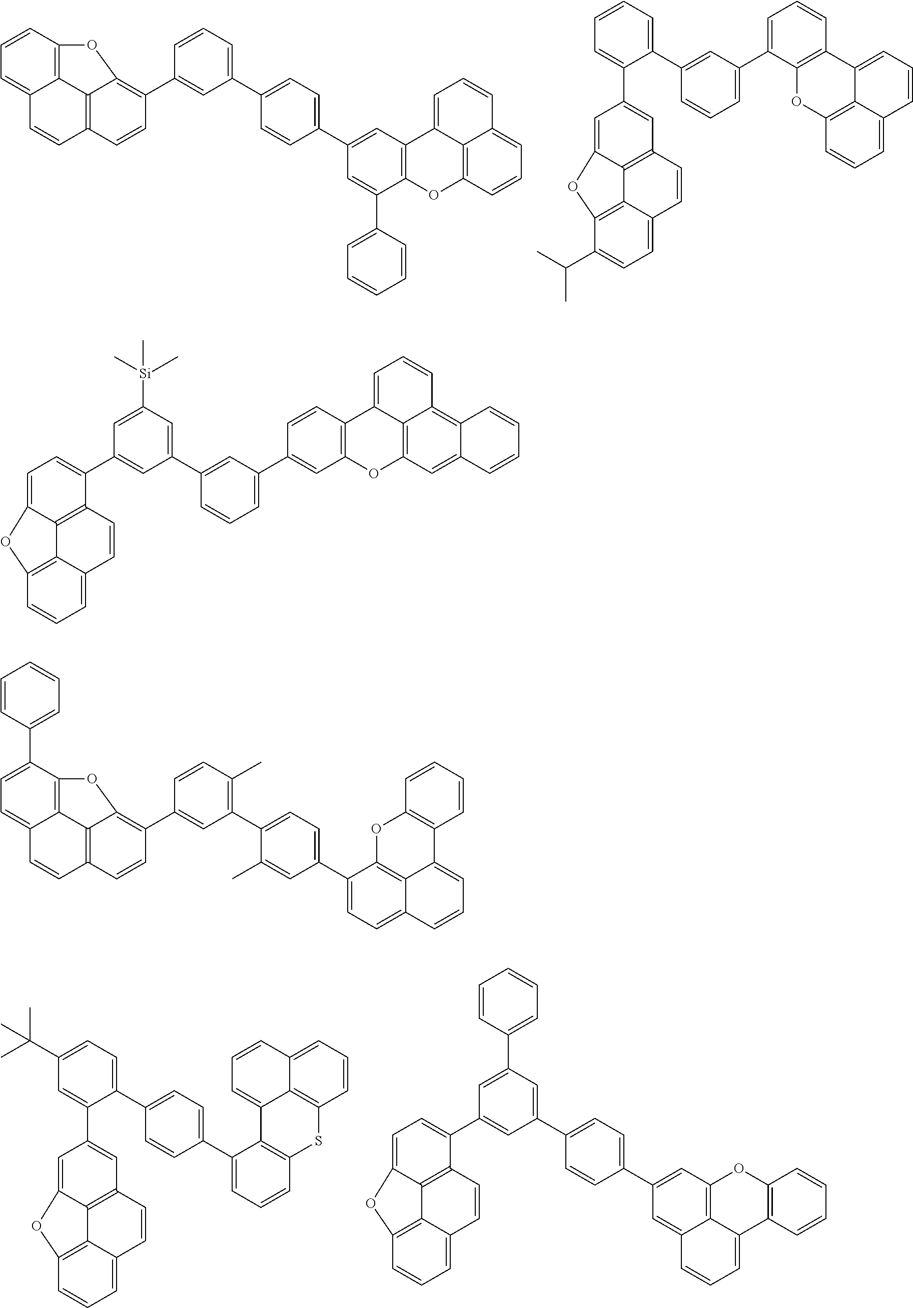

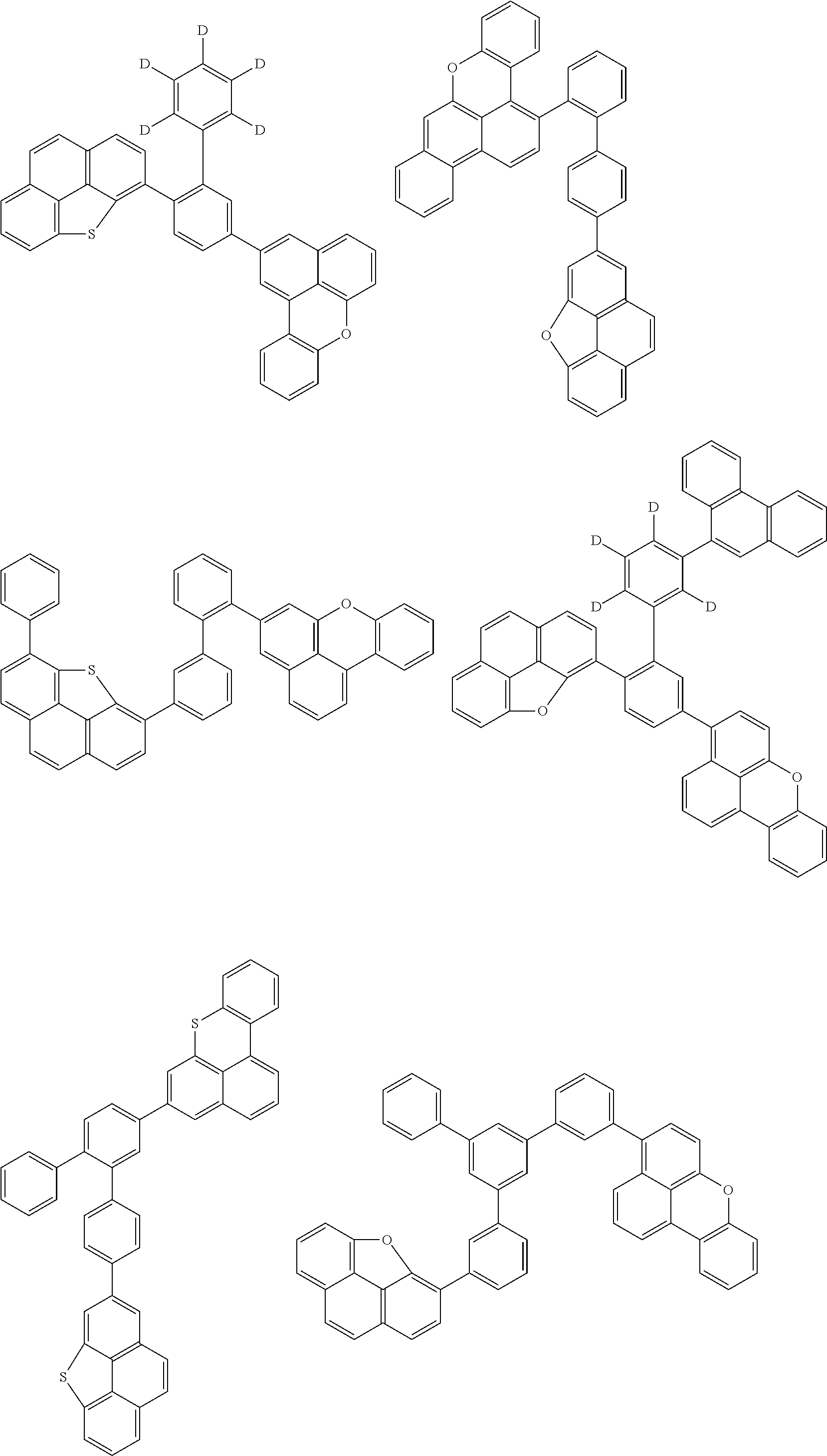

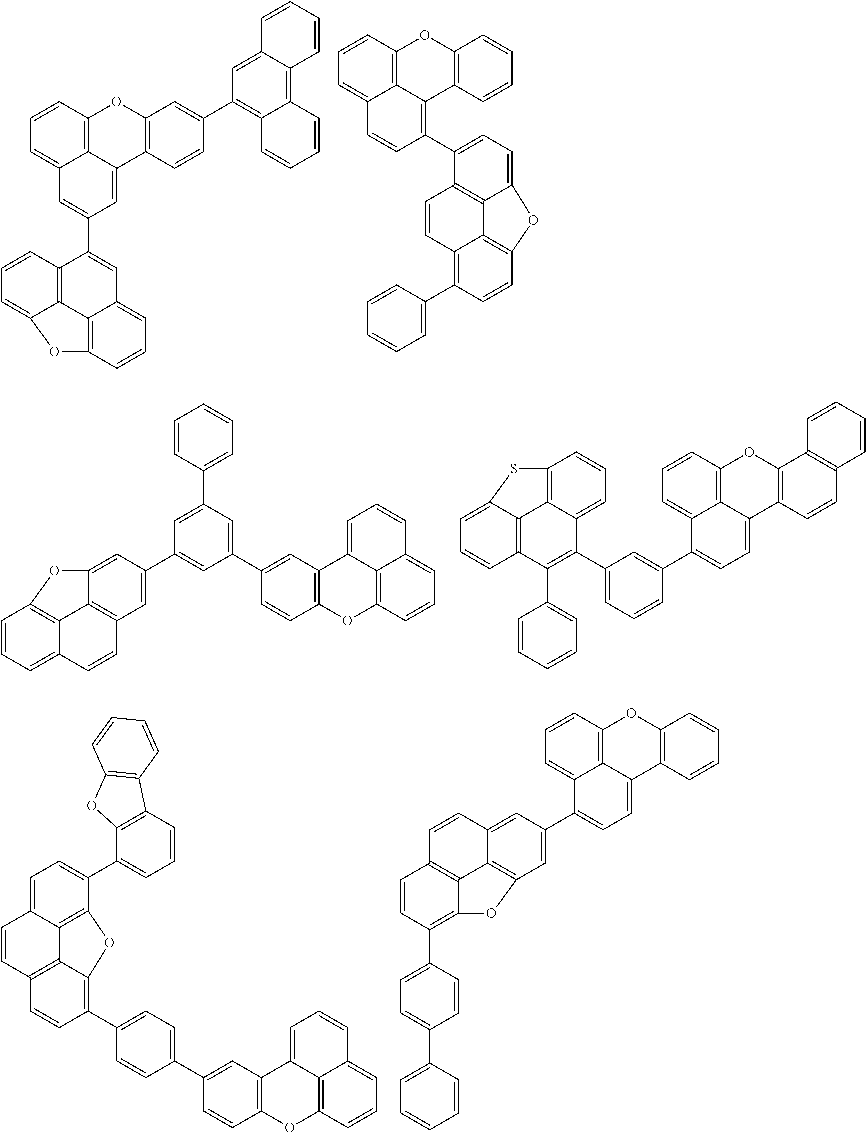

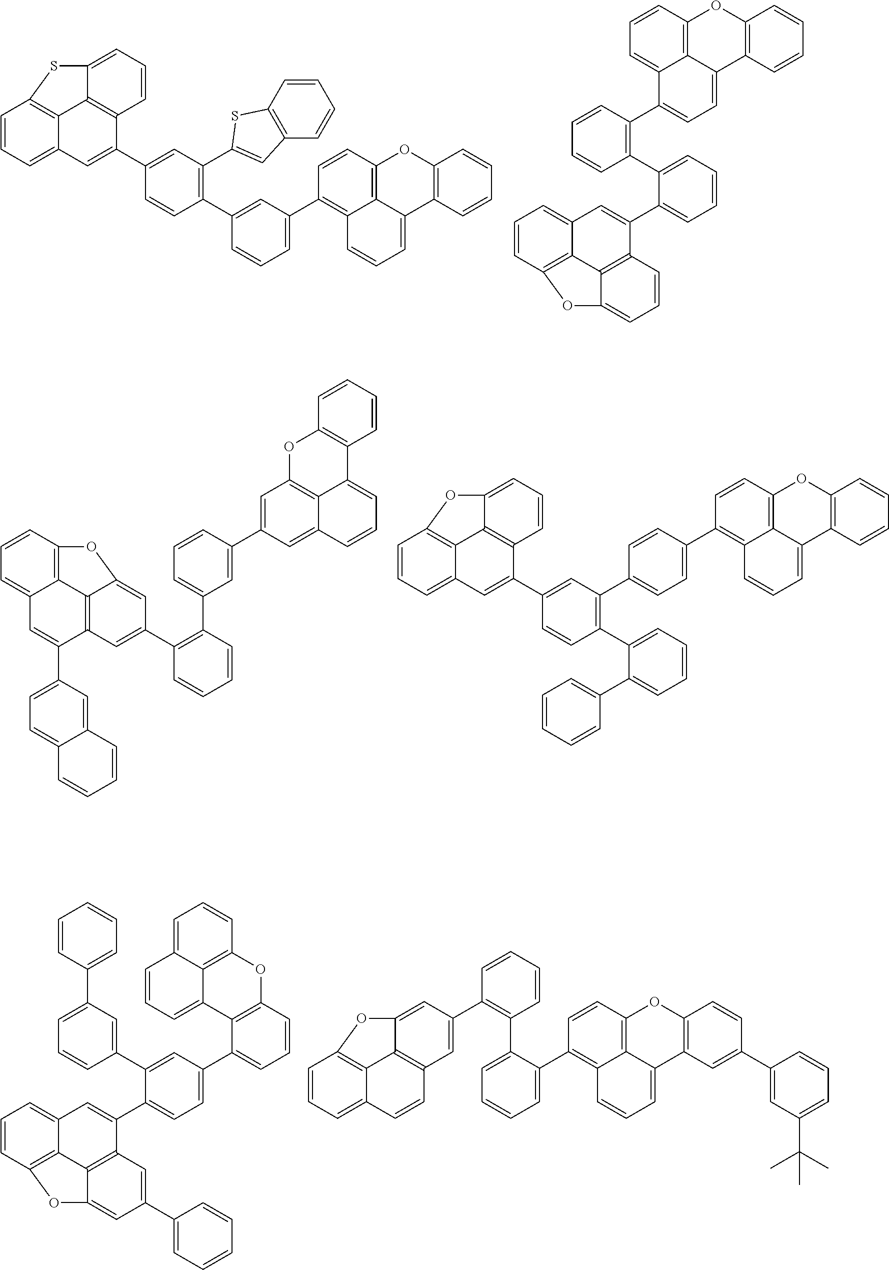

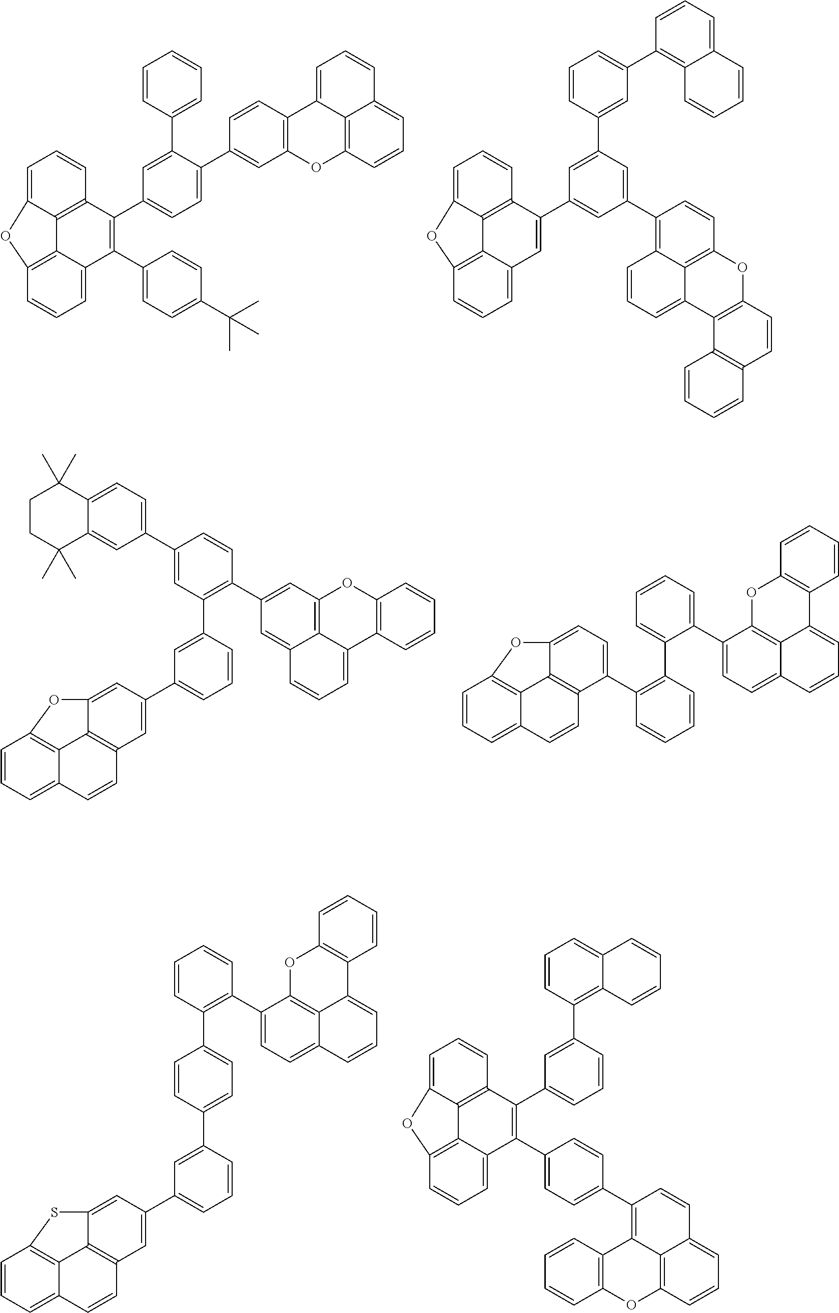

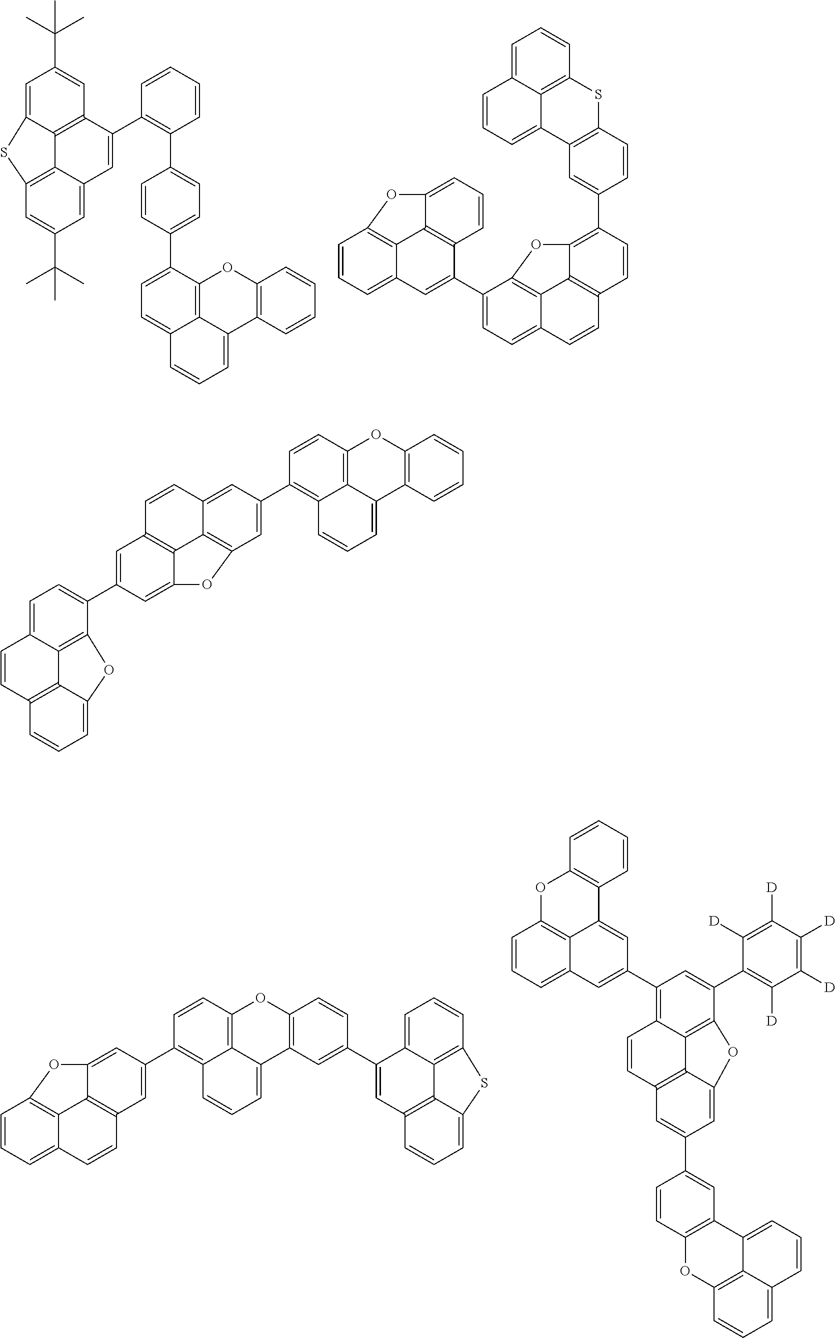

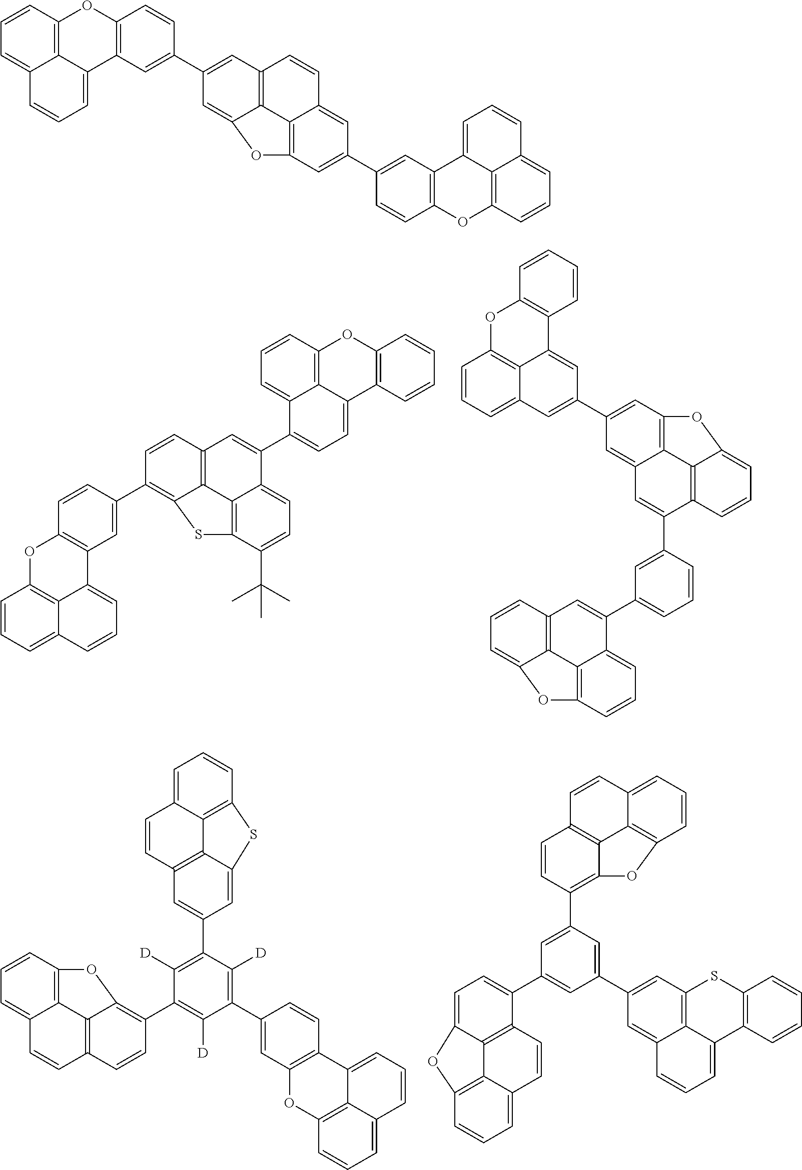

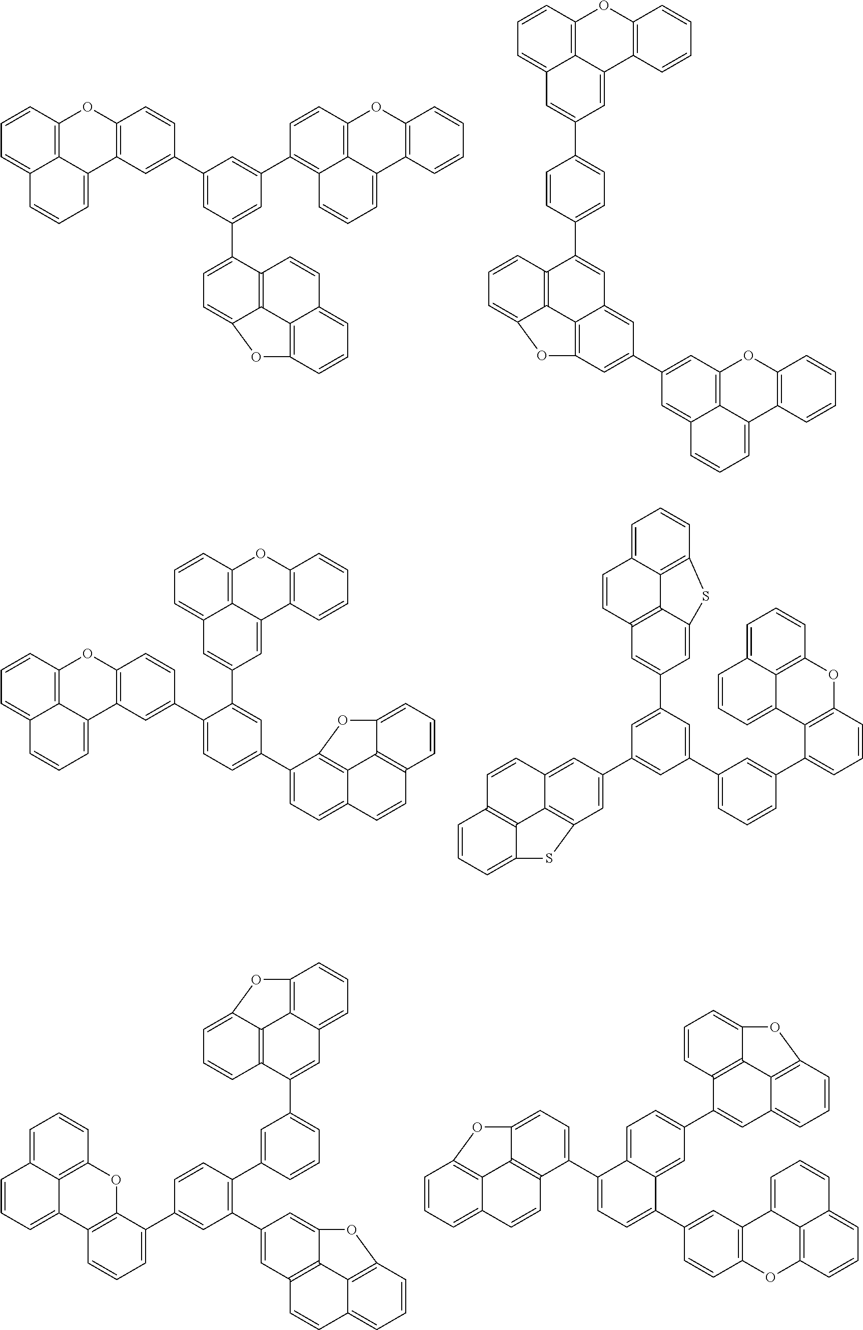

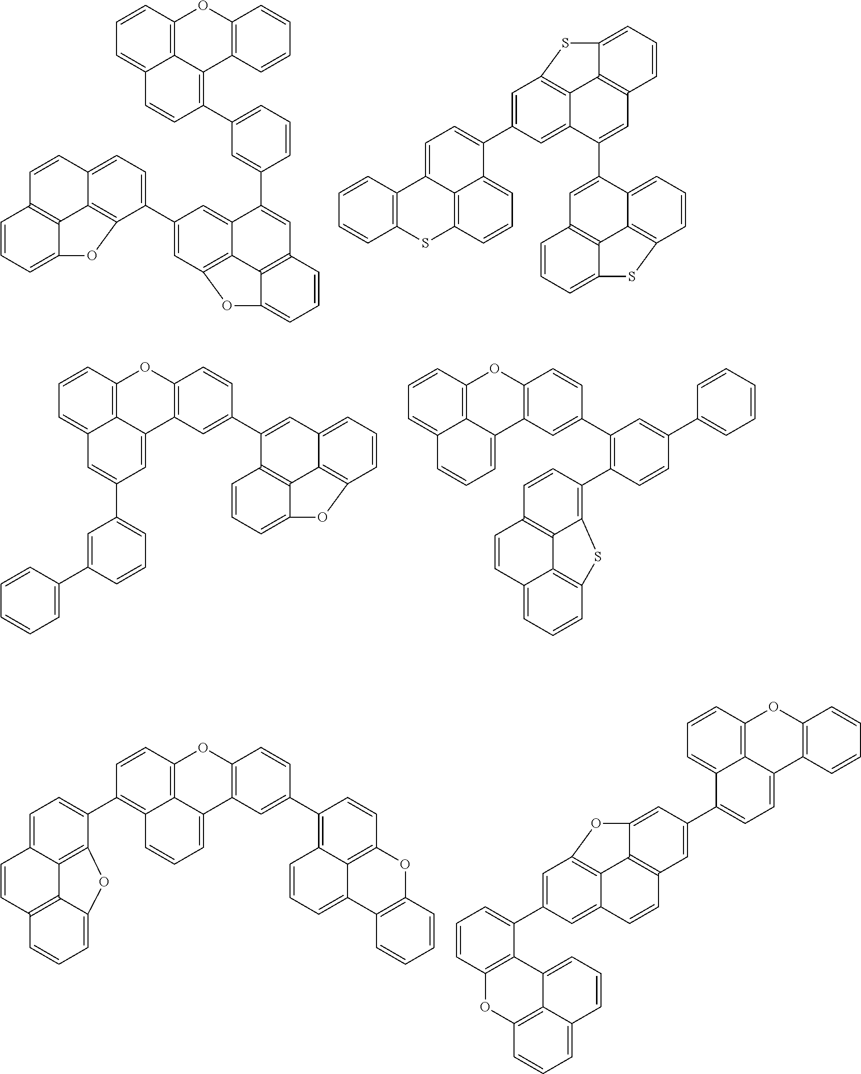

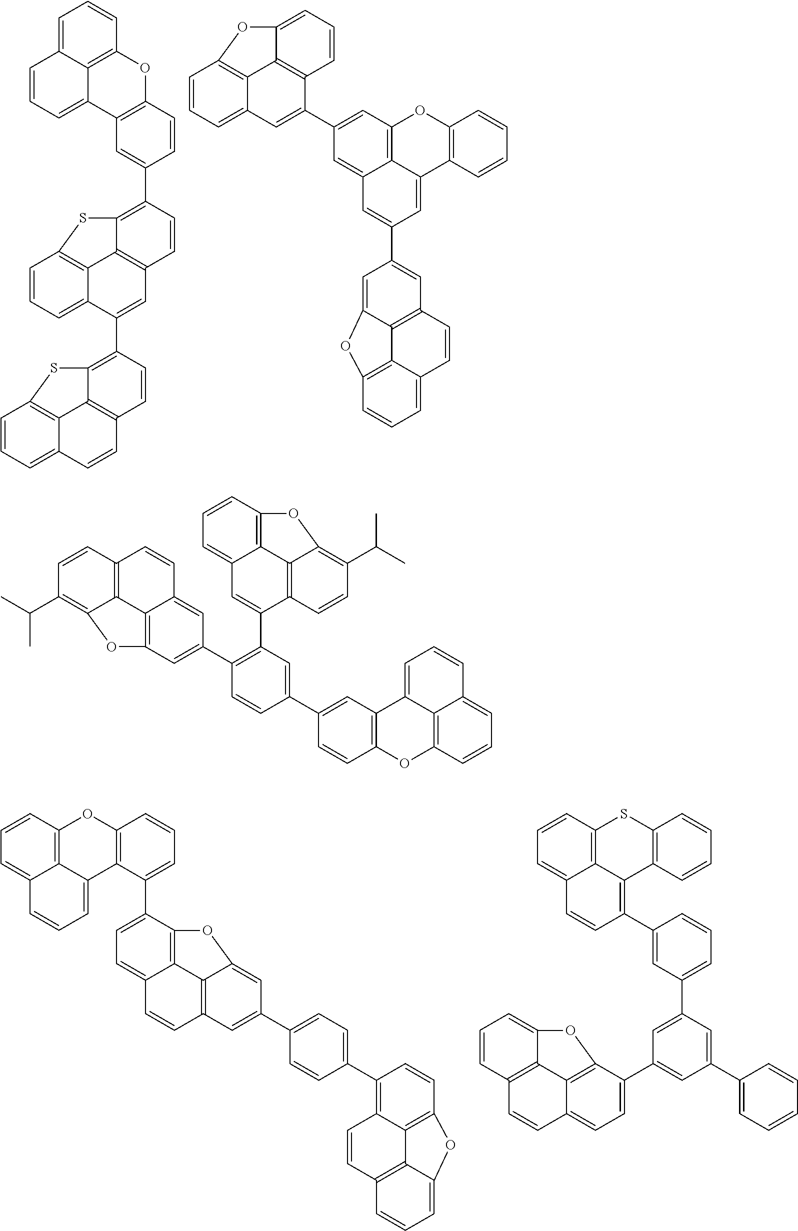

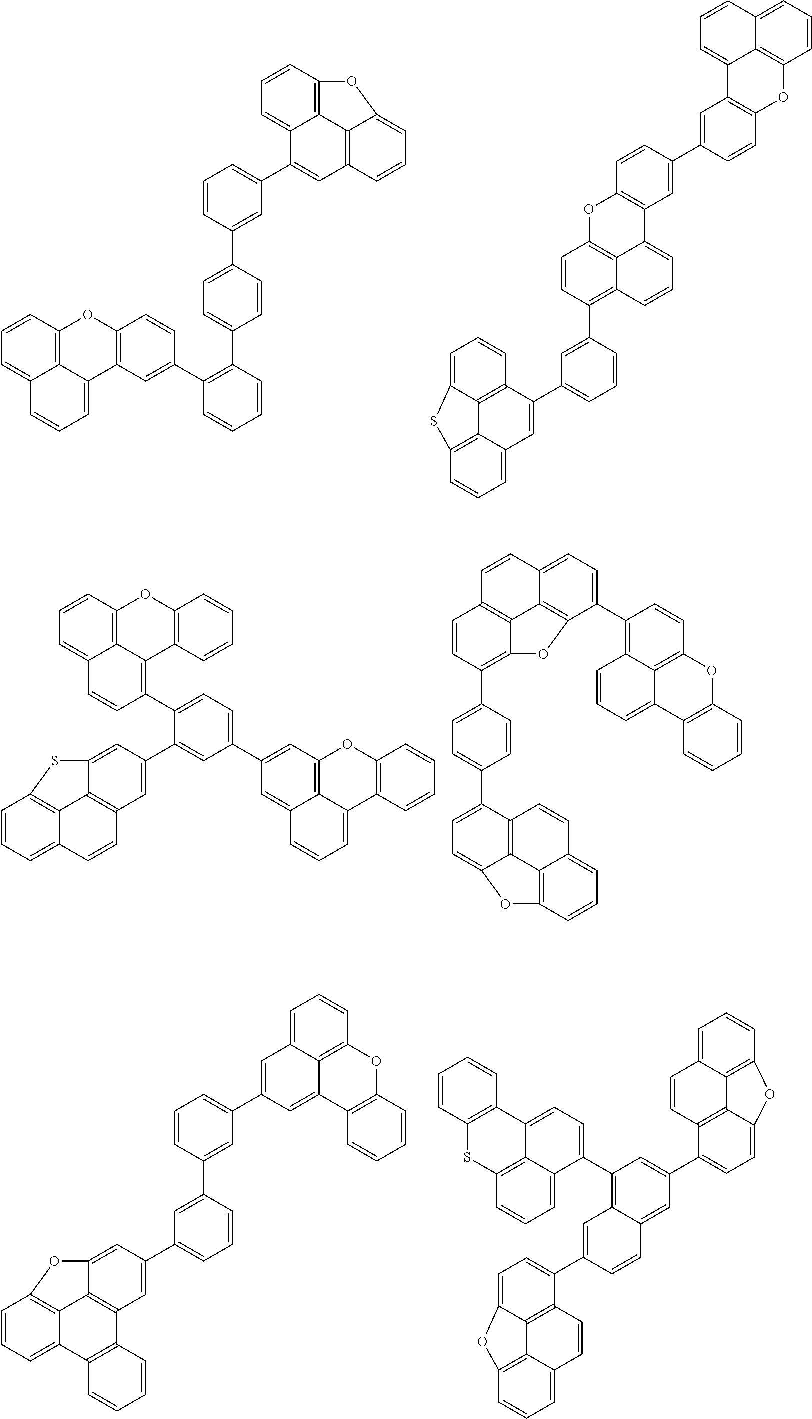

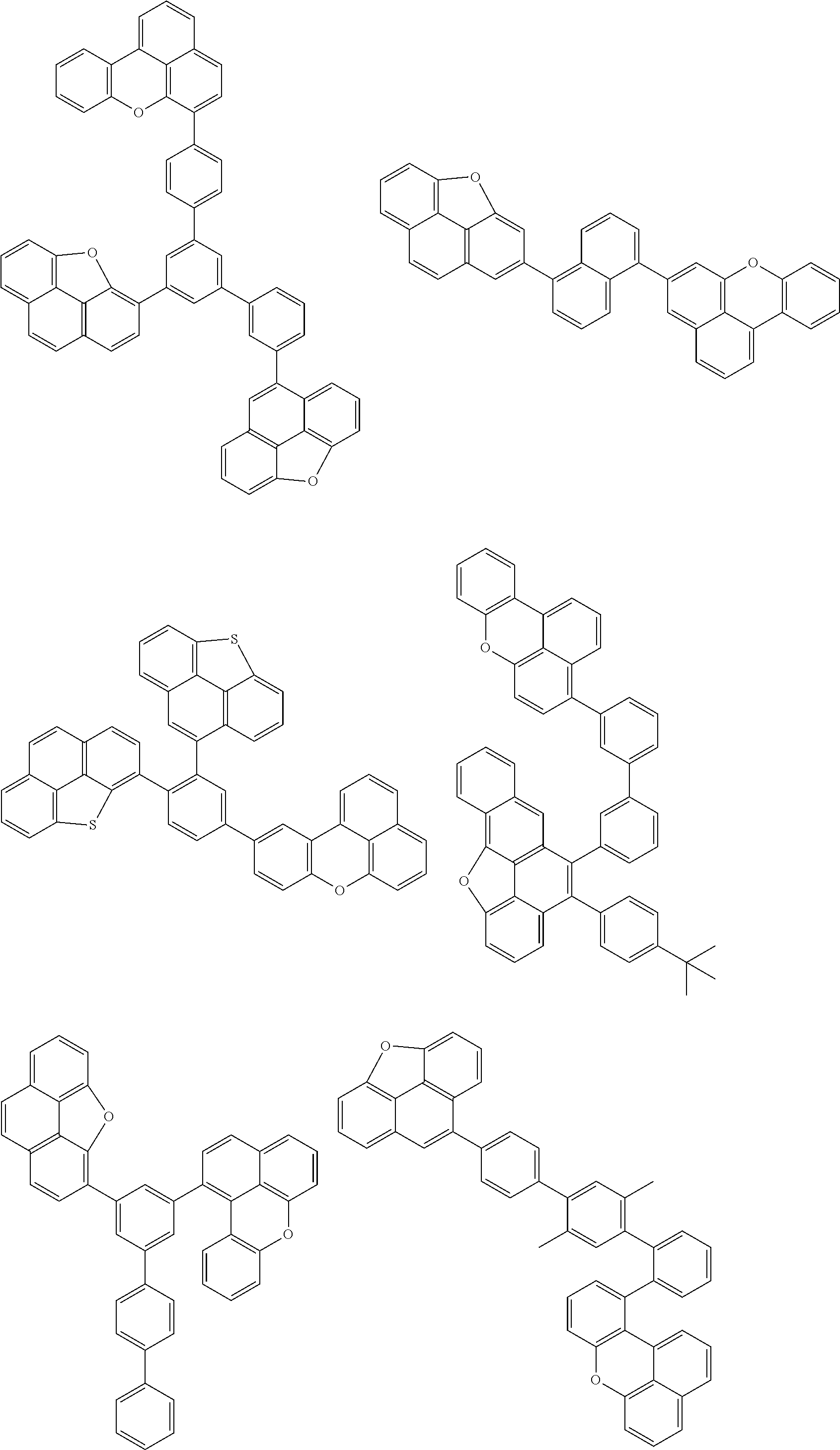

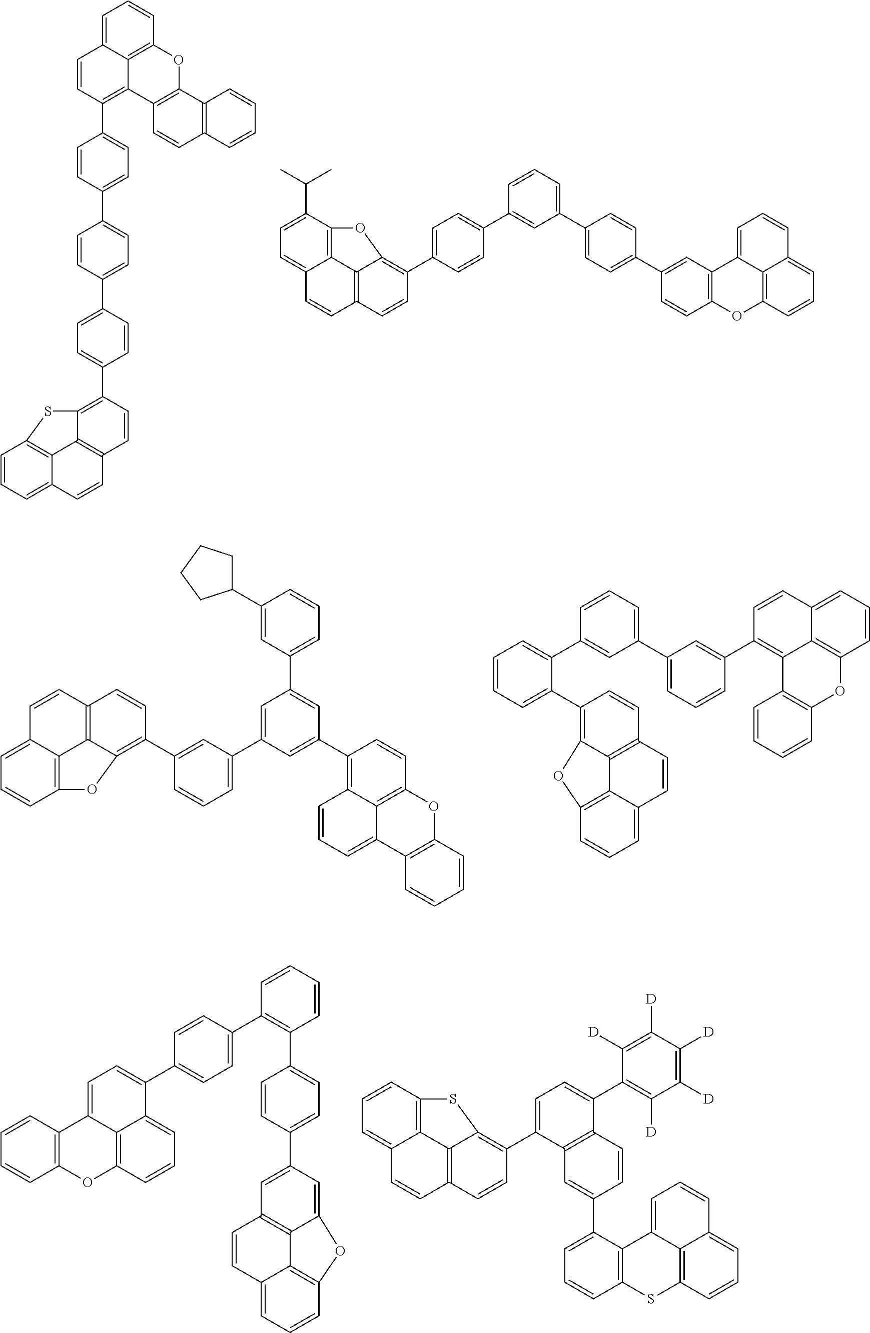

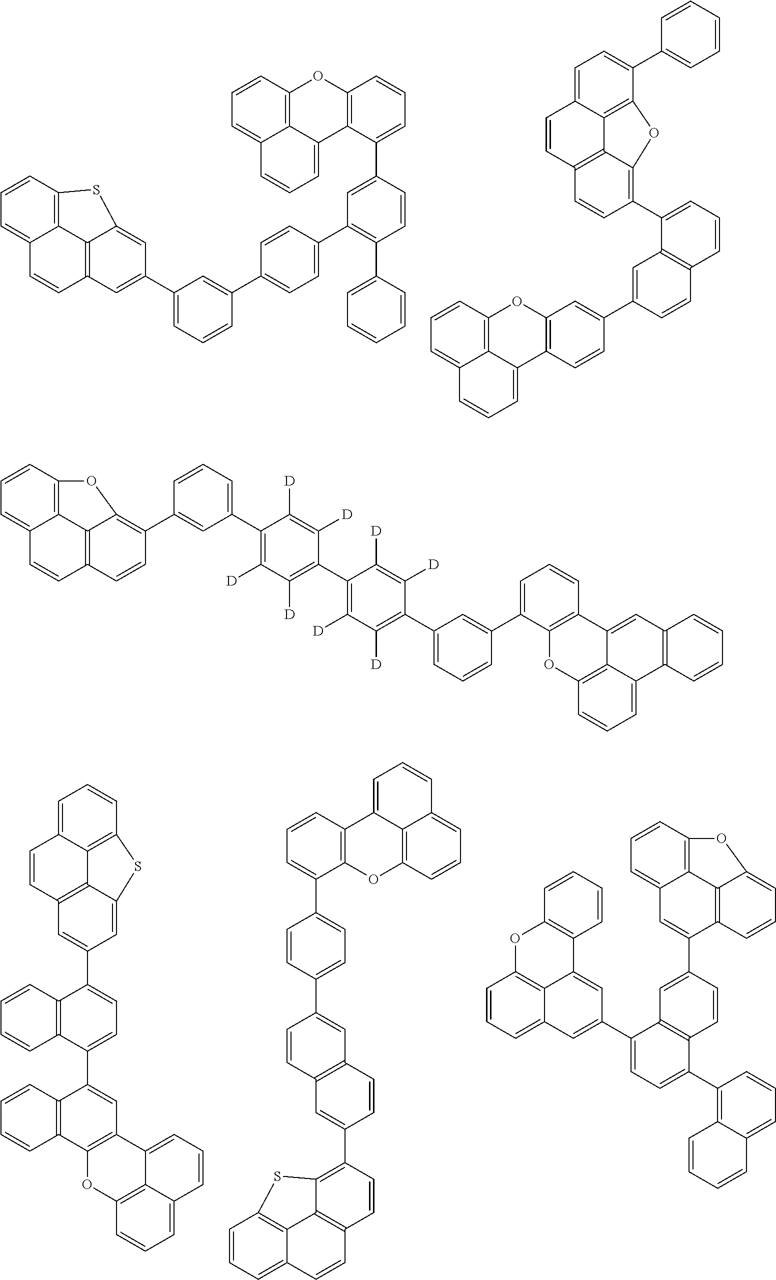

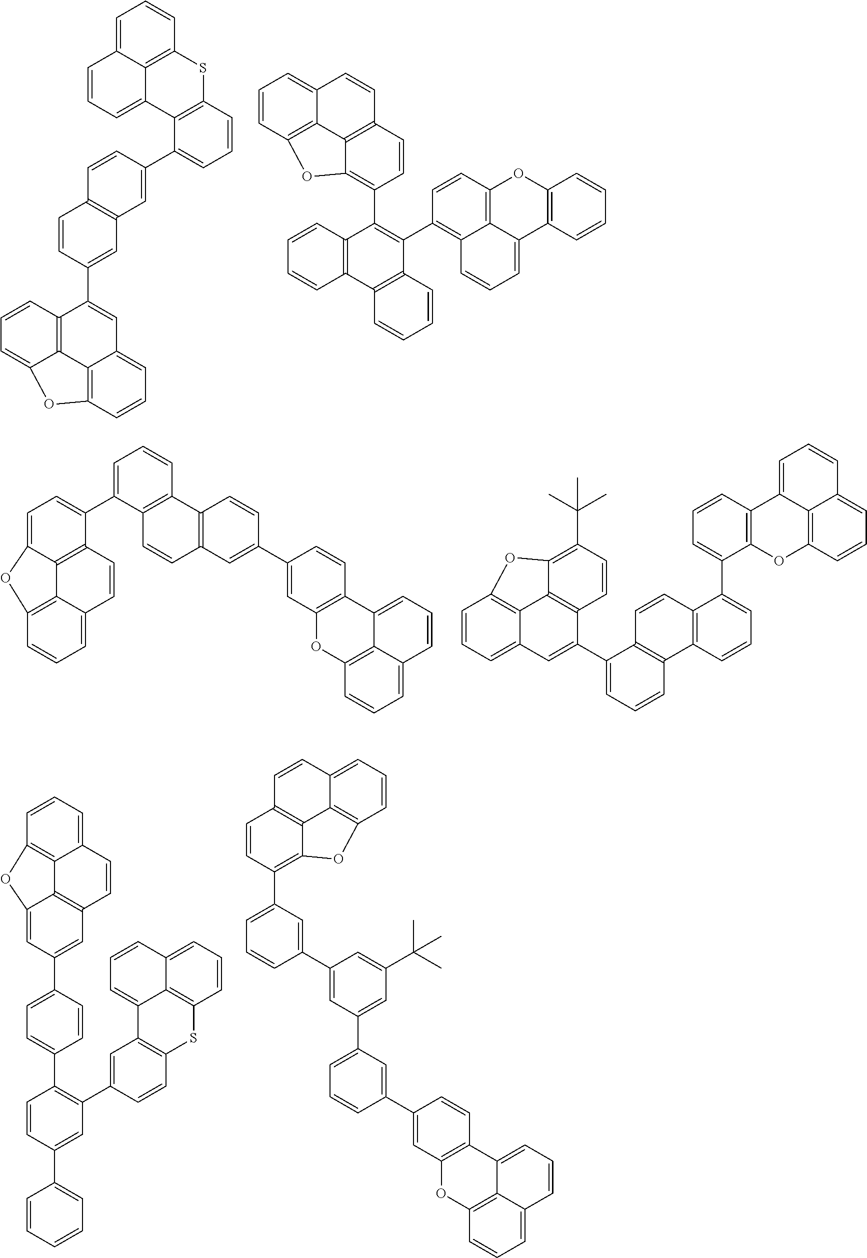

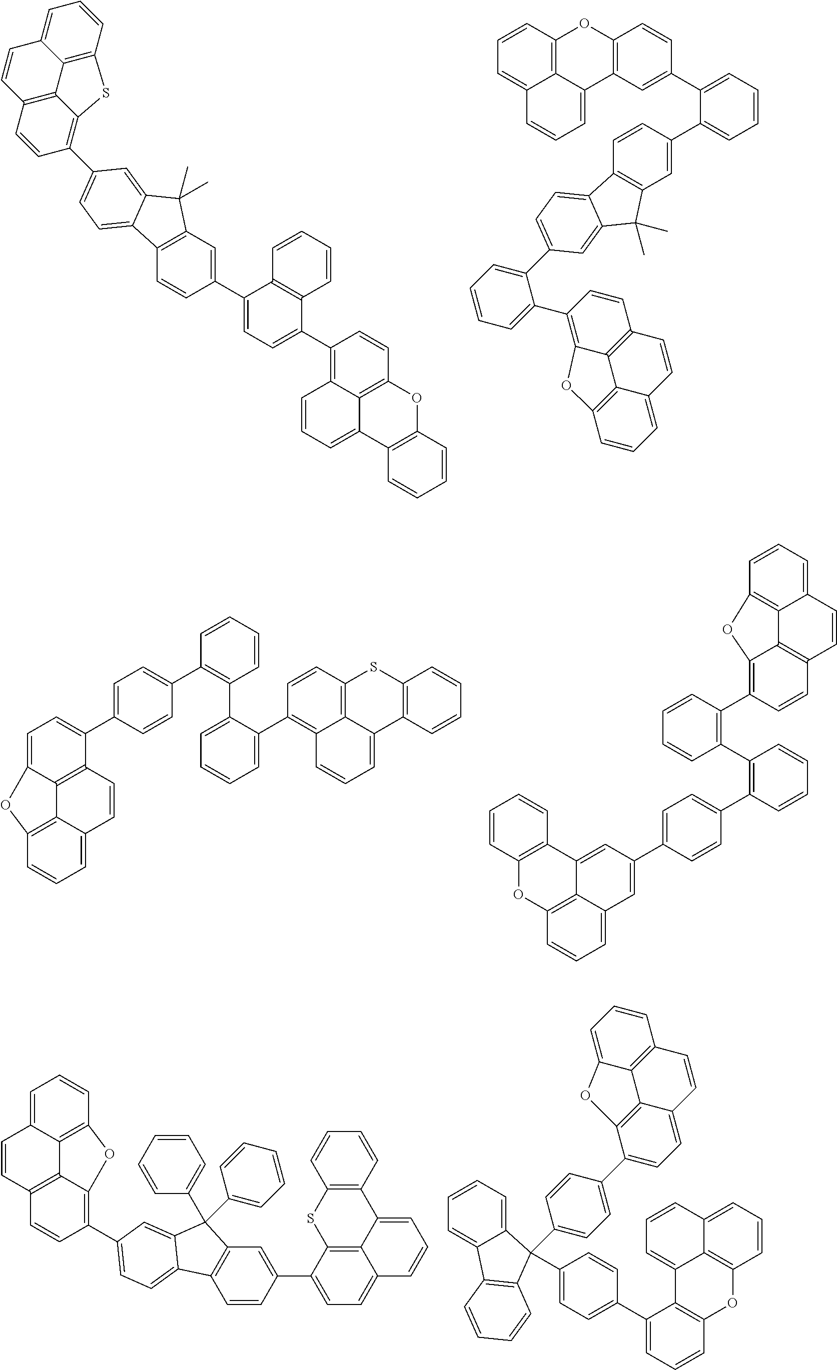

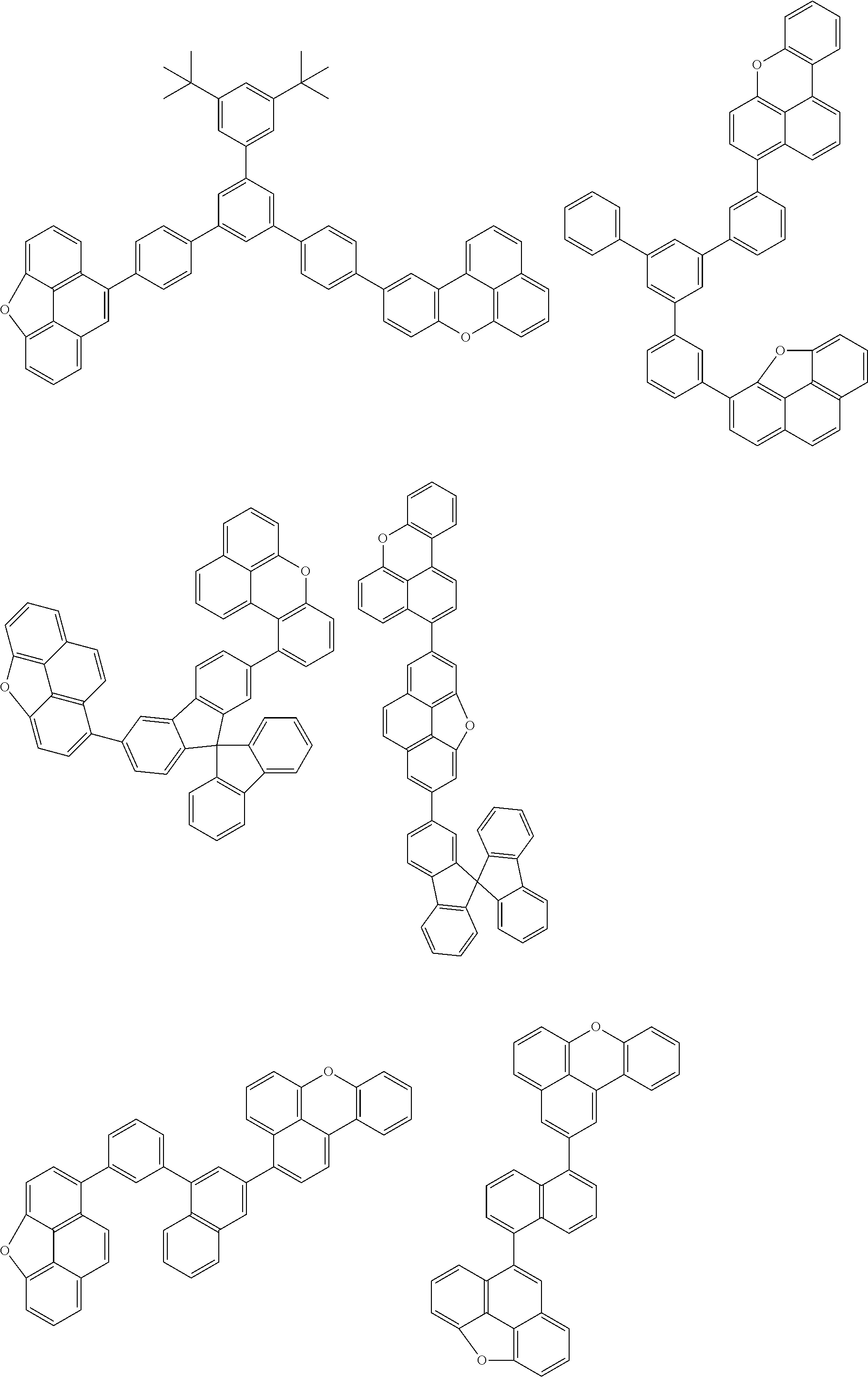

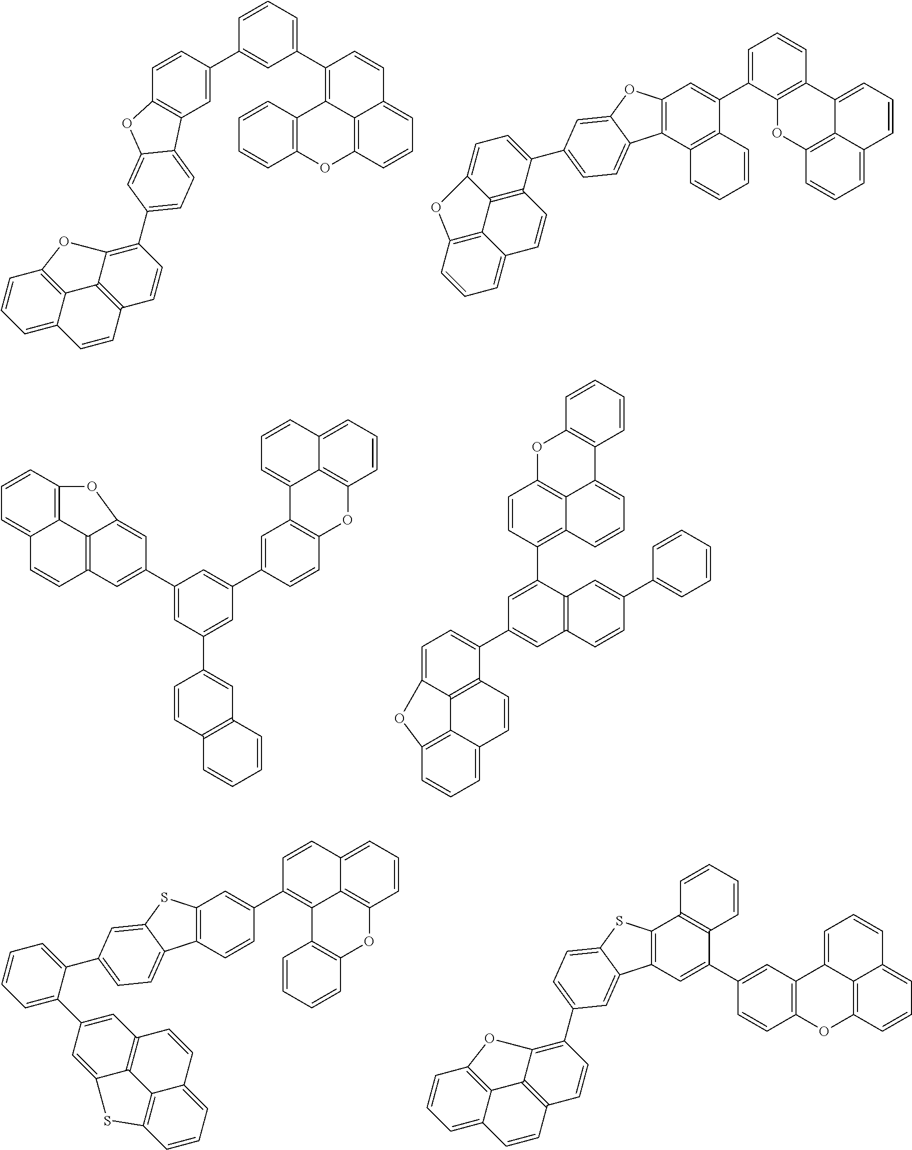

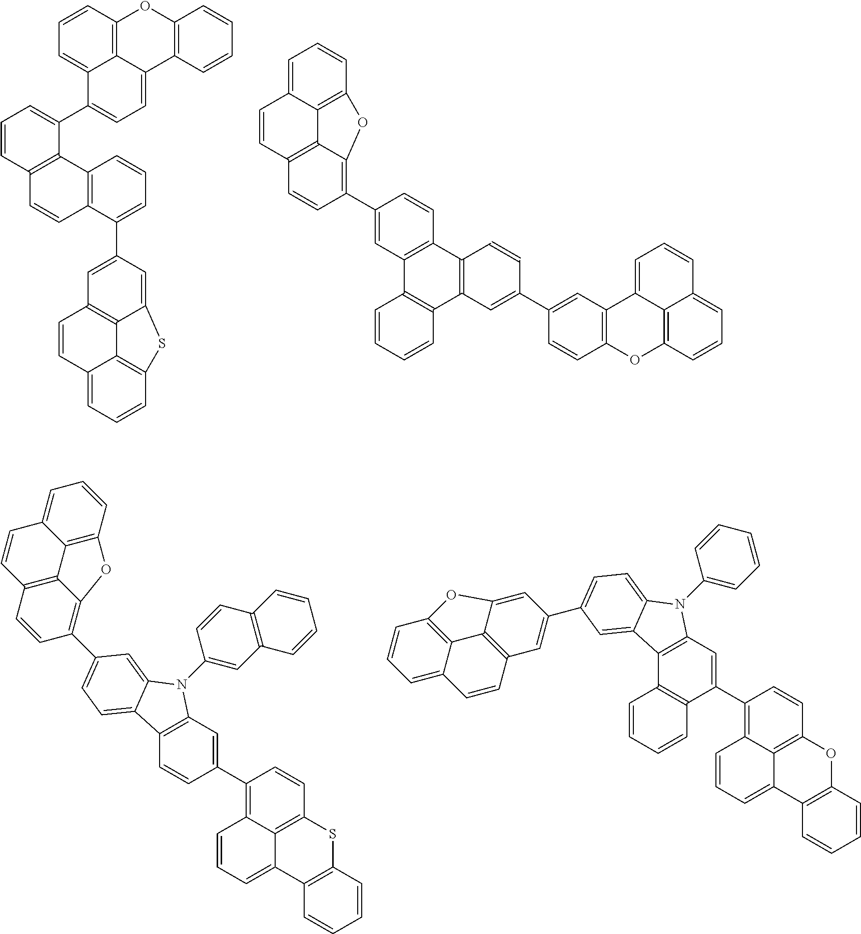 [SMILES: c1ccc2c(c1)Oc1cccc3c(-c4cccc5ccc6c(-c7cc8ccc9cccc%10sc(c7)c8c9%10)cccc6c45)ccc-2c13 ; c1cc2c3c(cccc3c1)-c1cc(-c3ccc4c5ccc(-c6ccc7ccc8cccc9oc6c7c89)cc5c5ccccc5c4c3)ccc1O2 ; c1ccc2c(c1)Sc1ccc(-c3ccc4c5ccc(-c6ccc7ccc8cccc9oc6c7c89)cc5n(-c5ccc6ccccc6c5)c4c3)c3cccc-2c13 ; c1ccc(-n2c3ccc(-c4cc5ccc6cccc7oc(c4)c5c67)cc3c3c4ccccc4c(-c4ccc5c6c(cccc46)-c4ccccc4O5)cc32)cc1]

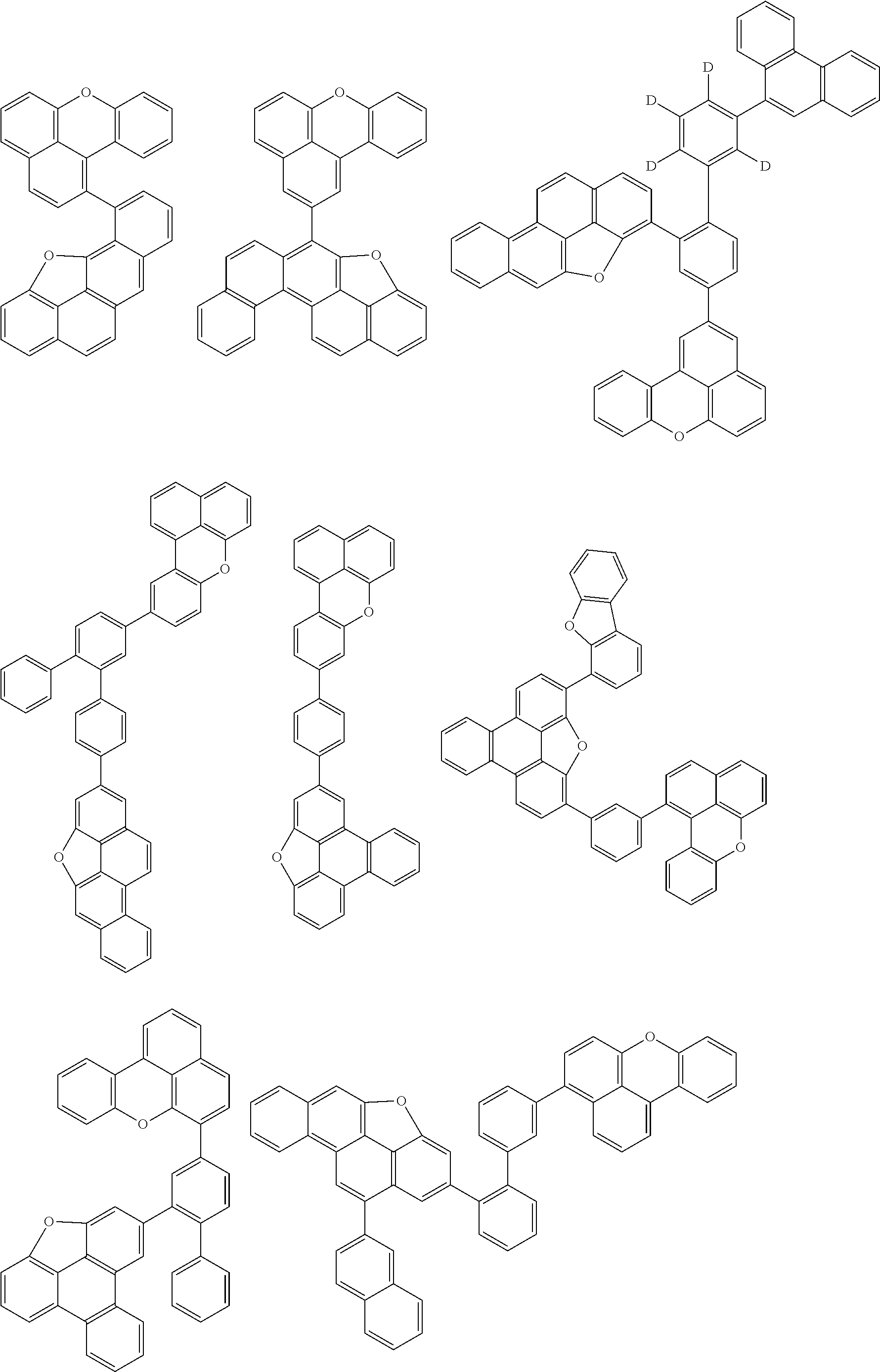

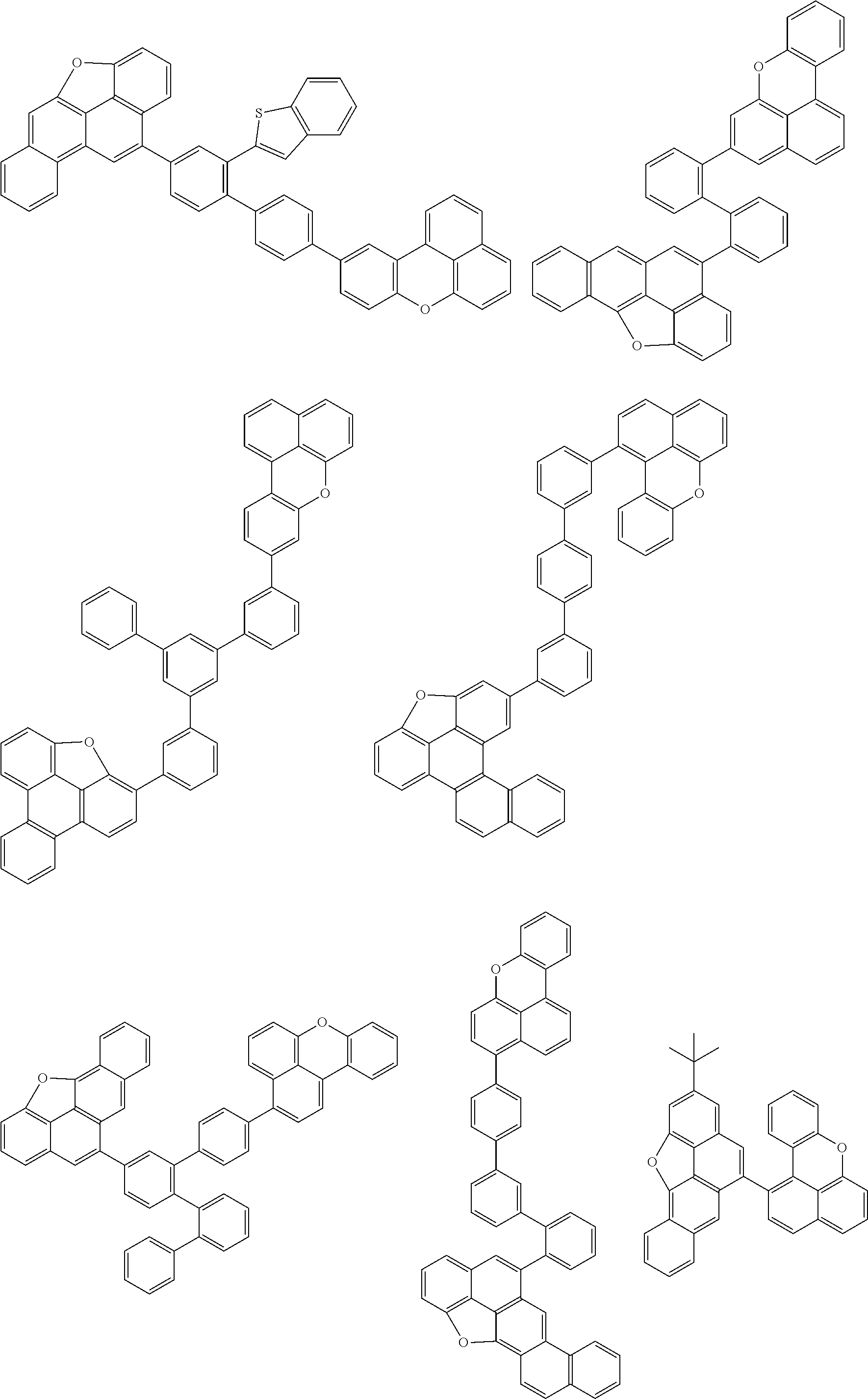

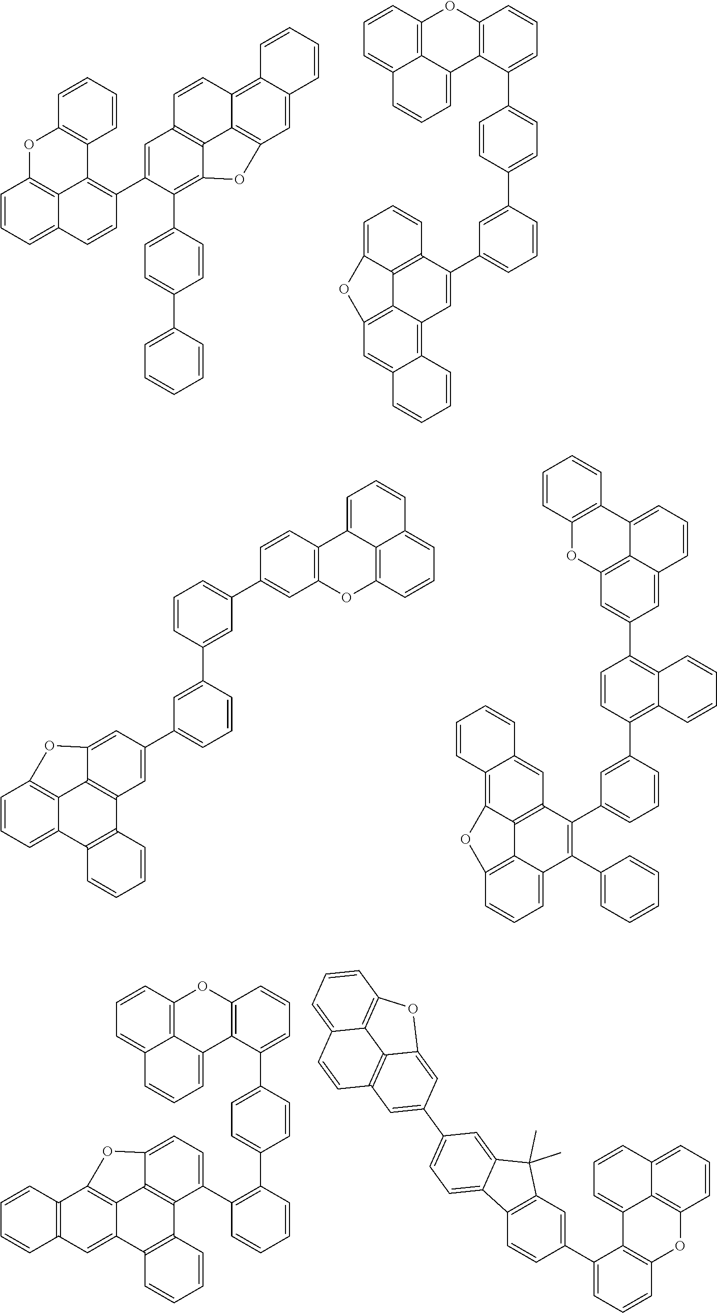

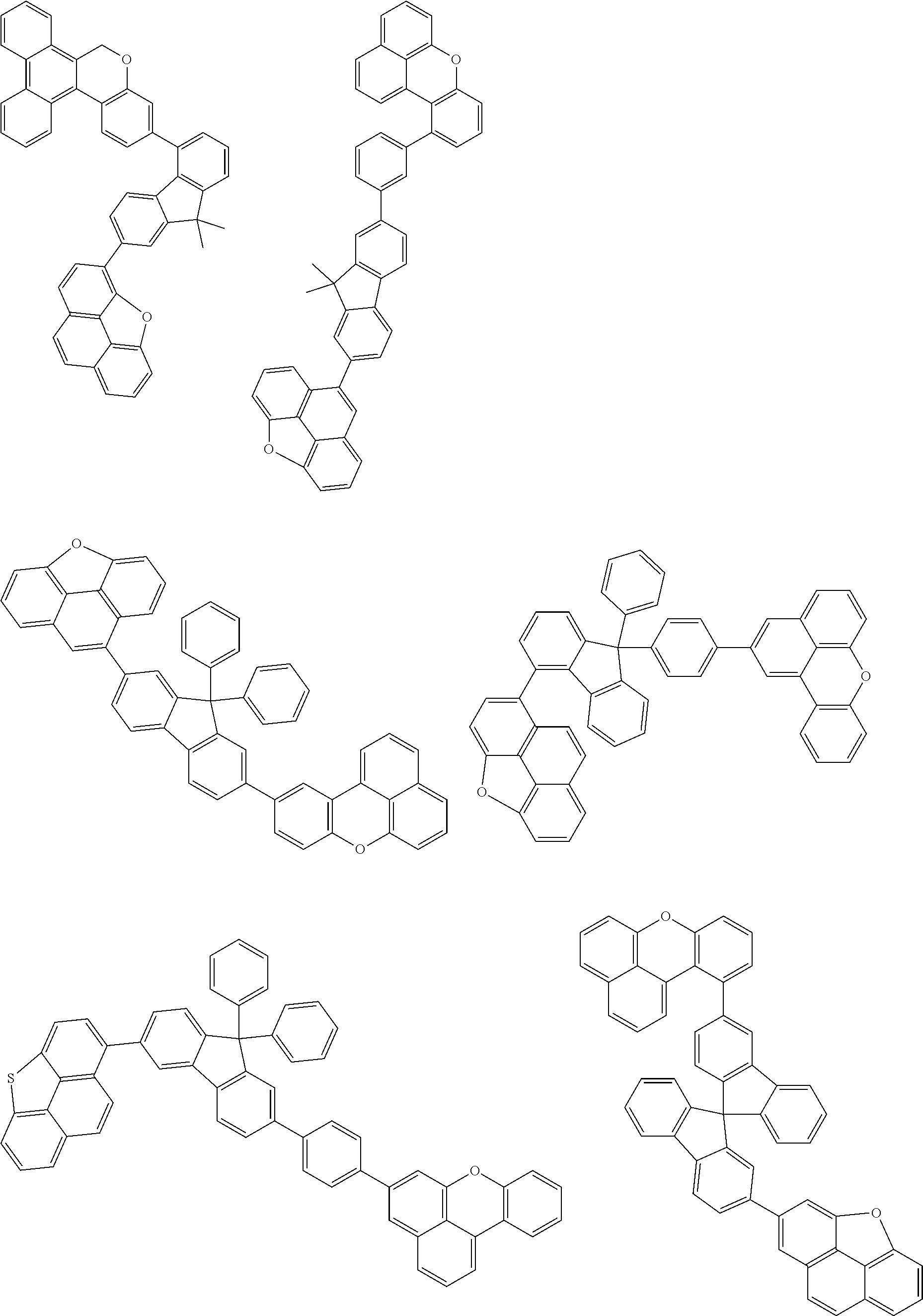

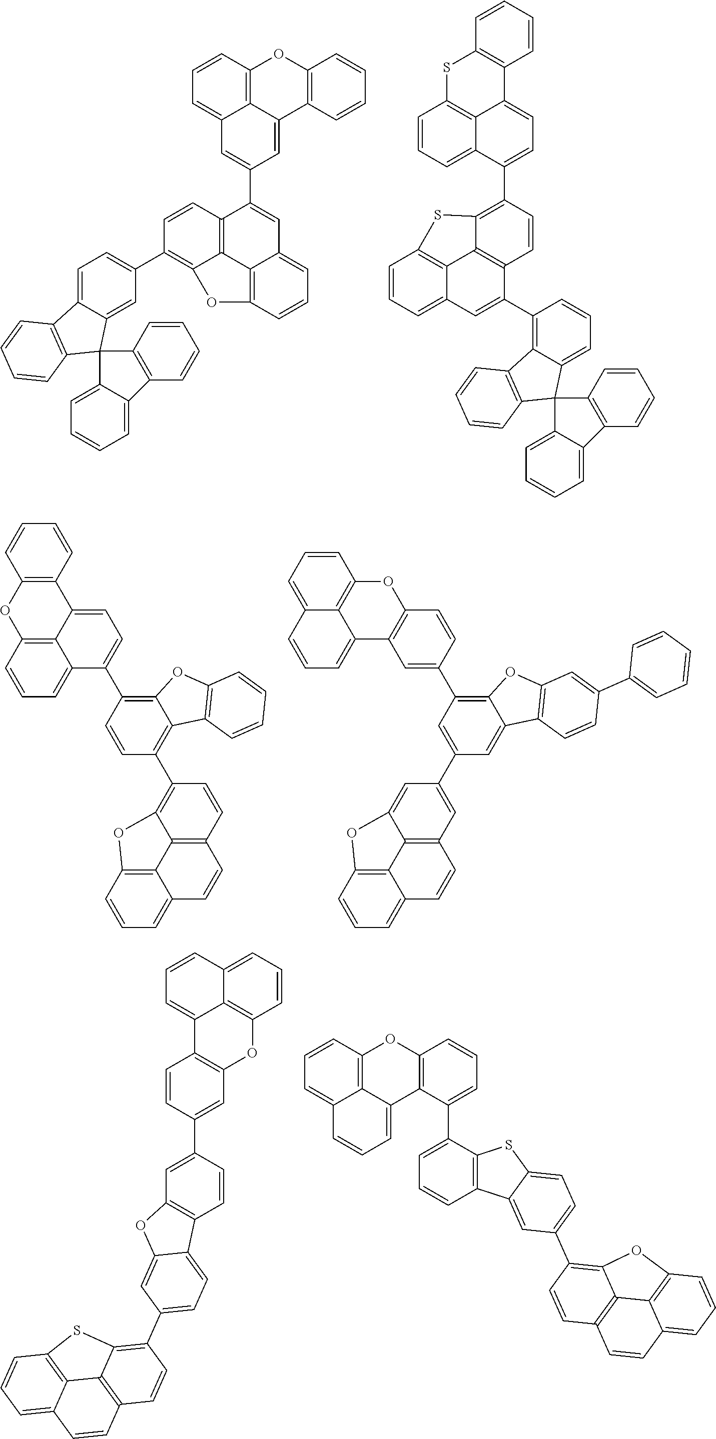

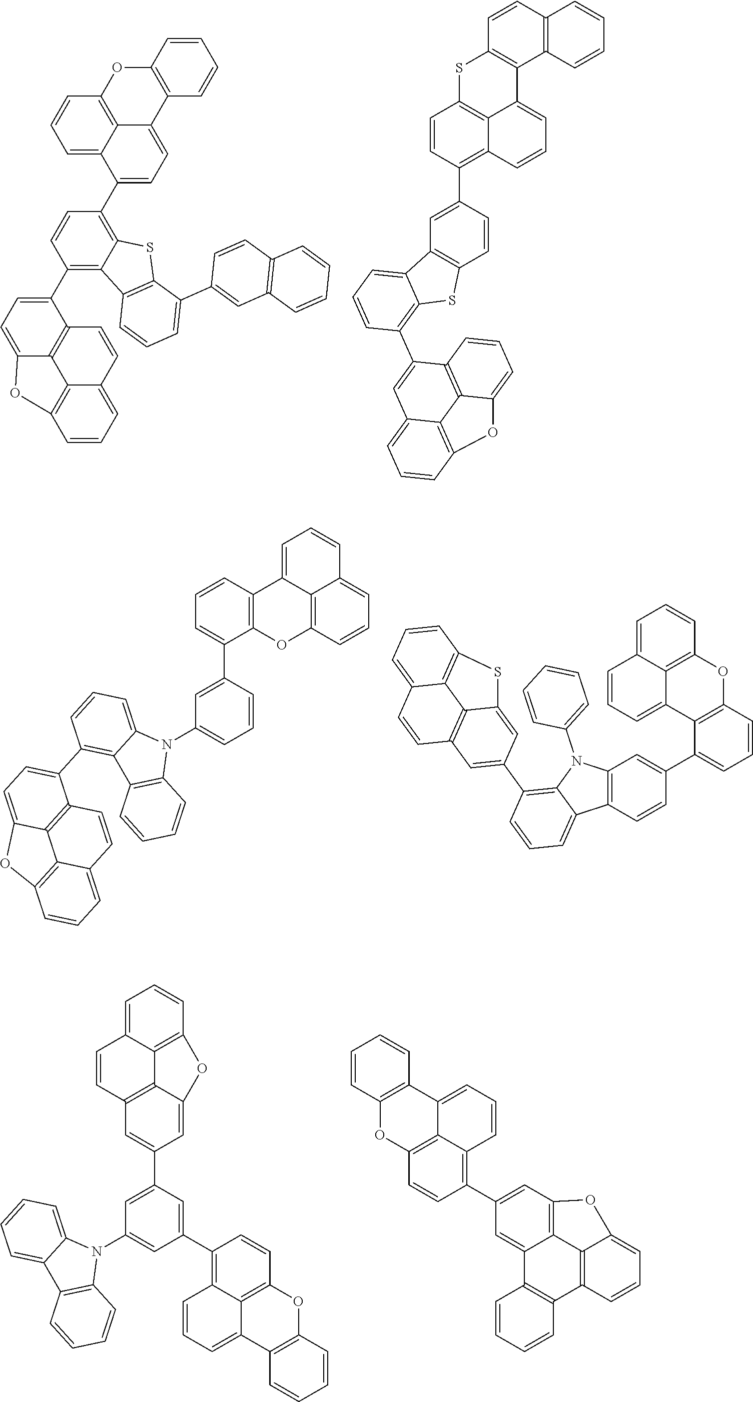

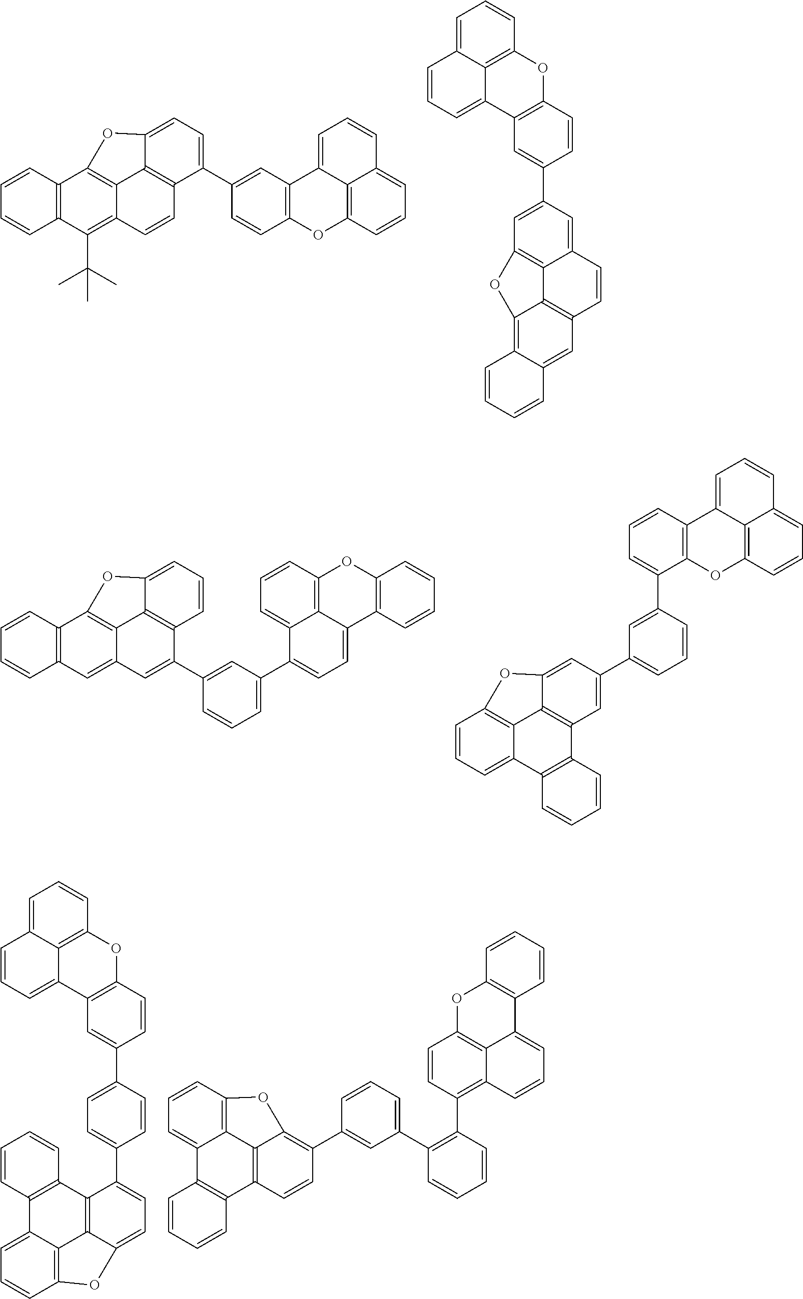

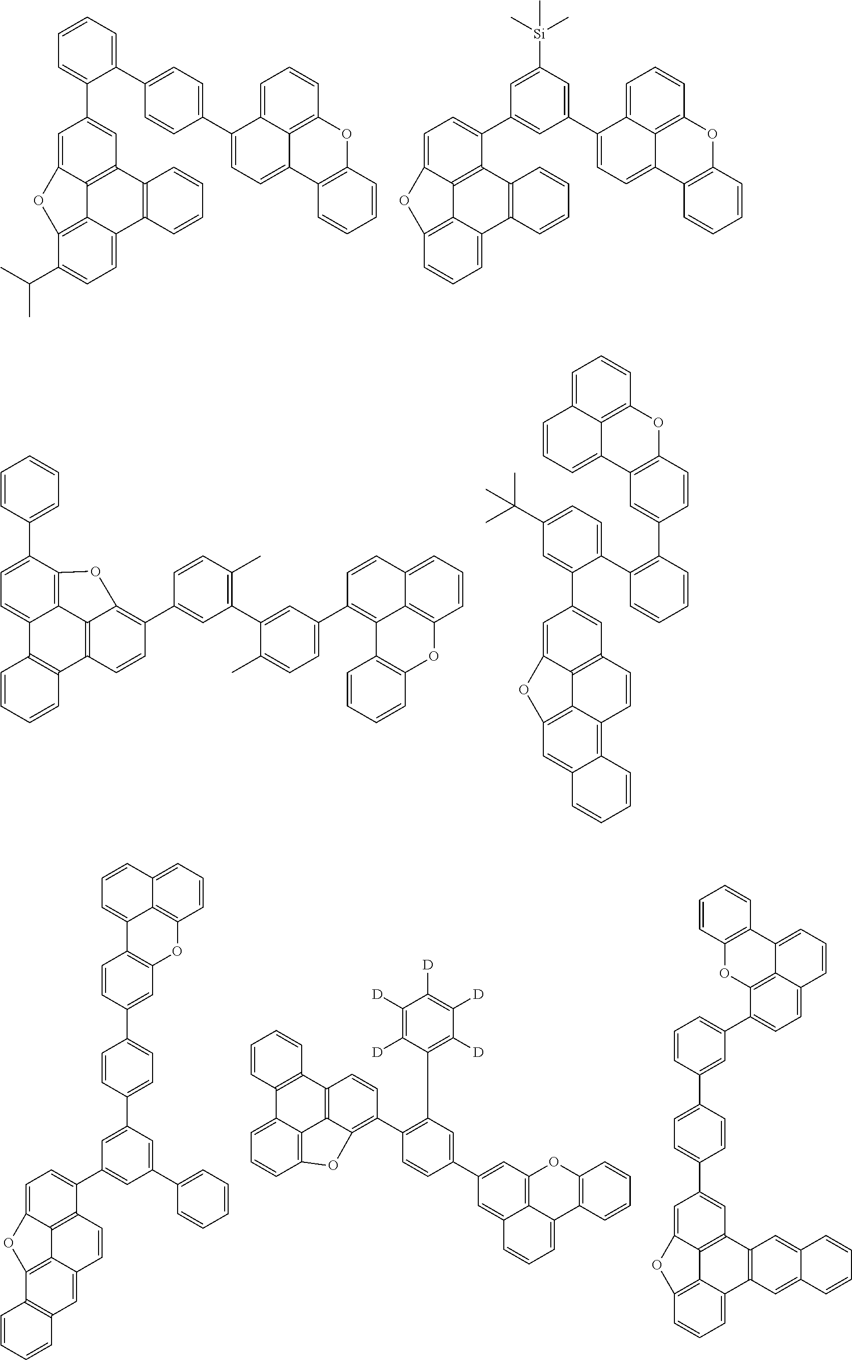

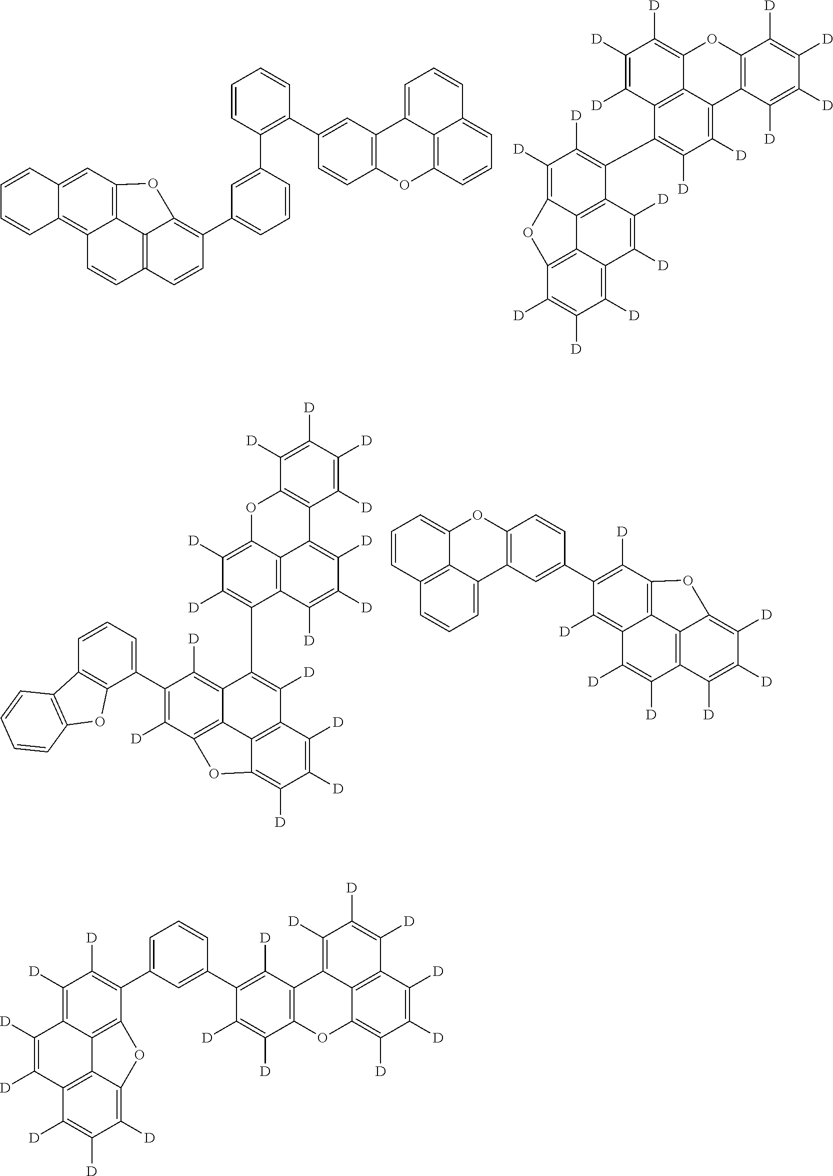

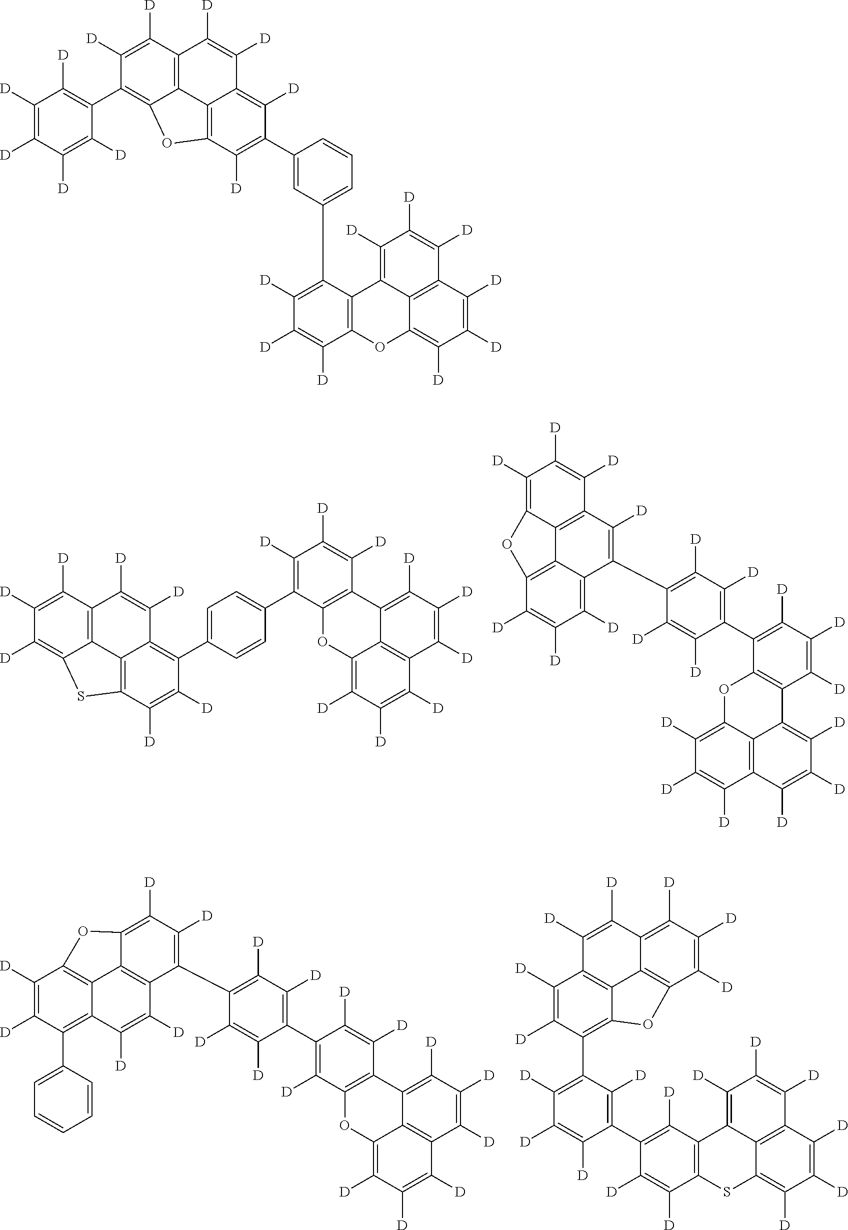

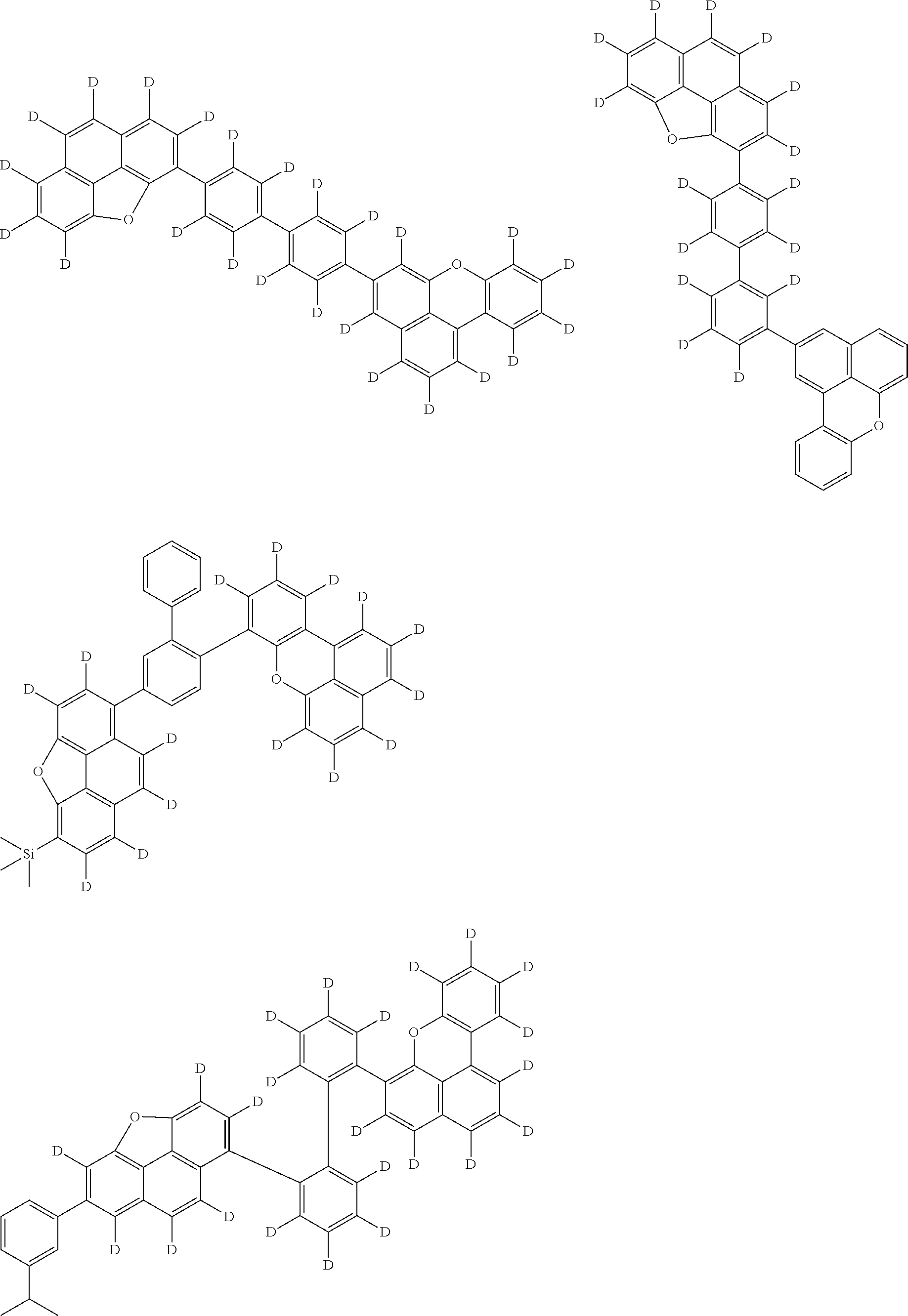

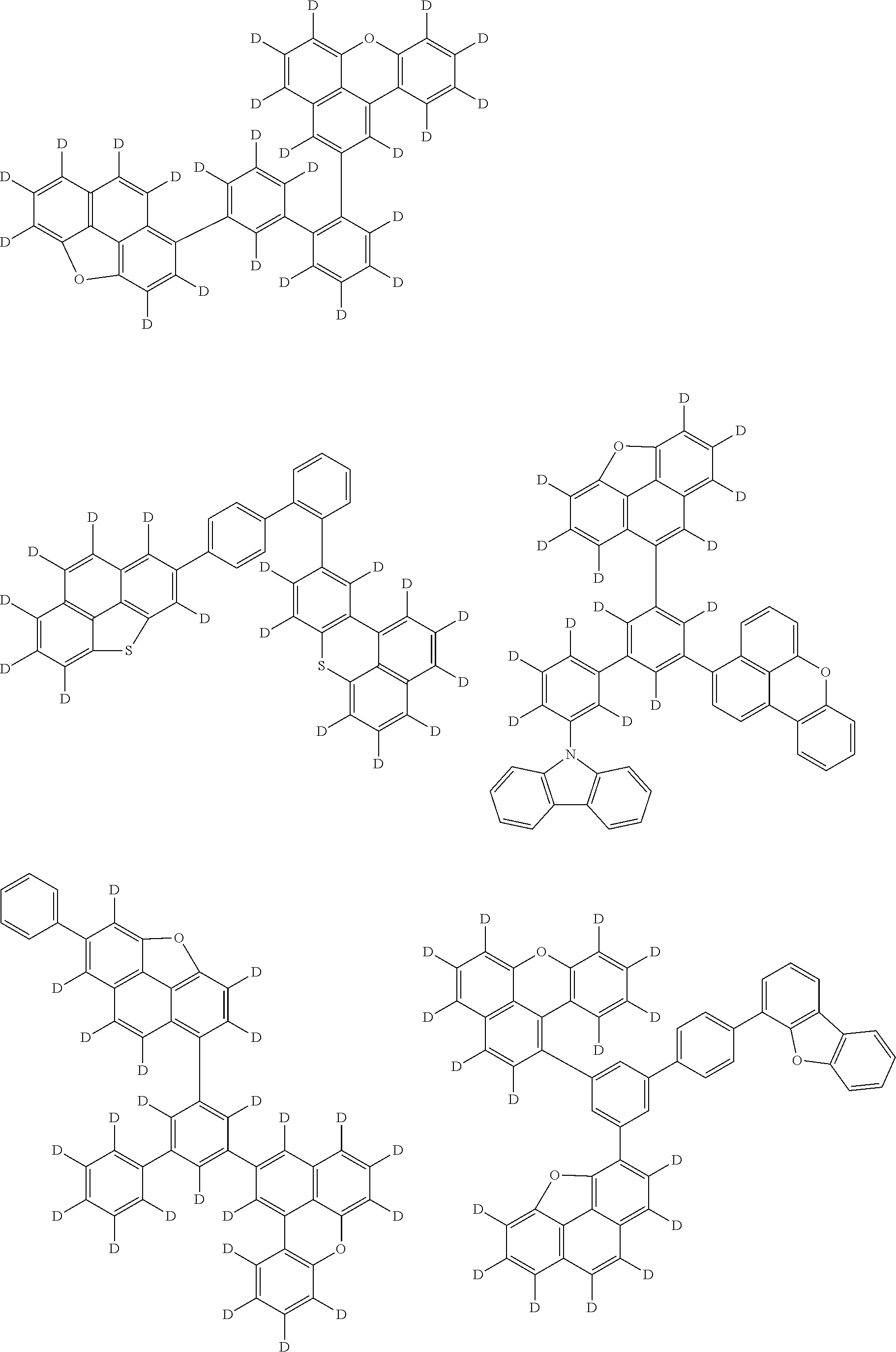

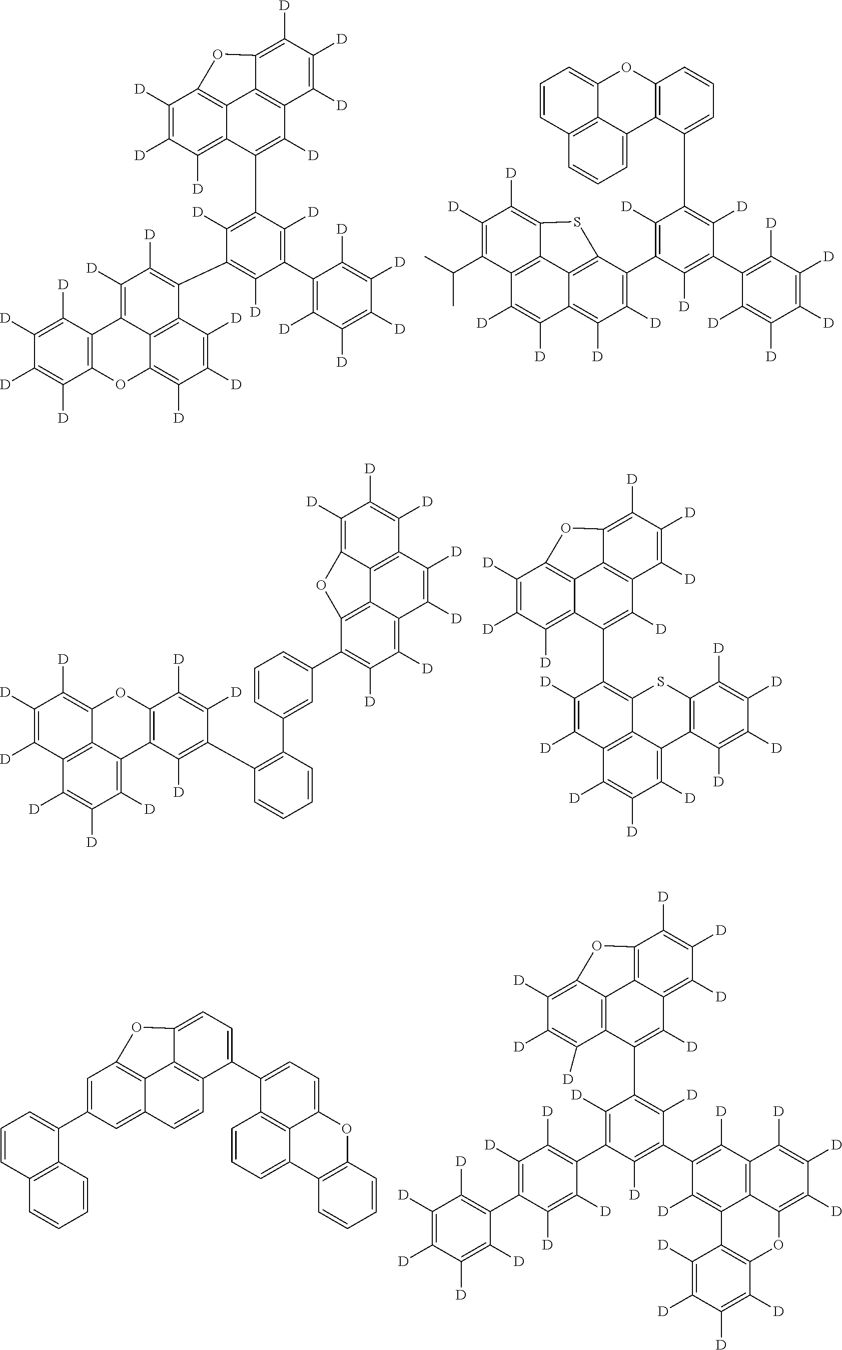

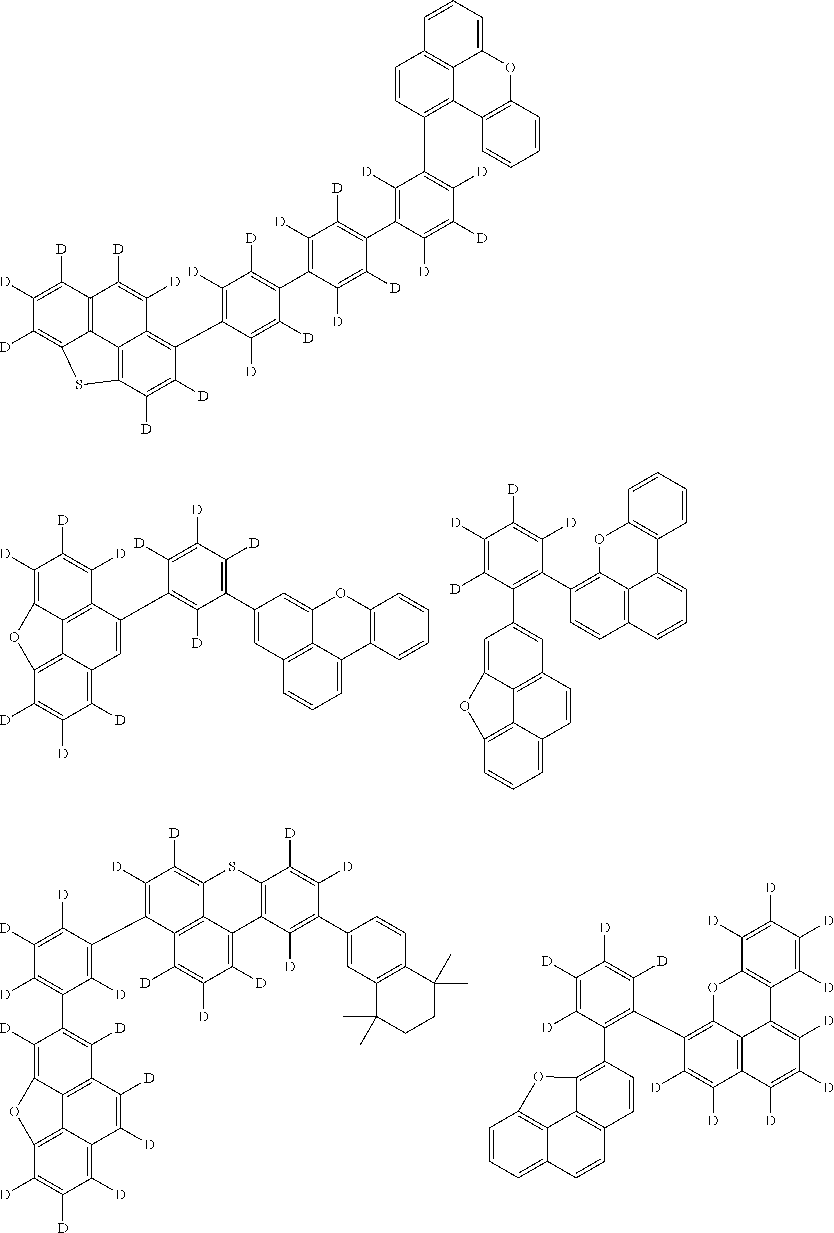

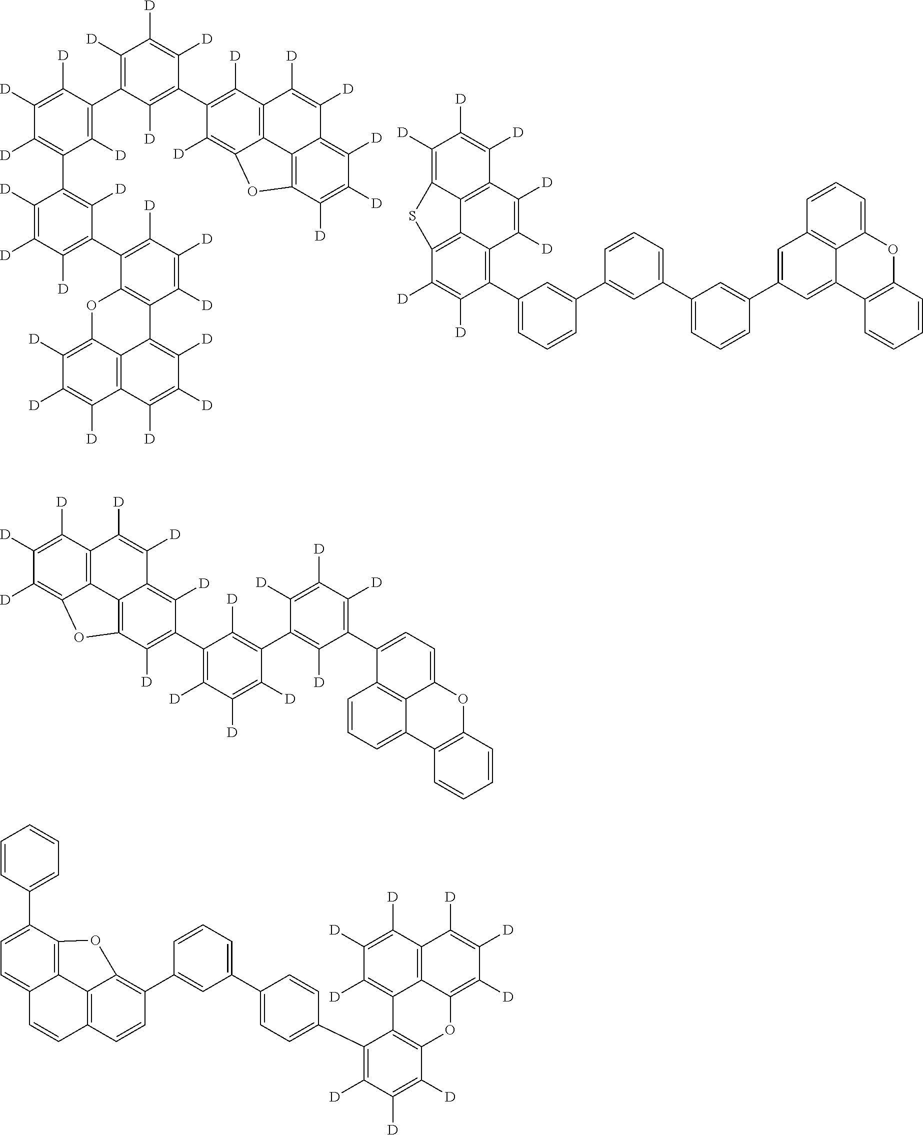

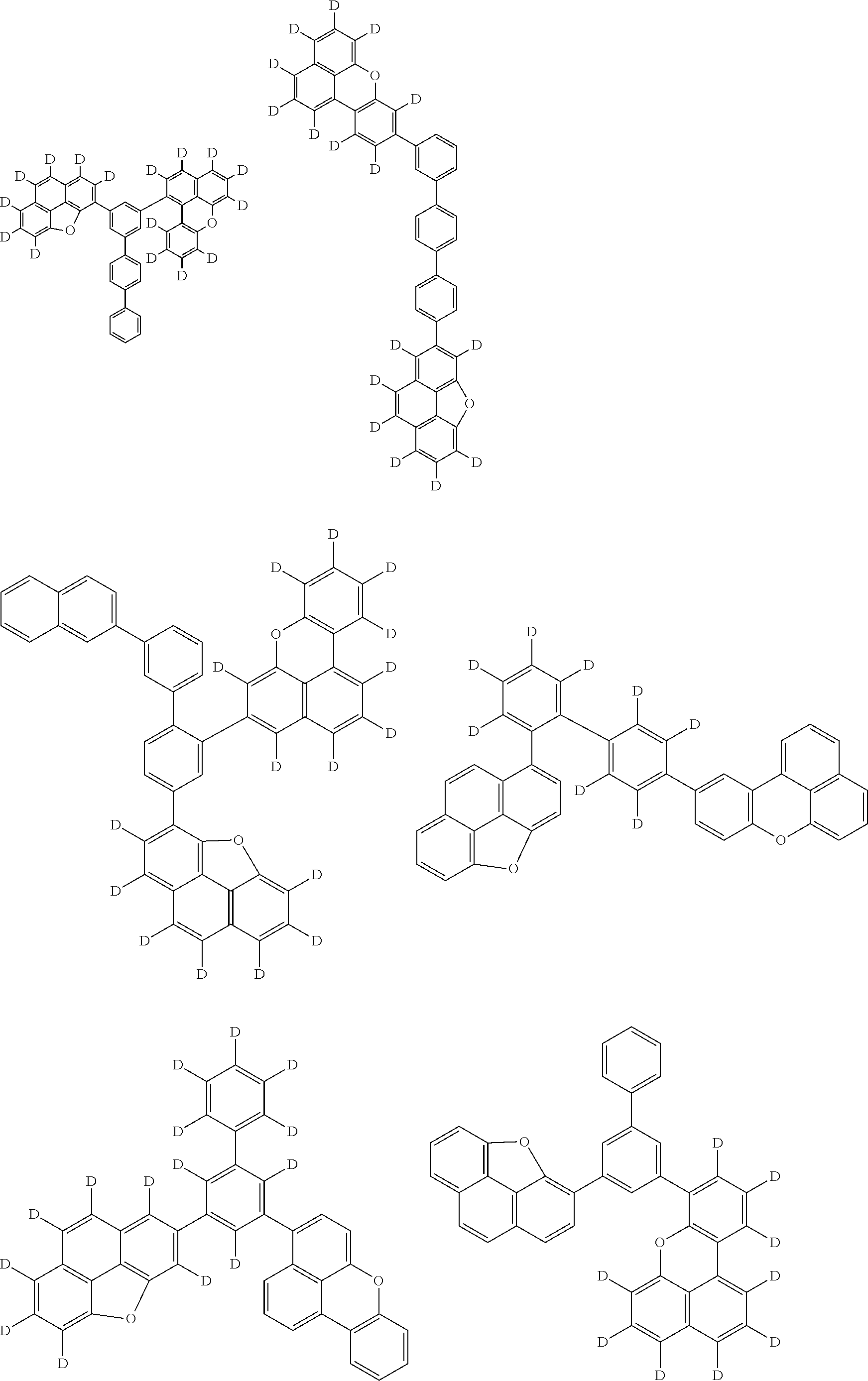

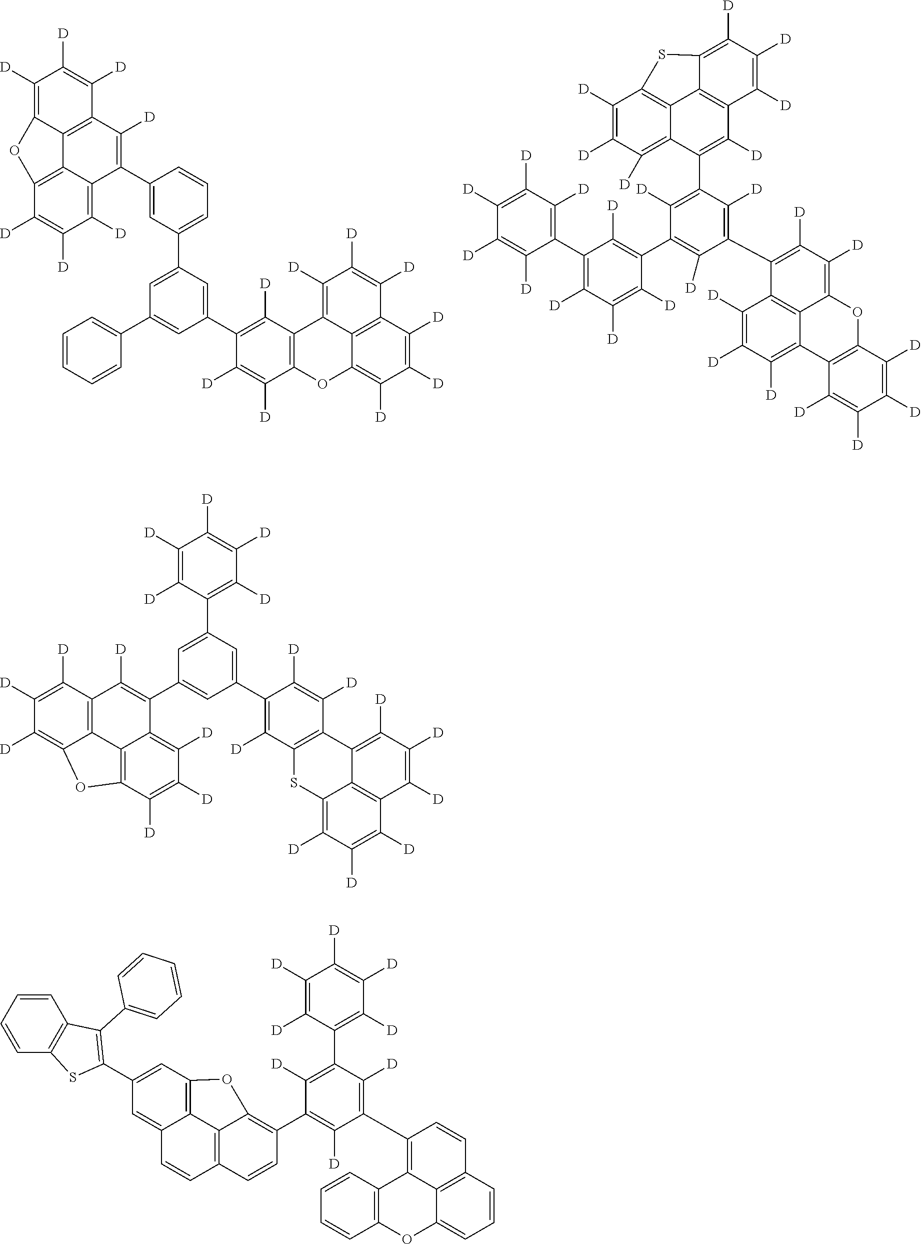

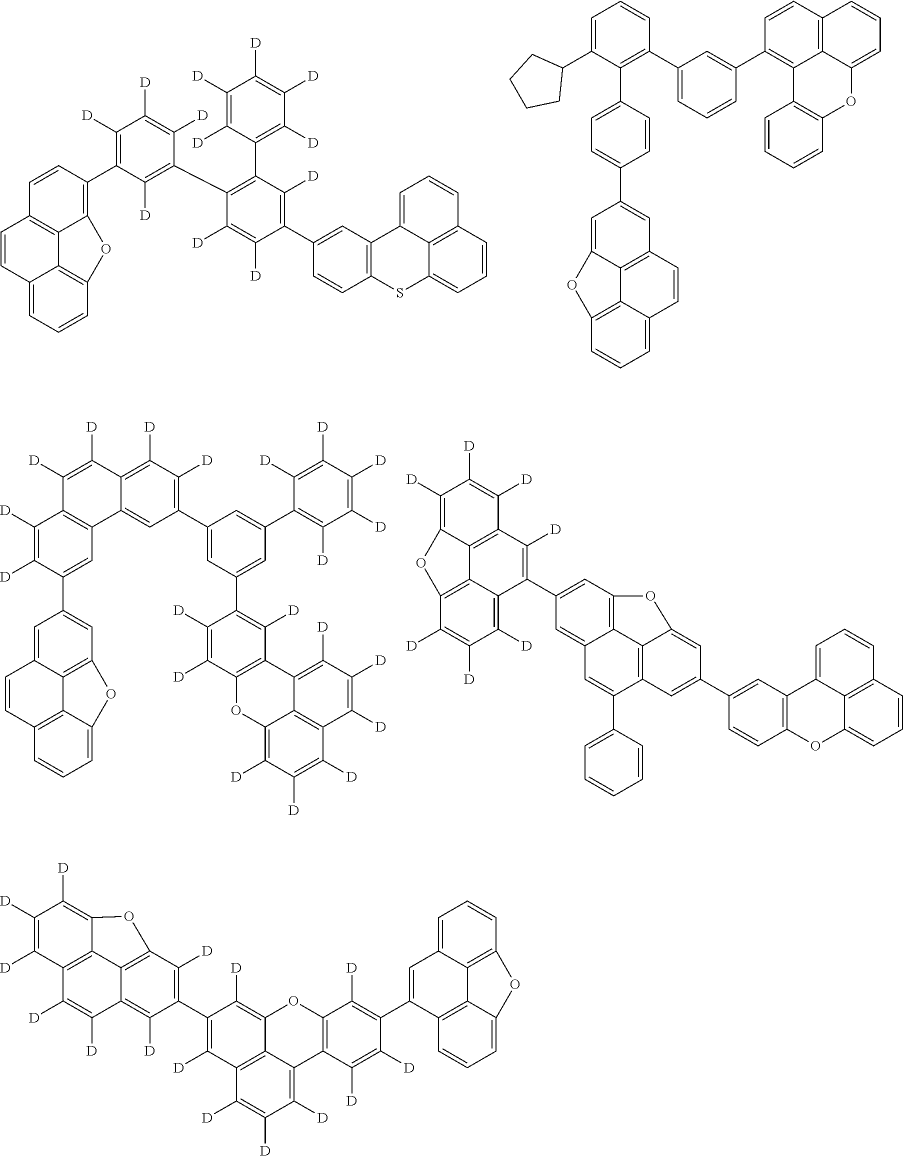

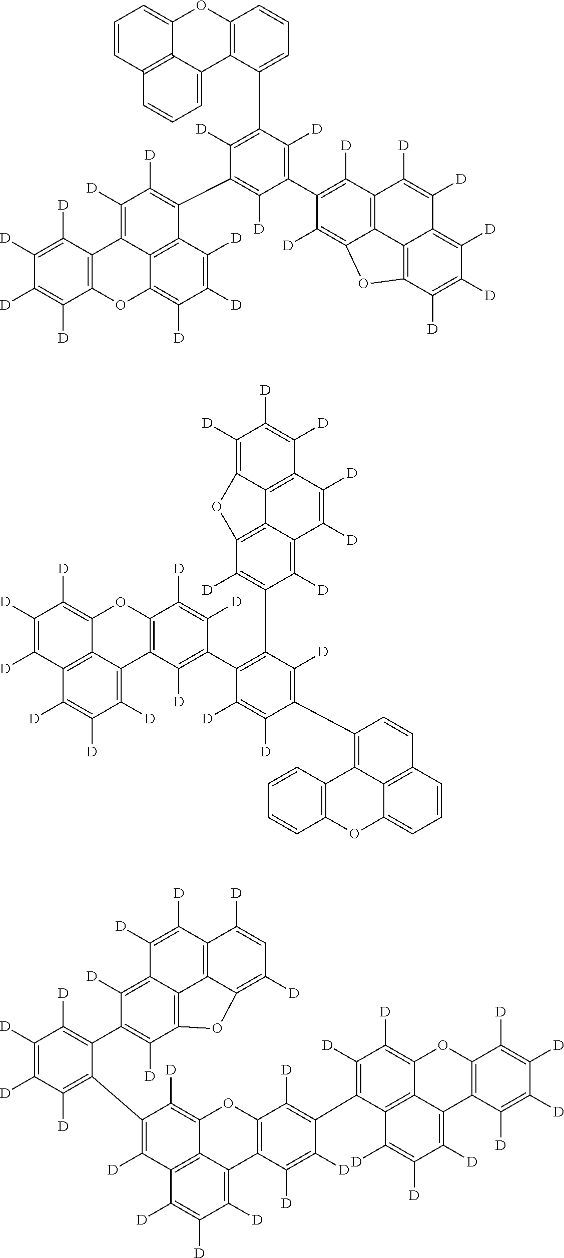

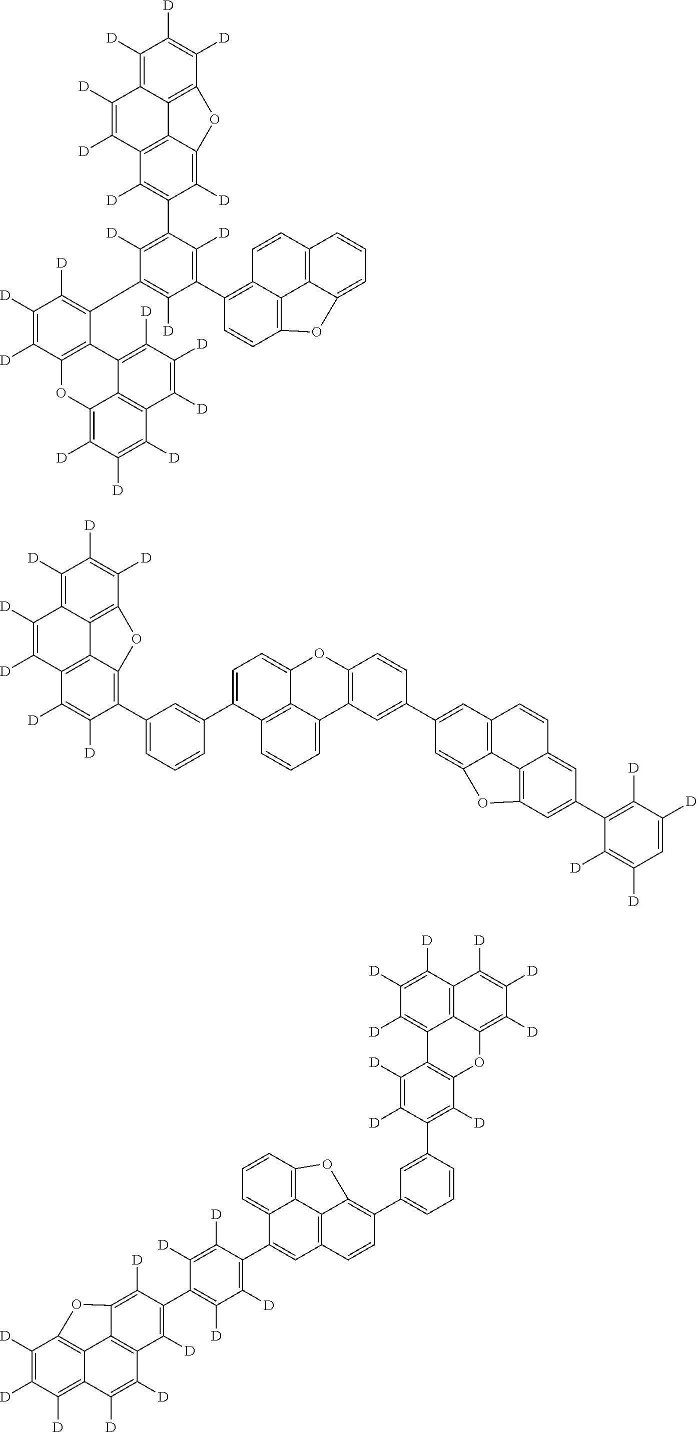

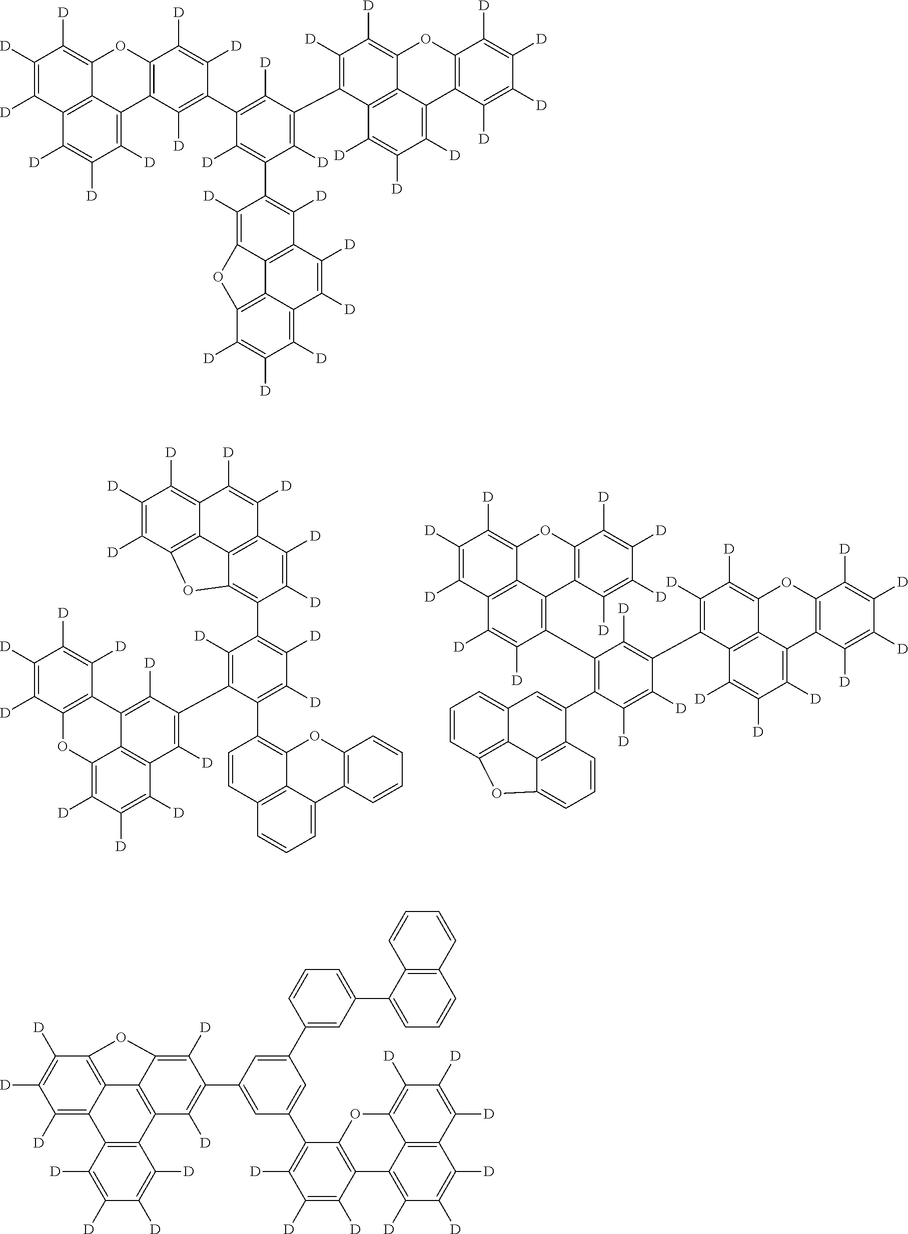

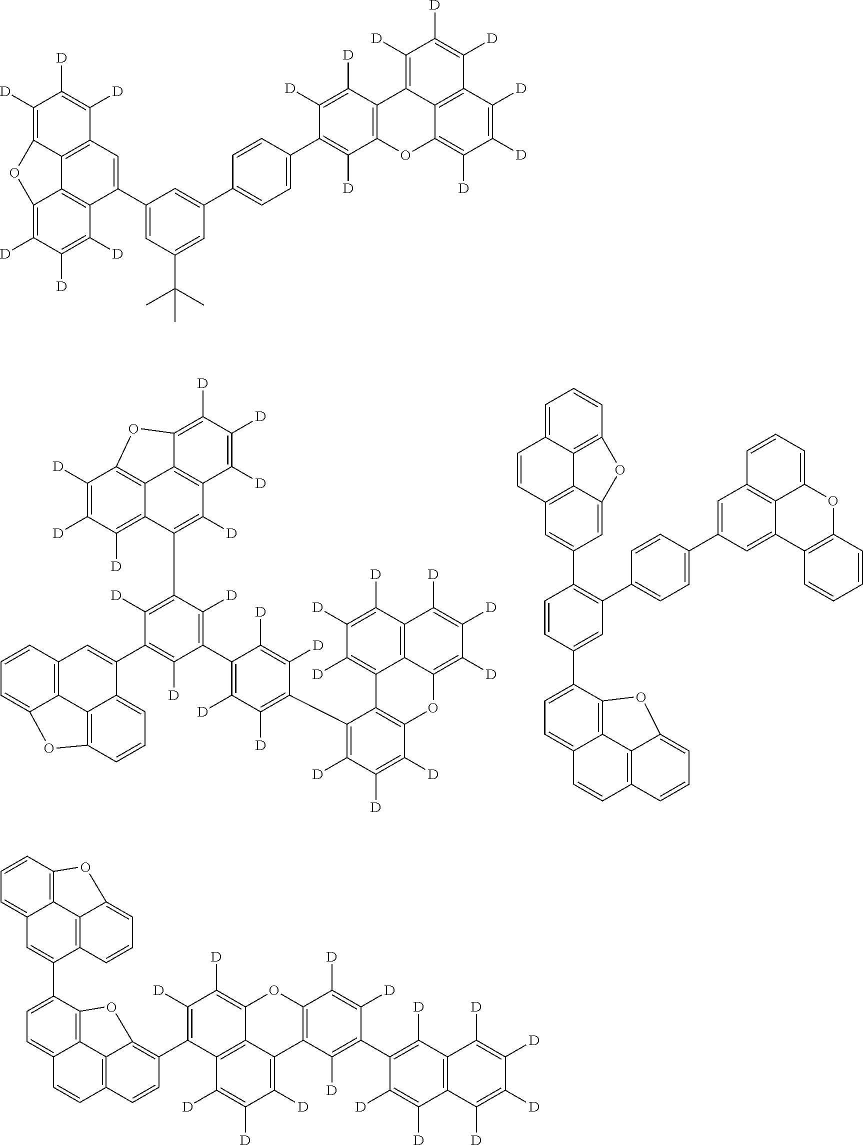

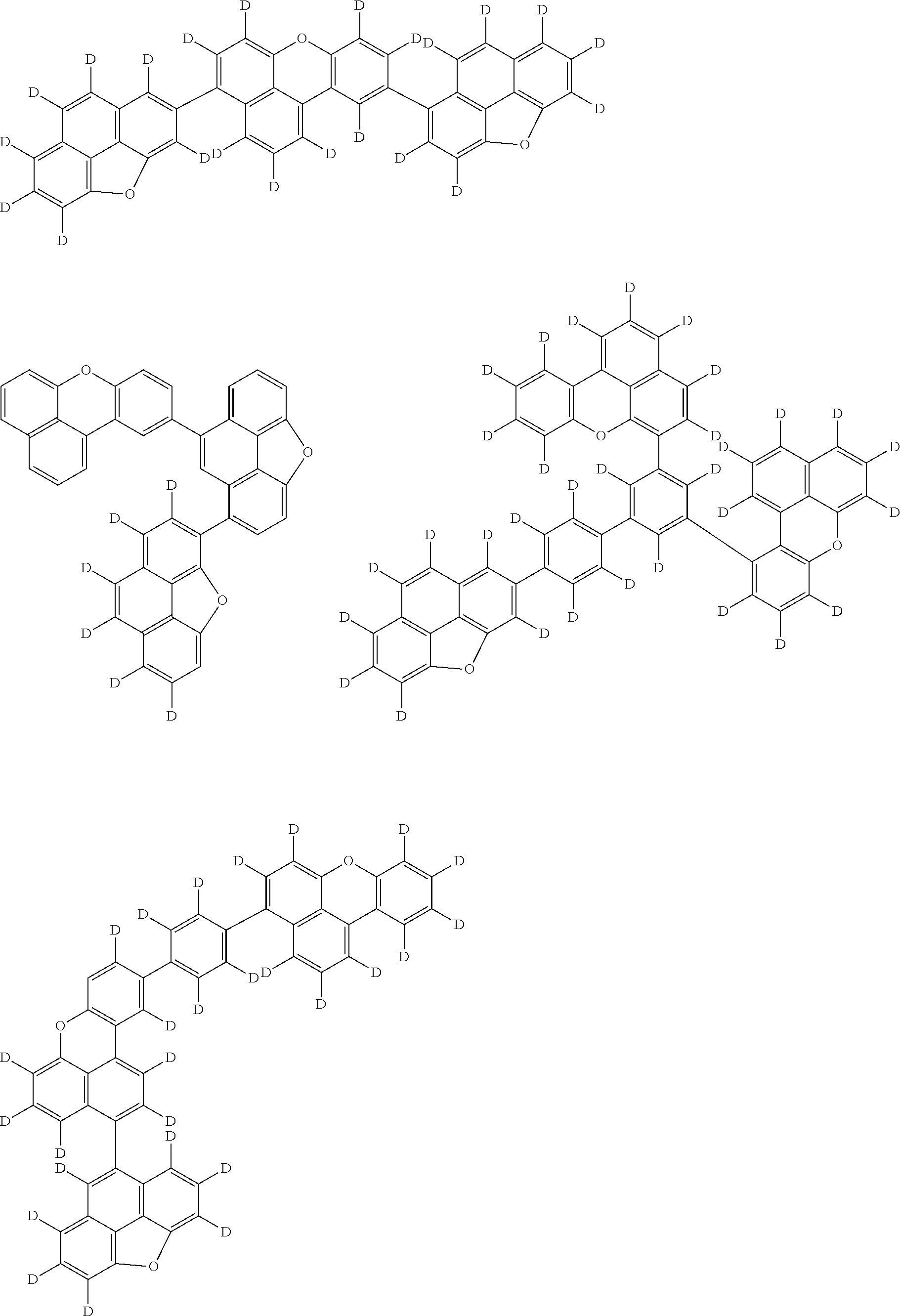

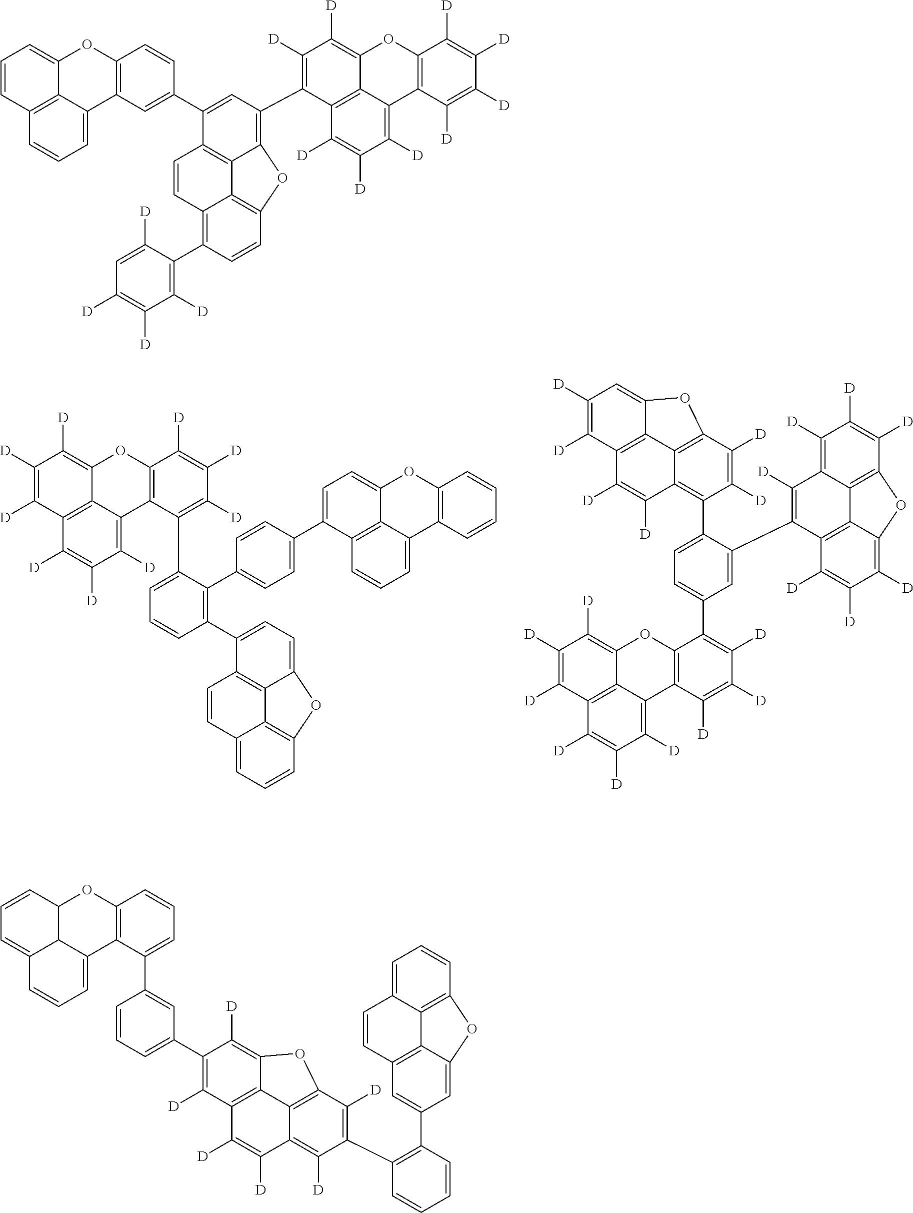

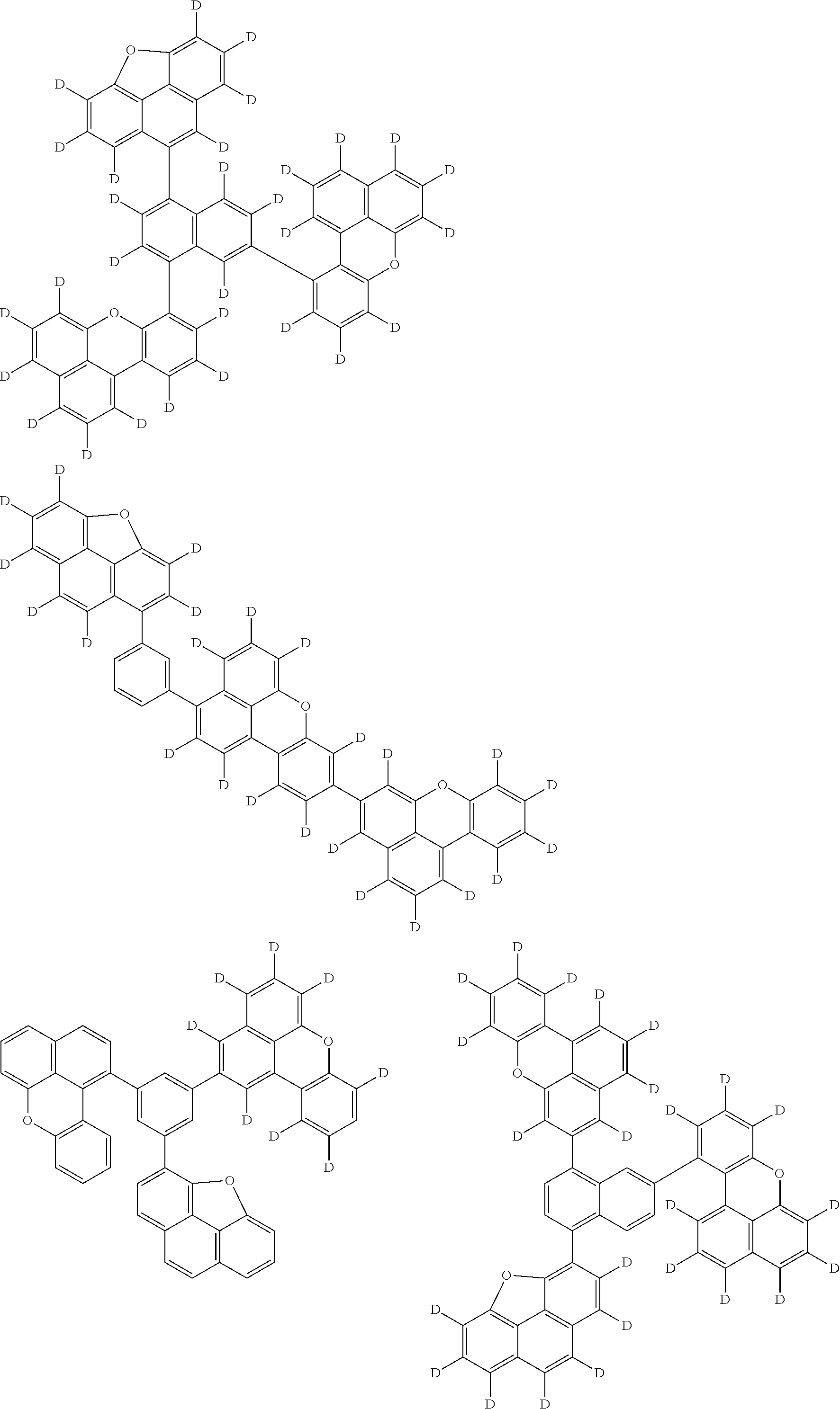

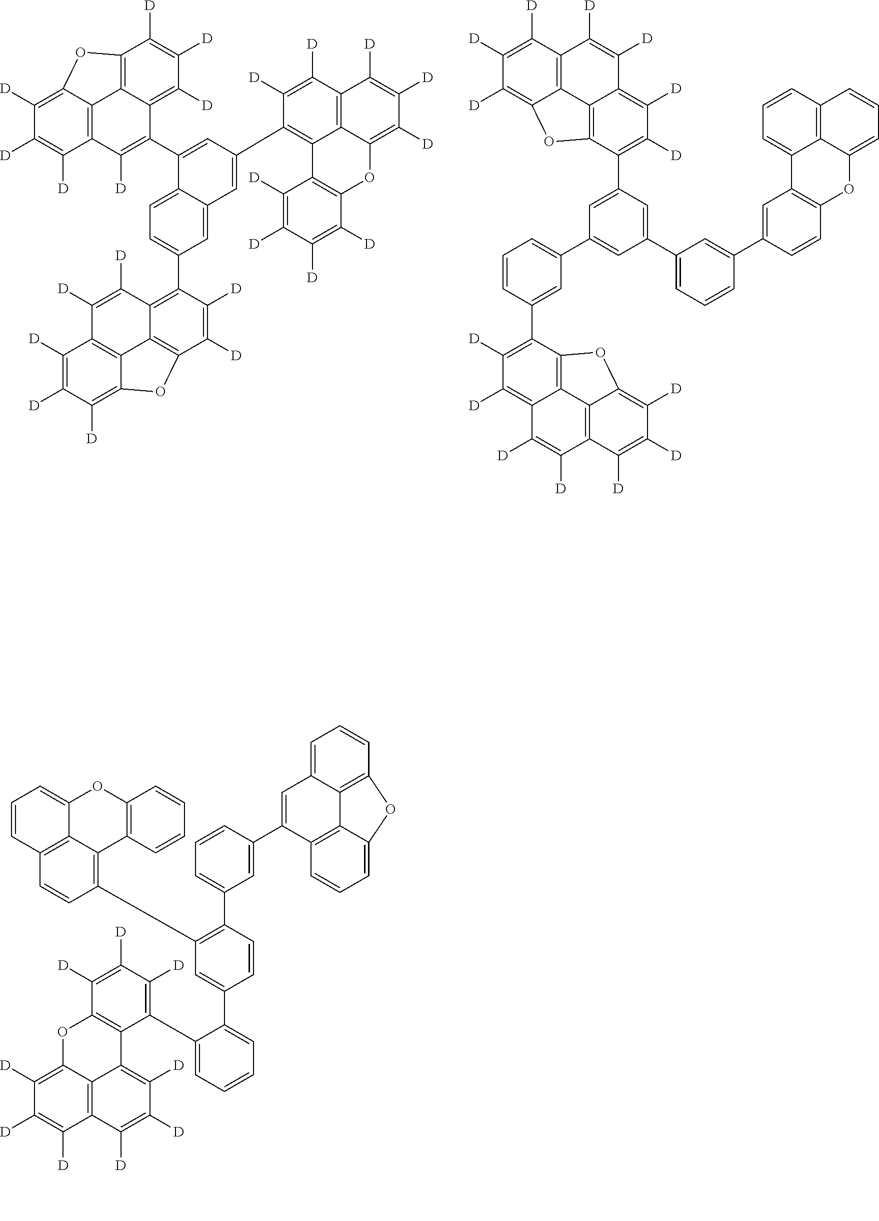

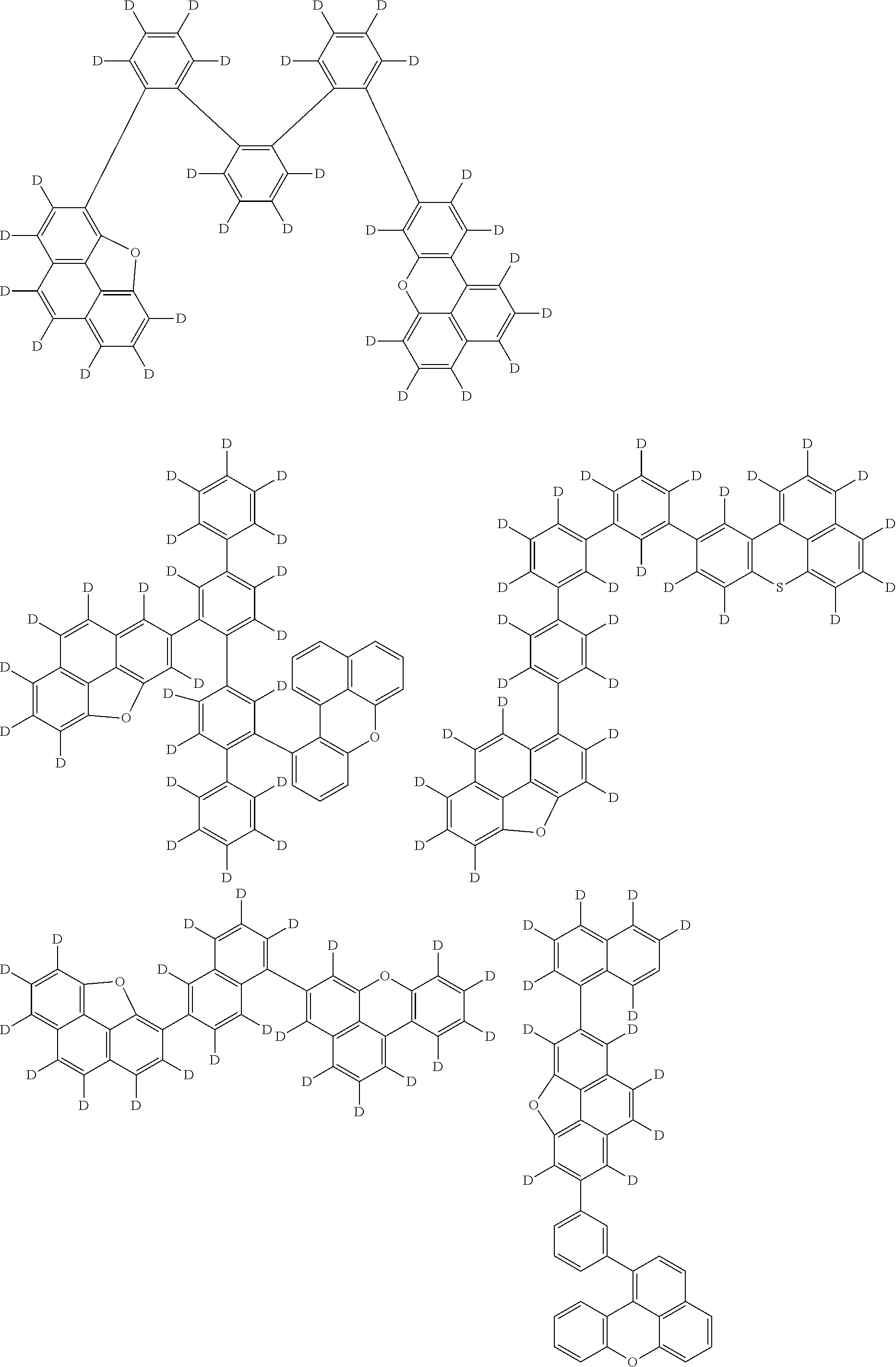

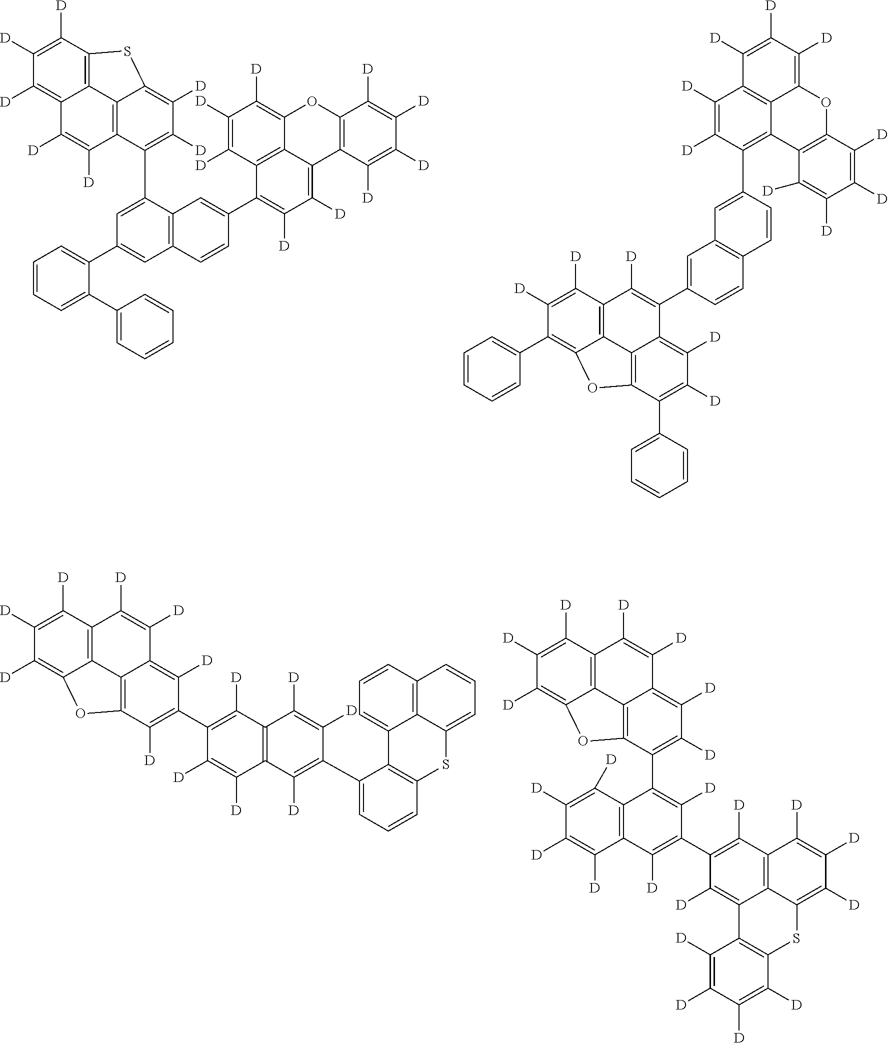

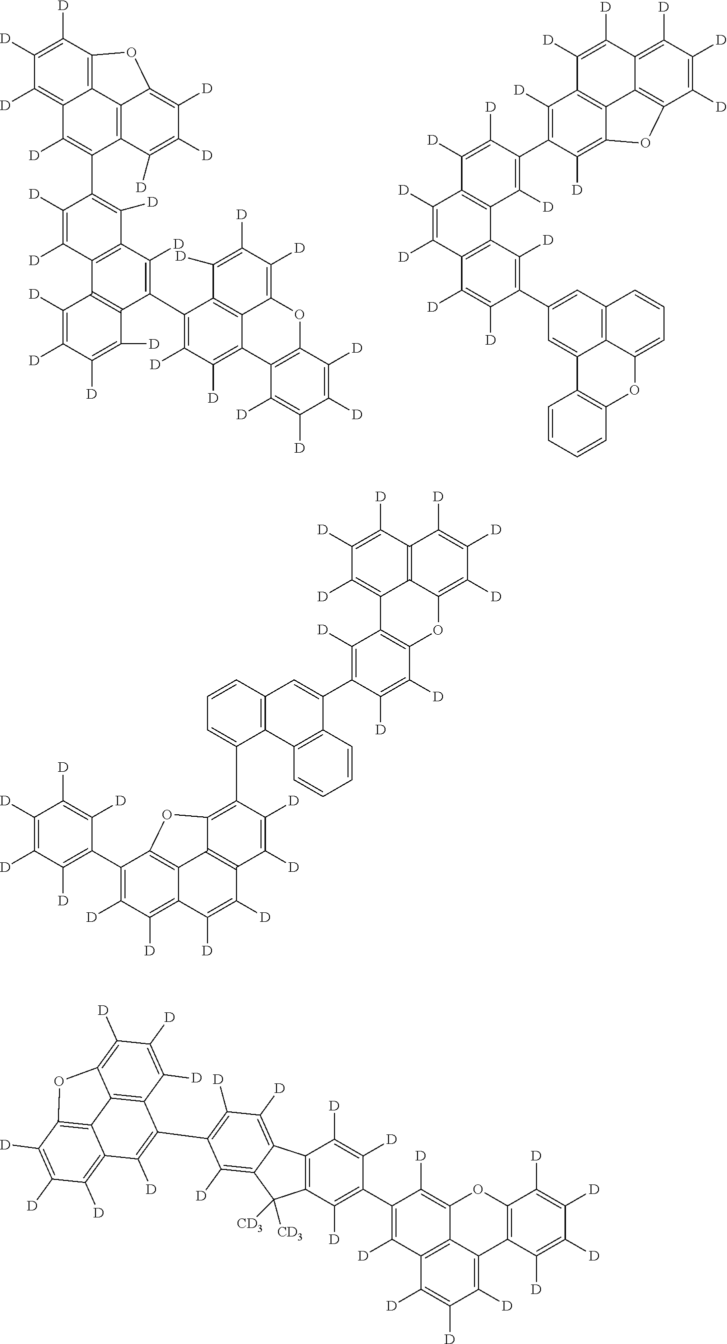

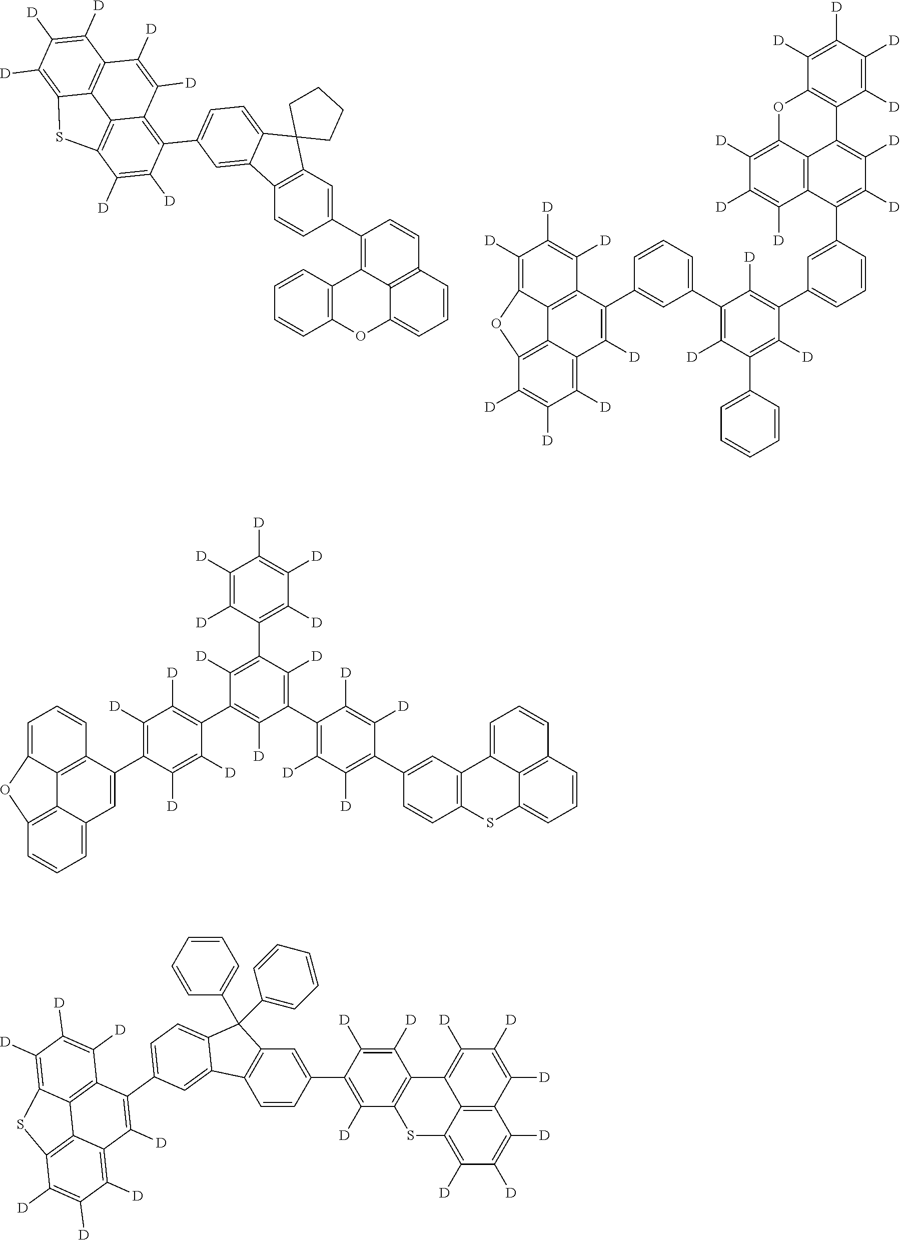

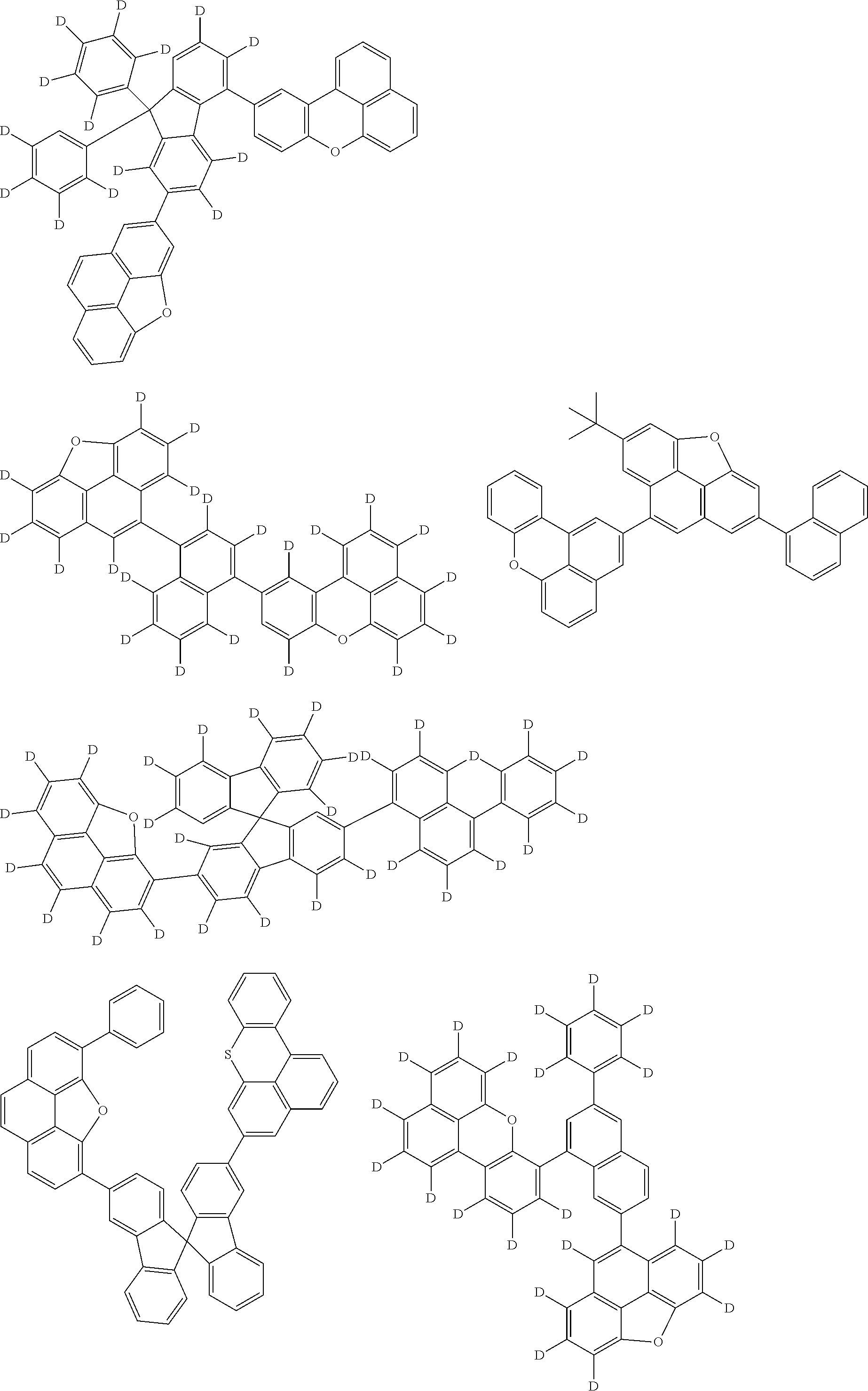

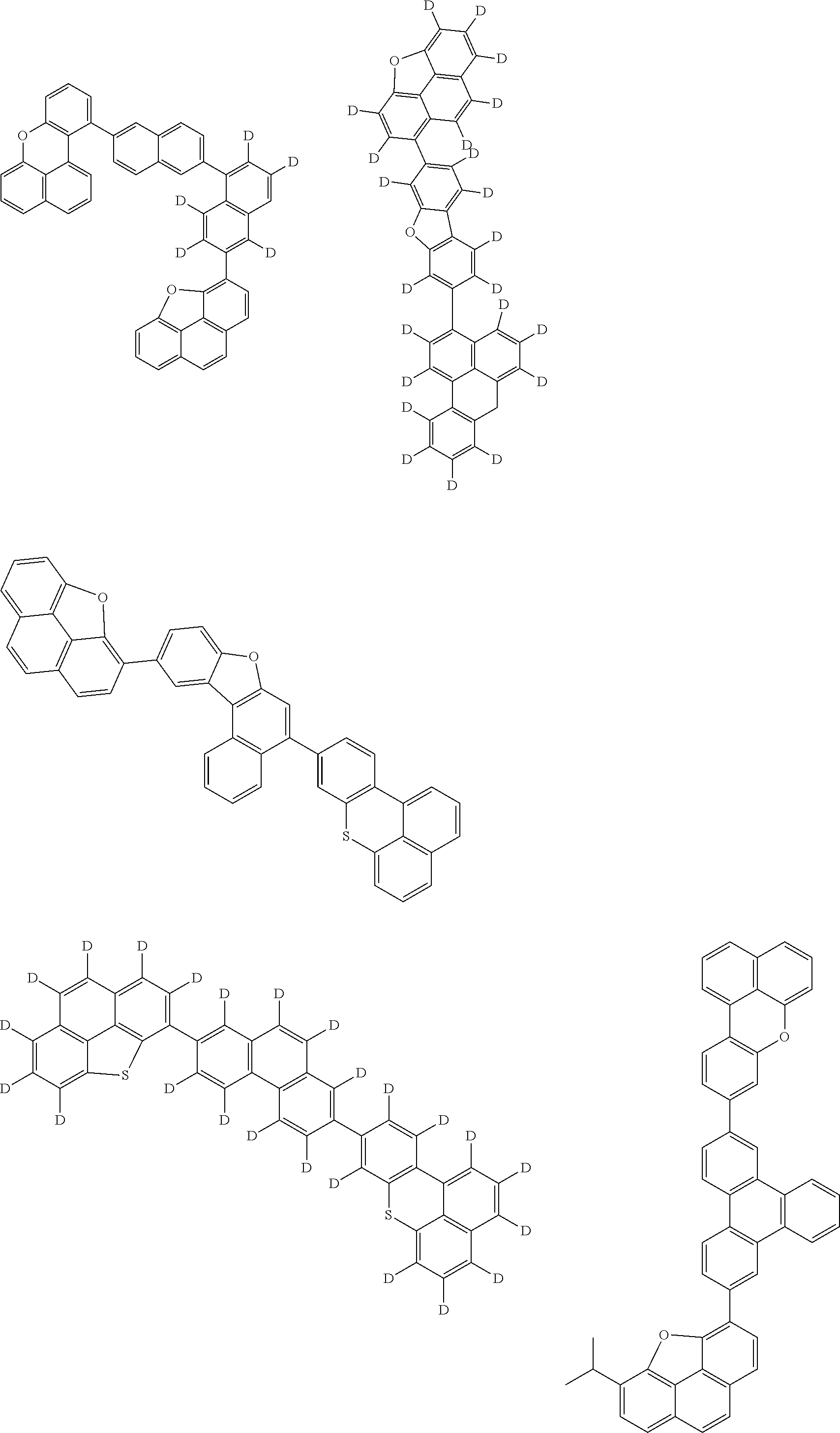

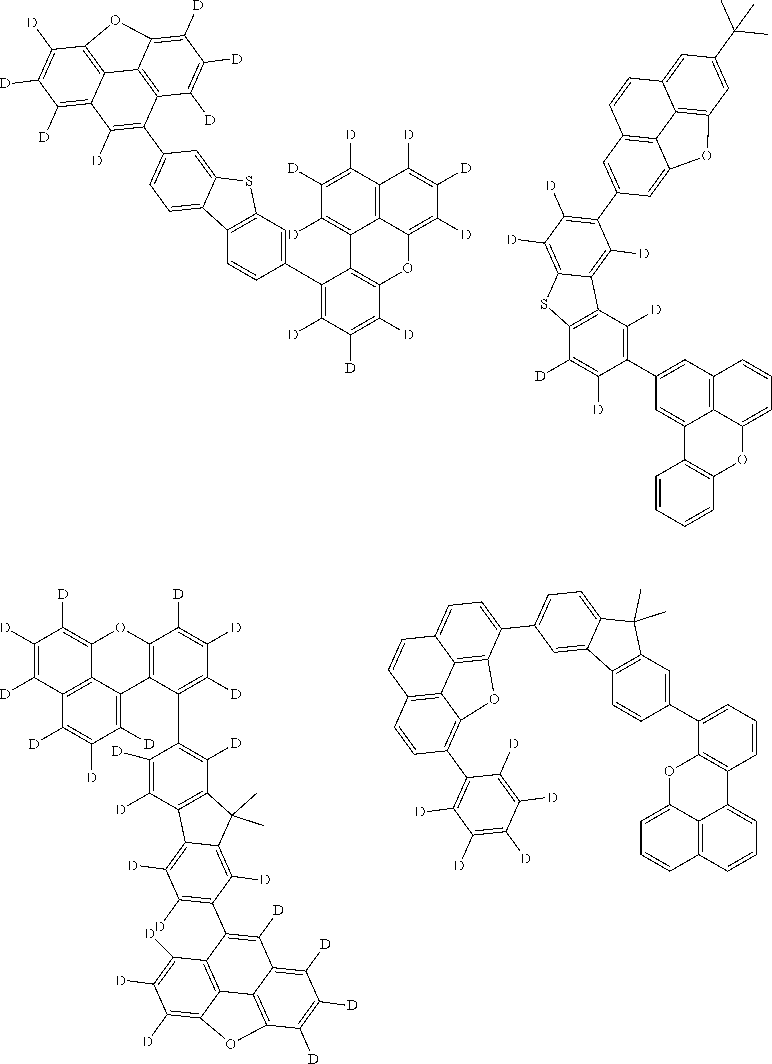

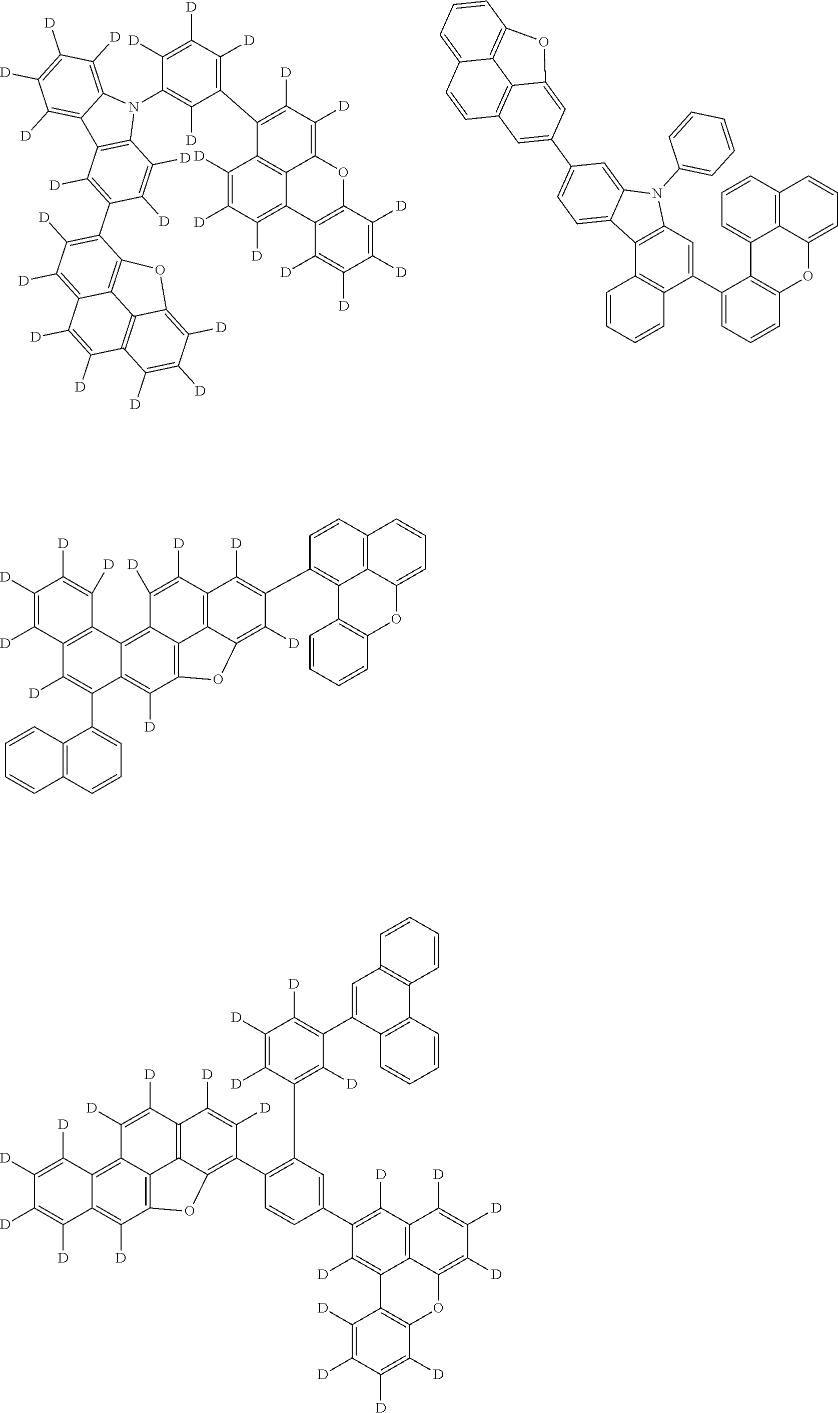

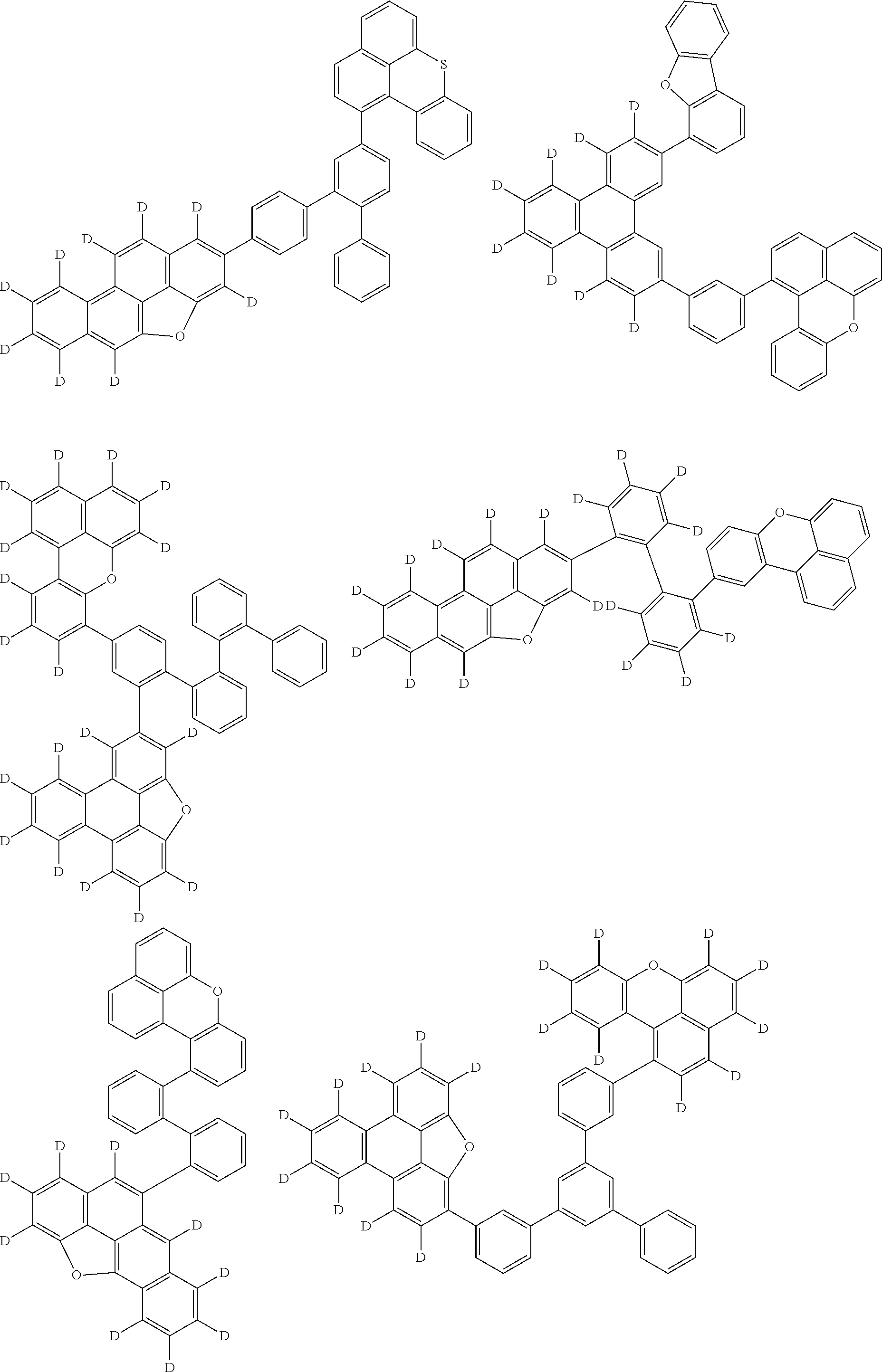

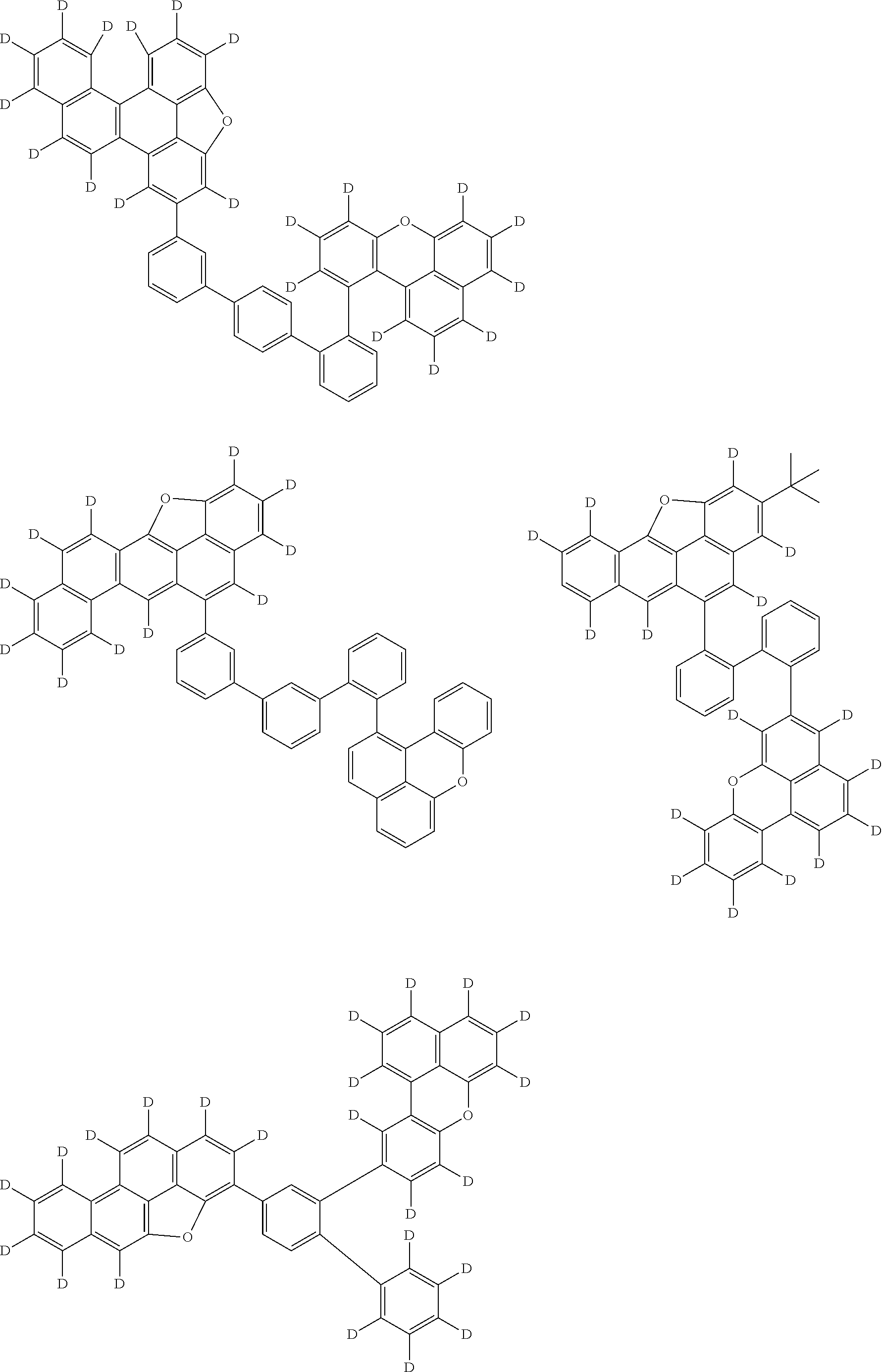

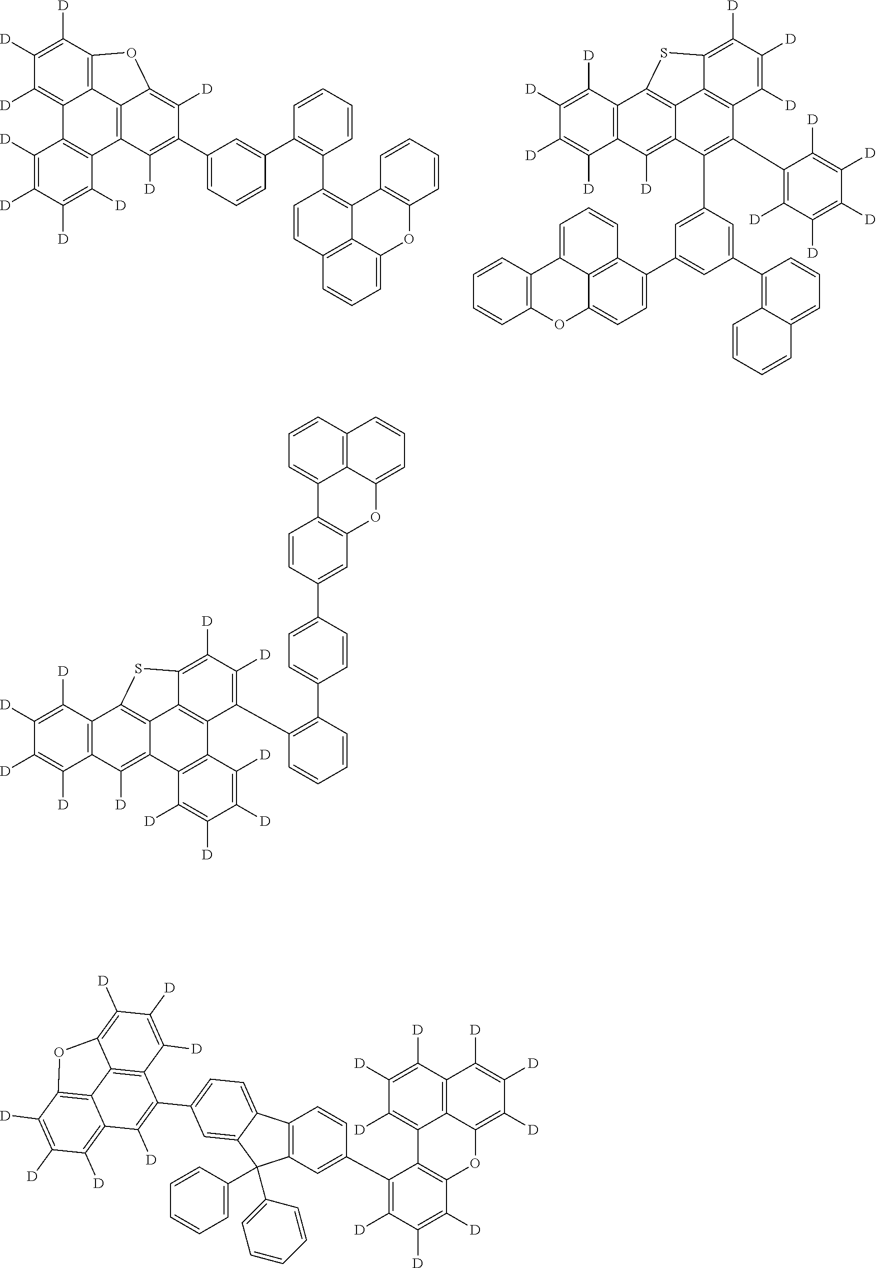

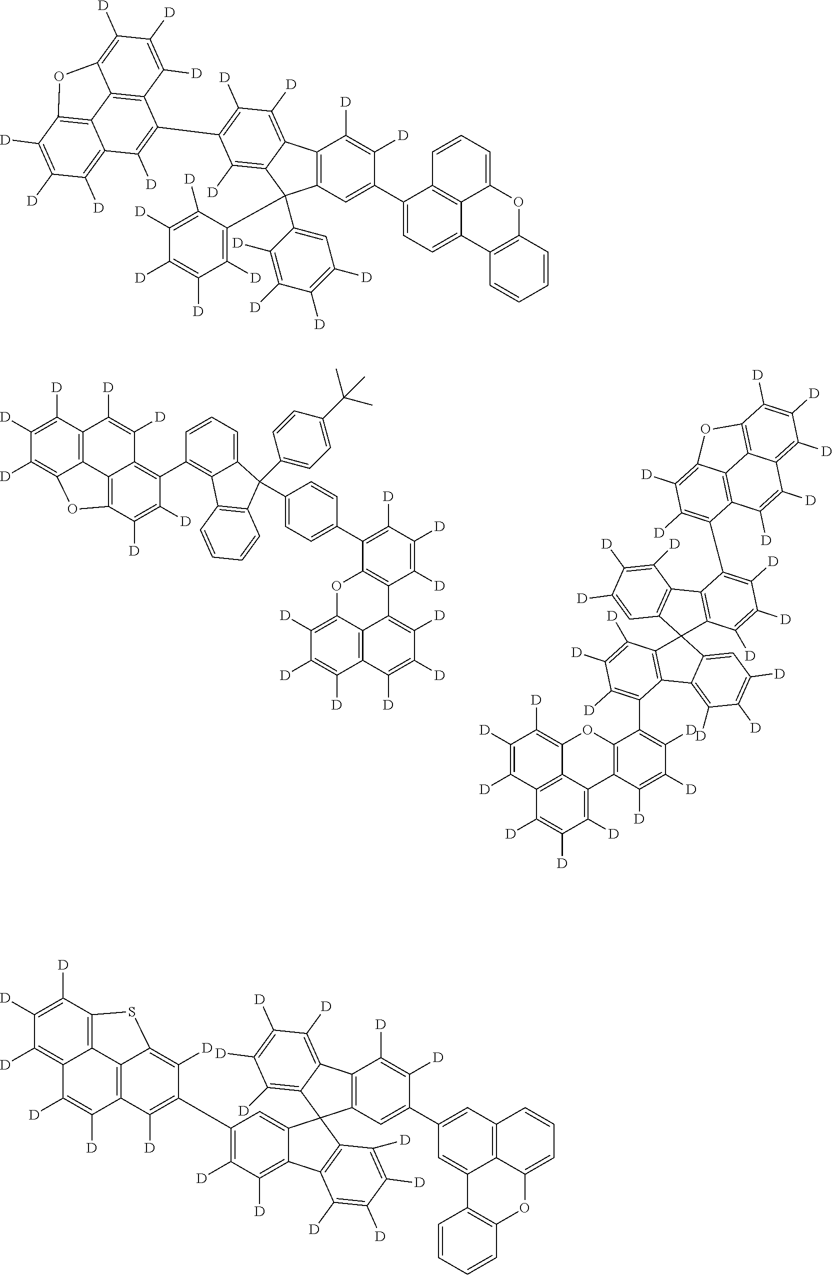

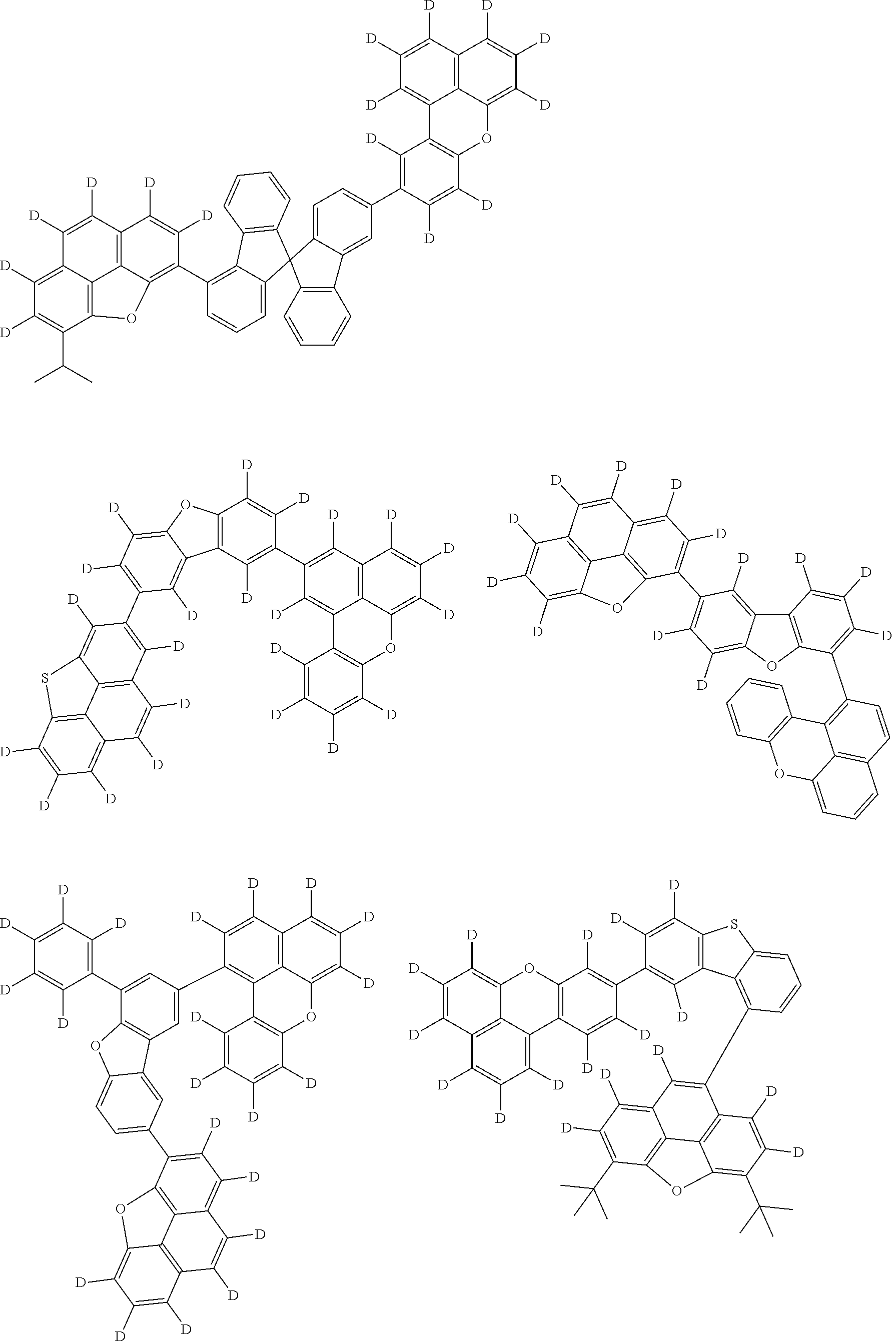

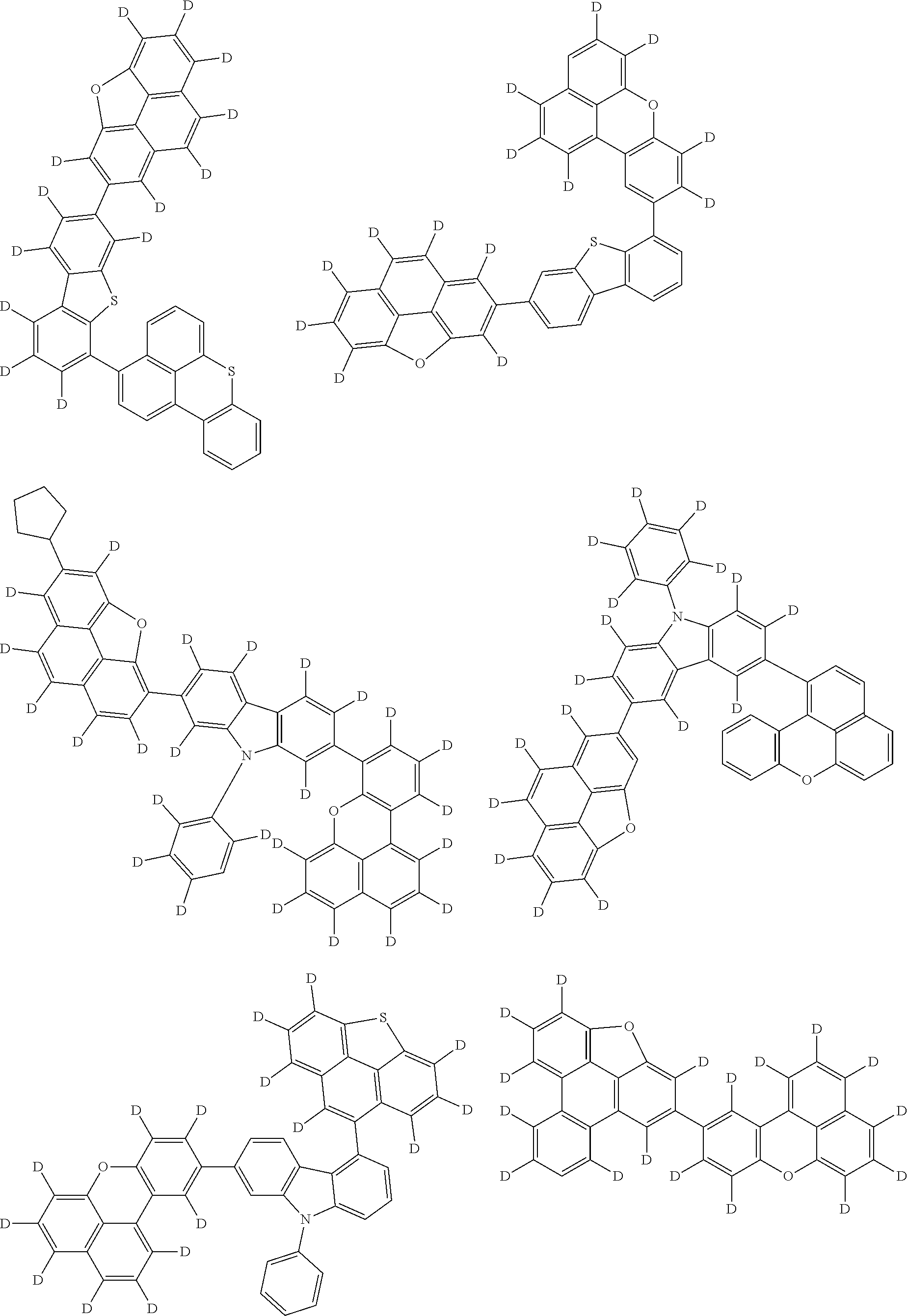

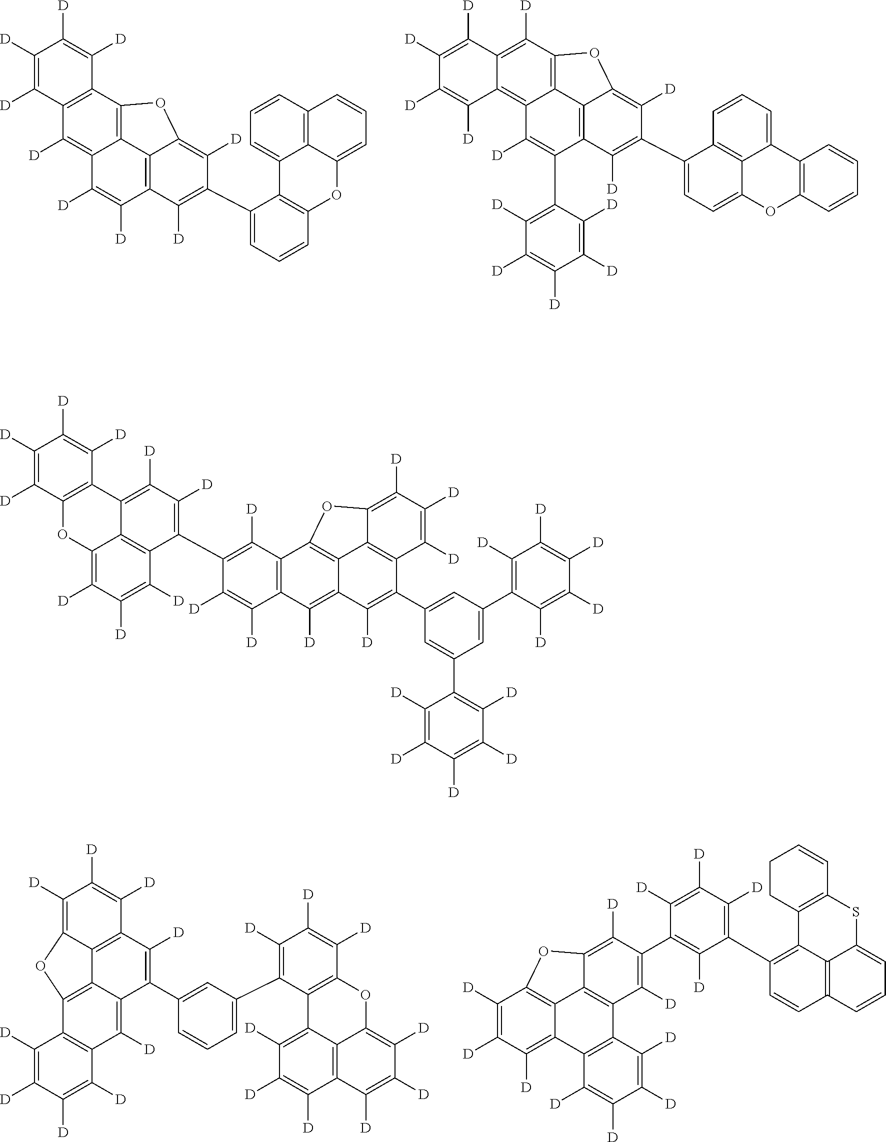

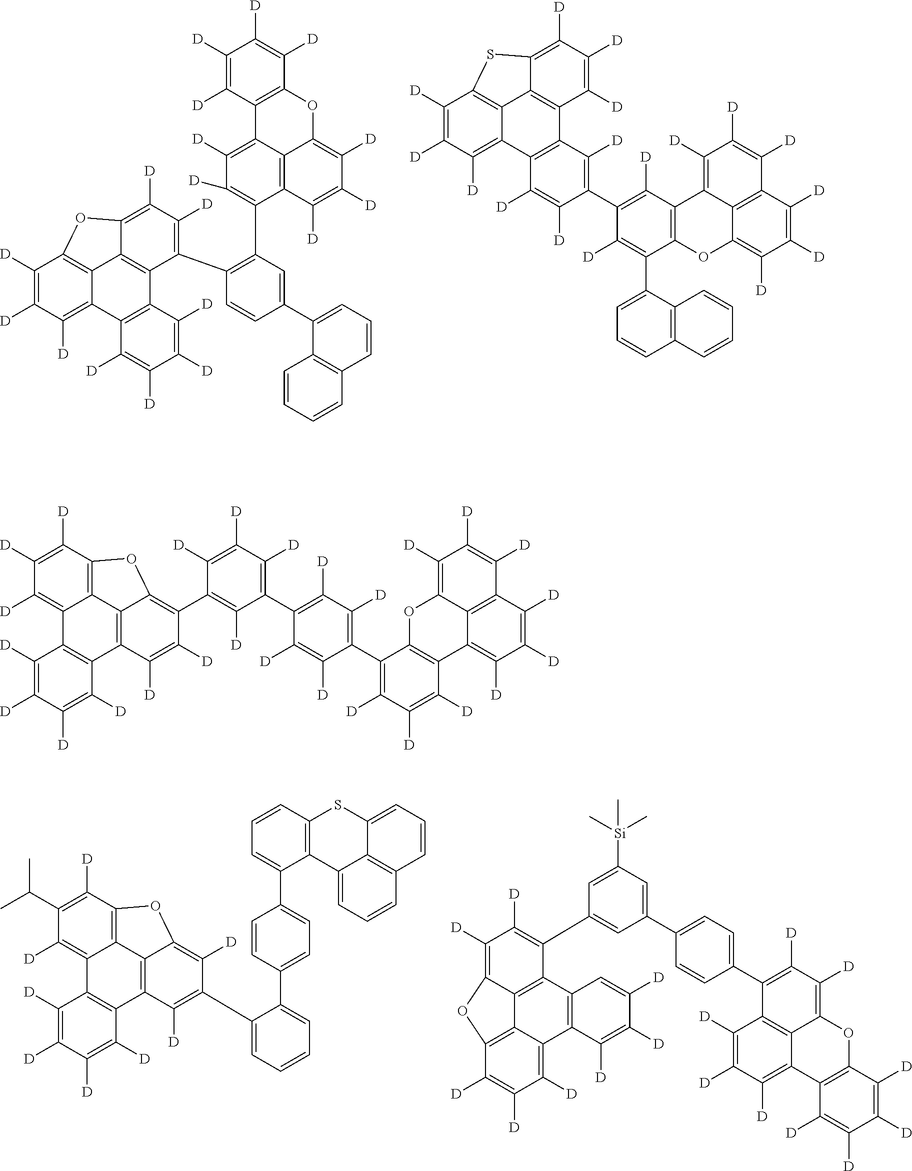

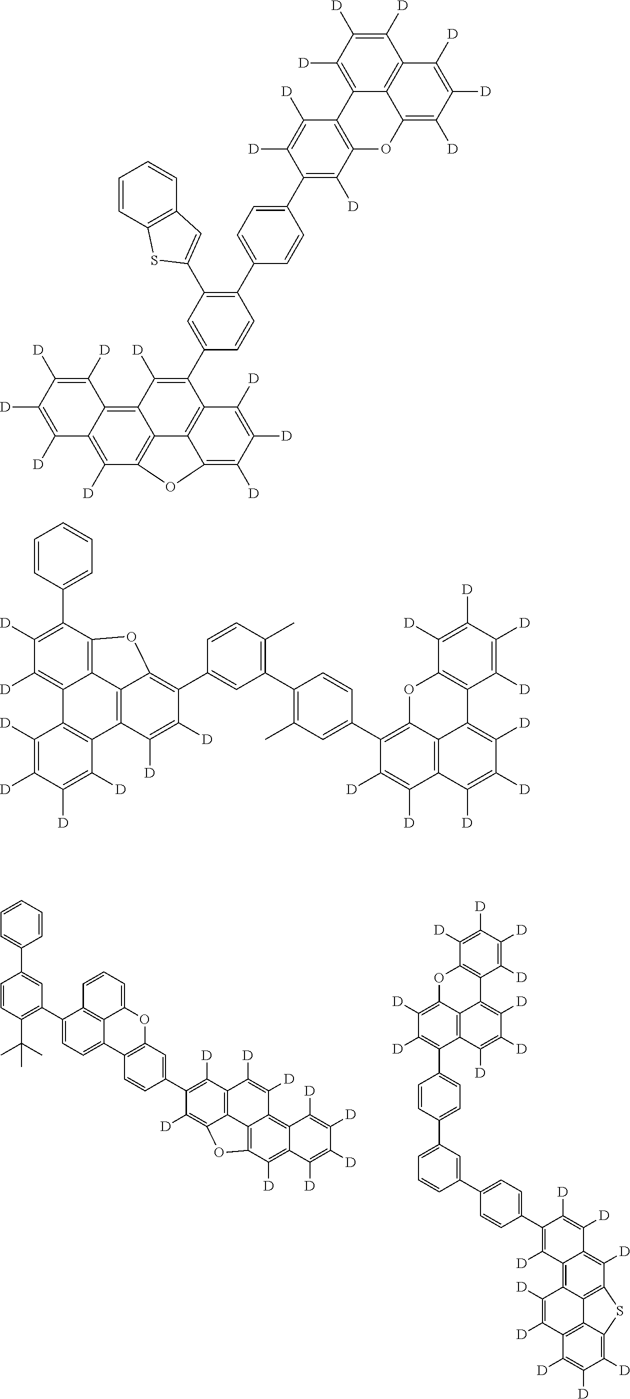

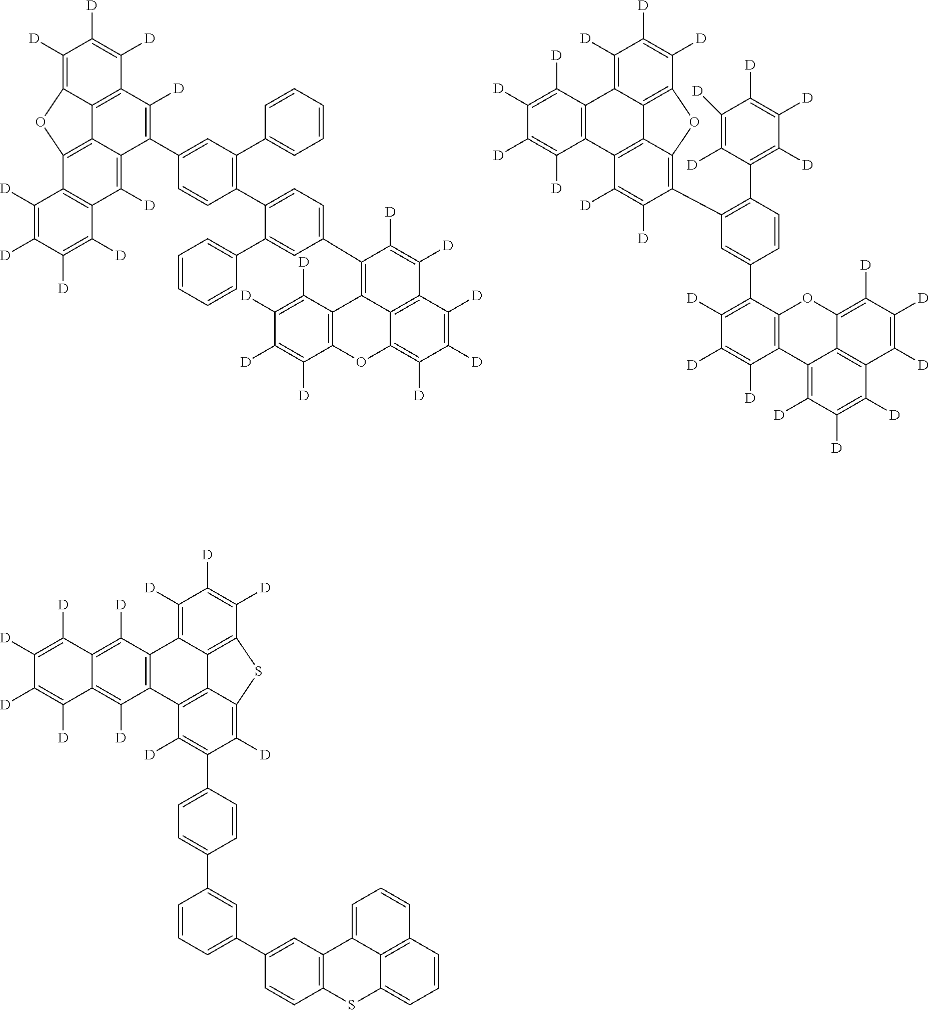

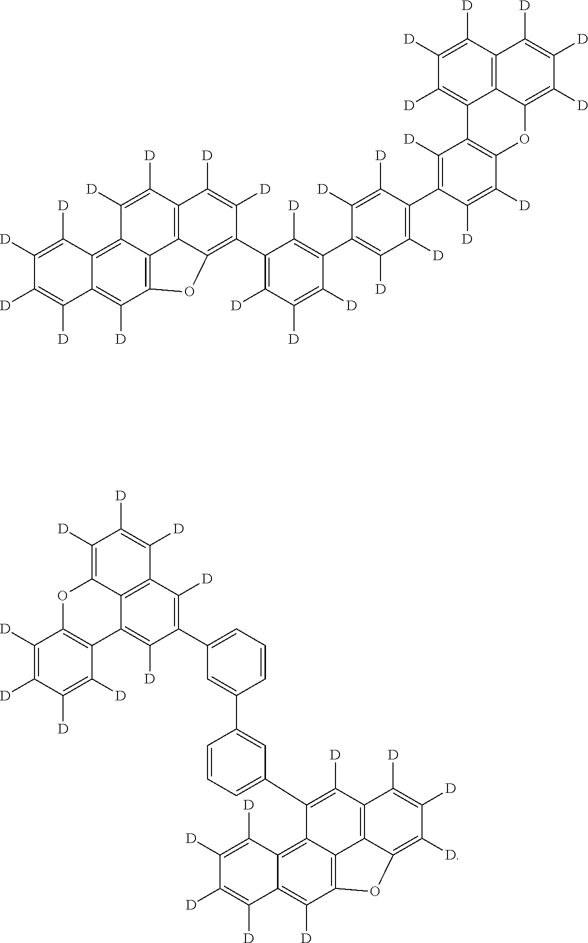

8. An organic light emitting device comprising:
an anode;
a cathode; and
an organic material layer having one or more layers provided between the anode and the cathode, wherein one or more layers of the organic material layer comprises the compound of Chemical Formula 1 of claim 1.

9. The organic light emitting device of claim 8, wherein the organic material layer comprises a light emitting layer provided between the anode and the cathode,
wherein the light emitting layer comprises: a first light emitting layer provided between the anode and the cathode; and a second light emitting layer provided between the first light emitting layer and the cathode and brought into contact with the first light emitting layer, and
the first light emitting layer comprises the compound of Chemical Formula 1.

10. The organic light emitting device of claim 9, wherein the second light emitting layer comprises at least one compound of a compound of the following Chemical Formula 2 and a compound of the following Chemical Formula 3: 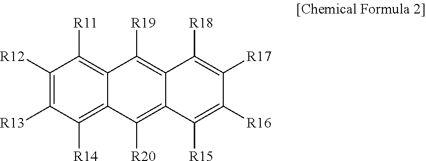
in Chemical Formula 2,
one or more of R₁₁ to R₂₀ are bonded to the * moiety of the following Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group, 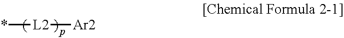
in Chemical Formula 2-1,
L2 is a direct bond; a substituted or unsubstituted arylene group; or a substituted or unsubstituted heteroarylene group,
Ar2 is a substituted or unsubstituted aryl group, and
p is an integer from 1 to 3, and when p is 2 or higher, L2's are the same as or different from each other, 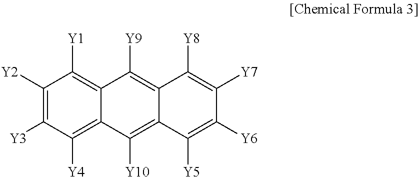
in Chemical Formula 3,
one or more of Y1 to Y10 are bonded to the * moiety of the following Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group;
a substituted or unsubstituted heteroaryl group; or a substituted or unsubstituted silyl group, 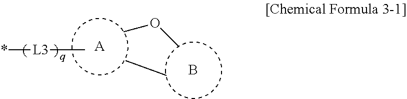
in Chemical Formula 3-1,
A and B are the same as or different from each other, and are each independently a substituted or unsubstituted aromatic hydrocarbon ring; or a substituted or unsubstituted aromatic hetero ring,
L3 is a direct bond; a substituted or unsubstituted arylene group; or a substituted or unsubstituted heteroarylene group, and
q is an integer from 1 to 3 and when q is 2 or higher, L3's are the same as or different from each other.

11. The organic light emitting device of claim 10, wherein for the light emitting layer, the first light emitting layer comprises the compound of Chemical Formula 1 as a first host material, and
the second light emitting layer comprises at least one compound of the compound of Chemical Formula 2 and the compound of Chemical Formula 3 as a second host material.

12. The organic light emitting device of claim 9, wherein each of the first light emitting layer and the second light emitting layer further comprises a dopant material.

13. The organic light emitting device of claim 12, wherein the dopant material is a fluorescent dopant.

14. The organic light emitting device of claim 13, wherein the fluorescent dopant is a pyrene-based compound or a non-pyrene-based compound.

15. The organic light emitting device of claim 14, wherein the non-pyrene-based compound is an arylamine-based compound or a boron-based compound.

16. An organic light emitting device comprising:
an anode;
a cathode; and
an organic material layer having one or more layers provided between the anode and the cathode,
wherein one or more layers of the organic material layer comprises the compound of any one of Chemical Formulae 11 to 13 of claim 6.

17. The organic light emitting device of claim 16, wherein the organic material layer comprises a light emitting layer provided between the anode and the cathode,
wherein the light emitting layer comprises: a first light emitting layer provided between the anode and the cathode; and a second light emitting layer provided between the first light emitting layer and the cathode and brought into contact with the first light emitting layer, and
the first light emitting layer comprises the compound of any one of Chemical Formulae 11 to 13.

18. The organic light emitting device of claim 17, wherein the second light emitting layer comprises at least one compound of a compound of the following Chemical Formula 2 and a compound of the following Chemical Formula 3: 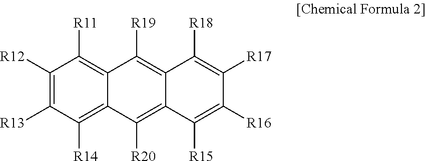
in Chemical Formula 2,
one or more of R11 to R20 are bonded to the * moiety of the following Chemical Formula 2-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group, 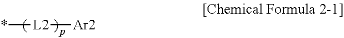
in Chemical Formula 2-1,
L2 is a direct bond; a substituted or unsubstituted arylene group; or a substituted or unsubstituted heteroarylene group,
Ar2 is a substituted or unsubstituted aryl group, and
p is an integer from 1 to 3, and when p is 2 or higher, L2's are the same as or different from each other, 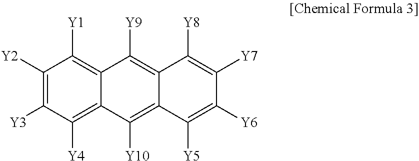
in Chemical Formula 3,
one or more of Y1 to Y10 are bonded to the * moiety of the following Chemical Formula 3-1, and the others are the same as or different from each other, and are each independently hydrogen; deuterium; a halogen group; a cyano group; a nitro group; a hydroxyl group; a substituted or unsubstituted alkyl group; a substituted or unsubstituted aryl group; a substituted or unsubstituted cycloalkyl group; a substituted or unsubstituted heteroaryl group; or a substituted or unsubstituted silyl group, 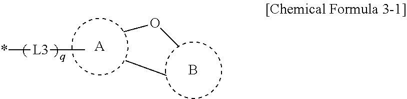
in Chemical Formula 3-1,
A and B are the same as or different from each other, and are each independently a substituted or unsubstituted aromatic hydrocarbon ring; or a substituted or unsubstituted aromatic hetero ring,
L3 is a direct bond; a substituted or unsubstituted arylene group; or a substituted or unsubstituted heteroarylene group, and
q is an integer from 1 to 3 and when q is 2 or higher, L3's are the same as or different from each other.
